(19) United States
(12) Patent Application Publication
(10) Pub. No.: US 2021/0022978 A1
(43) Pub. Date: Jan. 28, 2021

(54) BUMETANIDE DERIVATIVES FOR THE THERAPY OF HYPERHIDROSIS

(71) Applicant: ZILENTIN AG, Zug (CH)

(72) Inventors: Thomas Erker, Vienna (AT); Philipp Schreppel, Vienna (AT)

(21) Appl. No.: 17/045,738

(22) PCT Filed: Apr. 5, 2019
(86) PCT No.: PCT/EP2019/058653

§ 371 Date: Oct. 6, 2020

(30) Foreign Application Priority Data
Apr. 6, 2018 (EP) 18166173.7

(51) Int. Cl. A61K 8/49; C07C 311/46; C07D 295/135; C07D 233/84; C07D 239/38; C07D 211/52; C07D 211/70; C07D 333/20; C07D 295/155; C07D 235/08; C07D 277/16; A61Q 15/00; A61K 8/46

(52) U.S. Cl. CPC A61K 8/4986; A61K 8/466; C07D 295/135; C07D 233/84; C07D 239/38; C07D 211/52; C07D 211/70; C07D 333/20; C07D 295/155; C07D 235/08; C07D 277/16; A61Q 15/00; A61K 8/4973; A61K 8/4953; A61K 8/4946; A61K 8/4933; C07C 311/46

(57) ABSTRACT

The present invention relates to bumetanide derivatives of formula (I) as well as pharmaceutical compositions comprising these compounds for use in the treatment or prevention of diseases/disorders involving Na⁺—K⁺2Cl⁻-cotransporters (NKCCs), and particularly for use in the treatment or prevention of hyperhidrosis.

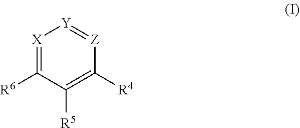

The present invention relates to bumetanide derivatives of formula (I) as well as pharmaceutical compositions comprising these compounds for use in the treatment or prevention of diseases/disorders involving Na⁺—K⁺-2Cl⁻-cotransporters (NKCCs), and particularly for use in the treatment or prevention of hyperhidrosis.

Na⁺—K⁺-Cl⁻-Co-Transporters

The intracellular chloride concentration ([Cl⁻]ᵢ) is mostly controlled by the chloride-cation-cotransporters (CCCs) of the SLC12 gene family (Maa et al., 2011). These transporters are among the most important ion transporters in multicellular organisms and are crucial for survival (Alessi et al., 2014). Na⁺—K⁺-2Cl⁻-cotransporters (NKCCs) transfer Cl⁻ into the cell and K⁺—Cl⁻-cotransporters (KCCs) are outwardly directed (Munoz, DeFelipe, & Alvarez-Leefmans, 2007). These CCCs are intrinsic membrane proteins that use the energetically favorable transmembrane gradients of potassium and sodium ions to transport Cl⁻ across membranes. These gradients are established by active primary transport of the ouabain-sensitive Na⁺—K⁺-ATPase (Alessi et al., 2014). The Cl⁻ transport is performed electroneutrally therefore without any net charge movement across the membrane (Payne et al., 2003). NKCC1 and NKCC2 use the inward sodium current to transport Cl⁻ into the cell above its equilibrium level. KCC1, KCC2, KCC3 and KCC4 use the potassium gradient to transport Cl⁻ out of the cell lowering Cl⁻-concentration below the equilibrium level (Maa et al., 2011). NKCC1 is widely distributed throughout the body and expressed in neurons, glial cells, the choroid plexus and vascular endothelium, whereas NKCC2 is primarily expressed in the kidney (Maa et al., 2011). NKCC1 and NKCC2 share 60% homology at the protein level (Markadieu, N., Delpire, E., 2014). The cellular Cl⁻-efflux and influx is also regulated by two serine-threonine kinases SPAK and OSR1 that phosphorylate critical N- and C-residues of NKCCs and KCCs. Thus, they activate NKCCs and cause Cl⁻-influx, but at the same time they inhibit KCCs and Cl⁻ efflux (Alessi et al., 2014). In the mammalian central nervous system (CNS) the intracellular Cl⁻ concentration ([Cl⁻]ᵢ) determines the strength and direction of GABAergic neurotransmission (Kahle & Staley, 2008). In the adult central nervous system there are very low levels of [Cl⁻]ᵢ and the activation of the GABA_{A} receptor leads to an influx of Cl⁻ into the cell, causing hyperpolarization and inhibition (Khanna, Walcott, & Kahle, 2013). The immature brain of neonates, on the other hand exhibits a much higher [Cl⁻]ᵢ, so that activating the GABA_{A} receptor causes an efflux of Cl⁻, which depolarizes the neuron and leads to synaptic excitation (Kahle & Staley, 2008).

NKCC2

NKCC2 is expressed at the apical membrane of the epithelial cells in the ascending limb of henle, which reabsorbs around 20-30% of the NaCl filtered by the glomerulus (Ares G., Caceres P., Ortiz P., 2011). The main function of the ascending limb of henle is the reabsorption of NaCl, but no water. This leads to a further dilution of the forming urine in the tubule lumen. NKCC2 is also expressed in the macula densa. The macula densa cells act as NaCl-sensors and are able to adjust the glomerular filtration by either vasoconstriction or vasodilation of the afferent arteriole. A decrease of the tubular NaCl concentration will lead to a vasodilation of the afferent arteriole and a release of renin by the granular cells. In contrast, an increase of the tubular NaCl concentration will lead to a vasoconstriction of the afferent arteriole and thereby a decrease of glomerular filtration. This mechanism is known as the tubuloglomerular feedback and NKCC2 has been shown to play a very important role in sensing high NaCl concentration (Peti-Peterdi, J., Harris, R., 2010).

NKCC1

In contrast to NKCC2, NKCC1 is widely distributed throughout the body and has a lot of different functions. It is highly expressed in the inner ear spiral and vestibular ganglia. Regulation of [Cl⁻]ᵢ seems to be an important function of NKCC1 in adult neurons, that are not located in the CNS. In the immature brain, however, there is an increased expression of NKCC1 and these elevated levels of [Cl⁻]ᵢ seem to have developmental effects (Dzhala et al., 2005). NKCC1 is also highly expressed in the salivary gland, where it participates in the secretion of fluid and mucine. It is also expressed in the intestine, where it is also involved in fluid excretion. The most striking deficit of NKCC1 knockout mice is deafness and imbalance, which originates from the fact that NKCC1 is highly expressed in the inner ear. It plays a major role in afferent neurons. In the CNS it is only elevated in immature neurons and plays an important role in neuronal maturation. NKCC1 knockout mice also suffer from hypotension and male infertility. The hypotension originates from a decreased vascular tone (Markadieu, N., Delpire, E., 2014).

GABA

Gamma-Aminobutyric acid (GABA) is the primary inhibitory neurotransmitter in the adult mammalian brain (Dzhala et al., 2005). However, GABA-mediated signaling also plays a key role in all important developmental steps such as cell proliferation (Owens & Kriegstein, 2002). The GABA_{A} receptor is a ligand-gated chloride channel that is opened by GABA docking to its binding-site. The receptor performs a conformational change, which allows Cl⁻ to passively flow either into the cell or out of the cell, depending on the chloride equilibrium concentration (Maa et al., 2011). The channel also allows bicarbonate to permeate the channel pore, but less efficiently than chloride (Owens & Kriegstein, 2002). Inflow of Cl⁻ causes hyperpolarization, outflow of Cl⁻ on the other hand, results in depolarization. In the immature brain GABA has a depolarizing effect, it excites neurons and can therefore cause seizures (Dzhala et al., 2005). The depolarizing effect of GABA is crucial for brain development. It has been shown that the GABA_{A} receptor influences DNA-synthesis, proliferation and neuronal migration (Owens & Kriegstein, 2002). In the immature neuron there is a delicate equilibrium between inhibition and excitation. This balance plays an important role during the early stages of brain development. Excessive inhibition leads to failure in neuronal growth and synaptic maturation, whereas excessive excitation can cause seizures and even excitotoxic death (Maa et al., 2011).

Anxiety Disorders

Anxiety disorders affect around 18% of adults and are the most prevalent class of psychiatric disorders (Le'pine, J., 2002) Anxiety disorders are currently treated with psychotherapy and medication, such as serotonine reuptake inhibitors (SSRIs), antidepressants, monoamine oxidase inhibitors, benzodiazepines and anticonvulsants. However, 20-40% of the patients do not respond to any of those medications mentioned above (Denys D., de Geus F. 2005). Another downside of these drugs are the severe side effects that these drugs can cause in the CNS. The long term use of SSRIs can cause sexual dysfunction and weight gain (Hirschfeld, R., 2003). Since a big part of patients with anxiolytic disorders are nonresponders and because of the severe side effects currently used drugs might cause, there is a great need for new drugs. It was shown that bumetanide has an anxiolytic effect in rat models of conditioned anxiety. Rats treated with bumetanide expressed a significantly smaller percentage of test period freezing in a model of contextual fear conditioning than with the vehicle alone. Bumetanide also had a significant effect in the model of fear-potentiated startle (Krystal A D, Sutherland J, Hochman D W 2012).

Autism Spectrum Disorder

Autism spectrum disorder (ASD) is a range of complex neurodevelopmental disorders that are characterized by repetitive and characteristic patterns of behavior and difficulties of social interaction and communication. ASD consists of a range of different disorders, with autism being the most severe form. Other forms are Asperger syndrome, childhood disintegrative disorder and pervasive developmental disorders as part of ASD. One in 68 children is affected by a ASD and boys are significantly more likely than girls to develop ASD. It occurs throughout all racial and ethnics groups and across all socioeconomic levels (NNDS Autism Spectrum Disorder Fact Sheet 2016). Delivery in rodents plays a very important role, causing neuroprotective and analgesic effects in newborns. These effects are caused by an oxytocin-mediated decrease of [Cl⁻]ᵢ. In two models of autism in rodents (VPA rats and FRX mice) this sequence is abolished in CA3 pyramidal neurons. It can be restored by administering bumetanide, which leads to a restoration of the GABA developmental sequence and those rats do not show any autistic phenotype in rodent offspring (Tyzio R. et al. 2014). Bumetanide has also been used in an open label trial with seven patients diagnosed with autism. The patients were treated with bumetanide for 10 months and bumetanide caused an improvement in emotion recognition and enhanced the activation of brain regions involved in social and emotional perception (Hadjikhani N., et al. 2015). Since there is no cure and only very limited treatment options available for ASD, there is a strong need for improved therapy.

Hyperhidrosis

Hyperhidrosis is a medical condition that causes excessive sweating. It is classified as either primary or secondary hyperhidrosis. Primary hyperhidrosis is idiopathic caused by over-activity of the sympathetic nerves. It only afflicts a limited body area, mostly underarms, palms, soles or head. While most of the body stays dry, one or two areas drip with sweat. Secondary hyperhidrosis results from side effects of certain medication or an underlying medical condition, such as diabetes or gout (Website of American Academy of Dermatology (2016)). Hyperhidrosis can have a substantial impact in everyday life, such as limitations in work, social interaction, physical activities, and mental and emotional health. Available treatments include topical aluminium chloride hexahydrate, oral anticholinergics, injectable botulinum toxin or surgery. They all vary in their efficacy, side effects, cost and ease of use. In the US 15.3 million people are affected by hyperhidrosis, which is 4.8% of the population. Only 51% of the affected people discussed their condition with a health care professional. 75% of the surveyed people report that their condition has had some negative impact on their social life, wellbeing, emotional or mental health. A large part agree that excessive sweating is embarrassing and leads to anxiety (Doolittle J., et al. 2016). Given the negative impact that patients report, there is clearly a need for better understanding and treatment of hyperhidrosis. By blocking the NKCC in sweat glands, excessive sweating can be treated. Bumetanide has shown to be partly effective in treating hyperhidrosis (Louie, et al., 2016).

Spinal Cord Injury

Between 250000 and 500000 people worldwide suffer spinal cord injury (SCI) each year. 90% of the causes for SCI are traumatic causes such as road traffic crashes, falls or violence. People affected by SCI have a two to five times increased risk to die prematurely, have lower school enrollment and economic participation. Symptoms of SCI depend on the severity of injury and its location, but most patients experience chronic pain (WHO (2013) Spinal Cord Injury Fact Sheet N°384). Injury and noxious input can lead to a long-lasting increase in spinal cord neural excitability that may cause chronic pain. Spinal cord injury (SCI) can transform the activation of GABA-channels from hyperpolarizing to depolarizing. The effect of SCI was linked to a downregulation of KCC2 leading to a high [Cl⁻]ᵢ leading to the shift of GABAergic activation. Neural injury seems to push the spinal systems towards a state of early development, where GABA has a depolarizing effect. This depolarizing effect might cause the development of chronic pain and spasticity. Bumetanide can restore normal GABAergic function by blocking the NKCC and restoring normal [Cl⁻]ᵢ concentration (Huang Y., et al. 2016). There is a big need of an improved therapy for those patients suffering from chronic pain caused by SCI.

Recovery after Stroke

Stroke is the fifth leading cause of death in the US and 795000 people in the US have a stroke each year. A block or rupture of a blood vessel supplying the brain with blood is causing a stroke. Stroke is also a major cause of disability, it reduces mobility in more than half of stroke survivors older than 65 (Centers for Disease Control and Prevention, (2016) Stroke Fact Sheet). Ischemic stroke promotes adult neurogenesis, but it seems that these new neurons have rather limited capabilities to survive in the long term. An ischemic stroke can cause an imbalance in the expression of NKCC and KCC, leading to an increased [Cl⁻]ᵢ and ultimately to a shift in GABAergic activation from hyperpolarizing to depolarizing. Chronic post-treatment with bumetanide can enhance the migration of neuroblasts towards the damaged striatum and can also enhance the survival of these new-born neurons. Furthermore, the behavioral assessment showed improved beam-walking performance. Therefore, bumetanide and its derivatives might cause a favorable microenvironment for newborn neurons that enhances their generation and survival (Xu W., et al. 2016). Thus, the derivatives can be used to enhance regeneration and reduce damage after ischemic stroke, but also in other diseases (e.g., Alzheimer's disease) for memory enhancement.

Schizophrenia

Schizophrenia is characterized by distortions in emotions, perceptions, thinking, behavior, sense of self and language. Many patients experience hearing voices and delusions. More than 21 million people are affected worldwide and it is associated with considerable disability. Patients are 2-2.5 times more likely to die prematurely and discrimination and stigma are very common (WHO (2016) Schizophrenia Fact Sheet). The neurophysiological basis of schizophrenia remains poorly understood, but many studies suggest that a dysregulation of cortical GABA transmission might be the cause of schizophrenia. In a recent study a gain-of-function missense variant in SLC12A2, encoding the bumetanide sensitive NKCC1 cotransporter, was identified in human schizophrenia. Functional experiments showed that this variant of NKCC1 is a gain-of-function variant, increasing Cl⁻-dependent activity even in conditions in which the transporter is normally functionally silent (hypotonicity) (Merner, N. D., et al. 2016). Another study found a KCC loss-of-function variant in human schizophrenia (Merner, N. D., et al. 2015). In both cases (gain-of-function of NKCC, loss-of-function of KCC) the [Cl⁻]ᵢ is increased, which might lead to a disruption of GABA neurotransmission. Blocking the NKCC would lead to a normalization of [Cl⁻]ᵢ and reverse GABA signaling back to hyperpolarizing.

Parasites

In a recent study it has been found that furosemide (another loop diuretic blocking the NKCC) exhibited submicromolar inhibition of rAceMIF tautomerase activity and was able to produce positive effects in various assays (Cho Y., et al. 2011). Since bumetanide and its derivatives are also inhibitors of NKCC, it is considered that they will express a similar, if not stronger effect than furosemide.

Soil-Transmitted Helminth Infections

More than 1.5 billion people worldwide are infected with soil-transmitted helminth infections. That is 24% of the world's population. These helminth infections are caused by three different species of parasitic worms: Hookworms, the roundworm and the whipworm. The adult worms live in the intestine of infected people, where they produce thousands of eggs per day. These eggs that are passed in the faeces of infected people, e.g. by being attached to vegetables that are neither cooked nor peeled. Another way of infection is drinking from a contaminated water source or children, who play in contaminated soil. Additionally, hookworm eggs hatch in the soil, releasing larvae that are able to penetrate through the skin and people get infected by walking bare foot through contaminated soil. Symptoms and morbidity highly depend on the number of worms people are infected with. Light infections do usually not show any symptoms, whereas heavier infections can cause diarrhea, abdominal pain, general weakness, impaired cognitive and physical development and blood loss that can lead to anemia (WHO (2016) Soil- transmitted helminth infections Fact Sheet). A recent study found that furosemide, another aryl-sulfonamide-derivative also used as a high ceiling loop diuretic that is blocking the NKCC, has anthelminthic activity. Human MIF is a proinflammatory protein with various functions. Furosemide targets AceMIF, which is similar to human MIF and is thought to help the worm evade the immune response of the host (Cho Y., et al. 2011). Bumetanide-derivatives might be potent inhibitors of Hookworm MIF and have superior worm killing potential. Especially lipophilic derivatives with enhanced pharmacokinetic properties are promising derivatives for a better treatment of helminth infections.

Other Parasites

The new compounds might also help treating other helminths such as the tapeworm, whipworm, guinea worm, pinworm, toxocara, Strongyloides stercoralis and Ascaris lumbricoides. Other than helminths it might be used to treat diseases caused by parasitic flukes such as Schistosomiasis, Gnathostomiasis, Paragonimiasis, Fascioliasis and Swimmer's itch. Additionally the compounds can be used to treat diseases caused by protozoa such as malaria, amoebiasis, Giardiasis, African sleeping sickness, Toxoplasmosis, Acanthamoeba keratitis, Leishmaniasis, Babesiosis, Granulomatous amoebic encephalitis, Cryptosporidiosis, Cyclosporiasis and Primary amoebic meningoencephalitis. The compounds can also be used to treat ectoparasites such as Sarcoptes scabiei, Pediculus humanus capitis, Phthirus pubis, Human botfly maggots, Tunga penetrans, lxodoidea and others. The compopunds can also be used against plant pests such as nematodes, arthropods, ectoparasites and mollusks.

Edema

Edema is the result of an imbalance between the capillary and intestinal spaces. The kidney regulates the extracellular volume by adjusting sodium and water excretion. Edema may be caused by venous obstruction, increased plasma volume and increased capillary permeability. Treatment of edema consists of sodium restriction, diuretic use and treatment of the underlying disorder. The edema may be localized only in the limb or generalized and massive in the whole body. As bumetanide is a loop diuretic and blocking the NKCC, it prevents sodium reuptake in the loop of Henle and leads to sodium and water excretion. Bumetanide and its derivatives may be used as a treatment for edema (O'Brien, O. G., et al. 2005).

Brain Edema

Cerebral edema (brain edema) causes intracranial hypertension (ICH) which leads to severe outcome of patients in the clinical setting. Effective anti-edema therapy may significantly decrease the mortality in a variety of neurological conditions. At present drug treatment is a cornerstone in the management of cerebral edema. Osmotherapy has been the mainstay of pharmacological therapy. Mannitol and hypertonic saline (HS) are the most commonly used osmotic agents. The inhibitors of Na/H exchanger, NKCC attenuate brain edema formation through inhibition of excessive transportation of ion and water from blood into the cerebral tissue (Deng Y., et al. 2016). NKCC inhibitors can thus be used in the therapy of cerebral edema.

Down Syndrome

Down syndrome is the most frequent genetic cause that leads to intellectual disability. Adults and children, who suffer from Down syndrome express lower than normal intelligence quotients, learning deficits and memory impairment. It is caused by extra genetic material in chromosome 21. This can be due to a process called nondisjunction, where the genetic material fails to separate resulting in an extra chromosome (trisomy 21). The prevalence is around 1 to 1000 births worldwide, which means that each year around 3000 to 5000 children are born with this disorder (WHO (2017), Genes and chromosomal diseases). Altered GABAergic transmission contributes considerably to the learning and memory deficits in mouse models. A recent publication has shown that bumetanide was able to restore normal GABAergic transmission and reduce cognitive impairments (Deidda, G., et al. 2015). Based on this study, bumetanide and derivatives thereof are a promising approach for the treatment of mental disability in patients with Down syndrome.

Glaucoma

Glaucoma is the second leading cause of blindness worldwide, as the age of the population increases. Glaucoma is a term for a group of different diseases: In primary open angle glaucoma the fluid in the eye is not able to drain, because the channels are blocked. This leads to an increased pressure in the eye and causes blindness. Since there are only few symptoms, patients do not recognize the slow loss of sight at first. The cause of angle closure glaucoma is similar to the open angle glaucoma, but the onset happens much faster and is accompanied by symptoms, such as headache, blurred vision and pain in the eye. With the aging of the worldwide population, the need for additional treatment is growing every year. (WHO (2004) Glaucoma is the second leading cause of blindness globally).

Bumetanide and its Derivatives

Bumetanide, i.e. 3-(butylamino)-4-phenoxy-5-sulfamoylbenzoic acid, is a loop diuretic (high ceiling diuretic) that blocks both NKCC1 and NKCC2 and has been approved by the FDA and the EMA for the treatment of edema, particularly edema associated with congestive heart failure, hepatic disease and renal disease, including the nephrotic syndrome; bumetanide and/or certain derivatives thereof have also been proposed for various other therapeutic applications (Lykke K et al., Br J Pharmacol. 2015, 172(18): 4469-80; Töllner K et al., Eur J Pharmacol. 2015, 746:78-88; Töllner K et al., Ann Neurol. 2014, 75(4):550-62; Erker T et al., Epilepsia. 2016, 57(5):698-705; Louie J C et al., Physiol Rep. 2016, 4(22). pii: e13024). However, due to its very polar carboxylic acid group, bumetanide can barely penetrate through cell membranes, which severely limits the therapeutic potential of this drug. In view of the weak therapeutic activity and the undesirably strong diuretic effect of bumetanide, there is thus an urgent and unmet need for novel and/or improved therapeutic agents that can be used for the therapy of NKCC-implicated disorders and do not suffer from the disadvantages associated with bumetanide.

Certain other (hetero)arylsulfonamides, benzoic acids, thiophenes, pyrazolopyrimidines or other derivatives are described, e.g., in Feit P W et al., J Med Chem. 1976, 19(3):402-6; Feit P W et al., J Med Chem. 1977, 20(12):1687-91; Consiglio G et al., ARKIVOC. 2002, 11:104-17; Hauck S et al., Bioorg Med Chem. 2016, 24(22):5717-29; Moni L et al., Synthesis. 2016, 48(23):4050-9; Moni L et al., Molecules. 2016, 21(9):1153/1-1153/9; Palfrey H C et al., American Journal of Physiology. 1984, 246(3, Pt. 1):C242-C246; Englert H et al., Archiv der Pharmazie (Weinheim, Germany). 1983, 316(5):460-3; Nielsen O T et al., Am Chem Soc Symp Ser, Diuretic Agents. 1978, 83:12-23; Petzinger E et al., Am J Physiol. 1993, 265(5 Pt 1):G942-54; AU-A-521892; CN-A-104926804, DE-A-1966878; DE-A-2654795; GB-A-1523632; U.S. Pat. Nos. 3,985,777; 4,010,273; US 2014/066504; WO 2008/052190; WO 2010/083442; WO 2010/085352; WO 2012/018635; WO 2013/087090; WO 2014/157635; WO 2014/196793; and WO 2014/039454.

It is thus an object of the present invention to provide novel and/or improved active agents for the therapy of hyperhidrosis or other diseases/disorders involving NKCCs, particularly NKCC1.

In the context of the present invention, it has been found that the bumetanide derivatives of formula (I) as described and defined herein can be used as inhibitors of Na⁺—K⁺-2Cl⁻-cotransporters (NKCCs), particularly as NKCC1 inhibitors. Moreover, it has been found that the bumetanide derivatives of formula (I) exhibit considerably improved properties, particularly with respect to penetration, diuresis and metabolic stability. The compounds provided herein thus show an increased lipophilicity and improved skin penetration, a significantly reduced diuretic activity, an improved metabolic stability and, overall, an enhanced therapeutic effectiveness. This makes the compounds according to the invention highly advantageous for therapeutic applications, including for the treatment or prevention of diseases or disorders involving NKCCs, particularly NKCC1. The compounds of formula (I) according to the present invention can hence advantageously be used for the treatment or prevention of hyperhidrosis (Louie J C et al., Physiol Rep. 2016, 4(22). pii: e13024; Bovell D L et al., Exp Dermatol. 2011, 20(12):1017-20; Cui C Y et al., J Dermatol Sci. 2016, 81(2):129-31) or other diseases/disorders involving NKCCs, including any of the diseases/disorders described herein above in the introduction of the present specification. The invention also provides compounds of formula (I) that are selective inhibitors of NKCC1, particularly compounds that inhibit NKCC1 more potently than NKCC2, which renders these compounds especially suitable for the treatment or prevention of NKCC1-implicated diseases/disorders as well as for therapeutic applications in which a high-ceiling diuretic effect is undesirable, including in particular the treatment or prevention of hyperhidrosis. Some of the compounds of the invention furthermore show an advantageous water-solubility. The invention also provides compounds of formula (I) that comprise a carboxylic ester group and can be hydrolyzed by esterases in the skin of a patient to release more polar therapeutically active compounds which, due to their increased polarity, will not easily be transported back to the skin surface and will thus accumulate at the desired target site (“metabolic trapping”), resulting in a more pronounced and/or prolonged therapeutic effect. All these properties render the compounds provided herein highly suitable as medicaments for inhibiting NKCCs, particularly NKCC1, and thus for the therapeutic intervention in diseases/disorders involving NKCCs and, in particular, for the treatment or prevention of hyperhidrosis.

Accordingly, the present invention provides a compound of the following formula (I) or a pharmaceutically acceptable salt or solvate thereof

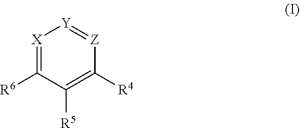

for use in the treatment or prevention of hyperhidrosis.

In formula (I), the ring moiety

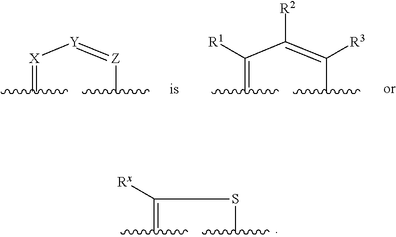

R^{x} is R¹ or R³.

R¹ is selected from —COOH, —COO—(C₁₋₁₅ alkyl), —COO—(C₀₋₁₅ alkylene)-carbocyclyl, —COO—(C₀₋₁₅ alkylene)-heterocyclyl, —O—CHO, —O—CO—(C₁₋₁₅ alkyl), —O—CO—(C₀₋₁₅ alkylene)-carbocyclyl, —O—CO—(C₀₋₁₅ alkylene)-heterocyclyl, —CHO, —CO—(C₁₋₁₅ alkyl), —CO—(C₀₋₁₅ alkylene)-carbocyclyl, —CO—(C₀₋₁₅ alkylene)-heterocyclyl, —CO—NH₂, —CO—N(R¹¹)—(C₁₋₁₅ alkyl), —CO—N(R¹¹)—(C₀₋₁₅ alkylene)-carbocyclyl, —CO—N(R¹¹)—(C₀₋₁₅ alkylene)-heterocyclyl, —N(R¹¹)—CHO, —N(R¹¹)—CO—(C₁₋₁₅ alkyl), —N(R¹¹)—CO—(C₀₋₁₅ alkylene)-carbocyclyl, —N(R¹¹)—CO—(C₀₋₁₅ alkylene)-heterocyclyl, C₁₋₁₅ alkyl, —(C₀₋₁₅ alkylene)-carbocyclyl, —(C₀₋₁₅ alkylene)-heterocyclyl, C₂₋₁₅ alkenyl, —(C₂₋₁₅ alkenylene)-carbocyclyl, —(C₂₋₁₅ alkenylene)-heterocyclyl, C₂₋₁₅ alkynyl, —(C₂₋₁₅ alkynylene)-carbocyclyl and —(C₂₋₁₅ alkynylene)-heterocyclyl,

wherein the alkyl moiety of any of the aforementioned groups, the alkylene moiety of any of the aforementioned groups, the alkenylene moiety of any of the aforementioned groups, the alkynylene moiety of any of the aforementioned groups, said C₁₋₁₅ alkyl, said C₂₋₁₅ alkenyl and said C₂₋₁₅ alkynyl are each optionally substituted with one or more groups independently selected from halogen, —CF₃, —CN, —NO₂, —N(R¹¹)(R¹¹), —O(R¹¹), —S(R¹¹) and —SO₃H, wherein one or more —CH₂— units comprised in the alkyl moiety of any of the aforementioned groups, in the alkylene moiety of any of the aforementioned groups, in the alkenylene moiety of any of the aforementioned groups, in the alkynylene moiety of any of the aforementioned groups, in said C₁₋₁₅ alkyl, in said C₂₋₁₅ alkenyl, or in said C₂₋₁₅ alkynyl are each optionally replaced by a group independently selected from —O—, —CO—, —COO—, —O—CO—, —N(R¹¹)—, —N(R¹¹)—CO—, —CO—N(R¹¹)—, —S—, —SO—, —SO₂—, —SO₂—N(R¹¹)- and —N(R¹¹)—SO₂—,

and further wherein the carbocyclyl moiety of any of the aforementioned groups and the heterocyclyl moiety of any of the aforementioned groups are each optionally substituted with one or more groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹¹), —S(R¹¹), —SO₃H, carbocyclyl and heterocyclyl.

Each R¹¹ is independently hydrogen or C₁₋₆ alkyl.

R² is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl).

R³ is selected from —SO₂—NH₂, —SO₂—NH(C₁₋₆ alkyl), —SO₂—N(C₁₋₆ alkyl)(C₁₋₆ alkyl), —SO₂—N═(C₁₋₆ alkylidene) and —SO₂-halogen, wherein the alkyl moiety of said —SO₂—NH(C₁₋₆ alkyl), one or both of the alkyl moieties of said —SO₂—N(C₁₋₆ alkyl)(C₁₋₆ alkyl), and the alkylidene moiety of said —SO₂—N═(C₁₋₆ alkylidene) are each optionally substituted with one or more groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl).

R⁴ is a group R^{4a}, and R⁵ is a group R^{5a}, or R⁴ and R⁵ are mutually linked to form a group —R^{5b}—.

R⁴ is selected from —O—R⁴¹, —S—R⁴¹, —NH—R⁴¹, —N(C₁₋₆ alkyl)-R⁴¹, halogen, hydrogen, carbocyclyl and heterocyclyl, wherein said carbocyclyl and said heterocyclyl are each optionally substituted with one or more groups R⁴².

R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₀₋₄ alkylene)-carbocyclyl and the heterocyclyl moiety of said —(C₀₋₄ alkylene)-heterocyclyl are each optionally substituted with one or more groups R⁴², and wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more groups R⁴³.

Each R⁴² is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆alkyl).

Each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃, —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl) and —N(C₁₋₆ alkyl)-CO—(C₁₋₆ alkyl).

R⁵ is selected from —NH₂, —NH(C₁₋₆ alkyl), —N(C₁₋₆ alkyl)(C₁₋₆ alkyl), —NO₂ and hydrogen, wherein the alkyl moiety of said —NH(C₁₋₆ alkyl) and one or both of the alkyl moieties of said —N(C₁₋₆ alkyl)(C₁₋₆ alkyl) are each optionally substituted with one or more groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH, —S(C₁₋₆ alkyl), carbocyclyl and heterocyclyl, wherein said carbocyclyl and said heterocyclyl are each optionally substituted with one or more groups R⁵¹.

Each R⁵¹ is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl).

R^{5b} is selected from —R^{5b1}—R^{5b2}—R^{5b1}—, —N═C(R⁵³)—R^{5b3}—R^{5b1}—, —R^{5b1}—R^{5b3}—C(R⁵³)═N—, and —N═C(R⁵³)—R^{5b4}—C(R⁵³)═N—.

Each R^{5b1} is independently selected from —N(R⁵²)—, —O— and —S—.

R^{5b2} is selected from —C(R⁵³)(R⁵³)—, —C(R⁵³)(R⁵³)—C(R⁵³)(R⁵³)—, —C(R⁵³)═C(R⁵³)—, —C(R⁵³)(R⁵³)—C(R⁵³)═C(R⁵³)— and —C(R⁵³)═C(R⁵³)—C(R⁵³)(R⁵³)—.

R^{5b3} is selected from a covalent bond, —C(R⁵³)(R⁵³)—, —C(R⁵³)(R⁵³)—C(R⁵³)(R⁵³)— and —C(R⁵³)═C(R⁵³)—.

R^{5b4} is selected from a covalent bond and —C(R⁵³)(R⁵³)—.

Each R⁵² is independently selected from hydrogen, C₁₋₆ alkyl, C₂₋₆ alkenyl, C₂₋₆ alkynyl, —(C₀₋₄ alkylene)-aryl and —(C₀₋₄ alkylene)-heteroaryl.

Each R⁵³ is independently selected from hydrogen, C₁₋₆ alkyl, C₂₋₆ alkenyl, C₂₋₆ alkynyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl), —N(C₁₋₆ alkyl)-SO₂—(C₁₋₆ alkyl), —(C₀₋₄ alkylene)-aryl and —(C₀₋₄ alkylene)-heteroaryl, and any two groups R⁵³ that are attached to the same carbon atom may also together form a group ═O, and any two groups R⁵³ that are attached to adjacent carbon atoms connected by a double bond may also be mutually linked to form a group —C(R⁵⁴)═C(R⁵⁴)—C(R⁵⁴)═C(R⁵⁴)—.

Each R⁵⁴ is independently selected from hydrogen, C₁₋₆ alkyl, C₂₋₆ alkenyl, C₂₋₆ alkynyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl).

R⁶ is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl).

Alternatively, R¹ and R⁶ are mutually linked to form a group —R¹⁶—, wherein:
R¹⁶ is a group —C(R¹⁶¹)(R¹⁶¹)—C(R¹⁶¹)(R¹⁶¹)—C(R¹⁶¹)(R¹⁶¹)—C(R¹⁶¹)(R¹⁶¹)—, wherein one or two —C(R¹⁶¹)(R¹⁶¹)— units comprised in said group are each replaced by —R¹⁶³—;
each R¹⁶¹ is independently selected from hydrogen, C₁₋₆ alkyl, C₂₋₆ alkenyl, C₂₋₆ alkynyl, halogen, C₁₋₆ haloalkyl, —(C₀₋₆ alkylene)-CF₃, —(C₀₋₆ alkylene)-CN, —(C₀₋₆ alkylene)-NO₂, —(C₀₋₆ alkylene)-N(R¹⁶²)(R¹⁶²), —(C₀₋₆ alkylene)-O(R¹⁶²), —(C₀₋₆ alkylene)-S(R¹⁶²), —(C₀₋₆ alkylene)-SO₃H, —(C₀₋₆ alkylene)-carbocyclyl and —(C₀₋₆ alkylene)-heterocyclyl;
each R¹⁶² is independently hydrogen or C₁₋₆ alkyl; and
each R¹⁶³ is independently selected from —N(R¹⁶¹)—, —O— and —S—.

In accordance with the invention, if the ring moiety

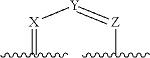

in formula (I) is

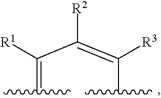

R² is hydrogen, R³ is —SO₂—NH₂, R⁴ is —O-phenyl, R⁵ is —NH—CH₂CH₂CH₂CH₃ and R⁶ is hydrogen, then R¹ is different from —COOH.

The present invention also relates to a pharmaceutical composition comprising a compound of formula (I) or a pharmaceutically acceptable salt or solvate thereof, in combination with a pharmaceutically acceptable excipient, for use in the treatment or prevention of hyperhidrosis.

Moreover, the present invention relates to the use of a compound of formula (I) or a pharmaceutically acceptable salt or solvate thereof in the preparation of a medicament for the treatment or prevention of hyperhidrosis.

The invention likewise relates to a method of treating or preventing hyperhidrosis, the method comprising administering a compound of formula (I) or a pharmaceutically acceptable salt or solvate thereof, or a pharmaceutical composition comprising any of the aforementioned entities in combination with a pharmaceutically acceptable excipient, to a subject (preferably a human) in need thereof. It will be understood that a therapeutically effective amount of the compound of formula (I) or the pharmaceutically acceptable salt or solvate thereof, or of the pharmaceutical composition, is to be administered in accordance with this method.

The present invention furthermore relates to the use of a compound of formula (I) or a pharmaceutically/physiologically acceptable salt or solvate thereof for suppressing or reducing the non-pathological sweating of a subject (e.g., a human). Accordingly, the invention relates to the non-therapeutic use of a compound of formula (I) or a pharmaceutically/physiologically acceptable salt or solvate thereof for suppressing or reducing the sweating of a subject (e.g., a human). The invention likewise refers to a method (particularly a non-therapeutic method) of suppressing or reducing the sweating of a subject (e.g., a human), wherein the method comprises administering a compound of formula (I) or a pharmaceutically/physiologically acceptable salt or solvate thereof to the subject. The subject whose sweating is to be suppressed or reduced is preferably a healthy subject, particularly a subject that does not suffer from hyperhidrosis. The invention particularly relates to suppressing or reducing the sweating of a subject, wherein the sweating is caused by or occurs during physical stress or physical exercise (e.g., sport).

For the treatment or prevention of hyperhidrosis but also for the suppression or reduction of non-pathological sweating, it is preferred that the compound of formula (I) or the pharmaceutically/physiologically acceptable salt or solvate thereof is administered topically, particularly to those skin sections that are affected (or strongly affected) by sweating and/or those skin sections where it is desired to reduce sweating. For topical application, the compound of formula (I) or the pharmaceutically/physiologically acceptable salt or solvate thereof can be provided as such or, for example, in the form of a composition (e.g., in the form of a pharmaceutical composition comprising said compound and a pharmaceutically acceptable excipient for use in the treatment of prevention of hyperhidrosis, or in the form of a cosmetic composition comprising said compound and a cosmetically/physiologically acceptable excipient for suppressing or reducing non-pathological sweating). The compound of formula (I) or the pharmaceutically/physiologically acceptable salt or solvate thereof can also be provided in the form of a wipe (or a wiping cloth) comprising said compound, in the form of an insole (or a shoe insert or an arch support; including, in particular, an orthopedic insole, an orthopedic shoe insert, or an orthopedic arch support) comprising said compound, or in the form of a garment (or a piece of clothing) comprising said compound. Each of the aforementioned articles (i.e., the wipe, wiping cloth, insole, shoe insert, arch support, garment, or piece of clothing) may, e.g., be treated, coated or impregnated with said compound.

Preferably, at least one surface (particularly at least one body-facing surface) of the aforementioned articles is (or has been) treated, coated or impregnated with a compound of formula (I) or a pharmaceutically/physiologically acceptable salt or solvate thereof. The present invention also relates to such articles containing a compound as provided herein. Thus, the invention encompasses, in particular, an article comprising a compound of formula (I) or a pharmaceutically/physiologically acceptable salt or solvate thereof, wherein the article is (i) a wipe (or a wiping cloth), (ii) an insole (or a shoe insert or an arch support; e.g., an orthopedic insole, an orthopedic shoe insert, or an orthopedic arch support), or (iii) a garment (or a piece of clothing).

In accordance with the invention, not only hyperhidrosis but also other diseases/disorders involving (or mediated by) NKCCs, particularly diseases/disorders involving (or mediated by) NKCC1, can be treated or prevented using a compound of formula (I).

Thus, the present invention also relates to a compound of formula (I) as described and defined herein above, or a pharmaceutically acceptable salt or solvate thereof, or a pharmaceutical composition comprising any of the aforementioned entities and a pharmaceutically acceptable excipient, for use in the treatment or prevention of a disease or disorder involving an NKCC, particularly a disease or disorder involving NKCC1.

The invention further relates to the use of a compound of formula (I) or a pharmaceutically acceptable salt or solvate thereof in the preparation of a medicament for the treatment or prevention of a disease or disorder involving an NKCC, particularly a disease or disorder involving NKCC1.

The invention likewise provides a method of treating or preventing a disease or disorder involving an NKCC (particularly a disease or disorder involving NKCC1), the method comprising administering a compound of formula (I) or a pharmaceutically acceptable salt or solvate thereof, or a pharmaceutical composition comprising any of the aforementioned entities in combination with a pharmaceutically acceptable excipient, to a subject (preferably a human) in need thereof. It will be understood that a therapeutically effective amount of the compound of formula (I) or the pharmaceutically acceptable salt or solvate thereof, or of the pharmaceutical composition, is to be administered in accordance with this method.

The disease or disorder to be treated or prevented in accordance with the present invention, specifically the disease or disorder involving an NKCC (preferably NKCC1), is not particularly limited, and is preferably selected from hyperhidrosis (e.g., primary hyperhidrosis, secondary hyperhidrosis, or nocturnal hyperhidrosis), an anxiety disorder, an autism spectrum disorder (e.g., autism, Asperger syndrome, childhood disintegrative disorder, or a pervasive developmental disorder as part of an autism spectrum disorder), traumatic brain injury, spinal cord injury (including also chronic pain caused by spinal cord injury), peripheral nerve injury, stroke (e.g., ischemic stroke; including, in particular, the use of the compounds according to the invention in promoting recovery after stroke, or the use of said compounds in reducing brain damage and/or neurological deficits after stroke), Alzheimer's disease, schizophrenia, asthma, edema (e.g., brain edema), Down syndrome (particularly mental disability in patients with Down syndrome), glaucoma (e.g., primary open angle glaucoma or angle closure glaucoma), or a parasitic infection. The parasitic infection may be, for example, (i) a helminth infection, including also a soil-transmitted helminth infection, particularly an infection with (or a disease caused by) a hookworm, roundworm, whipworm, tapeworm, guinea worm, pinworm, toxocara, Strongyloides stercoralis, or Ascaris lumbricoides; (ii) a parasitic fluke infection or a disease caused by a parasitic fluke, particularly Schistosomiasis, Gnathostomiasis, Paragonimiasis, Fascioliasis, or Swimmer's itch; (iii) a protozoan infection or a disease caused by protozoa such as, e.g., malaria, amoebiasis, giardiasis, African sleeping sickness, toxoplasmosis, Acanthamoeba keratitis, leishmaniasis, babesiosis, granulomatous amoebic encephalitis, cryptosporidiosis, cyclosporiasis, or primary amoebic meningoencephalitis; or (iv) an ectoparasitic infection, particularly an infection with (or a disease caused by) Sarcoptes scabiei, Pediculus humanus capitis, Phthirus pubis, human botfly maggots, Tunga penetrans, or lxodoidea.

Accordingly, it is preferred that the disease or disorder to be treated or prevented in accordance with the invention is selected from hyperhidrosis, primary hyperhidrosis, secondary hyperhidrosis, nocturnal hyperhidrosis, an anxiety disorder, an autism spectrum disorder, autism, Asperger syndrome, childhood disintegrative disorder, a pervasive developmental disorder as part of an autism spectrum disorder, traumatic brain injury, spinal cord injury (including also chronic pain caused by spinal cord injury), peripheral nerve injury, stroke (e.g., ischemic stroke), Alzheimer's disease, schizophrenia, asthma, edema (e.g., brain edema), Down syndrome, mental disability in patients with Down syndrome, glaucoma, primary open angle glaucoma, angle closure glaucoma, or a parasitic infection. The parasitic infection is preferably selected from: (i) a helminth infection, particularly a hookworm infection, a roundworm infection, a whipworm infection, a tapeworm infection, a guinea worm infection, a pinworm infection, a toxocara infection, a Strongyloides stercoralis infection, or an Ascaris lumbricoides infection; (ii) a parasitic fluke infection, particularly Schistosomiasis, Gnathostomiasis, Paragonimiasis, Fascioliasis, or Swimmer's itch; (iii) a protozoan infection, particularly malaria, amoebiasis, giardiasis, African sleeping sickness, toxoplasmosis, Acanthamoeba keratitis, leishmaniasis, babesiosis, granulomatous amoebic encephalitis, cryptosporidiosis, cyclosporiasis, or primary amoebic meningoencephalitis; or (iv) an ectoparasitic infection, particularly an infection with (or a disease caused by) Sarcoptes scabiei, Pediculus humanus capitis, Phthirus pubis, human botfly maggots, Tunga penetrans, or lxodoidea. It is particularly preferred that the disease or disorder to be treated or prevented in accordance with the invention is hyperhidrosis (e.g., primary hyperhidrosis, secondary hyperhidrosis, or nocturnal hyperhidrosis).

The disease or disorder to be treated or prevented in accordance with the present invention may also be, e.g., a neurological disease or disorder involving (or mediated by) an NKCC, particularly a neurological disease or disorder involving (or mediated by) NKCC1. Corresponding examples include, in particular, stroke (e.g., ischemic stroke), traumatic brain injury, spinal cord injury, peripheral nerve injury, or brain edema.

The compounds of formula (I) can further be used as pest control agents, particularly for controlling a plant pest (such as, e.g., a nematode, an arthropod, an ectoparasite and/or a mollusk), e.g., by applying a compound of formula (I) or a salt or solvate thereof to a plant or to soil.

Moreover, the present invention also provides novel compounds, as described in more detail further below, which can be used as medicaments, and a pharmaceutical composition comprising such a compound and a pharmaceutically acceptable excipient.

The present invention furthermore relates to the use of a compound of formula (I) or a pharmaceutically acceptable salt or solvate thereof as an NKCC inhibitor in research, particularly as a research tool compound for inhibiting NKCC (particularly NKCC1). Accordingly, the invention refers to the in vitro use of a compound of formula (I) or a pharmaceutically acceptable salt or solvate thereof as an NKCC inhibitor (particularly as an NKCC1 inhibitor) and, likewise, to the in vitro use of a compound of formula (I) or a pharmaceutically acceptable salt or solvate thereof as a research tool compound acting as an NKCC inhibitor (particularly as an NKCC1 inhibitor). The invention also relates to an in vitro method of inhibiting NKCC (particularly NKCC1), comprising the application of a compound of formula (I) or a pharmaceutically acceptable salt or solvate thereof. The present invention further provides an in vitro method of inhibiting NKCC (particularly NKCC1), comprising the use of a compound of formula (I) or a pharmaceutically acceptable salt or solvate thereof as an NKCC inhibitor (particularly as an NKCC1 inhibitor). It is to be understood that the term “in vitro” is used in this specific context in the sense of “outside a living human or animal body”, which includes, in particular, experiments performed with cells, cellular or subcellular extracts, and/or biological molecules in an artificial environment such as an aqueous solution or a culture medium which may be provided, e.g., in a flask, a test tube, a Petri dish, a microtiter plate, etc.

The compound of formula (I) as well as the pharmaceutically acceptable salt or solvate thereof will be described in more detail in the following:

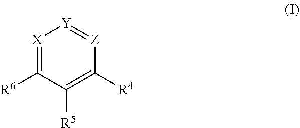

In formula (I), the ring moiety

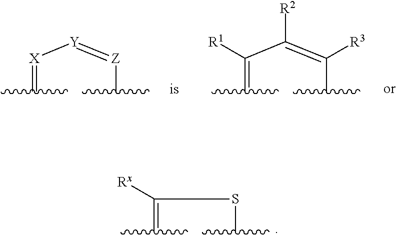

It will be understood that, if the ring moiety

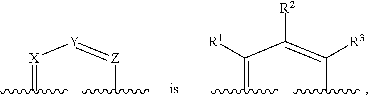

then the compound of formula (I) has the following structure:

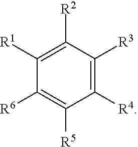

Conversely, if the ring moiety

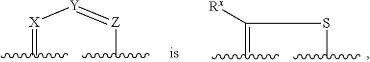

then the compound of formula (I) has the following structure:

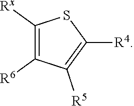

R¹ is selected from —COOH, —COO—(C₁₋₁₅ alkyl), —COO—(C₀₋₁₅ alkylene)-carbocyclyl, —COO—(C₀₋₁₅ alkylene)-heterocyclyl, —O—CHO, —O—CO—(C₁₋₁₅ alkyl), —O—CO—(C₀₋₁₅ alkylene)-carbocyclyl, —O—CO—(C₀₋₁₅ alkylene)-heterocyclyl, —CHO, —CO—(C₁₋₁₅ alkyl), —CO—(C₀₋₁₅ alkylene)-carbocyclyl, —CO—(C₀₋₁₅ alkylene)-heterocyclyl, —CO—NH₂, —CO—N(R¹)—(C₁₋₁₅ alkyl), —CO—N(R¹¹)—(C₀₋₁₅ alkylene)-carbocyclyl, —CO—N(R¹¹)—(C₀₋₁₅ alkylene)-heterocyclyl, —N(R¹¹)—CHO, —N(R¹¹)—CO—(C₁₋₁₅ alkyl), —N(R¹¹)—CO—(C₀₋₁₅ alkylene)-carbocyclyl, —N(R¹¹)—CO—(C₀₋₁₅ alkylene)-heterocyclyl, C₁₋₁₅ alkyl, —(C₀₋₁₅ alkylene)-carbocyclyl, —(C₀₋₁₅ alkylene)-heterocyclyl, C₂₋₁₅ alkenyl, —(C₂₋₁₅ alkenylene)-carbocyclyl, —(C₂₋₁₅ alkenylene)-heterocyclyl, C₂₋₁₅ alkynyl, —(C₂₋₁₅ alkynylene)-carbocyclyl and —(C₂₋₁₅ alkynylene)-heterocyclyl,

wherein the alkyl moiety of any of the aforementioned groups, the alkylene moiety of any of the aforementioned groups, the alkenylene moiety of any of the aforementioned groups, the alkynylene moiety of any of the aforementioned groups, said C₁₋₁₅ alkyl, said C₂₋₁₅ alkenyl and said C₂₋₁₅ alkynyl are each optionally substituted with one or more (e.g., one, two or three) groups independently selected from halogen, —CF₃, —CN, —NO₂, —N(R¹¹)(R¹¹), —O(R¹¹), —S(R¹¹) and —SO₃H,

wherein one or more (e.g., one or two) —CH₂— units comprised in the alkyl moiety of any of the aforementioned groups, in the alkylene moiety of any of the aforementioned groups, in the alkenylene moiety of any of the aforementioned groups, in the alkynylene moiety of any of the aforementioned groups, in said C₁₋₁₅ alkyl, in said C₂₋₁₅ alkenyl, or in said C₂₋₁₅ alkynyl are each optionally replaced by a group independently selected from —O—, —CO—, —COO—, —O—CO—, —N(R¹¹)—, —N(R¹¹)—CO—, —CO—N(R¹¹)—, —S—, —SO—, —SO₂—, —SO₂—N(R¹¹)— and —N(R¹¹)—SO₂—,

and further wherein the carbocyclyl moiety of any of the aforementioned groups and the heterocyclyl moiety of any of the aforementioned groups are each optionally substituted with one or more (e.g., one, two or three) groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹¹), —S(R¹¹), —SO₃H, carbocyclyl and heterocyclyl.

Each R¹¹ is independently hydrogen or C₁₋₆ alkyl. Preferably, each R¹¹ is independently hydrogen or C₁₋₄ alkyl (e.g., methyl or ethyl).

R¹ may be, for example, any one of the specific groups R¹ as comprised in any of the compounds described in the examples section.

In particular, R¹ may be, e.g., —COO—(C₁₋₁₅ alkyl), wherein the alkyl moiety of said —COO—(C₁₋₁₅ alkyl) is optionally substituted with one or more (e.g., one, two or three) groups independently selected from halogen, —CF₃, —CN, —NO₂, —NH₂, —NH(C₁₋₄ alkyl), —N(C₁₋₄ alkyl)(C₁₋₄ alkyl), —OH, —O(C₁₋₄ alkyl), —SH and —S(C₁₋₄ alkyl), and further wherein one or two —CH₂— units comprised in the alkyl moiety of said —COO—(C₁₋₁₅ alkyl) are each optionally replaced by a group independently selected from —O—, —CO—, —COO—, —O—CO—, —NH—, —N(C₁₋₄ alkyl)-, —NH—CO—, —N(C₁₋₄ alkyl)-CO—, —CO—NH—, —CO—N(C₁₋₄ alkyl)-, —S—, —SO—, —SO₂—, —SO₂—NH—, —SO₂—N(C₁₋₄ alkyl)-, —NH—SO₂— and —N(C₁₋₄ alkyl)-SO₂—. The alkyl moiety of said —COO—(C₁₋₁₅ alkyl) has 1 to 15 carbon atoms, particularly 1 to 10 carbon atoms. Preferred examples of such R¹ groups include the corresponding groups R¹ of the compounds described in the examples section (including, e.g., —COO—CH₃ or —COO—CH₂CH₃; particularly —COO—CH₃).

A further preferred example of R¹ is —COOH.

Further preferred examples of R¹ are —(C₁₋₄ alkylene)-NH—(C₁₋₄ alkylene)-R¹² (such as, e.g., —CH₂—NH—CH₂—R¹²), —COO—(C₁₋₄ alkylene)-R¹² (e.g., —COO—CH₂—R¹²), —O—CO—(C₁₋₄ alkylene)-R¹², —CO—(C₁₋₄ alkylene)-R¹², —CO—NH—(C₁₋₄ alkylene)-R¹² (e.g., —CO—NH—CH₂—R¹²), —CO—N(C₁₋₄ alkyl)-(C₁₋₄ alkylene)-R¹², —NH—CO—(C₁₋₄ alkylene)-R¹², or —N(C₁₋₄ alkyl)-CO—(C₁₋₄ alkylene)-R¹², wherein R¹² is independently selected from —CF₃, —CN and halogen (e.g., —F, —C, —Br or —I); preferably, R¹² is independently selected from —CF₃ and —CN; more preferably, R¹² is —CF₃. Specific examples of such R¹ groups include the corresponding groups R¹ of the compounds described in the examples section. A particularly preferred example of R¹ is —(C₁₋₄ alkylene)-NH—(C₁₋₄ alkylene)-CF₃, and even more preferred example is —CH₂—NH—CH₂—CF₃.

A further example of R¹ is —(C₁₋₄ alkylene)-S-heterocyclyl, particularly —(C₁₋₄ alkylene)-S-heteroaryl (such as, e.g., —CH₂—S-heteroaryl), wherein the heterocyclyl moiety of said —(C₁₋₄ alkylene)-S-heterocyclyl or the heteroaryl moiety of said —(C₁₋₄ alkylene)-S-heteroaryl or of said —CH₂—S-heteroaryl is optionally substituted with one or more (e.g., one, two or three) groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹¹), —S(R¹¹), —SO₃H, carbocyclyl and heterocyclyl. Specific examples of such R¹ groups include the corresponding groups R¹ of the compounds described in the examples section.

A further example of R¹ is —(C₁₋₄ alkylene)-O—(C₀₋₄ alkylene)-carbocyclyl, particularly —(C₁₋₄ alkylene)-O—(C₀₋₄ alkylene)-phenyl (such as, e.g., —CH₂—O-phenyl or —CH₂—O—CH₂-phenyl), wherein the carbocyclyl moiety of said —(C₁₋₄ alkylene)-O—(C₀₋₄ alkylene)-carbocyclyl or the phenyl moiety of said —(C₁₋₄ alkylene)-O—(C₀₋₄ alkylene)-phenyl (or of said —CH₂—O-phenyl or —CH₂—O—CH₂-phenyl) is optionally substituted with one or more (e.g., one, two or three) groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹¹), —S(R¹¹), —SO₃H, carbocyclyl and heterocyclyl. Specific examples of such R¹ groups include the corresponding groups R¹ of the compounds described in the examples section.

A further example of R¹ is —(C₁₋₄ alkylene)-heterocyclyl, particularly —CH₂-heterocyclyl, wherein the heterocyclyl moiety of said —(C₁₋₄ alkylene)-heterocyclyl or of said —CH₂-heterocyclyl is optionally substituted with one or more (e.g., one, two or three) groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹¹), —S(R¹¹), —SO₃H, carbocyclyl and heterocyclyl. The heterocyclyl moiety may be, e.g., a heterocycloalkyl (such as, e.g., pyrrolidinyl, piperidinyl, piperazinyl, or morpholinyl), a heterocycloalkenyl (such as, e.g., tetrahydropyridinyl), or a heteroaryl. Specific examples of such R¹ groups include the corresponding groups R¹ of the compounds described in the examples section.

R² is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl).

Preferably, R² is selected from hydrogen, C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN. More preferably, R² is hydrogen or C₁₋₄ alkyl. Even more preferably, R² is hydrogen.

R³ is selected from —SO₂—NH₂, —SO₂—NH(C₆ alkyl), —SO₂—N(C₆ alkyl)(C₆ alkyl), —SO₂—N═(C₁₋₆ alkylidene) and —SO₂-halogen, wherein the alkyl moiety of said —SO₂—NH(C₁₋₆ alkyl), one or both of the alkyl moieties of said —SO₂—N(C₁₋₆ alkyl)(C₁₋₆ alkyl), and the alkylidene moiety of said —SO₂—N═(C₁₋₆ alkylidene) are each optionally substituted with one or more (e.g., one, two or three) groups independently selected from halogen, —CF₃, —CN, —NO₂, —NH₂, —NH(C₆ alkyl), —N(C₁₋₆ alkyl)(C₁₋₆ alkyl), —OH, —O(C₁₋₆ alkyl), —SH and —S(C₁₋₆ alkyl).

Preferably, R³ is selected from —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C4 alkylidene) are each optionally substituted with one or more groups (particularly one group) independently selected from halogen, —CF₃, —CN, —NO₂, —NH₂, —NH(C₆ alkyl), —N(C₁₋₆ alkyl)(C₁₋₆ alkyl), —OH, —O(C₁₋₆ alkyl), —SH and —S(C₁₋₆ alkyl). More preferably, R³ is selected from —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one group selected from —NH₂, —NH(C₁₋₄ alkyl) and —N(C₁₋₄ alkyl)(C₁₋₄ alkyl). Even more preferably, R³ is selected from —SO₂—NH₂, —SO₂—NH(C₁₋₄ alkyl), —SO₂—N(C₁₋₄ alkyl)(C₁₋₄ alkyl), —SO₂—NH—(C₁₋₄ alkylene)-NH₂, —SO₂—NH—(C₁₋₄ alkylene)-NH(C₁₋₄ alkyl), —SO₂—NH—(C₁₋₄ alkylene)-N(C₁₋₄ alkyl)(C₁₋₄ alkyl), —SO₂—N═(C₁₋₄ alkylidene)-NH₂, —SO₂—N═(C₁₋₄ alkylidene)-NH(C₁₋₄ alkyl) and —SO₂—N═(C₁₋₄ alkylidene)-N(C₁₋₄ alkyl)(C₁₋₄ alkyl). Yet even more preferably, R³ is selected from —SO₂—NH₂, —SO₂—NH—CH₃, —SO₂—N(CH₃)₂, —SO₂—NH—(C₁₋₄ alkylene)-NH₂, —SO₂—NH—(C₁₋₄ alkylene)-NH—CH₃, —SO₂—NH—(C₁₋₄ alkylene)-N(CH₃)₂ (e.g., —SO₂—NH—CH₂CH₂—N(CH₃)₂), —SO₂—N═(C₁₋₄ alkylidene)-NH₂, —SO₂—N═(C₁₋₄ alkylidene)-NH—CH₃ and —SO₂—N═(C₁₋₄ alkylidene)-N(CH₃)₂ (e.g., —SO₂—N═CH—N(CH₃)₂). A particularly preferred example of R³ is —SO₂—NH₂. A further particularly preferred example of R³ is —SO₂—N═CH—N(CH₃)₂.

R^{x} is R¹ or R³ (as defined above, including also the preferred definitions of R¹ and R³).

Preferably, R^{x} is selected from —COOH, —COO—(C₁₋₆ alkyl), —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one group selected from —NH₂, —NH(C₁₋₄ alkyl) and —N(C₁₋₄ alkyl)(C₁₋₄ alkyl). More preferably, R^{x} is selected from —COOH, —COOCH₃, —SO₂—NH₂ and —SO₂—N═CH—N(CH₃)₂.

R⁴ is a group R^{4a}, and R⁵ is a group R^{5a}; or alternatively, R⁴ and R⁵ are mutually linked to form a group —R^{5b}—.

R^{4a} is selected from —O—R⁴¹, —S—R⁴¹, —NH—R⁴¹, —N(C₁₋₆ alkyl)-R⁴¹, halogen (e.g., —Cl), hydrogen, carbocyclyl and heterocyclyl, wherein said carbocyclyl and said heterocyclyl are each optionally substituted with one or more (e.g., one, two or three) groups R⁴²

Preferably, R^{4a} is selected from —O—R⁴¹, —S—R⁴¹, —NH—R⁴¹, —N(C₁₋₆ alkyl)-R⁴¹, halogen, carbocyclyl and heterocyclyl, wherein said carbocyclyl and said heterocyclyl are each optionally substituted with one or more groups R⁴². More preferably, R^{4a} is selected from —O—R⁴¹, —S—R⁴¹, —NH—R⁴¹, —N(C₁₋₆ alkyl)-R⁴¹, carbocyclyl (e.g., aryl, cycloalkyl, or cycloalkenyl) and heterocyclyl (e.g., heteroaryl, heterocycloalkyl, or heterocycloalkenyl), wherein said carbocyclyl and said heterocyclyl are each optionally substituted with one or more groups R⁴². Even more preferably, R^{4a} is selected from —O—R⁴¹, —S—R⁴¹, —NH—R⁴¹, —N(C₁₋₄ alkyl)-R⁴¹, aryl and heteroaryl, wherein said aryl and said heteroaryl are each optionally substituted with one or more groups R⁴².

R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₀₋₄ alkylene)-carbocyclyl and the heterocyclyl moiety of said —(C₀₋₄ alkylene)-heterocyclyl are each optionally substituted with one or more (e.g., one, two or three) groups R⁴², and wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more (e.g., one, two or three) groups R⁴³.

Preferably, R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₀₋₄ alkylene)-carbocyclyl is selected from cycloalkyl, cycloalkenyl and aryl, wherein the heterocyclyl moiety of said —(C₀₋₄ alkylene)-heterocyclyl is selected from heterocycloalkyl, heterocycloalkenyl and heteroaryl, wherein the carbocyclyl moiety of said —(C₀₋₄ alkylene)-carbocyclyl and the heterocyclyl moiety of said —(C₀₋₄ alkylene)-heterocyclyl are each optionally substituted with one or more (e.g., one, two or three) groups R⁴², and further wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more (e.g., one, two or three) groups R⁴³. More preferably, R⁴¹ is selected from —(C₀₋₄ alkylene)-aryl and —(C₀₋₄ alkylene)-heteroaryl, wherein the aryl moiety of said —(C₀₋₄ alkylene)-aryl and the heteroaryl moiety of said —(C₀₋₄ alkylene)-heteroaryl are each optionally substituted with one or more (e.g., one, two or three) groups R⁴², and further wherein the alkylene moiety of said —(C₀₋₄ alkylene)-aryl and the alkylene moiety of said —(C₀₋₄ alkylene)-heteroaryl are each optionally substituted with one or more (e.g., one, two or three) groups R⁴³. Even more preferably, R⁴¹ is selected from —(C₀₋₄ alkylene)-aryl and —(C₀₋₄ alkylene)-heteroaryl, wherein the aryl moiety of said —(C₀₋₄ alkylene)-aryl and the heteroaryl moiety of said —(C₀₋₄ alkylene)-heteroaryl are each optionally substituted with one or more (e.g., one, two or three) groups R⁴². A preferred example of the aryl moiety of said —(C₀₋₄ alkylene)-aryl is phenyl. A preferred example of the heteroaryl moiety of said —(C₀₋₄ alkylene)-heteroaryl is a 5- or 6-membered monocyclic heteroaryl having 1 or 2 ring heteroatoms independently selected from oxygen, nitrogen and sulfur (wherein the remaining ring atoms are carbon atoms), such as, e.g., imidazolyl, thiophenyl, or pyrimidinyl. Still more preferably, R⁴¹ is selected from phenyl and heteroaryl, wherein said heteroaryl is a 5- or 6-membered monocyclic heteroaryl having 1 or 2 ring heteroatoms independently selected from oxygen, nitrogen and sulfur (the remaining ring atoms of the monocyclic heteroaryl are carbon atoms), and further wherein said phenyl or said heteroaryl is optionally substituted with one or more (e.g., one, two or three) groups R⁴²

Each R⁴² is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆alkyl).

Preferably, each R⁴² is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

Each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃, —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl) and —N(C₁₋₆ alkyl)-CO—(C₁₋₆ alkyl).

Preferably, each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃ and —CN.

In accordance with the above definitions, it is particularly preferred that R^{4a} is selected from —O—(C₀₋₄ alkylene)-aryl, —O—(C₀₋₄ alkylene)-heteroaryl, —S—(C₀₋₄ alkylene)-aryl, —S—(C₀₋₄ alkylene)-heteroaryl, —NH—(C₀₋₄ alkylene)-aryl, —NH—(C₀₋₄ alkylene)-heteroaryl, —N(C₀₋₄ alkyl)-(C₀₋₄ alkylene)-aryl, —N(C₁₋₄ alkyl)-(C₀₋₄ alkylene)-heteroaryl, aryl and heteroaryl, wherein the aryl moiety of any of the aforementioned groups, the heteroaryl moiety of any of the aforementioned groups, said aryl and said heteroaryl are each optionally substituted with one or more (e.g., one, two or three) groups R⁴². Even more preferably, R^{4a} is selected from —O-aryl, —O-heteroaryl, —S-aryl, —S-heteroaryl, —NH-aryl, —NH-heteroaryl, —N(C₁₋₄ alkyl)-aryl, —N(C₁₋₄ alkyl)-heteroaryl, aryl and heteroaryl, wherein the aryl moiety of any of the aforementioned groups, the heteroaryl moiety of any of the aforementioned groups, said aryl and said heteroaryl are each optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN. Yet even more preferably, R^{4a} is selected from —O-phenyl, —O— heteroaryl, —S-phenyl, —S-heteroaryl, —NH-phenyl, —NH-heteroaryl, —N(C₁₋₄ alkyl)-phenyl, —N(C₁₋₄ alkyl)-heteroaryl, phenyl and heteroaryl, wherein said heteroaryl or the heteroaryl moiety of any of the aforementioned groups is a 5- or 6-membered monocyclic heteroaryl having 1 or 2 ring heteroatoms independently selected from oxygen, nitrogen and sulfur (the remaining ring atoms of the monocyclic heteroaryl are carbon atoms), and further wherein the phenyl moiety of any of the aforementioned groups, the heteroaryl moiety of any of the aforementioned groups, said phenyl and said heteroaryl are each optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN. A particularly preferred example of R^{4a} is —O-phenyl.

R^{5a} is selected from —NH₂, —NH(C₁₋₆ alkyl), —N(C₁₋₆ alkyl)(C₁₋₆ alkyl), —NO₂ and hydrogen, wherein the alkyl moiety of said —NH(C₁₋₆ alkyl) and one or both of the alkyl moieties of said —N(C₁₋₆ alkyl)(C₁₋₆ alkyl) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH, —S(C₁₋₆ alkyl), carbocyclyl and heterocyclyl, wherein said carbocyclyl and said heterocyclyl are each optionally substituted with one or more (e.g., one, two or three) groups R⁵¹.

Preferably, R^{5a} is selected from —NH₂, —NH(C₁₋₆ alkyl), —N(C₁₋₆ alkyl)(C₁₋₆ alkyl) and —NO₂, wherein the alkyl moiety of said —NH(C₁₋₆ alkyl) and one or both of the alkyl moieties of said —N(C₁₋₆ alkyl)(C₁₋₆ alkyl) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl). More preferably, R^{5a} is selected from —NH₂, —NH(C₁₋₆ alkyl), —N(C₁₋₆ alkyl)(C₁₋₆ alkyl), and —NO₂. Particularly preferred examples of R^{5a} are —NH₂, —NH—CH₂CH₂CH₂CH₃, or —NO₂.

Each R⁵¹ is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl).

Preferably, each R⁵¹ is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

R^{5b} is selected from —R^{5b1}—R^{5b2}—R^{5b1}—, —N═C(R⁵³)—R^{5b3}—R^{5b1}—, —R^{5b1}—R^{5b3}—C(R⁵³)═N—, and —N═C(R⁵³)—R^{5b4}—C(R⁵³)═N—.

Each R^{5b1} is independently selected from —N(R⁵²)—, —O— and —S—.

R^{5b2} is selected from —C(R⁵³)(R⁵³)—, —C(R⁵³)(R⁵³)—C(R⁵³)(R⁵³)—, —C(R⁵³)═C(R⁵³)—, —C(R⁵³)(R⁵³)—C(R⁵³)═C(R⁵³)— and —C(R⁵³)═C(R⁵³)—C(R⁵³)(R⁵³)—.

R^{5b3} is selected from a covalent bond, —C(R⁵³)(R⁵³)—, —C(R⁵³)(R⁵³)—C(R⁵³)(R⁵³)— and —C(R⁵³)═C(R⁵³)—.

R^{5b4} is selected from a covalent bond and —C(R⁵³)(R⁵³)—.

Each R⁵² is independently selected from hydrogen, C₁₋₆ alkyl, C₂₋₆ alkenyl, C₂₋₆ alkynyl, —(C₀₋₄ alkylene)-aryl and —(C₀₋₄ alkylene)-heteroaryl.

Preferably, each R⁵² is independently selected from hydrogen, C₁₋₄ alkyl (e.g., n-butyl), C₂₋₄ alkenyl and C₂₋₄ alkynyl (e.g., —CH₂—C═CH). Even more preferably, each R⁵² is hydrogen.

Each R⁵³ is independently selected from hydrogen, C₁₋₆ alkyl, C₂₋₆ alkenyl, C₂₋₆ alkynyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl), —N(C₁₋₆ alkyl)-SO₂—(C₁₋₆ alkyl), —(C₀₋₄ alkylene)-aryl and —(C₀₋₄ alkylene)-heteroaryl; moreover, any two groups R⁵³ that are attached to the same carbon atom may also together form a group ═O; and any two groups R⁵³ that are attached to adjacent carbon atoms connected by a double bond (i.e., any two groups R⁵³ comprised in a moiety —C(R⁵³)═C(R⁵³)—) may also be mutually linked to form a group —C(R⁵⁴)═C(R⁵⁴)—C(R⁵⁴)═C(R⁵⁴)—.

Preferably, each R⁵³ is independently selected from hydrogen, C₁₋₆ alkyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), and —CN, and any two groups R⁵³ that are attached to the same carbon atom may also together form a group ═O, and any two groups R⁵³ that are attached to adjacent carbon atoms connected by a double bond may also be mutually linked to form a group —C(R⁵⁴)═C(R⁵⁴)—C(R⁵⁴)═C(R⁵⁴)—. More preferably, each R⁵³ is independently selected from hydrogen, C₁₋₆ alkyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), and —CN, and any two groups R⁵³ that are attached to the same carbon atom may also together form a group ═O. Even more preferably, each R⁵³ is independently selected from hydrogen, C₁₋₄ alkyl, —OH, —O(C₁₋₄ alkyl), —NH₂, —NH(C₁₋₄ alkyl), —N(C₁₋₄ alkyl)(C₁₋₄ alkyl), halogen, C₁₋₄ haloalkyl, —O—(C₁₋₄ haloalkyl), and —CN, and any two groups R⁵³ that are attached to the same carbon atom may also together form a group ═O.

Each R⁵⁴ is independently selected from hydrogen, C₁₋₆ alkyl, C₂₋₆ alkenyl, C₂₋₆ alkynyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₆ alkyl)-SO₂—(C₁₋₆ alkyl).

Preferably, each R⁵⁴ is independently selected from hydrogen, C₁₋₆ alkyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN. More preferably, each R⁵⁴ is hydrogen.

It is particularly preferred that R^{5b} is selected from —N(R⁵²)—C(R⁵)(R⁵³)—N(R⁵²)—, —N═C(R⁵³)—N(R⁵²)—, —N(R⁵²)—C(R⁵³)═N—, —N(R⁵²)—C(R⁵³)(R⁵³)—C(R⁵³)(R⁵³)—N(R⁵²)—, —N═C(R⁵³)—C(R⁵³)(R⁵³)—N(R⁵²)—, —N(R⁵²)—C(R⁵³)═C(R⁵³)—N(R⁵²)—, —N(R⁵²)—C(R⁵³)(R⁵³)—C(R⁵³)═N—, —N═C(R⁵³)—C(R⁵³)═N—, —N(R⁵²)—C(R⁵³)(R⁵³)—C(R⁵³)(R⁵³)—C(R⁵³)(R⁵³)—N(R⁵²)—, —N═C(R⁵³)—C(R⁵³)(R⁵³)—C(R⁵³)(R⁵³)—N(R⁵²)—, —N(R⁵²)—C(R⁵³)(R⁵³)—C(R⁵³)═C(R⁵³)—N(R⁵²)—, —N(R⁵²)—C(R⁵³)═C(R⁵³)—C(R⁵³)(R⁵³)—N(R⁵²)—, —N(R⁵²) C(R⁵³)(R⁵³)—C(R⁵³)(R⁵³)—C(R⁵³)═N—, —N═C(R⁵³)—C(R⁵³)(R⁵³)—C(R⁵³)═N—, —O—C(R⁵³)(R⁵³)—N(R⁵²)—, —O—C(R⁵³)(R⁵³)—C(R⁵³)(R⁵³)—N(R⁵²)—, —O—C(R⁵³)═C(R⁵³)—N(R⁵²)—, —O—C(R⁵³)(R⁵³)—C(R⁵³)(R⁵³)—C(R⁵³)(R⁵³)—N(R⁵²)—, —O—C(R⁵³)(R⁵³)—C(R⁵³)═C(R⁵³)—N(R⁵²)—, —O—C(R⁵³)═C(R⁵³)—C(R⁵³)(R⁵³)—N(R⁵²)—, —N(R⁵²)—C(R⁵³)(R⁵³)—O—, —N(R⁵²)—C(R⁵³)(R⁵³)—C(R⁵³)(R⁵³)—O—, —N(R⁵²)—C(R⁵³)═C(R⁵³)—O—, —N(R⁵²)—C(R⁵³)(R⁵³)—C(R⁵³)(R⁵³)—C(R⁵³)(R⁵³)—O—, —N(R⁵²)—C(R⁵³)(R⁵³)—C(R⁵³)═C(R⁵³)—O—, —N(R⁵²)—C(R⁵³)═C(R⁵³)—C(R⁵³)(R⁵³)—O—, —S—C(R⁵³)(R⁵³)—N(R⁵²)—, —S—C(R⁵³)(R⁵³)—C(R⁵³)(R⁵³)—N(R⁵²)—, —S—C(R⁵³)═C(R⁵³)—N(R⁵²)—, —S—C(R⁵³)(R⁵³)—C(R⁵³)(R⁵³)—C(R⁵³)(R⁵³)—N(R⁵²)—, —S—C(R⁵³)(R⁵³)—C(R⁵³)═C(R⁵³)—N(R⁵²)—, —S—C(R⁵³)═C(R⁵³)—C(R⁵³)(R⁵³)—N(R⁵²)—, —N(R⁵²)—C(R⁵³)(R⁵³)—S—, —N(R⁵²) C(R⁵³)(R⁵³)—C(R⁵³)(R⁵³)—S—, —N(R⁵²)—C(R⁵³)═C(R⁵³)—S—, —N(R⁵²)—C(R⁵³)(R⁵³)—C(R⁵³)(R⁵³)—C(R⁵³)(R⁵³)—S—, —N(R⁵²)—C(R⁵³)(R⁵³)—C(R⁵³)═C(R⁵³)—S—, —N(R⁵²)—C(R⁵³)═C(R⁵³)—C(R⁵³)(R⁵³)—S—, —N(R⁵²)—C(R⁵³)(R⁵³)—N(R⁵²)—, —N(R⁵²)—C(R⁵³)(R⁵³)—C(R⁵³)(R⁵³)—N(R⁵²)—, —N(R⁵²)—C(R⁵³)═C(R⁵³)—N(R⁵²)—, —N(R⁵²)—C(R⁵³)(R⁵³)—C(R⁵³)(R⁵³)—C(R⁵³)(R⁵³)—N(R⁵²)—, —N(R⁵²)—C(R⁵³)(R⁵³)—C(R⁵³)═C(R⁵³)—N(R⁵²)— and —N(R⁵²)—C(R⁵³)═C(R⁵³)—C(R⁵³)(R⁵³)—N(R⁵²)—.

Moreover, if the ring moiety

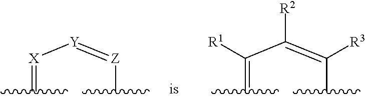

and if R⁴ and R⁵ are mutually linked to form a group —R^{5b}—, then it is even more preferred that R^{5b} is selected such that the compound of formula (I) has any one of the following structures:

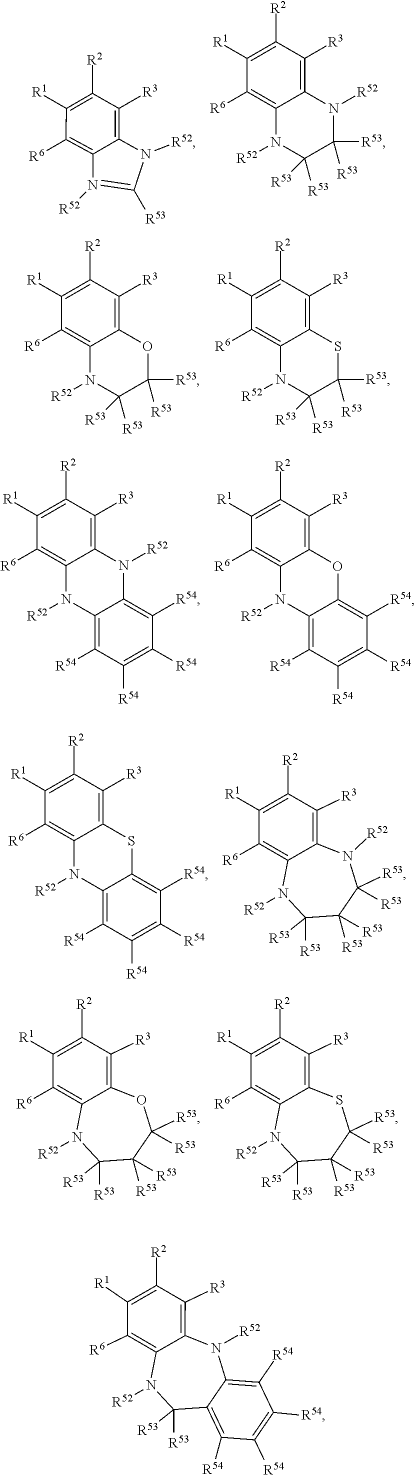

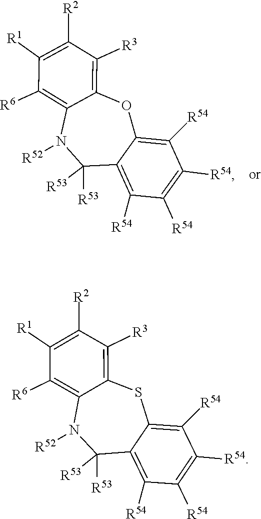

R⁶ is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl).

Preferably, R⁶ is selected from hydrogen, C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN. More preferably, R⁶ is hydrogen or C₁₋₄ alkyl. Even more preferably, R⁶ is hydrogen.

Alternatively (i.e., as an alternative to the above-described meanings of R¹ and R⁶), the groups R¹ and R⁶ are mutually linked to form a group —R¹⁶—, wherein:
R¹⁶ is a group —C(R¹⁶¹)(R¹⁶¹)—C(R¹⁶¹)(R¹⁶¹)—C(R¹⁶¹)(R¹⁶¹)—C(R¹⁶¹)(R¹⁶¹)—, wherein one or two —C(R¹⁶¹)(R¹⁶¹)— units comprised in said group are each replaced by —R¹⁶³—;
each R¹⁶¹ is independently selected from hydrogen, C₁₋₆ alkyl, C₂₋₆ alkenyl, C₂₋₆ alkynyl, halogen, C₁₋₆ haloalkyl, —(C₀₋₆ alkylene)-CF₃, —(C₀₋₆ alkylene)-CN, —(C₀₋₆ alkylene)-NO₂, —(C₀₋₆ alkylene)-N(R¹⁶²)(R¹⁶²), —(C₀₋₆ alkylene)-O(R¹⁶²), —(C₀₋₆ alkylene)-S(R¹⁶²), —(C₀₋₆ alkylene)-SO₃H, —(C₀₋₆ alkylene)-carbocyclyl and —(C₀₋₆ alkylene)-heterocyclyl;
each R¹⁶² is independently hydrogen or C₁₋₆ alkyl; and
each R¹⁶³ is independently selected from —N(R¹⁶¹)—, —O— and —S—.

Preferably, R¹⁶ is a group —CH₂—CH₂—CH₂—CH₂—, wherein one or two —CH₂— units comprised in said group are each replaced by —R¹⁶³—. More preferably, R¹⁶ is a group —CH₂—CH₂—CH₂—CH₂—, wherein one —CH₂— unit comprised in said group is replaced by —R¹⁶³—. Even more preferably, R¹⁶ is —CH₂—R¹⁶³—CH₂—CH₂— or —CH₂—CH₂—R¹⁶³—CH₂—.

Preferably, each R¹⁶³ is independently —N(R¹⁶¹)—. A preferred example of a corresponding group R¹⁶³ is —N(—CH₂—CF₃)—. Accordingly, it is particularly preferred that R¹⁶ is —CH₂—N(R¹⁶¹)—CH₂—CH₂— or —CH₂—CH₂—N(R¹⁶¹)—CH₂— (e.g., —CH₂—N(—CH₂—CF₃)—CH₂—CH₂— or —CH₂—CH₂—N(—CH₂—CF₃)—CH₂—).

In accordance with the invention, if the ring moiety

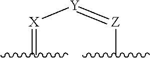

in formula (I) is

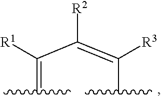

R² is hydrogen, R³ is —SO₂—NH₂, R⁴ is —O-phenyl, R⁵ is —NH—CH₂CH₂CH₂CH₃ and R⁶ is hydrogen, then R¹ is different from —COOH.

Thus, in other words, R¹ is different from —COOH (i.e., R¹ is not —COOH) if all of the following conditions are fulfilled: (i) the ring moiety

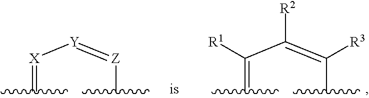

(ii) R² is hydrogen, (iii) R³ is —SO₂—NH₂, (iv) R⁴ is —O-phenyl, (v) R⁵ is —NH—CH₂CH₂CH₂CH₃, and (vi) R⁶ is hydrogen.

The compound of formula (I) may be, for example, any one of the specific compounds described in the examples section of this specification, either in non-salt form (e.g., free base/acid form) or as a pharmaceutically acceptable salt or solvate of the respective compound.

In particular, the compound of formula (I) may be a compound of any one of the following formulae, or a pharmaceutically acceptable salt or solvate thereof:

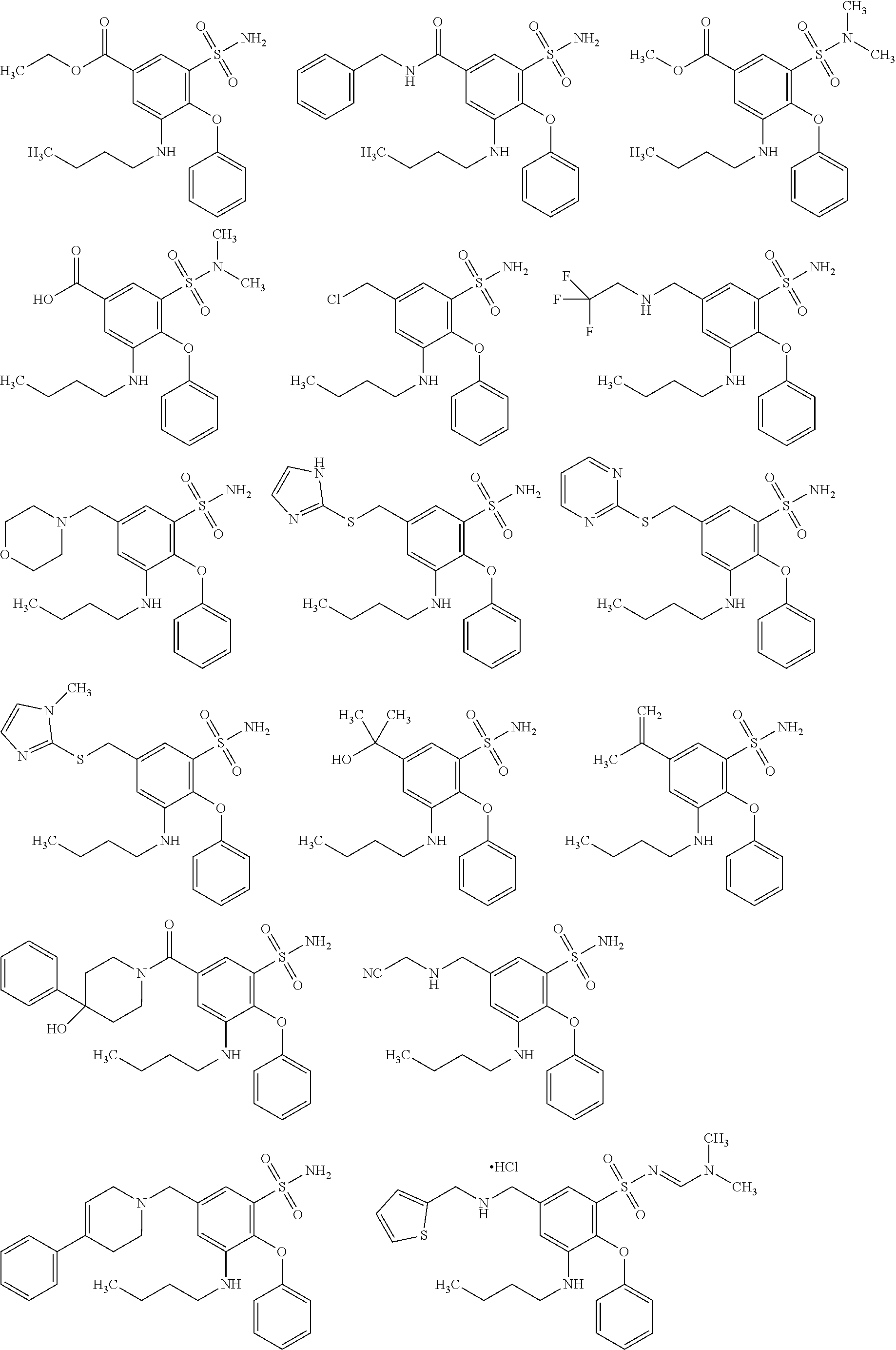

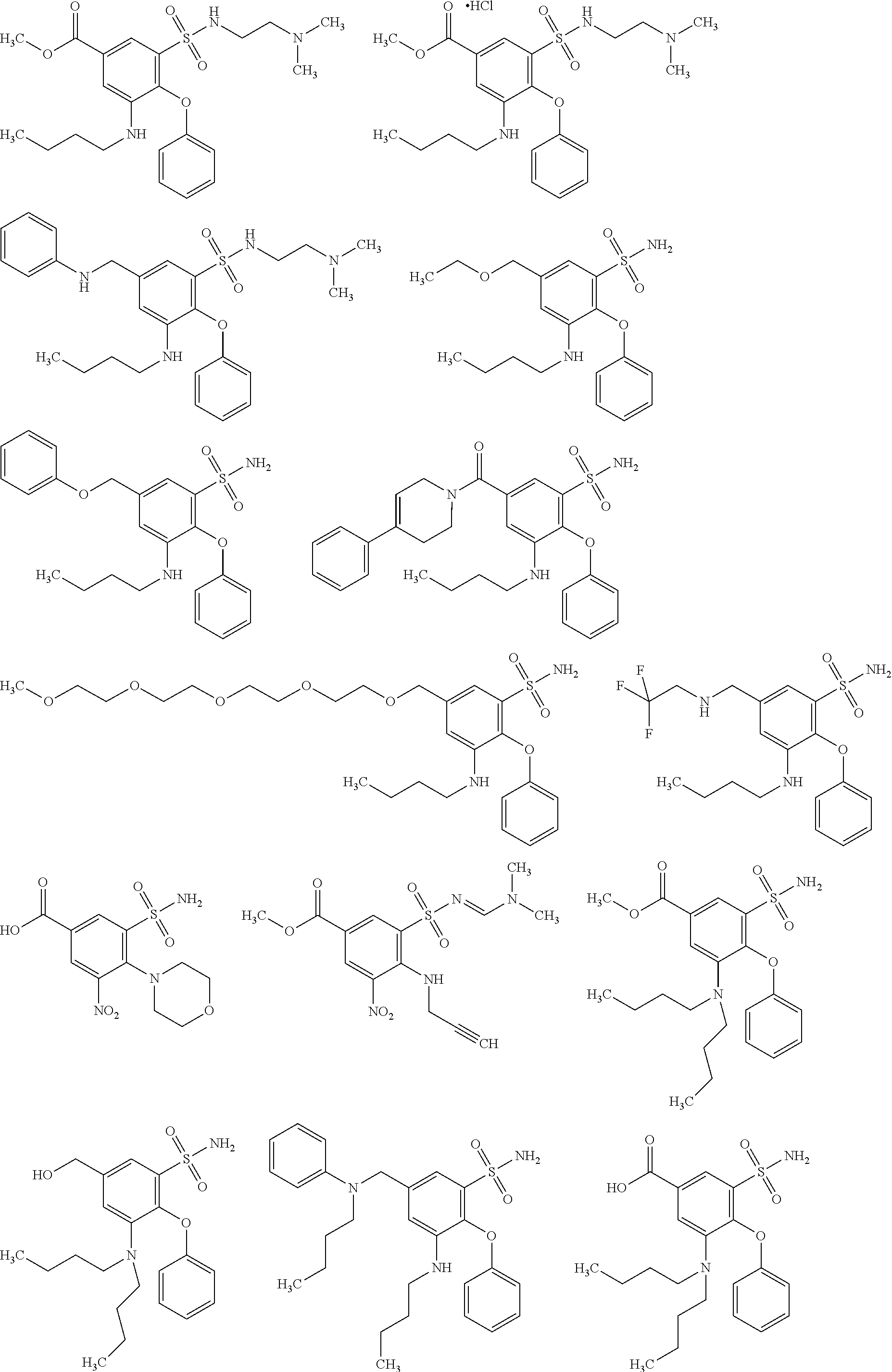

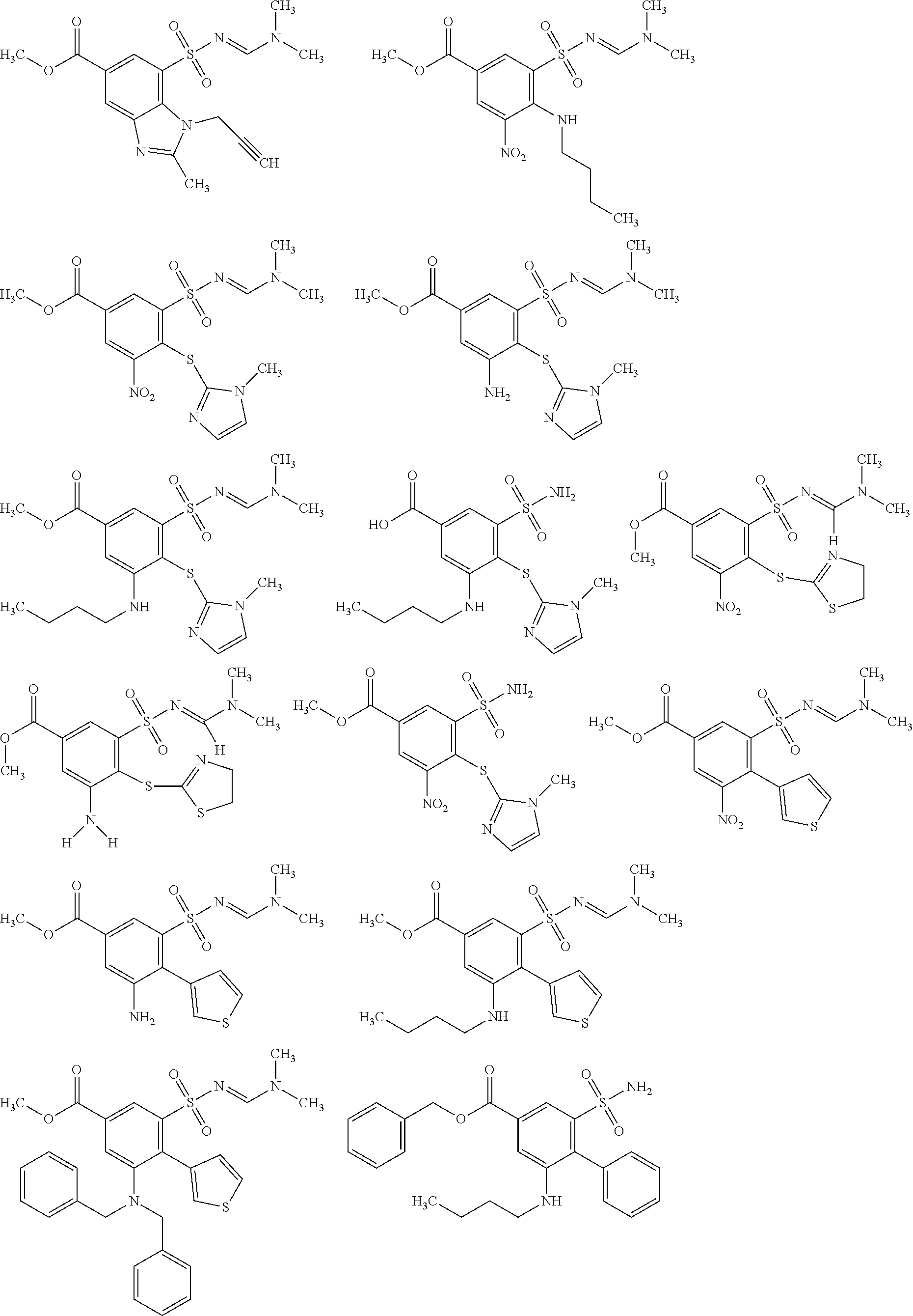

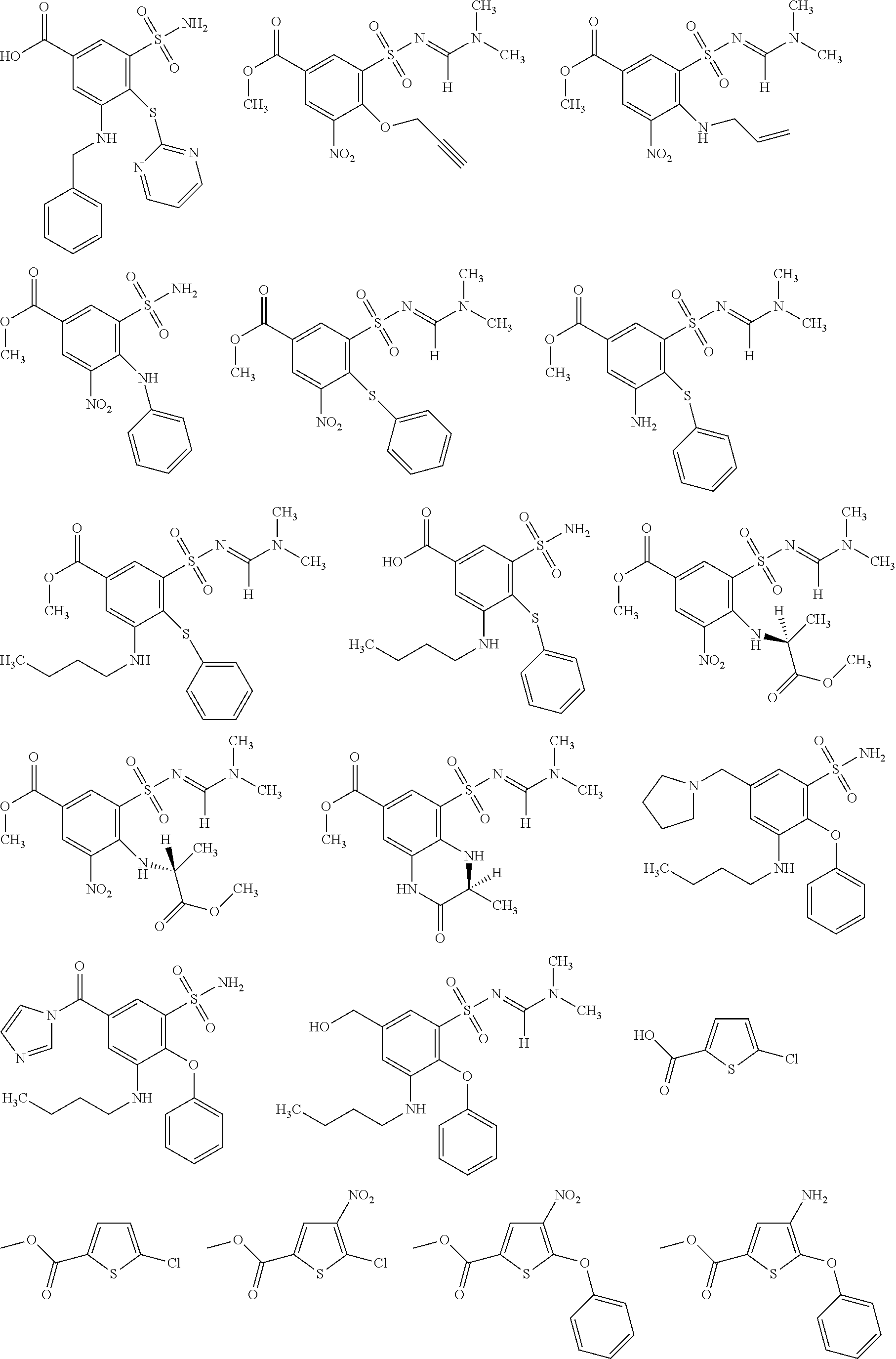

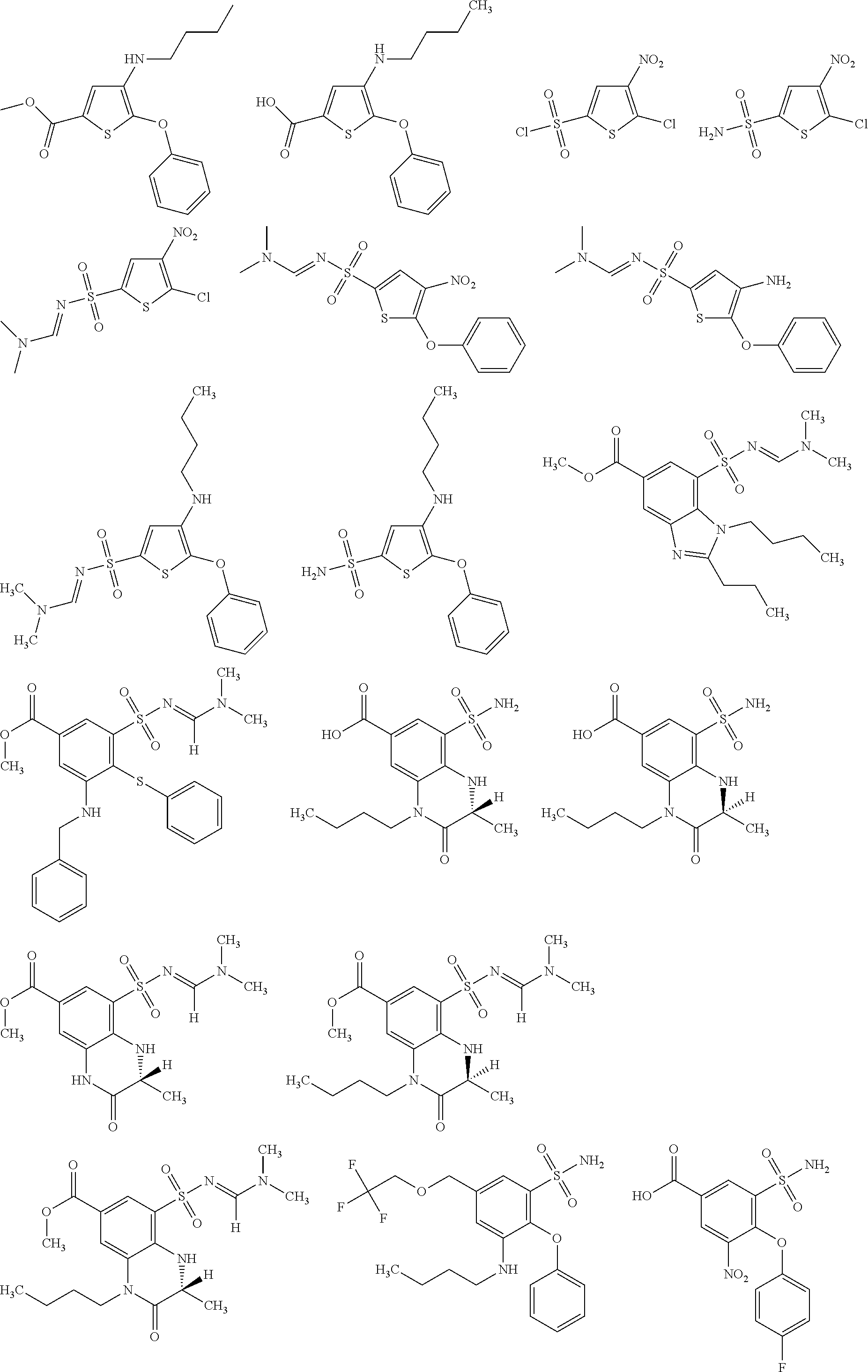

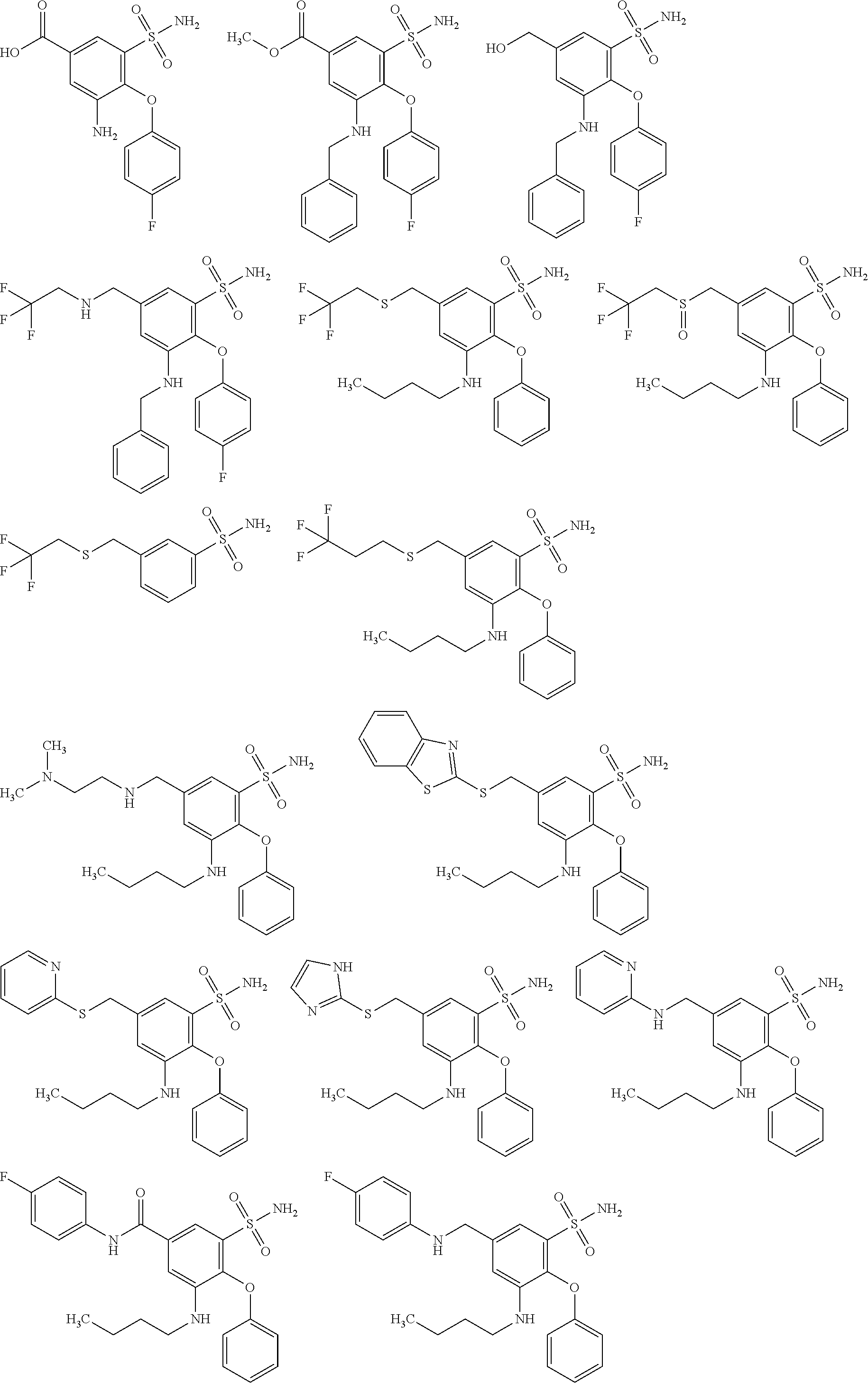

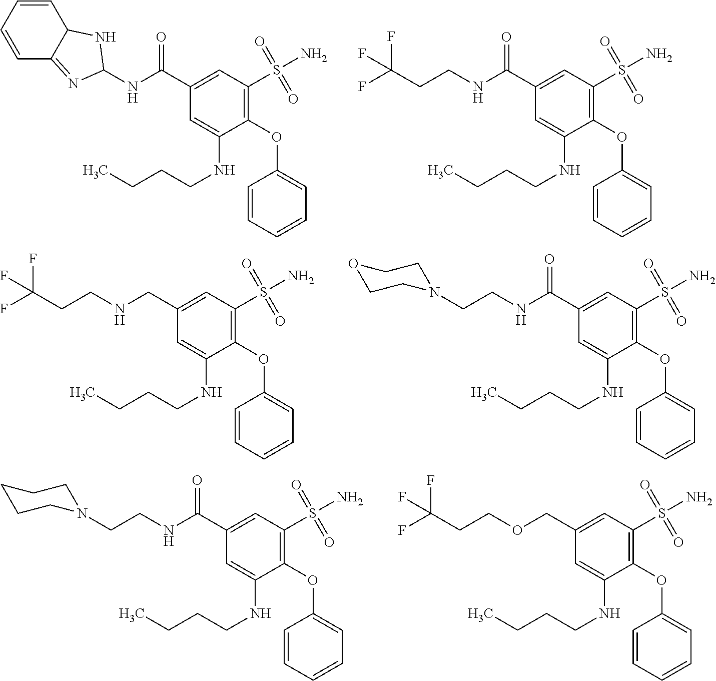

Error! Objects Cannot be Created from Editing Field Codes.

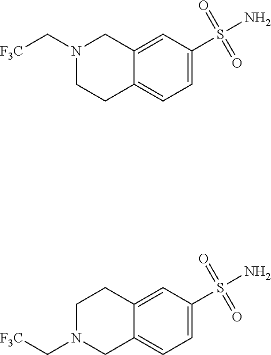 [SMILES: NS(=O)(=O)c1ccc2c(c1)CN(CC(F)(F)F)CC2 ; NS(=O)(=O)c1ccc2c(c1)CCN(CC(F)(F)F)C2]

In a first embodiment, the ring moiety

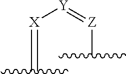

in the compound of formula (I) is

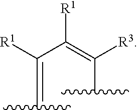

Accordingly, in this first embodiment, the compound of formula (I) is a compound of the following formula (Ia) or a pharmaceutically acceptable salt or solvate thereof:

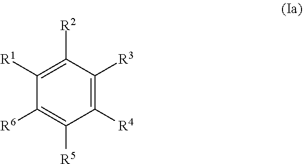

wherein the groups and variables in formula (Ia), including in particular R¹, R², R³, R⁴, R⁵ and R⁶, have the same meanings, including the same preferred meanings, as described and defined herein above for the compound of formula (I).

In a second embodiment, the ring moiety

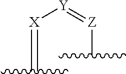

in the compound of formula (I) is

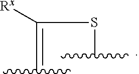

Accordingly, in this second embodiment, the compound of formula (I) is a compound of the following formula (Ib) or a pharmaceutically acceptable salt or solvate thereof:

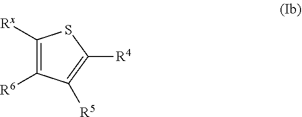

wherein the groups and variables in formula (Ib), including in particular R^{x}, R⁴, R⁵ and R⁶, have the same meanings, including the same preferred meanings, as described and defined herein above for the compound of formula (I).

In a third embodiment, the compound of formula (I) is a compound of the following formula (Ia) or a pharmaceutically acceptable salt or solvate thereof

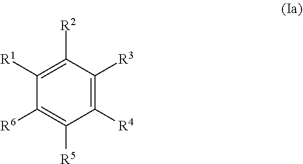

wherein R⁴ is a group R^{4a}, wherein R⁵ is a group R^{5a}, and wherein the further groups and variables in formula (Ia), including in particular R¹, R², R³ and R⁶, have the same meanings, including the same preferred meanings, as described and defined herein above for the compound of formula (I).

In this third embodiment, R^{4a} is selected from —O—R⁴¹, —S—R⁴¹, —NH—R⁴¹, —N(C₁₋₆ alkyl)-R⁴¹, halogen (e.g., —Cl), carbocyclyl and heterocyclyl, wherein said carbocyclyl and said heterocyclyl are each optionally substituted with one or more (e.g., one, two or three) groups R⁴². Preferably, R^{4a} is selected from —O—R⁴¹, —S—R⁴¹, —NH—R⁴¹, —N(C₁₋₆ alkyl)-R⁴¹, carbocyclyl (e.g., aryl, cycloalkyl, or cycloalkenyl) and heterocyclyl (e.g., heteroaryl, heterocycloalkyl, or heterocycloalkenyl), wherein said carbocyclyl and said heterocyclyl are each optionally substituted with one or more groups R⁴². More preferably, R^{4a} is selected from —O—R⁴¹, —S—R⁴¹, —NH—R⁴¹, —N(C₁₋₄ alkyl)-R⁴¹, aryl and heteroaryl, wherein said aryl and said heteroaryl are each optionally substituted with one or more groups R⁴²

R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₀₋₄ alkylene)-carbocyclyl and the heterocyclyl moiety of said —(C₀₋₄ alkylene)-heterocyclyl are each optionally substituted with one or more (e.g., one, two or three) groups R⁴², and wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more (e.g., one, two or three) groups R⁴³. Preferably, R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₀₋₄ alkylene)-carbocyclyl is selected from cycloalkyl, cycloalkenyl and aryl, wherein the heterocyclyl moiety of said —(C₀₋₄ alkylene)-heterocyclyl is selected from heterocycloalkyl, heterocycloalkenyl and heteroaryl, wherein the carbocyclyl moiety of said —(C₀₋₄ alkylene)-carbocyclyl and the heterocyclyl moiety of said —(C₀₋₄ alkylene)-heterocyclyl are each optionally substituted with one or more (e.g., one, two or three) groups R⁴², and further wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more (e.g., one, two or three) groups R⁴³. More preferably, R⁴¹ is selected from —(C₀₋₄ alkylene)-aryl and —(C₀₋₄ alkylene)-heteroaryl, wherein the aryl moiety of said —(C₀₋₄ alkylene)-aryl and the heteroaryl moiety of said —(C₀₋₄ alkylene)-heteroaryl are each optionally substituted with one or more (e.g., one, two or three) groups R⁴², and further wherein the alkylene moiety of said —(C₀₋₄ alkylene)-aryl and the alkylene moiety of said —(C₀₋₄ alkylene)-heteroaryl are each optionally substituted with one or more (e.g., one, two or three) groups R⁴³. Even more preferably, R⁴¹ is selected from —(C₀₋₄ alkylene)-aryl and —(C₀₋₄ alkylene)-heteroaryl, wherein the aryl moiety of said —(C₀₋₄ alkylene)-aryl and the heteroaryl moiety of said —(C₀₋₄ alkylene)-heteroaryl are each optionally substituted with one or more (e.g., one, two or three) groups R⁴². A preferred example of the aryl moiety of said —(C₀₋₄ alkylene)-aryl is phenyl. A preferred example of the heteroaryl moiety of said —(C₀₋₄ alkylene)-heteroaryl is a 5- or 6-membered monocyclic heteroaryl having 1 or 2 ring heteroatoms independently selected from oxygen, nitrogen and sulfur (wherein the remaining ring atoms are carbon atoms), such as, e.g., imidazolyl, thiophenyl, or pyrimidinyl. Still more preferably, R⁴¹ is selected from phenyl and heteroaryl, wherein said heteroaryl is a 5- or 6-membered monocyclic heteroaryl having 1 or 2 ring heteroatoms independently selected from oxygen, nitrogen and sulfur (the remaining ring atoms of the monocyclic heteroaryl are carbon atoms), and further wherein said phenyl or said heteroaryl is optionally substituted with one or more (e.g., one, two or three) groups R⁴²

Each R⁴² is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, each R⁴² is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

Each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃, —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl) and —N(C₁₋₆ alkyl)-CO—(C₁₋₆ alkyl). Preferably, each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃ and —CN.

In accordance with the above definitions, it is particularly preferred that R⁴ is selected from —O—(C₀₋₄ alkylene)-aryl, —O—(C₀₋₄ alkylene)-heteroaryl, —S—(C₀₋₄ alkylene)-aryl, —S—(C₀₋₄ alkylene)-heteroaryl, —NH—(C₀₋₄ alkylene)-aryl, —NH—(C₀₋₄ alkylene)-heteroaryl, —N(C₁₋₄ alkyl)-(C₀₋₄ alkylene)-aryl, —N(C₁₋₄ alkyl)-(C₀₋₄ alkylene)-heteroaryl, aryl and heteroaryl, wherein the aryl moiety of any of the aforementioned groups, the heteroaryl moiety of any of the aforementioned groups, said aryl and said heteroaryl are each optionally substituted with one or more (e.g., one, two or three) groups R⁴². Even more preferably, R^{4a} is selected from —O-aryl, —O-heteroaryl, —S-aryl, —S-heteroaryl, —NH-aryl, —NH-heteroaryl, —N(C₁₋₄ alkyl)-aryl, —N(C₁₋₄ alkyl)-heteroaryl, aryl and heteroaryl, wherein the aryl moiety of any of the aforementioned groups, the heteroaryl moiety of any of the aforementioned groups, said aryl and said heteroaryl are each optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN. Yet even more preferably, R^{4a} is selected from —O-phenyl, —O— heteroaryl, —S-phenyl, —S-heteroaryl, —NH-phenyl, —NH-heteroaryl, —N(C₁₋₄ alkyl)-phenyl, —N(C₁₋₄ alkyl)-heteroaryl, phenyl and heteroaryl, wherein said heteroaryl or the heteroaryl moiety of any of the aforementioned groups is a 5- or 6-membered monocyclic heteroaryl having 1 or 2 ring heteroatoms independently selected from oxygen, nitrogen and sulfur (the remaining ring atoms of the monocyclic heteroaryl are carbon atoms), and further wherein the phenyl moiety of any of the aforementioned groups, the heteroaryl moiety of any of the aforementioned groups, said phenyl and said heteroaryl are each optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN. A particularly preferred example of R^{4a} is —O-phenyl.

In this third embodiment, R^{5a} is selected from —NH₂, —NH(C₁₋₆ alkyl), —N(C₁₋₆ alkyl)(C₁₋₆ alkyl), —NO₂ and hydrogen, wherein the alkyl moiety of said —NH(C₁₋₆ alkyl) and one or both of the alkyl moieties of said —N(C₁₋₆ alkyl)(C₁₋₆ alkyl) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH, —S(C₁₋₆ alkyl), carbocyclyl and heterocyclyl, wherein said carbocyclyl and said heterocyclyl are each optionally substituted with one or more (e.g., one, two or three) groups R⁵¹. Preferably, R^{5a} is selected from —NH₂, —NH(C₁₋₆ alkyl), —N(C₁₋₆ alkyl)(C₁₋₆ alkyl) and —NO₂, wherein the alkyl moiety of said —NH(C₁₋₆ alkyl) and one or both of the alkyl moieties of said —N(C₁₋₆ alkyl)(C₁₋₆ alkyl) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl). More preferably, R^{5a} is selected from —NH₂, —NH(C₁₋₆ alkyl), —N(C₁₋₆ alkyl)(C₁₋₆ alkyl), and —NO₂. Particularly preferred examples of R^{5a} are —NH₂, —NH—CH₂CH₂CH₂CH₃, or —NO₂.

Each R⁵¹ is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, each R⁵¹ is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

In a fourth embodiment, the compound of formula (I) is a compound of the following formula (Ia) or a pharmaceutically acceptable salt or solvate thereof

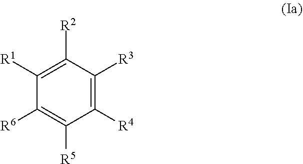

wherein R⁴ and R⁵ are mutually linked to form a group —R^{5b}—, and wherein the further groups and variables in formula (Ia), including in particular R¹, R², R³ and R⁶, have the same meanings, including the same preferred meanings, as described and defined herein above for the compound of formula (I).

In this fourth embodiment, R⁴ and R⁵ are mutually linked to form a group —R^{5b}—. The group R^{5b} is selected from —R^{5b1}—R^{5b2}—R^{5b1}, —N═C(R⁵³)—R^{5b3}—R^{5b1}—, —R^{5b1}—R^{5b3}—C(R⁵³)═N—, and —N═C(R⁵³)—R^{5b4}—C(R⁵³)═N—.

Each R^{5b1} is independently selected from —N(R⁵²)—, —O— and —S—. R^{5b2} is selected from —C(R⁵³)(R⁵³)—, —C(R⁵³)(R⁵³)—C(R⁵³)(R⁵³)—, —C(R⁵³)═C(R⁵³)—, —C(R⁵³)(R⁵³)—C(R⁵³)═C(R⁵³)— and —C(R⁵³)═C(R⁵³)—C(R⁵³)(R⁵³)—. R^{5b3} is selected from a covalent bond, —C(R⁵³)(R⁵³)—, —C(R⁵³)(R⁵³)—C(R⁵³)(R⁵³)— and —C(R⁵³)═C(R⁵³)—. R^{5b4} is selected from a covalent bond and —C(R⁵³)(R⁵³)—.

Each R⁵² is independently selected from hydrogen, C₁₋₆ alkyl, C₂₋₆ alkenyl, C₂₋₆ alkynyl, —(C₀₋₄ alkylene)-aryl and —(C₀₋₄ alkylene)-heteroaryl. Preferably, each R⁵² is independently selected from hydrogen, C₁₋₄ alkyl (e.g., n-butyl), C₂₋₄ alkenyl and C₂₋₄ alkynyl (e.g., —CH₂—C═CH). Even more preferably, each R⁵² is hydrogen.

Each R⁵³ is independently selected from hydrogen, C₁₋₆ alkyl, C₂₋₆ alkenyl, C₂₋₆ alkynyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl), —N(C₁₋₆ alkyl)-SO₂—(C₁₋₆ alkyl), —(C₀₋₄ alkylene)-aryl and —(C₀₋₄ alkylene)-heteroaryl; moreover, any two groups R⁵³ that are attached to the same carbon atom may also together form a group ═O; and any two groups R⁵³ that are attached to adjacent carbon atoms connected by a double bond (i.e., any two groups R⁵³ comprised in a moiety —C(R⁵³)═C(R⁵³)—) may also be mutually linked to form a group —C(R⁵⁴)═C(R⁵⁴)—C(R⁵⁴)═C(R⁵⁴)—. Preferably, each R⁵³ is independently selected from hydrogen, C₁₋₆ alkyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), and —CN, and any two groups R⁵³ that are attached to the same carbon atom may also together form a group ═O, and any two groups R⁵³ that are attached to adjacent carbon atoms connected by a double bond may also be mutually linked to form a group —C(R⁵⁴)═C(R⁵⁴)—C(R⁵⁴)═C(R⁵⁴)—. More preferably, each R⁵³ is independently selected from hydrogen, C₁₋₆ alkyl, —OH, —O(C₁₋₆ alkyl), —SH, —S(C₁₋₆ alkyl), —NH₂, —NH(C₁₋₆alkyl), —N(C₁₋₆ alkyl)(C₁₋₆ alkyl), halogen, C₁₋₆ haloalkyl, —O—(C₁₋₆ haloalkyl), and —CN, and any two groups R⁵³ that are attached to the same carbon atom may also together form a group ═O. Even more preferably, each R⁵³ is independently selected from hydrogen, C₁₋₄ alkyl, —OH, —O(C₁₋₄ alkyl), —NH₂, —NH(C₁₋₄ alkyl), —N(C₁₋₄ alkyl)(C₁₋₄ alkyl), halogen, C₁₋₄ haloalkyl, —O—(C₁₋₄ haloalkyl), and —CN, and any two groups R⁵³ that are attached to the same carbon atom may also together form a group ═O.

Each R⁵⁴ is independently selected from hydrogen, C₁₋₆ alkyl, C₂₋₆ alkenyl, C₂₋₆ alkynyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, each R⁵⁴ is independently selected from hydrogen, C₁₋₆ alkyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN. More preferably, each R⁵⁴ is hydrogen.

It is particularly preferred that R^{5b} is selected from —N(R⁵²)—C(R⁵³)(R⁵³)—N(R⁵²)—, —N═C(R⁵³)—N(R⁵²)—, —N(R⁵²)—C(R⁵³)═N—, —N(R⁵²)—C(R⁵³)(R⁵³)—C(R⁵³)(R⁵³)—N(R⁵²)—, —N═C(R⁵³)—C(R⁵³)(R⁵³)—N(R⁵²)—, —N(R⁵²)—C(R⁵³)═C(R⁵³)—N(R⁵²)—, —N(R⁵²)—C(R⁵³)(R⁵³)—C(R⁵³)═N—, —N═C(R⁵³)—C(R⁵³)═N—, —N(R⁵²)—C(R⁵³)(R⁵³)—C(R⁵³)(R⁵³)—C(R⁵³)(R⁵³)—N(R⁵²)—, —N═C(R⁵³)—C(R⁵³)(R⁵³)—C(R⁵³)(R⁵³)—N(R⁵²)—, —N(R⁵²)—C(R⁵³)(R⁵³)—C(R⁵³)═C(R⁵³)—N(R⁵²)—, —N(R⁵²)—C(R⁵³)═C(R⁵³)—C(R⁵³)(R⁵³)—N(R⁵²)—, —N(R⁵²)—C(R⁵³)(R⁵³)—C(R⁵³)(R⁵³)—C(R⁵³)═N—, —N═C(R⁵³)—C(R⁵³)(R⁵³)—C(R⁵³)═N—, —O—C(R⁵³)(R⁵³)—N(R⁵²)—, —O—C(R⁵³)(R⁵³)—C(R⁵³)(R⁵³)—N(R⁵²)—, —O—C(R⁵³)═C(R⁵³)—N(R⁵²)—, —O—C(R⁵³)(R⁵³)—C(R³)(R⁵³)—C(R⁵³)(R⁵³)—N(R⁵²)—, —O—C(R⁵³)(R⁵³)—C(R⁵³)═C(R⁵³)—N(R⁵²)—, —O—C(R⁵³)═C(R⁵³)—C(R⁵³)(R⁵³)—N(R⁵²)—, —N(R⁵²)—C(R⁵³)(R⁵³)—O—, —N(R⁵²)—C(R³)(R³)—C(R³)(R⁵³)—O—, —N(R⁵²)—C(R⁵³)═C(R⁵³)—O—, —N(R⁵²)—C(R⁵³)(R⁵³)—C(R⁵³)(R⁵³)—C(R⁵³)(R⁵³)—O—, —N(R⁵²)—C(R⁵³)(R⁵³)—C(R⁵³)═C(R⁵³)—O—, —N(R⁵²)—C(R⁵³)═C(R⁵³)—C(R⁵³)(R⁵³)—O—, —S—C(R⁵³)(R⁵³)—N(R⁵²)—, —S—C(R⁵³)(R⁵³)—C(R⁵³)(R⁵³)—N(R⁵²)—, —S—C(R⁵³)═C(R⁵³)—N(R⁵²)—, —S—C(R⁵³)(R⁵³)—C(R⁵³)(R⁵³)—C(R⁵³)(R⁵³)—N(R⁵²)—, —S—C(R⁵³)(R⁵³)—C(R⁵³)═C(R⁵³)—N(R⁵²)—, —S—C(R⁵³)═C(R⁵³)—C(R⁵³)(R⁵³)—N(R⁵²)—, —N(R⁵²)—C(R⁵³)(R⁵³)—S—, —N(R⁵²) C(R⁵³)(R⁵³)—C(R⁵³)(R⁵³)—S—, —N(R⁵²)—C(R⁵³)═C(R⁵³)—S—, —N(R⁵²)—C(R⁵³)(R⁵³)—C(R⁵³)(R⁵³)—C(R⁵³)(R⁵³)—S—, —N(R⁵²)—C(R⁵³)(R⁵³)—C(R⁵³)═C(R⁵³)—S—, —N(R⁵²)—C(R⁵³)═C(R⁵³)—C(R⁵³)(R⁵³)—S—, —N(R⁵²)—C(R⁵³)(R⁵³)—N(R⁵²)—, —N(R⁵²)—C(R⁵³)(R⁵³)—C(R⁵³)(R⁵³)—N(R⁵²)—, —N(R⁵²)—C(R⁵³)═C(R⁵³)—N(R⁵²)—, —N(R⁵²)—C(R³)(R⁵³)—C(R⁵³)(R⁵³)—C(R⁵³)(R⁵³)—N(R⁵²)—, —N(R⁵²)—C(R⁵³)(R⁵³)—C(R⁵³)═C(R⁵³)—N(R⁵²)— and —N(R⁵²)—C(R⁵³)═C(R⁵³)—C(R⁵³)(R⁵³)—N(R⁵²)—.

It is even more preferred in this fourth embodiment that R^{5b} is selected such that the compound of formula (Ia) has any one of the following structures:

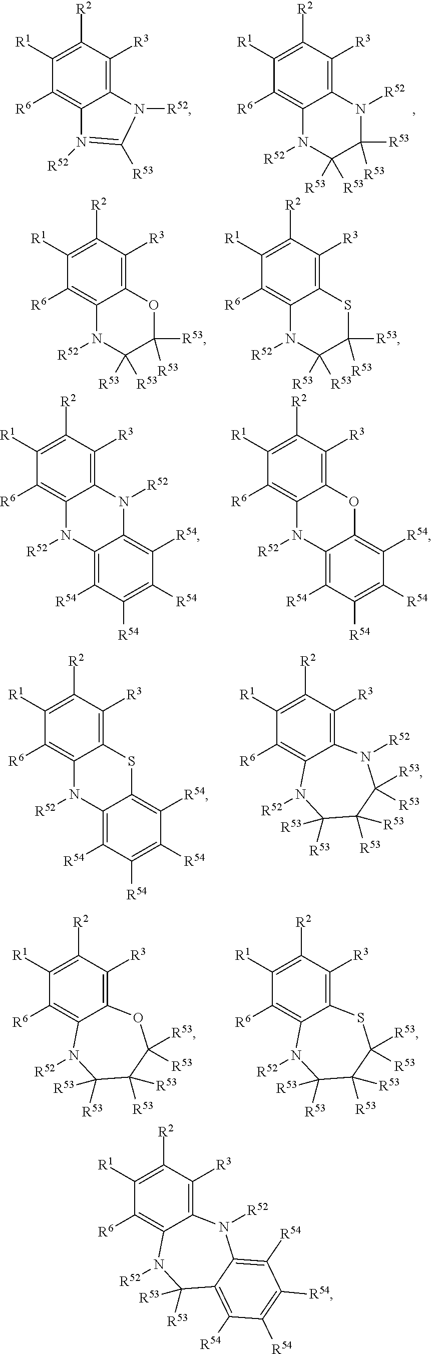

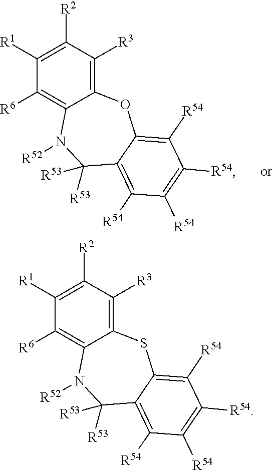

In a fifth embodiment, the compound of formula (I) is a compound of the following formula (Ia) or a pharmaceutically acceptable salt or solvate thereof

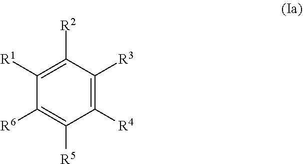

wherein R⁴ is a group R^{4a}, wherein R^{4a} is —O-phenyl, wherein R⁵ is a group R^{5a}, and wherein the further groups and variables in formula (Ia), including in particular R¹, R², R³, R^{5a} and R⁶, have the same meanings, including the same preferred meanings, as described and defined herein above for the compound of formula (I).

In a sixth embodiment, the compound of formula (I) is a compound of the following formula (Ia) or a pharmaceutically acceptable salt or solvate thereof

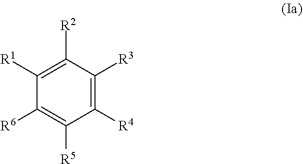

wherein R⁴ is a group R^{4a}, wherein R⁵ is a group R^{5a}, wherein R^{5a} is —NH₂, and wherein the further groups and variables in formula (Ia), including in particular R¹, R², R³, R^{4a} and R⁶, have the same meanings, including the same preferred meanings, as described and defined herein above for the compound of formula (I).

In a seventh embodiment, the compound of formula (I) is a compound of the following formula (Ia) or a pharmaceutically acceptable salt or solvate thereof

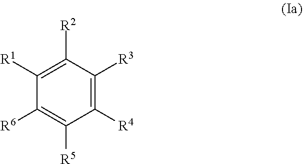

wherein R⁴ is a group R^{4a}, wherein R⁵ is a group R^{5a}, wherein R^{5a} is —NH—CH₂CH₂CH₂CH₃, and wherein the further groups and variables in formula (Ia), including in particular R¹, R², R³, R⁴ and R⁶, have the same meanings, including the same preferred meanings, as described and defined herein above for the compound of formula (I).

In an eighth embodiment, the compound of formula (I) is a compound of the following formula (Ia) or a pharmaceutically acceptable salt or solvate thereof

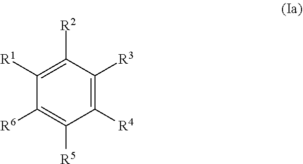

wherein R⁴ is a group R^{4a}, wherein R⁵ is a group R^{5a}, wherein R^{5a} is —NO₂, and wherein the further groups and variables in formula (Ia), including in particular R¹, R², R³, R^{4a} and R⁶, have the same meanings, including the same preferred meanings, as described and defined herein above for the compound of formula (I).

In a ninth embodiment, the compound of formula (I) is a compound of the following formula (Ia) or a pharmaceutically acceptable salt or solvate thereof

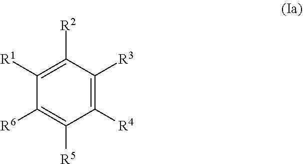

wherein R³ is —SO₂—NH₂, and wherein the further groups and variables in formula (Ia), including in particular R¹, R², R⁴, R⁵ and R⁶, have the same meanings, including the same preferred meanings, as described and defined herein above for the compound of formula (I).

In a tenth embodiment, the compound of formula (I) is a compound of the following formula (Ia) or a pharmaceutically acceptable salt or solvate thereof

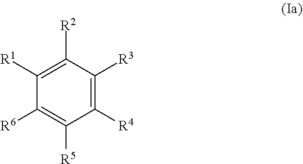

wherein R³ is —SO₂—N═CH—N(CH₃)₂, and wherein the further groups and variables in formula (Ia), including in particular R¹, R², R⁴, R⁵ and R⁶, have the same meanings, including the same preferred meanings, as described and defined herein above for the compound of formula (I).

In an eleventh embodiment, the compound of formula (I) is a compound of the following formula (Ia) or a pharmaceutically acceptable salt or solvate thereof

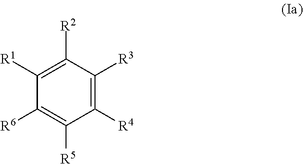

wherein R³ is —SO₂—NH₂, wherein R⁴ is a group R^{4a}, wherein R^{4a} is —O-phenyl, wherein R⁵ is a group R^{5a}, and wherein the further groups and variables in formula (Ia), including in particular R¹, R², R⁵ and R⁶, have the same meanings, including the same preferred meanings, as described and defined herein above for the compound of formula (I).

In a twelfth embodiment, the compound of formula (I) is a compound of the following formula (Ia) or a pharmaceutically acceptable salt or solvate thereof

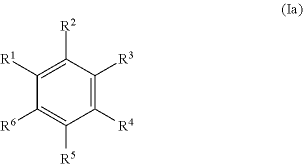

wherein R³ is —SO₂—NH₂, wherein R⁴ is a group R^{4a}, wherein R⁵ is a group R^{5a}, wherein R^{5a} is —NH₂, and wherein the further groups and variables in formula (Ia), including in particular R¹, R², R⁴ and R⁶, have the same meanings, including the same preferred meanings, as described and defined herein above for the compound of formula (I).

In a thirteenth embodiment, the compound of formula (I) is a compound of the following formula (Ia) or a pharmaceutically acceptable salt or solvate thereof

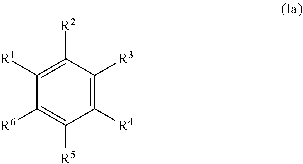

wherein R³ is —SO₂—NH₂, wherein R⁴ is a group R^{4a}, wherein R⁵ is a group R^{5a}, wherein R^{5a} is —NH—CH₂CH₂CH₂CH₃, and wherein the further groups and variables in formula (Ia), including in particular R¹, R², R⁴ and R⁶, have the same meanings, including the same preferred meanings, as described and defined herein above for the compound of formula (I).

In a fourteenth embodiment, the compound of formula (I) is a compound of the following formula (Ia) or a pharmaceutically acceptable salt or solvate thereof

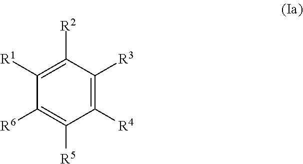

wherein R³ is —SO₂—NH₂, wherein R⁴ is a group R^{4a}, wherein R⁵ is a group R^{5a}, wherein R^{5a} is —NO₂, and wherein the further groups and variables in formula (Ia), including in particular R¹, R², R⁴ and R⁶, have the same meanings, including the same preferred meanings, as described and defined herein above for the compound of formula (I).

In a fifteenth embodiment, the compound of formula (I) is a compound of the following formula (Ia) or a pharmaceutically acceptable salt or solvate thereof

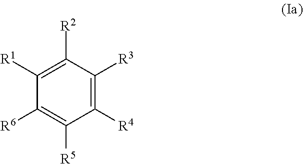

wherein R³ is —SO₂—N═CH—N(CH₃)₂, wherein R⁴ is a group R^{4a}, wherein R^{4a} is —O-phenyl, wherein R⁵ is a group R^{5a}, and wherein the further groups and variables in formula (Ia), including in particular R¹, R², R^{5a} and R⁶, have the same meanings, including the same preferred meanings, as described and defined herein above for the compound of formula (I).

In a sixteenth embodiment, the compound of formula (I) is a compound of the following formula (Ia) or a pharmaceutically acceptable salt or solvate thereof

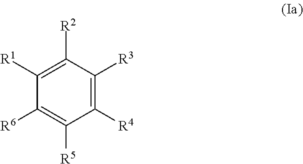

wherein R³ is —SO₂—N═CH—N(CH₃)₂, wherein R⁴ is a group R^{4a}, wherein R⁵ is a group R^{5a}, wherein R^{5a} is —NH₂, and wherein the further groups and variables in formula (Ia), including in particular R¹, R², R^{4a} and R⁶, have the same meanings, including the same preferred meanings, as described and defined herein above for the compound of formula (I).

In a seventeenth embodiment, the compound of formula (I) is a compound of the following formula (Ia) or a pharmaceutically acceptable salt or solvate thereof

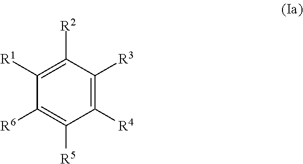

wherein R³ is —SO₂—N═CH—N(CH₃)₂, wherein R⁴ is a group R^{4a}, wherein R⁵ is a group R^{5a}, wherein R^{5a} is —NH—CH₂CH₂CH₂CH₃, and wherein the further groups and variables in formula (Ia), including in particular R¹, R², R^{4a} and R⁶, have the same meanings, including the same preferred meanings, as described and defined herein above for the compound of formula (I).

In an eighteenth embodiment, the compound of formula (I) is a compound of the following formula (Ia) or a pharmaceutically acceptable salt or solvate thereof

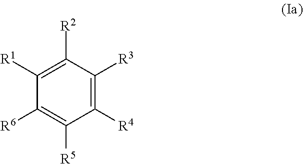

wherein R³ is —SO₂—N═CH—N(CH₃)₂, wherein R⁴ is a group R^{4a}, wherein R⁵ is a group R^{5a}, wherein R^{5a} is —NO₂, and wherein the further groups and variables in formula (Ia), including in particular R¹, R², R^{4a} and R⁶, have the same meanings, including the same preferred meanings, as described and defined herein above for the compound of formula (I).

In a nineteenth embodiment, the compound of formula (I) is a compound of the following formula (Ia) or a pharmaceutically acceptable salt or solvate thereof

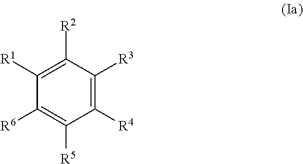

wherein R⁴ is —O-phenyl, wherein R⁵ is —NH₂, and wherein the further groups and variables in formula (Ia), including in particular R¹, R², R³ and R⁶, have the same meanings, including the same preferred meanings, as described and defined herein above for the compound of formula (I).

In a twentieth embodiment, the compound of formula (I) is a compound of the following formula (Ia) or a pharmaceutically acceptable salt or solvate thereof

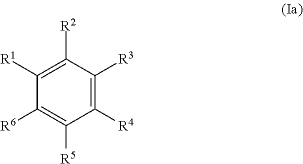

wherein R⁴ is —O-phenyl, wherein R⁵ is —NH—CH₂CH₂CH₂CH₃, and wherein the further groups and variables in formula (Ia), including in particular R¹, R², R³ and R⁶, have the same meanings, including the same preferred meanings, as described and defined herein above for the compound of formula (I).

In a twenty-first embodiment, the compound of formula (I) is a compound of the following formula (Ia) or a pharmaceutically acceptable salt or solvate thereof

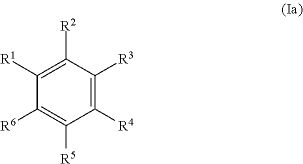

wherein R⁴ is —O-phenyl, wherein R⁵ is —NO₂, and wherein the further groups and variables in formula (Ia), including in particular R¹, R², R³ and R⁶, have the same meanings, including the same preferred meanings, as described and defined herein above for the compound of formula (I).

In a twenty-second embodiment, the compound of formula (I) is a compound of the following formula (Ia) or a pharmaceutically acceptable salt or solvate thereof

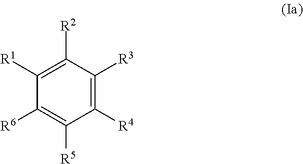

wherein R¹ is as defined below, and wherein the further groups and variables in formula (Ia), including in particular R², R³, R⁴, R⁵ and R⁶, have the same meanings, including the same preferred meanings, as described and defined herein above for the compound of formula (I).

In this twenty-second embodiment, R¹ is —COO—(C₁₋₁₅ alkyl), wherein the alkyl moiety of said —COO—(C₁₋₁₅ alkyl) is optionally substituted with one or more (e.g., one, two or three) groups independently selected from halogen, —CF₃, —CN, —NO₂, —NH₂, —NH(C₁₋₄ alkyl), —N(C₁₋₄ alkyl)(C₁₋₄ alkyl), —OH, —O(C₁₋₄ alkyl), —SH and —S(C₁₋₄ alkyl), and further wherein one or two —CH₂— units comprised in the alkyl moiety of said —COO—(C₁₋₁₅ alkyl) are each optionally replaced by a group independently selected from —O—, —CO—, —COO—, —O—CO—, —NH—, —N(C₁₋₄ alkyl)-, —NH—CO—, —N(C₁₋₄ alkyl)-CO—, —CO—NH—, —CO—N(C₁₋₄ alkyl)-, —S—, —SO—, —SO₂—, —SO₂—NH—, —SO₂—N(C₁₋₄ alkyl)-, —NH—SO₂— and —N(C4 alkyl)-SO₂—. The alkyl moiety of said —COO—(C₁₋₁₅ alkyl) has 1 to 15 carbon atoms, particularly 1 to 10 carbon atoms. Preferred examples of such R¹ groups include the corresponding groups R¹ of the compounds described in the examples section, including in particular —COO—CH₃ or —COO—CH₂CH₃. A particularly preferred group R¹ is —COO—CH₃.

In a twenty-third embodiment, the compound of formula (I) is a compound of the following formula (Ia) or a pharmaceutically acceptable salt or solvate thereof

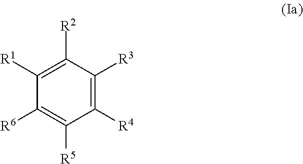

wherein R¹ is —COOH, and wherein the further groups and variables in formula (Ia), including in particular R², R³, R⁴, R⁵ and R⁶, have the same meanings, including the same preferred meanings, as described and defined herein above for the compound of formula (I).

In a twenty-fourth embodiment, the compound of formula (I) is a compound of the following formula (Ia) or a pharmaceutically acceptable salt or solvate thereof

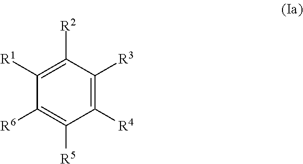

wherein R¹ is as defined below, and wherein the further groups and variables in formula (Ia), including in particular R², R³, R⁴, R⁵ and R⁶, have the same meanings, including the same preferred meanings, as described and defined herein above for the compound of formula (I).

In this twenty-fourth embodiment, R¹ is selected from —(C₁₋₄ alkylene)-NH—(C₁₋₄ alkylene)-R¹² (e.g., —CH₂—NH—CH₂—R¹²), —COO—(C₁₋₄ alkylene)-R¹² (e.g., —COO—CH₂—R¹²), —O—CO—(C₁₋₄ alkylene)-R¹², —CO—(C₁₋₄ alkylene)-R¹², —CO—NH—(C₁₋₄ alkylene)-R¹² (e.g., —CO—NH—CH₂—R¹²), —CO—N(C₁₋₄ alkyl)-(C₁₋₄ alkylene)-R¹², —NH—CO—(C₁₋₄ alkylene)-R¹² and —N(C₁₋₄ alkyl)-CO—(C₁₋₄ alkylene)-R¹², wherein R¹² is independently selected from —CF₃, —CN and halogen (e.g., —F, —Cl, —Br or —I). Preferably, R¹² is independently selected from —CF₃ and —CN; more preferably, R¹² is —CF₃. Specific examples of such R¹ groups include the corresponding groups R¹ of the compounds described in the examples section. More preferably, R¹ is —(C₁₋₄ alkylene)-NH—(C₁₋₄ alkylene)-CF₃, and even more preferably R¹ is —CH₂—NH—CH₂—CF₃.

Thus, in accordance with this twenty-fourth embodiment, the present invention provides a compound of the following formula (Ia) or a pharmaceutically acceptable salt or solvate thereof

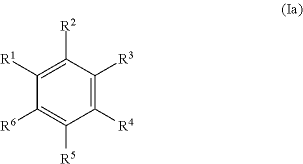

In this twenty-fourth embodiment, R¹ in formula (Ia) is selected from —(C₁₋₄ alkylene)-NH—(C₁₋₄ alkylene)-R¹² (e.g., —CH₂—NH—CH₂—R¹²), —COO—(C₁₋₄ alkylene)-R¹² (e.g., —COO—CH₂—R¹²), —O—CO—(C₁₋₄ alkylene)-R¹², —CO—(C₁₋₄ alkylene)-R¹², —CO—NH—(C₁₋₄ alkylene)-R¹² (e.g., —CO—NH—CH₂—R¹²), —CO—N(C₁₋₄ alkyl)-(C₁₋₄ alkylene)-R¹², —NH—CO—(C₁₋₄ alkylene)-R¹² and —N(C₁₋₄ alkyl)-CO—(C₁₋₄ alkylene)-R¹², wherein R¹² is independently selected from —CF₃, —CN and halogen (e.g., —F, —Cl, —Br or —I). Preferably, R¹² is independently selected from —CF₃ and —CN; more preferably, R¹² is —CF₃. Specific examples of such R¹ groups include the corresponding groups R¹ of the compounds described in the examples section. It is particularly preferred that R¹ is —(C₁₋₄ alkylene)-NH—(C₁₋₄ alkylene)-CF₃, and even more preferably R¹ is —CH₂—NH—CH₂—CF₃.

In the twenty-fourth embodiment, R² is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, R² is selected from hydrogen, C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN. More preferably, R² is hydrogen or C₁₋₄ alkyl. Even more preferably, R² is hydrogen.

In the twenty-fourth embodiment, R³ is selected from —SO₂—NH₂, —SO₂—NH(C₁₋₆ alkyl), —SO₂—N(C₁₋₆ alkyl)(C₁₋₆ alkyl), —SO₂—N═(C₁₋₆ alkylidene) and —SO₂-halogen, wherein the alkyl moiety of said —SO₂—NH(C₁₋₆ alkyl), one or both of the alkyl moieties of said —SO₂—N(C₁₋₆ alkyl)(C₁₋₆ alkyl), and the alkylidene moiety of said —SO₂—N═(C₁₋₆ alkylidene) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl). Preferably, R³ is selected from —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one or more groups (particularly one group) independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl). More preferably, R³ is selected from —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one group selected from —NH₂, —NH(C₁₋₄ alkyl) and —N(C₁₋₄ alkyl)(C₁₋₄ alkyl). Even more preferably, R³ is selected from —SO₂—NH₂, —SO₂—NH(C₁₋₄ alkyl), —SO₂—N(C₁₋₄ alkyl)(C₁₋₄ alkyl), —SO₂—NH—(C₁₋₄ alkylene)-NH₂, —SO₂—NH—(C₁₋₄ alkylene)-NH(C₁₋₄ alkyl), —SO₂—NH—(C₁₋₄ alkylene)-N(C₁₋₄ alkyl)(C₁₋₄ alkyl), —SO₂—N═(C₁₋₄ alkylidene)-NH₂, —SO₂—N═(C₁₋₄ alkylidene)-NH(C₁₋₄ alkyl) and —SO₂—N═(C₁₋₄ alkylidene)-N(C₁₋₄ alkyl)(C₁₋₄ alkyl). Yet even more preferably, R³ is selected from —SO₂—NH₂, —SO₂—NH—CH₃, —SO₂—N(CH₃)₂, —SO₂—NH—(C₁₋₄ alkylene)-NH₂, —SO₂—NH—(C₁₋₄ alkylene)-NH—CH₃, —SO₂—NH—(C₁₋₄ alkylene)-N(CH₃)₂ (e.g., —SO₂—NH—CH₂CH₂—N(CH₃)₂), —SO₂—N═(C₁₋₄ alkylidene)-NH₂, —SO₂—N═(C₁₋₄ alkylidene)-NH—CH₃ and —SO₂—N═(C₁₋₄ alkylidene)-N(CH₃)₂ (e.g., —SO₂—N═CH—N(CH₃)₂). Still more preferably, R³ is —SO₂—NH₂.

In the twenty-fourth embodiment, R⁴ is selected from —O—R⁴¹, —S—R⁴¹, —NH—R⁴¹, —N(C₁₋₆ alkyl)-R⁴¹, halogen (e.g., —Cl), hydrogen, carbocyclyl and heterocyclyl, wherein said carbocyclyl and said heterocyclyl are each optionally substituted with one or more (e.g., one, two or three) groups R⁴². Preferably, R⁴ is selected from —O—R⁴¹, —S—R⁴¹, —NH—R⁴¹, —N(C₁₋₆ alkyl)-R⁴¹, halogen, carbocyclyl and heterocyclyl, wherein said carbocyclyl and said heterocyclyl are each optionally substituted with one or more groups R⁴². More preferably, R⁴ is selected from —O—R⁴¹, —S—R⁴¹, —NH—R⁴¹, —N(C₁₋₆ alkyl)-R⁴¹, carbocyclyl (e.g., aryl, cycloalkyl, or cycloalkenyl) and heterocyclyl (e.g., heteroaryl, heterocycloalkyl, or heterocycloalkenyl), wherein said carbocyclyl and said heterocyclyl are each optionally substituted with one or more groups R⁴². Even more preferably, R⁴ is selected from —O—R⁴¹, —S—R⁴¹, —NH—R⁴¹, —N(C₁₋₄ alkyl)-R⁴¹, aryl and heteroaryl, wherein said aryl and said heteroaryl are each optionally substituted with one or more groups R⁴².

In the twenty-fourth embodiment, R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₁₋₄ alkylene)-carbocyclyl and the heterocyclyl moiety of said —(C₁₋₄ alkylene)-heterocyclyl are each optionally substituted with one or more (e.g., one, two or three) groups R⁴², and wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₁₋₄ alkylene)-heterocyclyl are each optionally substituted with one or more (e.g., one, two or three) groups R⁴³. Preferably, R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₀₋₄ alkylene)-carbocyclyl is selected from cycloalkyl, cycloalkenyl and aryl, wherein the heterocyclyl moiety of said —(C₀₋₄ alkylene)-heterocyclyl is selected from heterocycloalkyl, heterocycloalkenyl and heteroaryl, wherein the carbocyclyl moiety of said —(C₀₋₄ alkylene)-carbocyclyl and the heterocyclyl moiety of said —(C₀₋₄ alkylene)-heterocyclyl are each optionally substituted with one or more (e.g., one, two or three) groups R⁴², and further wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more (e.g., one, two or three) groups R⁴³. More preferably, R⁴¹ is selected from —(C₀₋₄ alkylene)-aryl and —(C₀₋₄ alkylene)-heteroaryl, wherein the aryl moiety of said —(C₀₋₄ alkylene)-aryl and the heteroaryl moiety of said —(C₀₋₄ alkylene)-heteroaryl are each optionally substituted with one or more (e.g., one, two or three) groups R⁴², and further wherein the alkylene moiety of said —(C₀₋₄ alkylene)-aryl and the alkylene moiety of said —(C₀₋₄ alkylene)-heteroaryl are each optionally substituted with one or more (e.g., one, two or three) groups R⁴³. Even more preferably, R⁴¹ is selected from —(C₀₋₄ alkylene)-aryl and —(C₀₋₄ alkylene)-heteroaryl, wherein the aryl moiety of said —(C₁₋₄ alkylene)-aryl and the heteroaryl moiety of said —(C₀₋₄ alkylene)-heteroaryl are each optionally substituted with one or more (e.g., one, two or three) groups R⁴². A preferred example of the aryl moiety of said —(C₀₋₄ alkylene)-aryl is phenyl. A preferred example of the heteroaryl moiety of said —(C₀₋₄ alkylene)-heteroaryl is a 5- or 6-membered monocyclic heteroaryl having 1 or 2 ring heteroatoms independently selected from oxygen, nitrogen and sulfur (wherein the remaining ring atoms are carbon atoms), such as, e.g., imidazolyl, thiophenyl, or pyrimidinyl. Still more preferably, R⁴¹ is selected from phenyl and heteroaryl, wherein said heteroaryl is a 5- or 6-membered monocyclic heteroaryl having 1 or 2 ring heteroatoms independently selected from oxygen, nitrogen and sulfur (the remaining ring atoms of the monocyclic heteroaryl are carbon atoms), and further wherein said phenyl or said heteroaryl is optionally substituted with one or more (e.g., one, two or three) groups R⁴².

In the twenty-fourth embodiment, each R⁴² is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, each R⁴² is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

In the twenty-fourth embodiment, each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃, —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl) and —N(C₁₋₆ alkyl)-CO—(C₁₋₆ alkyl). Preferably, each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃ and —CN.

In the twenty-fourth embodiment, in accordance with the above definitions, it is particularly preferred that R⁴ is selected from —O—(C₁₋₄ alkylene)-aryl, —O—(C₁₋₄ alkylene)-heteroaryl, —S—(C₀₋₄ alkylene)-aryl, —S—(C₀₋₄ alkylene)-heteroaryl, —NH—(C₀₋₄ alkylene)-aryl, —NH—(C₀₋₄ alkylene)-heteroaryl, —N(C₁₋₄ alkyl)-(C₀₋₄ alkylene)-aryl, —N(C₁₋₄ alkyl)-(C₀₋₄ alkylene)-heteroaryl, aryl and heteroaryl, wherein the aryl moiety of any of the aforementioned groups, the heteroaryl moiety of any of the aforementioned groups, said aryl and said heteroaryl are each optionally substituted with one or more (e.g., one, two or three) groups R⁴². Even more preferably, R⁴ is selected from —O-aryl, —O-heteroaryl, —S-aryl, —S-heteroaryl, —NH-aryl, —NH-heteroaryl, —N(C₁₋₄ alkyl)-aryl, —N(C₁₋₄ alkyl)-heteroaryl, aryl and heteroaryl, wherein the aryl moiety of any of the aforementioned groups, the heteroaryl moiety of any of the aforementioned groups, said aryl and said heteroaryl are each optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN. Yet even more preferably, R⁴ is selected from —O— phenyl, —O-heteroaryl, —S-phenyl, —S-heteroaryl, —NH-phenyl, —NH-heteroaryl, —N(C₁₋₄ alkyl)-phenyl, —N(C₁₋₄ alkyl)-heteroaryl, phenyl and heteroaryl, wherein said heteroaryl or the heteroaryl moiety of any of the aforementioned groups is a 5- or 6-membered monocyclic heteroaryl having 1 or 2 ring heteroatoms independently selected from oxygen, nitrogen and sulfur (the remaining ring atoms of the monocyclic heteroaryl are carbon atoms), and further wherein the phenyl moiety of any of the aforementioned groups, the heteroaryl moiety of any of the aforementioned groups, said phenyl and said heteroaryl are each optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN. Still more preferably, R⁴ is —O-phenyl.

In the twenty-fourth embodiment, R⁵ is selected from —NH₂, —NH(C₁₋₆ alkyl), —N(C₁₋₆ alkyl)(C₁₋₆ alkyl), —NO₂ and hydrogen, wherein the alkyl moiety of said —NH(C₁₋₆ alkyl) and one or both of the alkyl moieties of said —N(C₁₋₆ alkyl)(C₁₋₆ alkyl) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH, —S(C₁₋₆ alkyl), carbocyclyl and heterocyclyl, wherein said carbocyclyl and said heterocyclyl are each optionally substituted with one or more (e.g., one, two or three) groups R⁵¹. Preferably, R⁵ is selected from —NH₂, —NH(C₁₋₆ alkyl), —N(C₁₋₆ alkyl)(C₁₋₆ alkyl) and —NO₂, wherein the alkyl moiety of said —NH(C₁₋₆ alkyl) and one or both of the alkyl moieties of said —N(C₁₋₆ alkyl)(C₁₋₆ alkyl) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl). More preferably, R⁵ is selected from —NH₂, —NH(C₁₋₆ alkyl), —N(C₁₋₆ alkyl)(C₁₋₆ alkyl), and —NO₂. Even more preferably, R⁵ is selected from —NH₂, —NH(C₁₋₆ alkyl), and —N(C₁₋₆ alkyl)(C₁₋₆ alkyl). Yet even more preferably, R⁵ is —NH(C₁₋₆ alkyl). Still more preferably, R⁵ is —NH—CH₂CH₂CH₂CH₃.

In the twenty-fourth embodiment, each R⁵¹ is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, each R⁵¹ is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

In the twenty-fourth embodiment, R⁶ is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, R⁶ is selected from hydrogen, C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN. More preferably, R⁶ is hydrogen or C₁₋₄ alkyl. Even more preferably, R⁶ is hydrogen.

In accordance with the twenty-fourth embodiment, the compound of formula (Ia) may be, in particular, a compound of any one of the following formulae, or a pharmaceutically acceptable salt or solvate thereof:
Error! Objects cannot be created from editing field codes.Error! Objects cannot be created from editing field codes.Error! Objects cannot be created from editing field codes.
Error! Objects cannot be created from editing field codes.Error! Objects cannot be created from editing field codes.

The present invention also relates to the compound of formula (Ia) as defined in the twenty-fourth embodiment, or a pharmaceutically acceptable salt or solvate thereof, for use as a medicament. Likewise the invention relates to a pharmaceutical composition comprising said compound of formula (Ia) or a pharmaceutically acceptable salt or solvate thereof, and a pharmaceutically acceptable excipient. The invention furthermore refers to said compound of formula (Ia) or a pharmaceutically acceptable salt or solvate thereof, or a pharmaceutical composition comprising any of the aforementioned entities and a pharmaceutically acceptable excipient, for use in the treatment or prevention of a disease or disorder involving an NKCC (particularly a disease or disorder involving NKCC1), including in particular any one of the specific diseases/disorders mentioned in the present specification. The invention also relates to the use of said compound of formula (Ia) or a pharmaceutically acceptable salt or solvate thereof in the preparation of a medicament for the treatment or prevention of a disease or disorder involving an NKCC (particularly a disease or disorder involving NKCC1), including in particular any one of the specific diseases/disorders mentioned in the present specification. Moreover, the present invention provides a method of treating or preventing a disease or disorder involving an NKCC (particularly a disease or disorder involving NKCC1), including in particular any one of the specific diseases/disorders mentioned in the present specification, the method comprising administering a compound of formula (Ia) as defined in the twenty-fourth embodiment, or a pharmaceutically acceptable salt or solvate thereof, or a pharmaceutical composition comprising any of the aforementioned entities in combination with a pharmaceutically acceptable excipient, to a subject (preferably a human) in need thereof. It will be understood that a therapeutically effective amount of the compound of formula (Ia) or the pharmaceutically acceptable salt or solvate thereof, or of the pharmaceutical composition, is to be administered in accordance with this method.

In a twenty-fifth embodiment, the compound of formula (I) is a compound of the following formula (Ia) or a pharmaceutically acceptable salt or solvate thereof

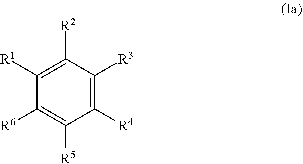

wherein R¹ is as defined below, and wherein the further groups and variables in formula (Ia), including in particular R², R³, R⁴, R⁵ and R⁶, have the same meanings, including the same preferred meanings, as described and defined herein above for the compound of formula (I).

In this twenty-fifth embodiment, R¹ is —(C₁₋₄ alkylene)-S-heterocyclyl, preferably —(C₁₋₄ alkylene)-S-heteroaryl (such as, e.g., —CH₂—S-heteroaryl), wherein the heterocyclyl moiety of said —(C₁₋₄ alkylene)-S-heterocyclyl or the heteroaryl moiety of said —(C₁₋₄ alkylene)-S-heteroaryl or of said —CH₂—S-heteroaryl is optionally substituted with one or more (e.g., one, two or three) groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹¹), —S(R¹¹), —SO₃H, carbocyclyl and heterocyclyl. Specific examples of such R¹ groups include the corresponding groups R¹ of the compounds described in the examples section.

In a twenty-sixth embodiment, the compound of formula (I) is a compound of the following formula (Ia) or a pharmaceutically acceptable salt or solvate thereof

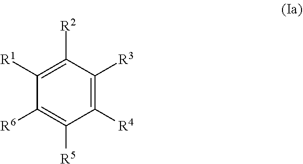

wherein R¹ is as defined below, and wherein the further groups and variables in formula (Ia), including in particular R², R³, R⁴, R⁵ and R⁶, have the same meanings, including the same preferred meanings, as described and defined herein above for the compound of formula (I).

In this twenty-sixth embodiment, R¹ is —(C₁₋₄ alkylene)-O—(C₀₋₄ alkylene)-carbocyclyl, preferably —(C₁₋₄ alkylene)-O—(C₀₋₄ alkylene)-phenyl (such as, e.g., —CH₂—O-phenyl or —CH₂—O—CH₂-phenyl), wherein the carbocyclyl moiety of said —(C₁₋₄ alkylene)-O—(C₀₋₄ alkylene)-carbocyclyl or the phenyl moiety of said —(C₁₋₄ alkylene)-O—(C₀₋₄ alkylene)-phenyl (or of said —CH₂—O-phenyl or —CH₂—O—CH₂-phenyl) is optionally substituted with one or more (e.g., one, two or three) groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹¹), —S(R¹¹), —SO₃H, carbocyclyl and heterocyclyl. Specific examples of such R¹ groups include the corresponding groups R¹ of the compounds described in the examples section.

In a twenty-seventh embodiment, the compound of formula (I) is a compound of the following formula (Ia) or a pharmaceutically acceptable salt or solvate thereof

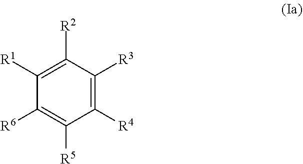

wherein R¹ is as defined below, and wherein the further groups and variables in formula (Ia), including in particular R², R³, R⁴, R⁵ and R⁶, have the same meanings, including the same preferred meanings, as described and defined herein above for the compound of formula (I).

In this twenty-seventh embodiment, R¹ is —(C₁₋₄ alkylene)-heterocyclyl, preferably —CH₂-heterocyclyl, wherein the heterocyclyl moiety of said —(C₁₋₄ alkylene)-heterocyclyl or of said —CH₂-heterocyclyl is optionally substituted with one or more (e.g., one, two or three) groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹¹), —S(R¹¹), —SO₃H, carbocyclyl and heterocyclyl. The heterocyclyl moiety may be, e.g., a heterocycloalkyl (such as, e.g., pyrrolidinyl, piperidinyl, piperazinyl, or morpholinyl), a heterocycloalkenyl (such as, e.g., tetrahydropyridinyl), or a heteroaryl. Specific examples of such R¹ groups include the corresponding groups R¹ of the compounds described in the examples section.

In a twenty-eighth embodiment, the compound of formula (I) is a compound of the following formula (Ia) or a pharmaceutically acceptable salt or solvate thereof

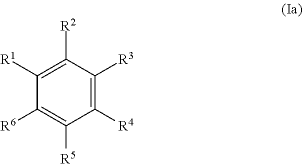

wherein R¹ is —COOH, wherein R³ is —SO₂—NH₂, wherein R⁴ is a group R^{4a}, wherein R⁵ is a group R^{5a}, wherein R⁵ is —NH₂, and wherein the further groups and variables in formula (Ia), including in particular R², R^{4a} and R⁶, have the same meanings, including the same preferred meanings, as described and defined herein above for the compound of formula (I).

In a twenty-ninth embodiment, the compound of formula (I) is a compound of the following formula (Ia) or a pharmaceutically acceptable salt or solvate thereof

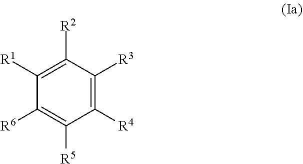

wherein R¹ is —COOH, wherein R³ is —SO₂—NH₂, wherein R⁴ is a group R^{4a}, wherein R⁵ is a group R^{5a}, wherein R^{5a} is —NH—CH₂CH₂CH₂CH₃, and wherein the further groups and variables in formula (Ia), including in particular R², R^{4a} and R⁶, have the same meanings, including the same preferred meanings, as described and defined herein above for the compound of formula (I).

In a thirtieth embodiment, the compound of formula (I) is a compound of the following formula (Ia) or a pharmaceutically acceptable salt or solvate thereof

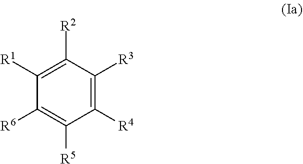

wherein R¹ is —COOH, wherein R³ is —SO₂—NH₂, wherein R⁴ is a group R^{4a}, wherein R⁵ is a group R^{5a}, wherein R⁵ is —NO₂, and wherein the further groups and variables in formula (Ia), including in particular R², R^{4a} and R⁶, have the same meanings, including the same preferred meanings, as described and defined herein above for the compound of formula (I).

In a thirty-first embodiment, the compound of formula (I) is a compound of the following formula (Ia) or a pharmaceutically acceptable salt or solvate thereof

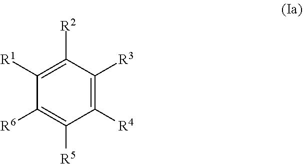

wherein R¹ is —COOH, wherein R³ is —SO₂—N═CH—N(CH₃)₂, wherein R⁴ is a group R^{4a}, wherein R⁵ is a group R^{5a}, wherein R^{5a} is —NH₂, and wherein the further groups and variables in formula (Ia), including in particular R², R^{4a} and R⁶, have the same meanings, including the same preferred meanings, as described and defined herein above for the compound of formula (I).

In a thirty-second embodiment, the compound of formula (I) is a compound of the following formula (Ia) or a pharmaceutically acceptable salt or solvate thereof

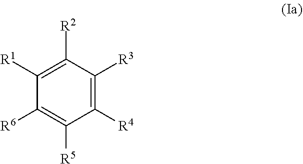

wherein R¹ is —COOH, wherein R³ is —SO₂—N═CH—N(CH₃)₂, wherein R⁴ is a group R^{4a}, wherein R⁵ is a group R^{5a}, wherein R^{5a} is —NH—CH₂CH₂CH₂CH₃, and wherein the further groups and variables in formula (Ia), including in particular R², R^{4a} and R⁶, have the same meanings, including the same preferred meanings, as described and defined herein above for the compound of formula (I).

In a thirty-third embodiment, the compound of formula (I) is a compound of the following formula (Ia) or a pharmaceutically acceptable salt or solvate thereof

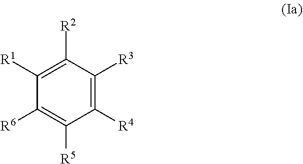

wherein R¹ is —COOH, wherein R³ is —SO₂—N═CH—N(CH₃)₂, wherein R⁴ is a group R^{4a}, wherein R⁵ is a group R^{5a}, wherein R^{5a} is —NO₂, and wherein the further groups and variables in formula (Ia), including in particular R², R^{4a} and R⁶, have the same meanings, including the same preferred meanings, as described and defined herein above for the compound of formula (I).

In a thirty-fourth embodiment, the compound of formula (I) is a compound of the following formula (Ia) or a pharmaceutically acceptable salt or solvate thereof

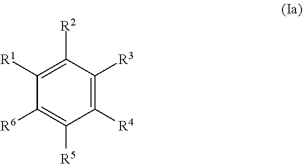

wherein R¹ is —COO—CH₃, wherein R³ is —SO₂—NH₂, wherein R⁴ is a group R^{4a}, wherein R⁵ is a group R^{5a}, wherein R⁵ is —NH₂, and wherein the further groups and variables in formula (Ia), including in particular R², R^{4a} and R⁶, have the same meanings, including the same preferred meanings, as described and defined herein above for the compound of formula (I).

In a thirty-fifth embodiment, the compound of formula (I) is a compound of the following formula (Ia) or a pharmaceutically acceptable salt or solvate thereof

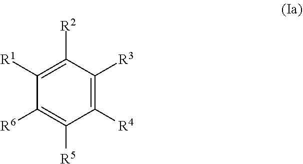

wherein R¹ is —COO—CH₃, wherein R³ is —SO₂—NH₂, wherein R⁴ is a group R^{4a}, wherein R⁵ is a group R^{5a}, wherein R^{5a} is —NH—CH₂CH₂CH₂CH₃, and wherein the further groups and variables in formula (Ia), including in particular R², R^{4a} and R⁶, have the same meanings, including the same preferred meanings, as described and defined herein above for the compound of formula (I).

In a thirty-sixth embodiment, the compound of formula (I) is a compound of the following formula (Ia) or a pharmaceutically acceptable salt or solvate thereof

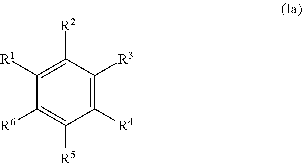

wherein R¹ is —COO—CH₃, wherein R³ is —SO₂—NH₂, wherein R⁴ is a group R^{4a}, wherein R⁵ is a group R^{5a}, wherein R⁵ is —NO₂, and wherein the further groups and variables in formula (Ia), including in particular R², R^{4a} and R⁶, have the same meanings, including the same preferred meanings, as described and defined herein above for the compound of formula (I).

In a thirty-seventh embodiment, the compound of formula (I) is a compound of the following formula (Ia) or a pharmaceutically acceptable salt or solvate thereof

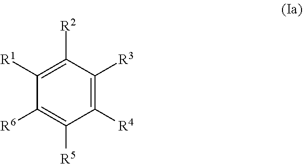

wherein R¹ is —COO—CH₃, wherein R³ is —SO₂—N═CH—N(CH₃)₂, wherein R⁴ is a group R⁴, wherein R⁵ is a group R^{5a}, wherein R^{5a} is —NH₂, and wherein the further groups and variables in formula (Ia), including in particular R², R⁴ and R⁶, have the same meanings, including the same preferred meanings, as described and defined herein above for the compound of formula (I).

In a thirty-eighth embodiment, the compound of formula (I) is a compound of the following formula (Ia) or a pharmaceutically acceptable salt or solvate thereof

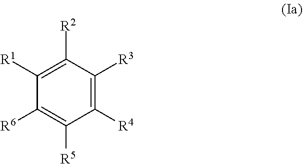

wherein R¹ is —COO—CH₃, wherein R³ is —SO₂—N═CH—N(CH₃)₂, wherein R⁴ is a group R⁴, wherein R⁵ is a group R^{5a}, wherein R^{5a} is —NH—CH₂CH₂CH₂CH₃, and wherein the further groups and variables in formula (Ia), including in particular R², R⁴ and R⁶, have the same meanings, including the same preferred meanings, as described and defined herein above for the compound of formula (I).

In a thirty-ninth embodiment, the compound of formula (I) is a compound of the following formula (Ia) or a pharmaceutically acceptable salt or solvate thereof

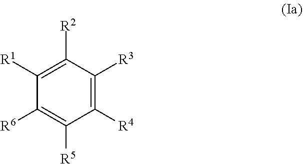

wherein R¹ is —COO—CH₃, wherein R³ is —SO₂—N═CH—N(CH₃)₂, wherein R⁴ is a group R⁴, wherein R⁵ is a group R^{5a}, wherein R^{5a} is —NO₂, and wherein the further groups and variables in formula (Ia), including in particular R², R⁴ and R⁶, have the same meanings, including the same preferred meanings, as described and defined herein above for the compound of formula (I).

In a fortieth embodiment, the compound of formula (I) is a compound of the following formula (Ib) or a pharmaceutically acceptable salt or solvate thereof

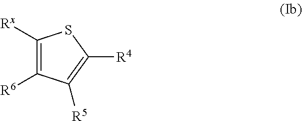

wherein R⁴ is a group R^{4a}, wherein R⁵ is a group R^{5a}, and wherein the further groups and variables in formula (Ib), including in particular R^{X} and R⁶, have the same meanings, including the same preferred meanings, as described and defined herein above for the compound of formula (I).

In this fortieth embodiment, R^{4a} is selected from —O—R⁴¹, —S—R⁴¹, —NH—R⁴¹, —N(C₁₋₆ alkyl)-R⁴¹, halogen (e.g., —Cl), carbocyclyl and heterocyclyl, wherein said carbocyclyl and said heterocyclyl are each optionally substituted with one or more (e.g., one, two or three) groups R⁴². Preferably, R^{4a} is selected from —O—R⁴¹, —S—R⁴¹, —NH—R⁴¹, —N(C₁₋₆ alkyl)-R⁴¹, halogen, carbocyclyl (e.g., aryl, cycloalkyl, or cycloalkenyl) and heterocyclyl (e.g., heteroaryl, heterocycloalkyl, or heterocycloalkenyl), wherein said carbocyclyl and said heterocyclyl are each optionally substituted with one or more groups R⁴². More preferably, R^{4a} is selected from —O—R⁴¹, —S—R⁴¹, —NH—R⁴¹, —N(C₁₋₄ alkyl)-R⁴¹, halogen, aryl and heteroaryl, wherein said aryl and said heteroaryl are each optionally substituted with one or more groups R⁴². Even more preferably, R^{4a} is selected from —O—R⁴¹, —S—R⁴¹, —NH—R⁴¹, —N(C₁₋₄ alkyl)-R⁴¹ and halogen. Still more preferably, R^{4a} is —O—R⁴¹ or halogen.

R⁴¹ is selected from —(C₁₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₁₋₄ alkylene)-carbocyclyl and the heterocyclyl moiety of said —(C₀₋₄ alkylene)-heterocyclyl are each optionally substituted with one or more (e.g., one, two or three) groups R⁴², and wherein said C₁₋₆ alkyl, said C₂₋₆ alkenyl, said C₂₋₆ alkynyl, the alkylene moiety of said —(C₁₋₄ alkylene)-carbocyclyl, and the alkylene moiety of said —(C₀₋₄ alkylene)-heterocyclyl are each optionally substituted with one or more (e.g., one, two or three) groups R⁴³. Preferably, R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₀₋₄ alkylene)-carbocyclyl is selected from cycloalkyl, cycloalkenyl and aryl, wherein the heterocyclyl moiety of said —(C₀₋₄ alkylene)-heterocyclyl is selected from heterocycloalkyl, heterocycloalkenyl and heteroaryl, wherein the carbocyclyl moiety of said —(C₀₋₄ alkylene)-carbocyclyl and the heterocyclyl moiety of said —(C₀₋₄ alkylene)-heterocyclyl are each optionally substituted with one or more (e.g., one, two or three) groups R⁴², and further wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more (e.g., one, two or three) groups R⁴³. More preferably, R⁴¹ is selected from —(C₀₋₄ alkylene)-aryl and —(C₀₋₄ alkylene)-heteroaryl, wherein the aryl moiety of said —(C₀₋₄ alkylene)-aryl and the heteroaryl moiety of said —(C₀₋₄ alkylene)-heteroaryl are each optionally substituted with one or more (e.g., one, two or three) groups R⁴², and further wherein the alkylene moiety of said —(C₀₋₄ alkylene)-aryl and the alkylene moiety of said —(C₀₋₄ alkylene)-heteroaryl are each optionally substituted with one or more (e.g., one, two or three) groups R⁴³. Even more preferably, R⁴¹ is selected from —(C₀₋₄ alkylene)-aryl and —(C₀₋₄ alkylene)-heteroaryl, wherein the aryl moiety of said —(C₀₋₄ alkylene)-aryl and the heteroaryl moiety of said —(C₀₋₄ alkylene)-heteroaryl are each optionally substituted with one or more (e.g., one, two or three) groups R⁴². A preferred example of the aryl moiety of said —(C₀₋₄ alkylene)-aryl is phenyl. A preferred example of the heteroaryl moiety of said —(C₀₋₄ alkylene)-heteroaryl is a 5- or 6-membered monocyclic heteroaryl having 1 or 2 ring heteroatoms independently selected from oxygen, nitrogen and sulfur (wherein the remaining ring atoms are carbon atoms), such as, e.g., imidazolyl, thiophenyl, or pyrimidinyl. Still more preferably, R⁴¹ is selected from phenyl and heteroaryl, wherein said heteroaryl is a 5- or 6-membered monocyclic heteroaryl having 1 or 2 ring heteroatoms independently selected from oxygen, nitrogen and sulfur (the remaining ring atoms of the monocyclic heteroaryl are carbon atoms), and further wherein said phenyl or said heteroaryl is optionally substituted with one or more (e.g., one, two or three) groups R⁴².

Each R⁴² is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, each R⁴² is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

Each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃, —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl) and —N(C₁₋₆ alkyl)-CO—(C₁₋₆ alkyl). Preferably, each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃ and —CN.

In accordance with the above definitions, it is particularly preferred that R^{4a} is —O-aryl or halogen, wherein the aryl moiety of said —O-aryl is optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN. Even more preferably, R^{4a} is —O— phenyl or halogen (such as, in particular, —Cl), wherein the phenyl moiety of said —O-phenyl is optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN.

In this fortieth embodiment, R^{5a} is selected from —NH₂, —NH(C₁₋₆ alkyl), —N(C₁₋₆ alkyl)(C₁₋₆ alkyl), —NO₂ and hydrogen, wherein the alkyl moiety of said —NH(C₁₋₆ alkyl) and one or both of the alkyl moieties of said —N(C₁₋₆ alkyl)(C₁₋₆ alkyl) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH, —S(C₁₋₆ alkyl), carbocyclyl and heterocyclyl, wherein said carbocyclyl and said heterocyclyl are each optionally substituted with one or more (e.g., one, two or three) groups R⁵¹. Preferably, R^{5a} is selected from —NH₂, —NH(C₁₋₆ alkyl), —N(C₁₋₆ alkyl)(C₁₋₆ alkyl), —NO₂ and hydrogen, wherein the alkyl moiety of said —NH(C₁₋₆ alkyl) and one or both of the alkyl moieties of said —N(C₁₋₆ alkyl)(C₁₋₆ alkyl) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl). More preferably, R^{5a} is selected from —NH₂, —NH(C₁₋₆ alkyl), —N(C₁₋₆ alkyl)(C₁₋₆ alkyl), —NO₂ and hydrogen. Particularly preferred examples of R^{5a} are —NH₂, —NH—CH₂CH₂CH₂CH₃, —NO₂ or hydrogen.

Each R⁵¹ is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, each R⁵¹ is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

In a forty-first embodiment, the compound of formula (I) is a compound of the following formula (Ib) or a pharmaceutically acceptable salt or solvate thereof

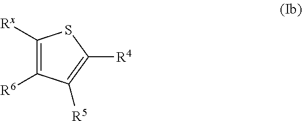

wherein the groups and variables in formula (Ib) have the following meanings:

In this forty-first embodiment, R^{x} is selected from —COOH, —COO—(C₆ alkyl), —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one group selected from —NH₂, —NH(C₄ alkyl) and —N(C₄ alkyl)(C₄ alkyl). Preferably, R^{x} is selected from —COOH, —COOCH₃, —SO₂—NH₂ and —SO₂—N═CH—N(CH₃)₂.

In this forty-first embodiment, R⁴ is a group R⁴.

R^{4a} is selected from —O—R⁴¹, —S—R⁴¹, —NH—R⁴¹, —N(C₁₋₆ alkyl)-R⁴¹, halogen (e.g., —Cl), carbocyclyl and heterocyclyl, wherein said carbocyclyl and said heterocyclyl are each optionally substituted with one or more (e.g., one, two or three) groups R⁴². Preferably, R^{4a} is selected from —O—R⁴¹, —S—R⁴¹, —NH—R⁴¹, —N(C₁₋₆ alkyl)-R⁴¹, halogen, carbocyclyl (e.g., aryl, cycloalkyl, or cycloalkenyl) and heterocyclyl (e.g., heteroaryl, heterocycloalkyl, or heterocycloalkenyl), wherein said carbocyclyl and said heterocyclyl are each optionally substituted with one or more groups R⁴². More preferably, R^{4a} is selected from —O—R⁴¹, —S—R⁴¹, —NH—R⁴¹, —N(C₄ alkyl)-R⁴¹, halogen, aryl and heteroaryl, wherein said aryl and said heteroaryl are each optionally substituted with one or more groups R⁴². Even more preferably, R^{4a} is selected from —O—R⁴¹, —S—R⁴¹, —NH—R⁴¹, —N(C₁₋₄ alkyl)-R⁴¹ and halogen. Still more preferably, R^{4a} is —O—R⁴¹ or halogen.

R⁴¹ is selected from —(C₁₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₁₋₄ alkylene)-carbocyclyl and the heterocyclyl moiety of said —(C₀₋₄ alkylene)-heterocyclyl are each optionally substituted with one or more (e.g., one, two or three) groups R⁴², and wherein said C₁₋₆ alkyl, said C₂₋₆ alkenyl, said C₂₋₆ alkynyl, the alkylene moiety of said —(C₁₋₄ alkylene)-carbocyclyl, and the alkylene moiety of said —(C₀₋₄ alkylene)-heterocyclyl are each optionally substituted with one or more (e.g., one, two or three) groups R⁴³. Preferably, R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₁₋₄ alkylene)-carbocyclyl is selected from cycloalkyl, cycloalkenyl and aryl, wherein the heterocyclyl moiety of said —(C₀₋₄ alkylene)-heterocyclyl is selected from heterocycloalkyl, heterocycloalkenyl and heteroaryl, wherein the carbocyclyl moiety of said —(C₀₋₄ alkylene)-carbocyclyl and the heterocyclyl moiety of said —(C₁₋₄ alkylene)-heterocyclyl are each optionally substituted with one or more (e.g., one, two or three) groups R⁴², and further wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more (e.g., one, two or three) groups R⁴³. More preferably, R⁴¹ is selected from —(C₀₋₄ alkylene)-aryl and —(C₀₋₄ alkylene)-heteroaryl, wherein the aryl moiety of said —(C₀₋₄ alkylene)-aryl and the heteroaryl moiety of said —(C₀₋₄ alkylene)-heteroaryl are each optionally substituted with one or more (e.g., one, two or three) groups R⁴², and further wherein the alkylene moiety of said —(C₀₋₄ alkylene)-aryl and the alkylene moiety of said —(C₀₋₄ alkylene)-heteroaryl are each optionally substituted with one or more (e.g., one, two or three) groups R⁴³. Even more preferably, R⁴¹ is selected from —(C₀₋₄ alkylene)-aryl and —(C₀₋₄ alkylene)-heteroaryl, wherein the aryl moiety of said —(C₀₋₄ alkylene)-aryl and the heteroaryl moiety of said —(C₀₋₄ alkylene)-heteroaryl are each optionally substituted with one or more (e.g., one, two or three) groups R⁴². A preferred example of the aryl moiety of said —(C₀₋₄ alkylene)-aryl is phenyl. A preferred example of the heteroaryl moiety of said —(C₀₋₄ alkylene)-heteroaryl is a 5- or 6-membered monocyclic heteroaryl having 1 or 2 ring heteroatoms independently selected from oxygen, nitrogen and sulfur (wherein the remaining ring atoms are carbon atoms), such as, e.g., imidazolyl, thiophenyl, or pyrimidinyl. Still more preferably, R⁴¹ is selected from phenyl and heteroaryl, wherein said heteroaryl is a 5- or 6-membered monocyclic heteroaryl having 1 or 2 ring heteroatoms independently selected from oxygen, nitrogen and sulfur (the remaining ring atoms of the monocyclic heteroaryl are carbon atoms), and further wherein said phenyl or said heteroaryl is optionally substituted with one or more (e.g., one, two or three) groups R⁴².

Each R⁴² is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, each R⁴² is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

Each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃, —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl) and —N(C₁₋₆ alkyl)-CO—(C₁₋₆ alkyl). Preferably, each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃ and —CN.

In accordance with the above definitions, it is particularly preferred that R^{4a} is —O-aryl or halogen, wherein the aryl moiety of said —O-aryl is optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN. Even more preferably, R^{4a} is —O— phenyl or halogen (such as, in particular, —Cl), wherein the phenyl moiety of said —O-phenyl is optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN.

In this forty-first embodiment, R⁵ is a group R^{5a}.

R^{5a} is selected from —NH₂, —NH(C₁₋₆ alkyl), —N(C₁₋₆ alkyl)(C₁₋₆ alkyl), —NO₂ and hydrogen, wherein the alkyl moiety of said —NH(C₁₋₆ alkyl) and one or both of the alkyl moieties of said —N(C₁₋₆ alkyl)(C₁₋₆ alkyl) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH, —S(C₁₋₆ alkyl), carbocyclyl and heterocyclyl, wherein said carbocyclyl and said heterocyclyl are each optionally substituted with one or more (e.g., one, two or three) groups R⁵¹. Preferably, R^{5a} is selected from —NH₂, —NH(C₁₋₆ alkyl), —N(C₁₋₆ alkyl)(C₁₋₆ alkyl), —NO₂ and hydrogen, wherein the alkyl moiety of said —NH(C₁₋₆ alkyl) and one or both of the alkyl moieties of said —N(C₁₋₆ alkyl)(C₁₋₆ alkyl) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl). More preferably, R^{5a} is selected from —NH₂, —NH(C₁₋₆ alkyl), —N(C₁₋₆ alkyl)(C₁₋₆ alkyl), —NO₂ and hydrogen. Particularly preferred examples of R^{5a} are —NH₂, —NH—CH₂CH₂CH₂CH₃, —NO₂ or hydrogen.

Each R⁵¹ is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, each R⁵¹ is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

R⁶ is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, R⁶ is selected from hydrogen, C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN. More preferably, R⁶ is hydrogen or C₁₋₄ alkyl. Even more preferably, R⁶ is hydrogen.

In a forty-second embodiment, the compound of formula (I) is a compound of the following formula (Ib) or a pharmaceutically acceptable salt or solvate thereof

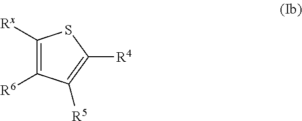

wherein R^{x} is selected from —COOH, —COOCH₃, —SO₂—NH₂ and —SO₂—N═CH—N(CH₃)₂, and wherein the further groups and variables in formula (Ib), including in particular R⁴, R⁵ and R⁶, have the same meanings, including the same preferred meanings, as described and defined in the forty-first embodiment.

In a forty-third embodiment, the compound of formula (I) is a compound of the following formula (Ib) or a pharmaceutically acceptable salt or solvate thereof

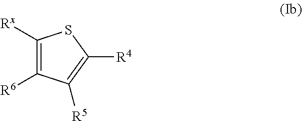

wherein R^{x} is —COOH, and wherein the further groups and variables in formula (Ib), including in particular R⁴, R⁵ and R⁶, have the same meanings, including the same preferred meanings, as described and defined in the forty-first embodiment.

In a forty-fourth embodiment, the compound of formula (I) is a compound of the following formula (Ib) or a pharmaceutically acceptable salt or solvate thereof

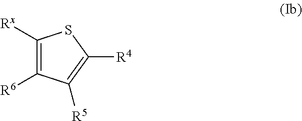

wherein R^{x} is —COOCH₃, and wherein the further groups and variables in formula (Ib), including in particular R⁴, R⁵ and R⁶, have the same meanings, including the same preferred meanings, as described and defined in the forty-first embodiment.

In a forty-fifth embodiment, the compound of formula (I) is a compound of the following formula (Ib) or a pharmaceutically acceptable salt or solvate thereof

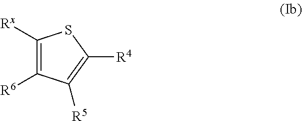

wherein R^{x} is —SO₂—NH₂, and wherein the further groups and variables in formula (Ib), including in particular R⁴, R⁵ and R⁶, have the same meanings, including the same preferred meanings, as described and defined in the forty-first embodiment.

In a forty-sixth embodiment, the compound of formula (I) is a compound of the following formula (Ib) or a pharmaceutically acceptable salt or solvate thereof

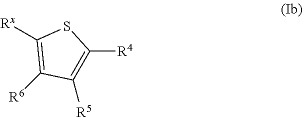

wherein R^{x} is —SO₂—N═CH—N(CH₃)₂, and wherein the further groups and variables in formula (Ib), including in particular R⁴, R⁵ and R⁶, have the same meanings, including the same preferred meanings, as described and defined in the forty-first embodiment.

In a forty-seventh embodiment, the compound of formula (I) is a compound of the following formula (Ib) or a pharmaceutically acceptable salt or solvate thereof

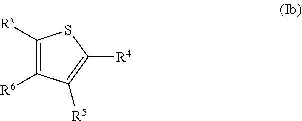

wherein R⁴ is —O-phenyl, wherein the phenyl moiety of said —O-phenyl is optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN; and wherein the further groups and variables in formula (Ib), including in particular R^{x}, R⁵ and R⁶, have the same meanings, including the same preferred meanings, as described and defined in the forty-first embodiment.

In a forty-eighth embodiment, the compound of formula (I) is a compound of the following formula (Ib) or a pharmaceutically acceptable salt or solvate thereof

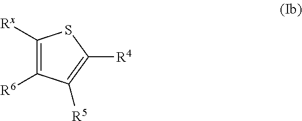

wherein R⁴ is halogen (such as, in particular, —Cl), and wherein the further groups and variables in formula (Ib), including in particular R^{x}, R⁵ and R⁶, have the same meanings, including the same preferred meanings, as described and defined in the forty-first embodiment.

In a forty-ninth embodiment, the compound of formula (I) is a compound of the following formula (Ib) or a pharmaceutically acceptable salt or solvate thereof

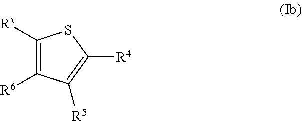

wherein R^{x} is —COOH, wherein R⁵ is —NH₂, and wherein the further groups and variables in formula (Ib), including in particular R⁴ and R⁶, have the same meanings, including the same preferred meanings, as described and defined in the forty-first embodiment.

In a fiftieth embodiment, the compound of formula (I) is a compound of the following formula (Ib) or a pharmaceutically acceptable salt or solvate thereof

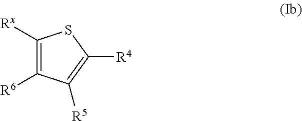

wherein R^{x} is —COOCH₃, wherein R⁵ is —NH₂, and wherein the further groups and variables in formula (Ib), including in particular R⁴ and R⁶, have the same meanings, including the same preferred meanings, as described and defined in the forty-first embodiment.

In a fifty-first embodiment, the compound of formula (I) is a compound of the following formula (Ib) or a pharmaceutically acceptable salt or solvate thereof

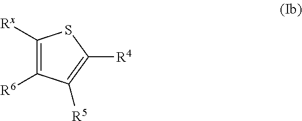

wherein R^{x} is —SO₂—NH₂, wherein R⁵ is —NH₂, and wherein the further groups and variables in formula (Ib), including in particular R⁴ and R⁶, have the same meanings, including the same preferred meanings, as described and defined in the forty-first embodiment.

In a fifty-second embodiment, the compound of formula (I) is a compound of the following formula (Ib) or a pharmaceutically acceptable salt or solvate thereof

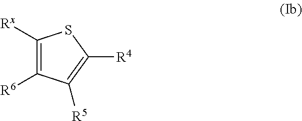

wherein R^{x} is —SO₂—N═CH—N(CH₃)₂, wherein R⁵ is —NH₂, and wherein the further groups and variables in formula (Ib), including in particular R⁴ and R⁶, have the same meanings, including the same preferred meanings, as described and defined in the forty-first embodiment.

In a fifty-third embodiment, the compound of formula (I) is a compound of the following formula (Ib) or a pharmaceutically acceptable salt or solvate thereof

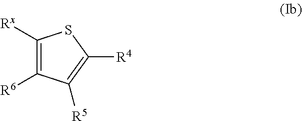

wherein R^{x} is —COOH, wherein R⁵ is —NH—CH₂CH₂CH₂CH₃, and wherein the further groups and variables in formula (Ib), including in particular R⁴ and R⁶, have the same meanings, including the same preferred meanings, as described and defined in the forty-first embodiment.

In a fifty-fourth embodiment, the compound of formula (I) is a compound of the following formula (Ib) or a pharmaceutically acceptable salt or solvate thereof

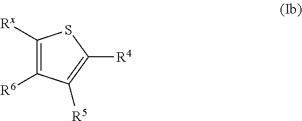

wherein R^{x} is —COOCH₃, wherein R⁵ is —NH—CH₂CH₂CH₂CH₃, and wherein the further groups and variables in formula (Ib), including in particular R⁴ and R⁶, have the same meanings, including the same preferred meanings, as described and defined in the forty-first embodiment.

In a fifty-fifth embodiment, the compound of formula (I) is a compound of the following formula (Ib) or a pharmaceutically acceptable salt or solvate thereof

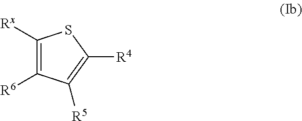

wherein R^{x} is —SO₂—NH₂, wherein R⁵ is —NH—CH₂CH₂CH₂CH₃, and wherein the further groups and variables in formula (Ib), including in particular R⁴ and R⁶, have the same meanings, including the same preferred meanings, as described and defined in the forty-first embodiment.

In a fifty-sixth embodiment, the compound of formula (I) is a compound of the following formula (Ib) or a pharmaceutically acceptable salt or solvate thereof

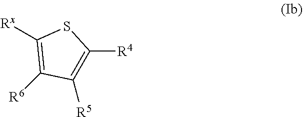

wherein R^{x} is —SO₂—N═CH—N(CH₃)₂, wherein R⁵ is —NH—CH₂CH₂CH₂CH₃, and wherein the further groups and variables in formula (Ib), including in particular R⁴ and R⁶, have the same meanings, including the same preferred meanings, as described and defined in the forty-first embodiment.

In a fifty-seventh embodiment, the compound of formula (I) is a compound of the following formula (Ib) or a pharmaceutically acceptable salt or solvate thereof

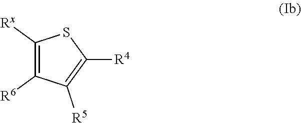

wherein R^{x} is —COOH, wherein R⁵ is —NO₂, and wherein the further groups and variables in formula (Ib), including in particular R⁴ and R⁶, have the same meanings, including the same preferred meanings, as described and defined in the forty-first embodiment.

In a fifty-eighth embodiment, the compound of formula (I) is a compound of the following formula (Ib) or a pharmaceutically acceptable salt or solvate thereof

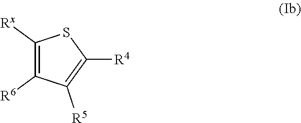

wherein R^{x} is —COOCH₃, wherein R⁵ is —NO₂, and wherein the further groups and variables in formula (Ib), including in particular R⁴ and R⁶, have the same meanings, including the same preferred meanings, as described and defined in the forty-first embodiment.

In a fifty-ninth embodiment, the compound of formula (I) is a compound of the following formula (Ib) or a pharmaceutically acceptable salt or solvate thereof

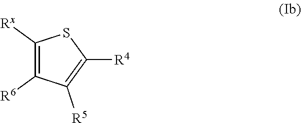

wherein R^{x} is —SO₂—NH₂, wherein R⁵ is —NO₂, and wherein the further groups and variables in formula (Ib), including in particular R⁴ and R⁶, have the same meanings, including the same preferred meanings, as described and defined in the forty-first embodiment.

In a sixtieth embodiment, the compound of formula (I) is a compound of the following formula (Ib) or a pharmaceutically acceptable salt or solvate thereof

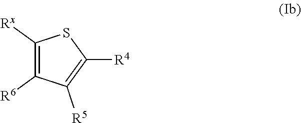

wherein R^{x} is —SO₂—N═CH—N(CH₃)₂, wherein R⁵ is —NO₂, and wherein the further groups and variables in formula (Ib), including in particular R⁴ and R⁶, have the same meanings, including the same preferred meanings, as described and defined in the forty-first embodiment.

In a sixty-first embodiment, the compound of formula (I) is a compound of the following formula (Ib) or a pharmaceutically acceptable salt or solvate thereof

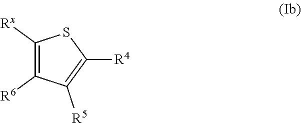

wherein R^{x} is —COOH, wherein R⁵ is hydrogen, and wherein the further groups and variables in formula (Ib), including in particular R⁴ and R⁶, have the same meanings, including the same preferred meanings, as described and defined in the forty-first embodiment.

In a sixty-second embodiment, the compound of formula (I) is a compound of the following formula (Ib) or a pharmaceutically acceptable salt or solvate thereof

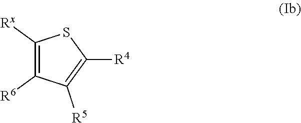

wherein R^{x} is —COOCH₃, wherein R⁵ is hydrogen, and wherein the further groups and variables in formula (Ib), including in particular R⁴ and R⁶, have the same meanings, including the same preferred meanings, as described and defined in the forty-first embodiment.

In a sixty-third embodiment, the compound of formula (I) is a compound of the following formula (Ib) or a pharmaceutically acceptable salt or solvate thereof

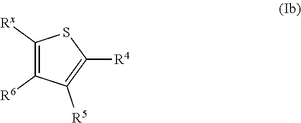

wherein R^{x} is —SO₂—NH₂, wherein R⁵ is hydrogen, and wherein the further groups and variables in formula (Ib), including in particular R⁴ and R⁶, have the same meanings, including the same preferred meanings, as described and defined in the forty-first embodiment.

In a sixty-fourth embodiment, the compound of formula (I) is a compound of the following formula (Ib) or a pharmaceutically acceptable salt or solvate thereof

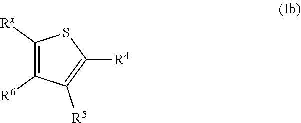

wherein R^{x} is —SO₂—N═CH—N(CH₃)₂, wherein R⁵ is hydrogen, and wherein the further groups and variables in formula (Ib), including in particular R⁴ and R⁶, have the same meanings, including the same preferred meanings, as described and defined in the forty-first embodiment.

In a sixty-fifth embodiment, the compound of formula (I) is a compound of the following formula (Ia) or a pharmaceutically acceptable salt or solvate thereof

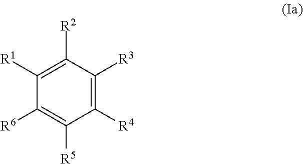

wherein R¹ and R⁶ are mutually linked to form a group —R¹⁶—, wherein R⁴ is a group R^{4a} and wherein R^{4a} is hydrogen, wherein R⁵ is a group R^{5a}, and wherein the further groups and variables in formula (Ia), including in particular R¹⁶, R², R³ and R⁵, have the same meanings, including the same preferred meanings, as described and defined herein above for the compound of formula (I).

In each one of the embodiments described herein above, the respective compound can be used as a medicament, particularly for the treatment or prevention of a disease or disorder involving an NKCC (preferably NKCC1), including for the treatment or prevention of any of the corresponding specific diseases/disorders referred to in the present specification. It is particularly preferred that the respective compound is used for the treatment or prevention of hyperhidrosis.

The present invention also relates to novel compounds. In particular, the invention relates to a compound of formula (I) as described and defined herein, including any one of the examples and embodiments of the compound of formula (I) described in this specification, or a pharmaceutically acceptable salt or solvate thereof. The invention further relates to any such compound for use as a medicament, and provides a pharmaceutical composition comprising a compound of formula (I) or a pharmaceutically acceptable salt or solvate thereof in combination with a pharmaceutically acceptable excipient.

In particular, the present invention relates to a compound of the following formula (a) or a pharmaceutically acceptable salt or solvate thereof (and, furthermore, the invention also relates to said compound for use as a medicament, and to a pharmaceutical composition comprising said compound and a pharmaceutically acceptable excipient):

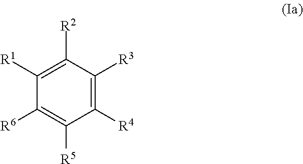

In formula (Ia), R¹ is selected from —COOH, —COO—(C₁₋₁₅ alkyl), —COO—(C₀₋₁₅ alkylene)-carbocyclyl, —COO—(C₀₋₁₅ alkylene)-heterocyclyl, —O—CHO, —O—CO—(C₁₋₁₅ alkyl), —O—CO—(C₀₋₁₅ alkylene)-carbocyclyl, —O—CO—(C₀₋₁₅ alkylene)-heterocyclyl, —CHO, —CO—(C₀₋₁₅ alkyl), —CO—(C₀₋₁₅ alkylene)-carbocyclyl, —CO—(C₀₋₁₅ alkylene)-heterocyclyl, —CO—NH₂, —CO—N(R¹¹)—(C₁₋₁₅ alkyl), —CO—N(R¹¹)—(C₀₋₁₅ alkylene)-carbocyclyl, —CO—N(R¹¹)—(C₀₋₁₅ alkylene)-heterocyclyl, —N(R¹¹)—CHO, —N(R¹¹)—CO—(C₁₋₁₅ alkyl), —N(R¹¹)—CO—(C₀₋₁₅ alkylene)-carbocyclyl, —N(R¹¹)—CO—(C₀₋₁₅ alkylene)-heterocyclyl, C₁₋₁₅ alkyl, —(C₀₋₁₅ alkylene)-carbocyclyl, —(C₀₋₁₅ alkylene)-heterocyclyl, C₂₋₁₅ alkenyl, —(C₂₋₁₅ alkenylene)-carbocyclyl, —(C₂₋₁₅ alkenylene)-heterocyclyl, C₂₋₁₅ alkynyl, —(C₂₋₁₅ alkynylene)-carbocyclyl and —(C₂₋₁₅ alkynylene)-heterocyclyl,

wherein the alkyl moiety of any of the aforementioned groups, the alkylene moiety of any of the aforementioned groups, the alkenylene moiety of any of the aforementioned groups, the alkynylene moiety of any of the aforementioned groups, said C₁₋₁₅ alkyl, said C₂₋₁₅ alkenyl and said C₂₋₁₅ alkynyl are each optionally substituted with one or more groups independently selected from halogen, —CF₃, —CN, —NO₂, —N(R¹¹)(R¹¹), —O(R¹¹), —S(R¹¹) and —SO₃H,

wherein one or more —CH₂— units comprised in the alkyl moiety of any of the aforementioned groups, in the alkylene moiety of any of the aforementioned groups, in the alkenylene moiety of any of the aforementioned groups, in the alkynylene moiety of any of the aforementioned groups, in said C₁₋₁₅ alkyl, in said C₂₋₁₅ alkenyl, or in said C₂₋₁₅ alkynyl are each optionally replaced by a group independently selected from —O—, —CO—, —COO—, —O—CO—, —N(R¹¹)—, —N(R¹¹)—CO—, —CO—N(R¹¹)—, —S—, —SO—, —SO₂—, —SO₂—N(R¹¹)— and —N(R¹¹)—SO₂—, and further wherein the carbocyclyl moiety of any of the aforementioned groups and the heterocyclyl moiety of any of the aforementioned groups are each optionally substituted with one or more groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹¹), —S(R¹¹), —SO₃H, carbocyclyl and heterocyclyl.

Each R¹¹ is independently hydrogen or C₁₋₆ alkyl. Preferably, each R¹¹ is independently hydrogen or C₁₋₄ alkyl (e.g., methyl or ethyl).

R¹ may be, for example, any one of the specific groups R¹ as comprised in any of the compounds described in the examples section.

In particular, R¹ may be, e.g., —COO—(C₁₋₁₅ alkyl), wherein the alkyl moiety of said —COO—(C₁₋₁₅ alkyl) is optionally substituted with one or more (e.g., one, two or three) groups independently selected from halogen, —CF₃, —CN, —NO₂, —NH₂, —NH(C₁₋₄ alkyl), —N(C₁₋₄ alkyl)(C₁₋₄ alkyl), —OH, —O(C₁₋₄ alkyl), —SH and —S(C₁₋₄ alkyl), and further wherein one or two —CH₂— units comprised in the alkyl moiety of said —COO—(C₁₋₁₅ alkyl) are each optionally replaced by a group independently selected from —O—, —CO—, —COO—, —O—CO—, —NH—, —N(C₁₋₄ alkyl)-, —NH—CO—, —N(C₁₋₄ alkyl)-CO—, —CO—NH—, —CO—N(C₁₋₄ alkyl)-, —S—, —SO—, —SO₂—, —SO₂—NH—, —SO₂—N(C₁₋₄ alkyl)-, —NH—SO₂— and —N(C₁₋₄ alkyl)-SO₂—. The alkyl moiety of said —COO—(C₁₋₁₅ alkyl) has 1 to 15 carbon atoms, particularly 1 to 10 carbon atoms. Preferred examples of such R¹ groups include the corresponding groups R¹ of the compounds described in the examples section (including, e.g., —COO—CH₃ or —COO—CH₂CH₃; particularly —COO—CH₃).

A further preferred example of R¹ is —COOH.

Further preferred examples of R¹ are —(C₁₋₄ alkylene)-NH—(C₁₋₄ alkylene)-R¹² (such as, e.g., —CH₂—NH—CH₂—R¹²), —COO—(C₁₋₄ alkylene)-R¹² (e.g., —COO—CH₂—R¹²), —O—CO—(C₁₋₄ alkylene)-R¹², —CO—(C₁₋₄ alkylene)-R¹², —CO—NH—(C₁₋₄ alkylene)-R¹² (e.g., —CO—NH—CH₂—R¹²), —CO—N(C₁₋₄ alkyl)-(C₁₋₄ alkylene)-R¹², —NH—CO—(C₁₋₄ alkylene)-R¹², or —N(C₁₋₄ alkyl)-CO—(C₁₋₄ alkylene)-R¹², wherein R¹² is independently selected from —CF₃, —CN and halogen (e.g., —F, —Cl, —Br or —I); preferably, R¹² is independently selected from —CF₃ and —CN; more preferably, R¹² is —CF₃. Specific examples of such R¹ groups include the corresponding groups R¹ of the compounds described in the examples section. A particularly preferred example of R¹ is —(C₁₋₄ alkylene)-NH—(C₁₋₄ alkylene)-CF₃, and even more preferred example is —CH₂—NH—CH₂—CF₃.

A further example of R¹ is —(C₁₋₄ alkylene)-S-heterocyclyl, particularly —(C₁₋₄ alkylene)-S-heteroaryl (such as, e.g., —CH₂—S-heteroaryl), wherein the heterocyclyl moiety of said —(C₁₋₄ alkylene)-S-heterocyclyl or the heteroaryl moiety of said —(C₁₋₄ alkylene)-S-heteroaryl or of said —CH₂—S-heteroaryl is optionally substituted with one or more (e.g., one, two or three) groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹¹), —S(R¹¹), —SO₃H, carbocyclyl and heterocyclyl. Specific examples of such R¹ groups include the corresponding groups R¹ of the compounds described in the examples section.

A further example of R¹ is —(C₁₋₄ alkylene)-O—(C₀₋₄ alkylene)-carbocyclyl, particularly —(C₁₋₄ alkylene)-O—(C₀₋₄ alkylene)-phenyl (such as, e.g., —CH₂—O-phenyl or —CH₂—O—CH₂-phenyl), wherein the carbocyclyl moiety of said —(C₁₋₄ alkylene)-O—(C₀₋₄ alkylene)-carbocyclyl or the phenyl moiety of said —(C₁₋₄ alkylene)-O—(C₀₋₄ alkylene)-phenyl (or of said —CH₂—O-phenyl or —CH₂—O—CH₂-phenyl) is optionally substituted with one or more (e.g., one, two or three) groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹¹), —S(R¹¹), —SO₃H, carbocyclyl and heterocyclyl. Specific examples of such R¹ groups include the corresponding groups R¹ of the compounds described in the examples section.

A further example of R¹ is —(C₁₋₄ alkylene)-heterocyclyl, particularly —CH₂-heterocyclyl, wherein the heterocyclyl moiety of said —(C₁₋₄ alkylene)-heterocyclyl or of said —CH₂-heterocyclyl is optionally substituted with one or more (e.g., one, two or three) groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹¹), —S(R¹¹), —SO₃H, carbocyclyl and heterocyclyl. The heterocyclyl moiety may be, e.g., a heterocycloalkyl (such as, e.g., pyrrolidinyl, piperidinyl, piperazinyl, or morpholinyl), a heterocycloalkenyl (such as, e.g., tetrahydropyridinyl), or a heteroaryl. Specific examples of such R¹ groups include the corresponding groups R¹ of the compounds described in the examples section.

R² is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, R² is selected from hydrogen, C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN. More preferably, R² is hydrogen or C₁₋₄ alkyl. Even more preferably, R² is hydrogen.

R³ is selected from —SO₂—NH₂, —SO₂—NH(C₆ alkyl), —SO₂—N(C₆ alkyl)(C₆ alkyl), —SO₂—N═(C₆ alkylidene) and —SO₂-halogen, wherein the alkyl moiety of said —SO₂—NH(C₁₋₆ alkyl), one or both of the alkyl moieties of said —SO₂—N(C₁₋₆ alkyl)(C₁₋₆ alkyl), and the alkylidene moiety of said —SO₂—N═(C₁₋₆ alkylidene) are each optionally substituted with one or more groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl). Preferably, R³ is selected from —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one or more groups (particularly one group) independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl). More preferably, R³ is selected from —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one group selected from —NH₂, —NH(C₁₋₄ alkyl) and —N(C₁₋₄ alkyl)(C₁₋₄ alkyl). Even more preferably, R³ is selected from —SO₂—NH₂, —SO₂—NH(C₁₋₄ alkyl), —SO₂—N(C₁₋₄ alkyl)(C₁₋₄ alkyl), —SO₂—NH—(C₁₋₄ alkylene)-NH₂, —SO₂—NH—(C₁₋₄ alkylene)-NH(C₁₋₄ alkyl), —SO₂—NH—(C₁₋₄ alkylene)-N(C₁₋₄ alkyl)(C₁₋₄ alkyl), —SO₂—N═(C₁₋₄ alkylidene)-NH₂, —SO₂—N═(C₁₋₄ alkylidene)-NH(C₁₋₄ alkyl) and —SO₂—N═(C₁₋₄ alkylidene)-N(C₁₋₄ alkyl)(C₁₋₄ alkyl). Yet even more preferably, R³ is selected from —SO₂—NH₂, —SO₂—NH—CH₃, —SO₂—N(CH₃)₂, —SO₂—NH—(C₁₋₄ alkylene)-NH₂, —SO₂—NH—(C₁₋₄ alkylene)-NH—CH₃, —SO₂—NH—(C₁₋₄ alkylene)-N(CH₃)₂ (e.g., —SO₂—NH—CH₂CH₂—N(CH₃)₂), —SO₂—N═(C₁₋₄ alkylidene)-NH₂, —SO₂—N═(C₁₋₄ alkylidene)-NH—CH₃ and —SO₂—N═(C₁₋₄ alkylidene)-N(CH₃)₂ (e.g., —SO₂—N═CH—N(CH₃)₂). A particularly preferred example of R³ is —SO₂—NH₂. A further particularly preferred example of R³ is —SO₂—N═CH—N(CH₃)₂.

R⁴ and R⁵ are mutually linked to form a group —R^{5b}—.

R^{5b} is selected from —R^{5b1}—R^{5b2}—R^{5b1}—, —N═C(R⁵³)—R^{5b3}—R^{5b1}—, —R^{5b1}—R^{5b3}—C(R⁵³)═N—, and —N═C(R⁵³)—R^{5b4}—C(R⁵³)═N—. Each R^{5b1} is independently selected from —N(R⁵²)—, —O— and —S—. R^{5b2} is selected from —C(R⁵³)(R⁵³)—, —C(R⁵³)(R⁵³)—C(R⁵³)(R⁵³)—, —C(R⁵³)═C(R⁵³)—, —C(R⁵³)(R⁵³)—C(R⁵³)═C(R⁵³)— and —C(R⁵³)═C(R⁵³)—C(R⁵³)(R⁵³)—. R^{5b3} is selected from a covalent bond, —C(R⁵³)(R⁵³)—, —C(R⁵³)(R⁵³)—C(R⁵³)(R⁵³)— and —C(R⁵³)═C(R⁵³)—. R^{5b4} is selected from a covalent bond and —C(R⁵³)(R⁵³)—.

Each R⁵² is independently selected from hydrogen, C₁₋₆ alkyl, C₂₋₆ alkenyl, C₂₋₆ alkynyl, —(C₀₋₄ alkylene)-aryl and —(C₀₋₄ alkylene)-heteroaryl. Preferably, each R⁵² is independently selected from hydrogen, C₁₋₄ alkyl (e.g., n-butyl), C₂₋₄ alkenyl and C₂₋₄ alkynyl (e.g., —CH₂—C═CH). Even more preferably, each R⁵² is hydrogen.

Each R⁵³ is independently selected from hydrogen, C₁₋₆ alkyl, C₂₋₆ alkenyl, C₂₋₆ alkynyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl), —N(C₁₋₆ alkyl)-SO₂—(C₁₋₆ alkyl), —(C₀₋₄ alkylene)-aryl and —(C₀₋₄ alkylene)-heteroaryl, and any two groups R⁵³ that are attached to the same carbon atom may also together form a group ═O, and any two groups R⁵³ that are attached to adjacent carbon atoms connected by a double bond may also be mutually linked to form a group —C(R⁵⁴)═C(R⁵⁴)—C(R⁵⁴)═C(R⁵⁴)—. Preferably, each R⁵³ is independently selected from hydrogen, C₁₋₆ alkyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), and —CN, and any two groups R⁵³ that are attached to the same carbon atom may also together form a group ═O, and any two groups R⁵³ that are attached to adjacent carbon atoms connected by a double bond may also be mutually linked to form a group —C(R⁵⁴)═C(R⁵⁴)—C(R⁵⁴)═C(R⁵⁴)—. More preferably, each R⁵³ is independently selected from hydrogen, C₁₋₆ alkyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), and —CN, and any two groups R⁵³ that are attached to the same carbon atom may also together form a group ═O. Even more preferably, each R⁵³ is independently selected from hydrogen, C₁₋₄ alkyl, —OH, —O(C₁₋₄ alkyl), —NH₂, —NH(C₁₋₄ alkyl), —N(C₁₋₄ alkyl)(C₁₋₄ alkyl), halogen, C₁₋₄ haloalkyl, —O—(C₁₋₄ haloalkyl), and —CN, and any two groups R⁵³ that are attached to the same carbon atom may also together form a group ═O.

Each R⁵⁴ is independently selected from hydrogen, C₁₋₆ alkyl, C₂₋₆ alkenyl, C₂₋₆ alkynyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, each R⁵⁴ is independently selected from hydrogen, C₁₋₆ alkyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN. More preferably, each R⁵⁴ is hydrogen.

It is particularly preferred that R^{5b} is selected from —N(R⁵²)—C(R⁵³)(R⁵³)—N(R⁵²)—, —N═C(R⁵³)—N(R⁵²)—, —N(R⁵²)—C(R⁵³)═N—, —N(R⁵²)—C(R⁵³)(R⁵³)—C(R⁵³)(R⁵³)—N(R⁵²)—, —N═C(R⁵³)—C(R⁵³)(R⁵³)—N(R⁵²)—, —N(R⁵²)—C(R⁵³)═C(R⁵³)—N(R⁵²)—, —N(R⁵²)—C(R⁵³)(R⁵³)—C(R⁵³)═N—, —N═C(R⁵³)—C(R⁵³)═N—, —N(R⁵²)—C(R⁵³)(R⁵³)—C(R⁵³)(R⁵³)—C(R⁵³)(R⁵³)—N(R⁵²)—, —N═C(R⁵³)—C(R⁵³)(R⁵³)—C(R⁵³)(R⁵³)—N(R⁵²)—, —N(R⁵²)—C(R⁵³)(R⁵³)—C(R⁵³)═C(R⁵³)—N(R⁵²)—, —N(R⁵²)—C(R⁵³)═C(R⁵³)—C(R⁵³)(R⁵³)—N(R⁵²)—, —N(R⁵²) C(R⁵³)(R⁵³)—C(R⁵³)(R⁵³)—C(R⁵³)═N—, —N═C(R⁵³)—C(R⁵³)(R⁵³)—C(R⁵³)═N—, —O—C(R⁵³)(R⁵³)—N(R⁵²)—, —O—C(R⁵³)(R⁵³)—C(R⁵³)(R⁵³)—N(R⁵²)—, —O—C(R⁵³)═C(R⁵³)—N(R⁵²)—, —O—C(R⁵³)(R⁵³)—C(R⁵³)(R⁵³)—C(R⁵³)(R⁵³)—N(R⁵²)—, —O—C(R⁵³)(R⁵³)—C(R⁵³)═C(R⁵³)—N(R⁵²)—, —O—C(R⁵³)═C(R⁵³)—C(R⁵³)(R⁵³)—N(R⁵²)—, —N(R⁵²)—C(R⁵³)(R⁵³)—O—, —N(R⁵²)—C(R⁵³)(R⁵³)—C(R⁵³)(R⁵³)—O—, —N(R⁵²)—C(R⁵³)═C(R⁵³)—O—, —N(R⁵²)—C(R⁵³)(R⁵³)—C(R⁵³)(R⁵³)—C(R⁵³)(R⁵³)—O—, —N(R⁵²)—C(R⁵³)(R⁵³)—C(R⁵³)═C(R⁵³)—O—, —N(R⁵²)—C(R⁵³)═C(R⁵³)—C(R⁵³)(R⁵³)—O—, —S—C(R⁵³)(R⁵³)—N(R⁵²)—, —S—C(R⁵³)(R⁵³)—C(R⁵³)(R⁵³)—N(R⁵²)—, —S—C(R⁵³)═C(R⁵³)—N(R⁵²)—, —S—C(R⁵³)(R⁵³)—C(R⁵³)(R⁵³)—C(R⁵³)(R⁵³)—N(R⁵²)—, —S—C(R⁵³)(R⁵³)—C(R⁵³)═C(R⁵³)—N(R⁵²)—, —S—C(R⁵³)═C(R⁵³)—C(R⁵³)(R⁵³)—N(R⁵²)—, —N(R⁵²)—C(R⁵³)(R⁵³)—S—, —N(R⁵²) C(R⁵³)(R⁵³)—C(R⁵³)(R⁵³)—S—, —N(R⁵²)—C(R⁵³)═C(R⁵³)—S—, —N(R⁵²)—C(R⁵³)(R⁵³)—C(R⁵³)(R⁵³)—C(R⁵³)(R⁵³)—S—, —N(R⁵²)—C(R⁵³)(R⁵³)—C(R⁵³)═C(R⁵³)—S—, —N(R⁵²)—C(R⁵³)═C(R⁵³)—C(R⁵³)(R⁵³)—S—, —N(R⁵²)—C(R⁵³)(R⁵³)—N(R⁵²)—, —N(R⁵²)—C(R⁵³)(R⁵³)—C(R⁵³)(R⁵³)—N(R⁵²)—, —N(R⁵²)—C(R⁵³)═C(R⁵³)—N(R⁵²)—, —N(R⁵²)—C(R⁵³)(R⁵³)—C(R⁵³)(R⁵³)—C(R⁵³)(R⁵³)—N(R⁵²)—, —N(R⁵²)—C(R⁵³)(R⁵³)—C(R⁵³)═C(R⁵³)—N(R⁵²)— and —N(R⁵²)—C(R⁵³)═C(R⁵³)—C(R⁵³)(R⁵³)—N(R⁵²)—.

Even more preferably, R^{5b} is selected such that the compound of formula (Ia) has any one of the following structures:

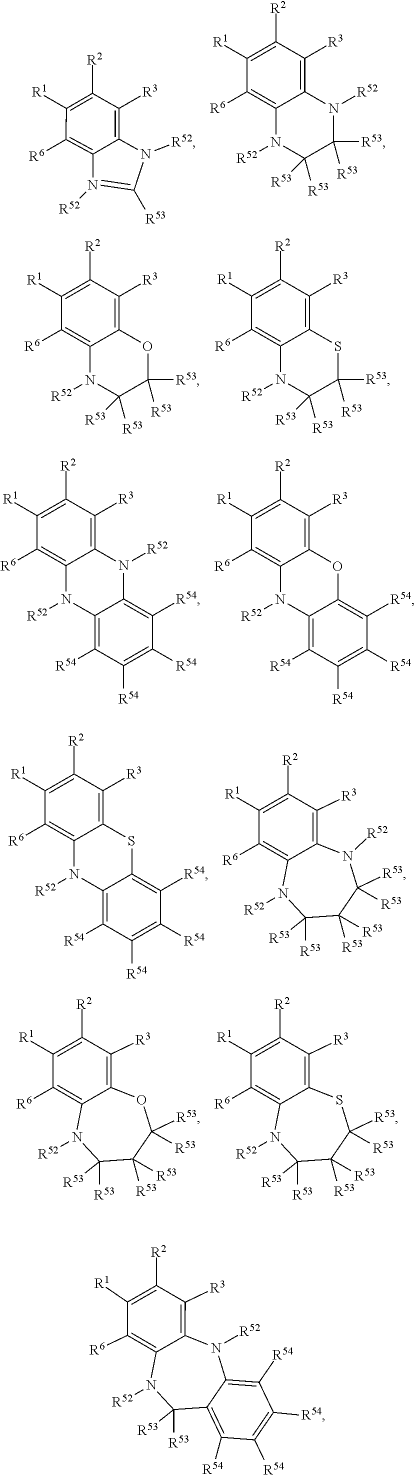

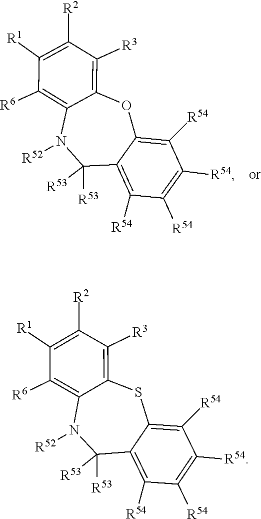

R⁶ is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, R⁶ is selected from hydrogen, C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN. More preferably, R⁶ is hydrogen or C₁₋₄ alkyl. Even more preferably, R⁶ is hydrogen.

Moreover, the invention provides a compound of the following formula (Ib) or a pharmaceutically acceptable salt or solvate thereof (and it also relates to said compound for use as a medicament, and to a pharmaceutical composition comprising said compound and a pharmaceutically acceptable excipient):

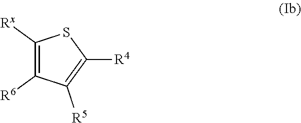

In formula (Ib), R^{x} is selected from —COOH, —COO—(C₁₋₆ alkyl), —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one group selected from —NH₂, —NH(C₁₋₄ alkyl) and —N(C₁₋₄ alkyl)(C₁₋₄ alkyl). Preferably, R^{x} is selected from —COOH, —COOCH₃, —SO₂—NH₂ and —SO₂—N═CH—N(CH₃)₂.

R⁴ is a group R^{4a}, and R⁵ is a group R^{5a}.

R^{4a} is selected from —O—R⁴¹, —S—R⁴¹, —NH—R⁴¹, —N(C₁₋₆ alkyl)-R⁴¹, halogen (e.g., —Cl), carbocyclyl and heterocyclyl, wherein said carbocyclyl and said heterocyclyl are each optionally substituted with one or more groups R⁴². Preferably, R^{4a} is selected from —O—R⁴¹, —S—R⁴¹, —NH—R⁴¹, —N(C₁₋₆ alkyl)-R⁴¹, halogen, carbocyclyl (e.g., aryl, cycloalkyl, or cycloalkenyl) and heterocyclyl (e.g., heteroaryl, heterocycloalkyl, or heterocycloalkenyl), wherein said carbocyclyl and said heterocyclyl are each optionally substituted with one or more groups R⁴². More preferably, R^{4a} is selected from —O—R⁴¹, —S—R⁴¹, —NH—R⁴¹, —N(C₁₋₄ alkyl)-R⁴¹, halogen, aryl and heteroaryl, wherein said aryl and said heteroaryl are each optionally substituted with one or more groups R⁴². Even more preferably, R^{4a} is selected from —O—R⁴¹, —S—R⁴¹, —NH—R⁴¹, —N(C₁₋₄ alkyl)-R⁴¹ and halogen. Still more preferably, R^{4a} is —O—R⁴¹ or halogen.

R⁴¹ is selected from —(C₁₋₄ alkylene)-carbocyclyl, —(C₁₋₄ alkylene)-heterocyclyl, C₁₋₆ alkyl, C₂₋₆ alkenyl and C₂₋₆ alkynyl, wherein the carbocyclyl moiety of said —(C₁₋₄ alkylene)-carbocyclyl and the heterocyclyl moiety of said —(C₀₋₄ alkylene)-heterocyclyl are each optionally substituted with one or more groups R⁴², and wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more groups R⁴³. Preferably, R⁴¹ is selected from —(C₁₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₁₋₄ alkylene)-carbocyclyl is selected from cycloalkyl, cycloalkenyl and aryl, wherein the heterocyclyl moiety of said —(C₀₋₄ alkylene)-heterocyclyl is selected from heterocycloalkyl, heterocycloalkenyl and heteroaryl, wherein the carbocyclyl moiety of said —(C₀₋₄ alkylene)-carbocyclyl and the heterocyclyl moiety of said —(C₀₋₄ alkylene)-heterocyclyl are each optionally substituted with one or more (e.g., one, two or three) groups R⁴², and further wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₀₋₄ alkylene)-heterocyclyl are each optionally substituted with one or more (e.g., one, two or three) groups R⁴³. More preferably, R⁴¹ is selected from —(C₀₋₄ alkylene)-aryl and —(C₀₋₄ alkylene)-heteroaryl, wherein the aryl moiety of said —(C₀₋₄ alkylene)-aryl and the heteroaryl moiety of said —(C₀₋₄ alkylene)-heteroaryl are each optionally substituted with one or more (e.g., one, two or three) groups R⁴², and further wherein the alkylene moiety of said —(C₀₋₄ alkylene)-aryl and the alkylene moiety of said —(C₀₋₄ alkylene)-heteroaryl are each optionally substituted with one or more (e.g., one, two or three) groups R⁴³. Even more preferably, R⁴¹ is selected from —(C₀₋₄ alkylene)-aryl and —(C₀₋₄ alkylene)-heteroaryl, wherein the aryl moiety of said —(C₀₋₄ alkylene)-aryl and the heteroaryl moiety of said —(C₀₋₄ alkylene)-heteroaryl are each optionally substituted with one or more (e.g., one, two or three) groups R⁴². A preferred example of the aryl moiety of said —(C₀₋₄ alkylene)-aryl is phenyl. A preferred example of the heteroaryl moiety of said —(C₀₋₄ alkylene)-heteroaryl is a 5- or 6-membered monocyclic heteroaryl having 1 or 2 ring heteroatoms independently selected from oxygen, nitrogen and sulfur (wherein the remaining ring atoms are carbon atoms), such as, e.g., imidazolyl, thiophenyl, or pyrimidinyl. Still more preferably, R⁴¹ is selected from phenyl and heteroaryl, wherein said heteroaryl is a 5- or 6-membered monocyclic heteroaryl having 1 or 2 ring heteroatoms independently selected from oxygen, nitrogen and sulfur (the remaining ring atoms of the monocyclic heteroaryl are carbon atoms), and further wherein said phenyl or said heteroaryl is optionally substituted with one or more (e.g., one, two or three) groups R⁴².

Each R⁴² is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, each R⁴² is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

Each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃, —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl) and —N(C₁₋₆ alkyl)-CO—(C₁₋₆ alkyl). Preferably, each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃ and —CN.

In accordance with the above definitions, it is particularly preferred that R^{4a} is —O-aryl or halogen, wherein the aryl moiety of said —O-aryl is optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN. Even more preferably, R^{4a} is —O— phenyl or halogen (such as, in particular, —Cl), wherein the phenyl moiety of said —O-phenyl is optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN.

R^{5a} is selected from —NH₂, —NH(C₁₋₆ alkyl) and —N(C₁₋₆ alkyl)(C₁₋₆ alkyl), wherein the alkyl moiety of said —NH(C₁₋₆ alkyl) and one or both of the alkyl moieties of said —N(C₁₋₆ alkyl)(C₁₋₆ alkyl) are each optionally substituted with one or more groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH, —S(C₁₋₆ alkyl), carbocyclyl and heterocyclyl, wherein said carbocyclyl and said heterocyclyl are each optionally substituted with one or more groups R⁵¹. Preferably, R^{5a} is selected from —NH₂, —NH(C₁₋₆ alkyl) and —N(C₁₋₆ alkyl)(C₁₋₆ alkyl), wherein the alkyl moiety of said —NH(C₁₋₆ alkyl) and one or both of the alkyl moieties of said —N(C₁₋₆ alkyl)(C₁₋₆ alkyl) are each optionally substituted with one or more (e.g., one, two or three) groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl). More preferably, R^{5a} is selected from —NH₂, —NH(C₁₋₆ alkyl) and —N(C₁₋₆ alkyl)(C₁₋₆ alkyl). Particularly preferred examples of R^{5a} are —NH₂ or —NH—CH₂CH₂CH₂CH₃.

Each R⁵¹ is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁ alkyl). Preferably, each R⁵¹ is independently selected from C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN.

R⁶ is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl). Preferably, R⁶ is selected from hydrogen, C₁₋₆ alkyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl) and —CN. More preferably, R⁶ is hydrogen or C₁₋₄ alkyl. Even more preferably, R⁶ is hydrogen.

The invention further relates to a compound of any one of the following formulae, or a pharmaceutically acceptable salt or solvate thereof (and it also relates to said compound for use as a medicament, and to a pharmaceutical composition comprising said compound and a pharmaceutically acceptable excipient):

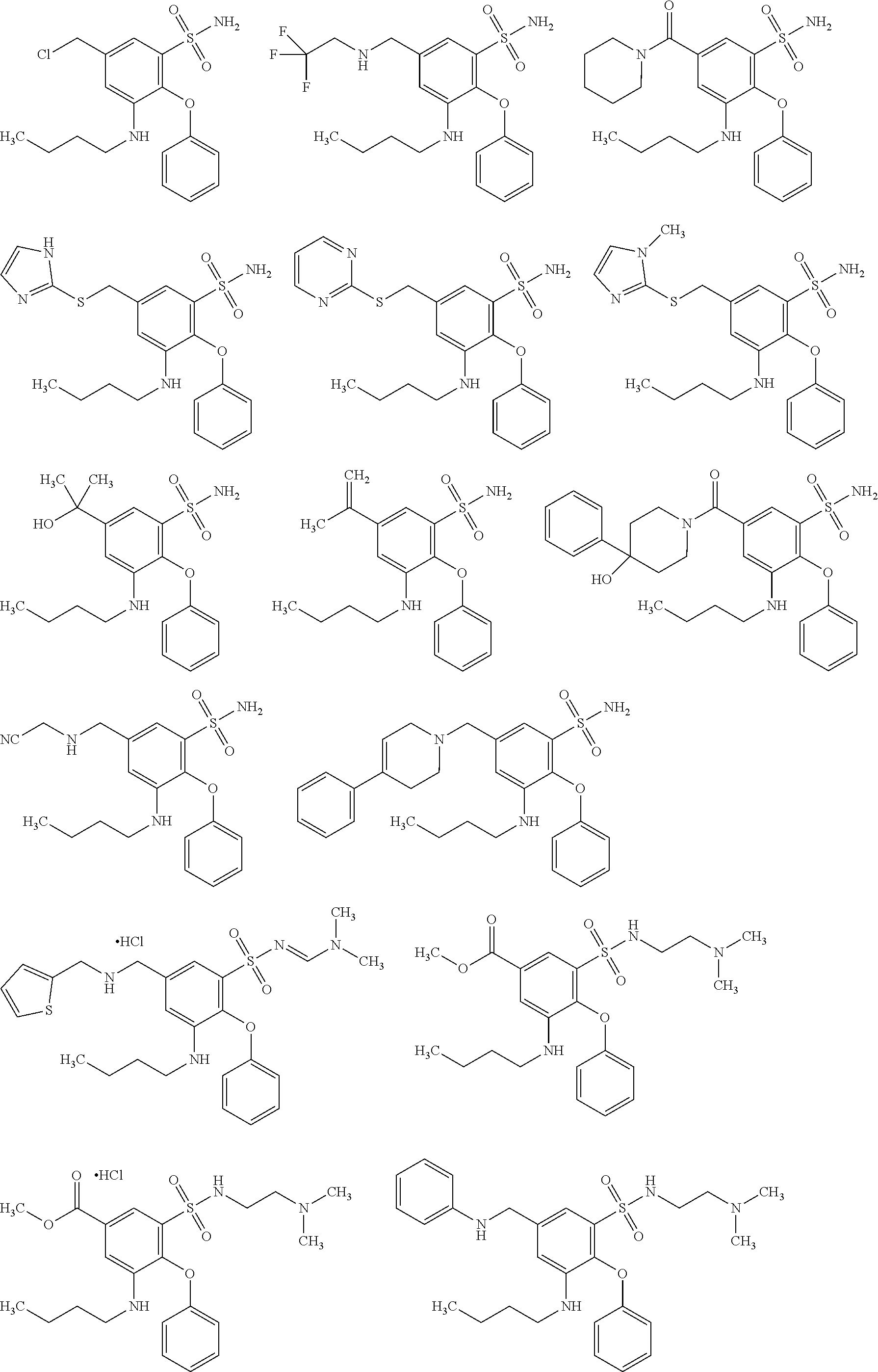

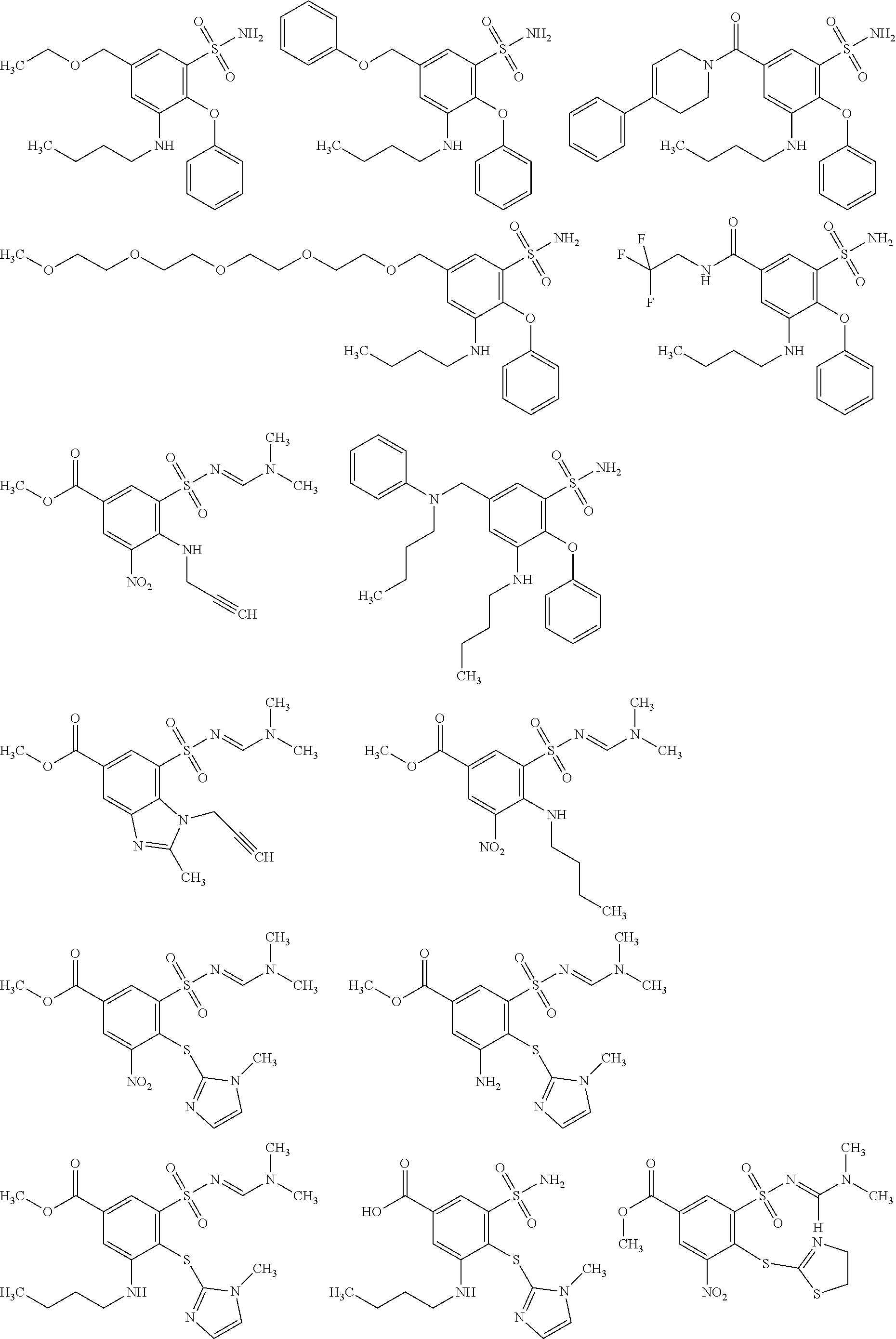

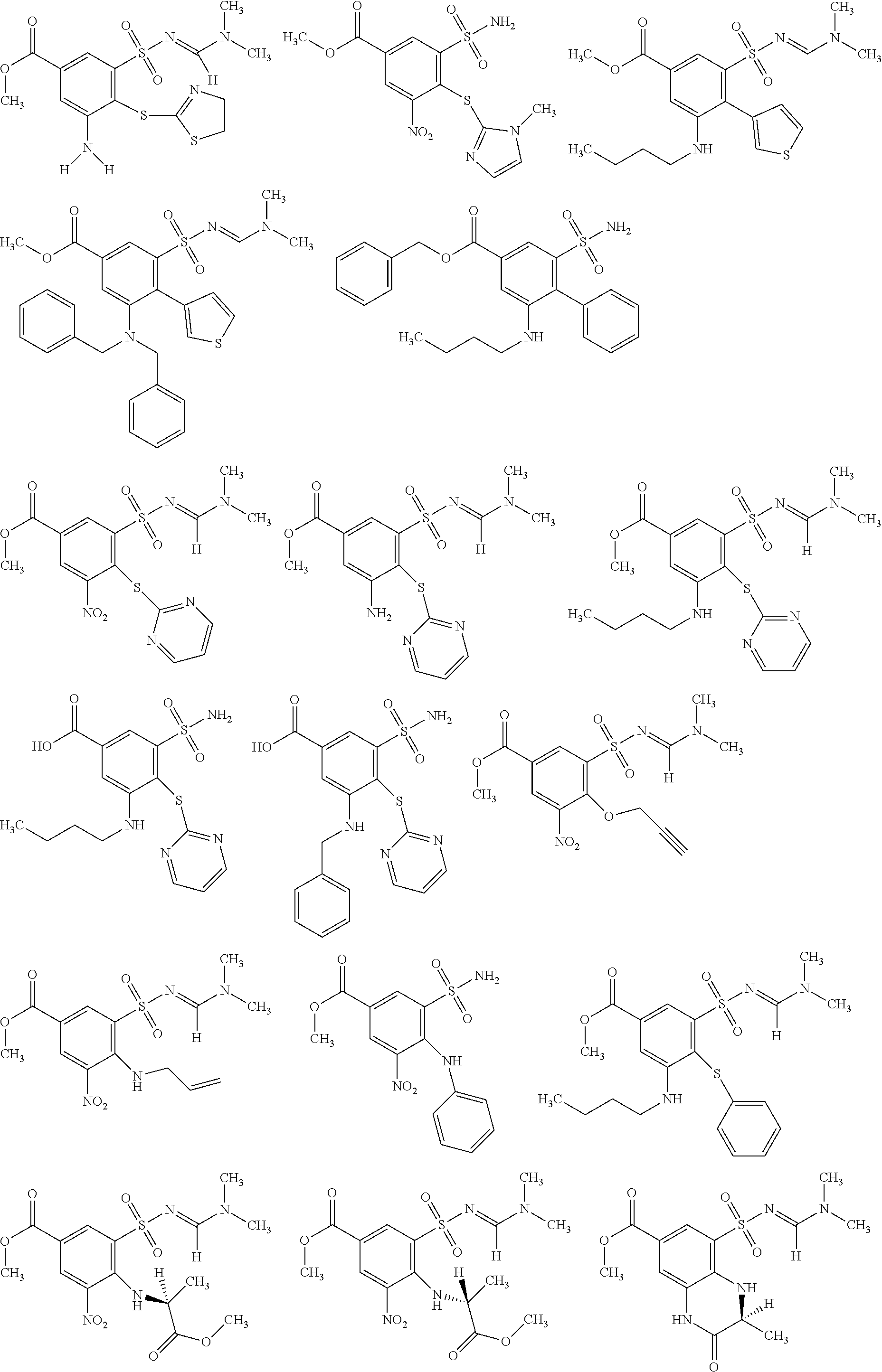

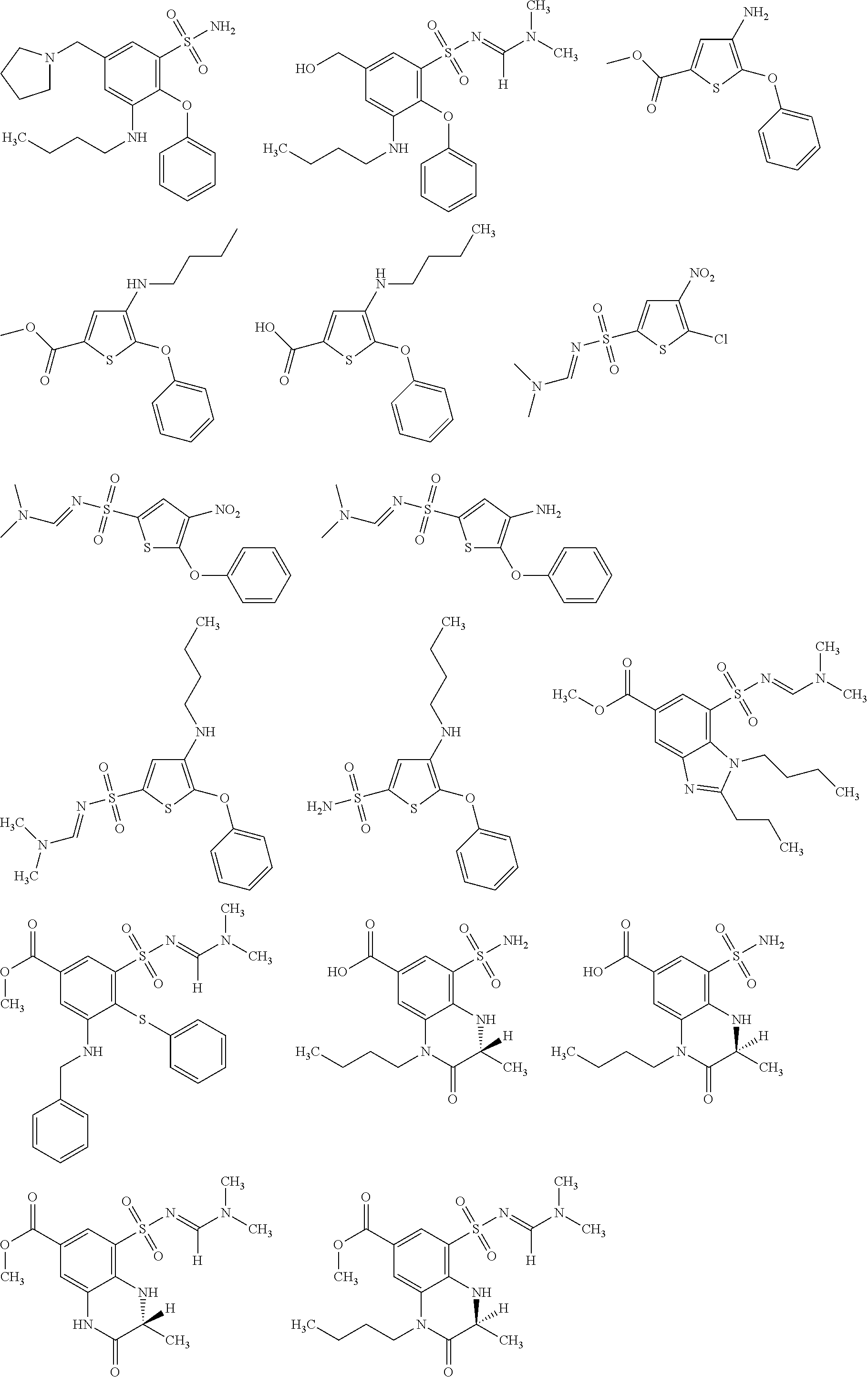

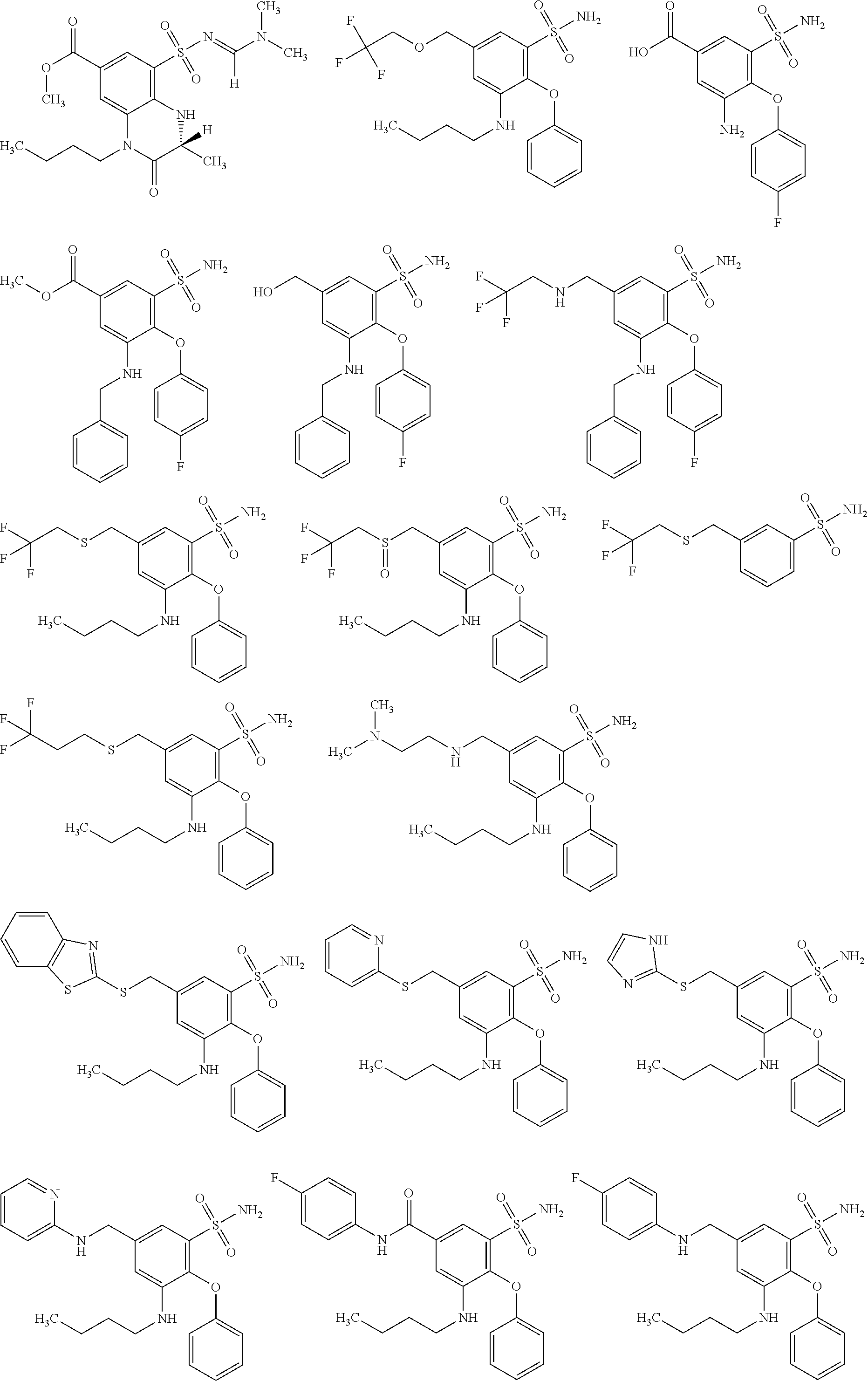

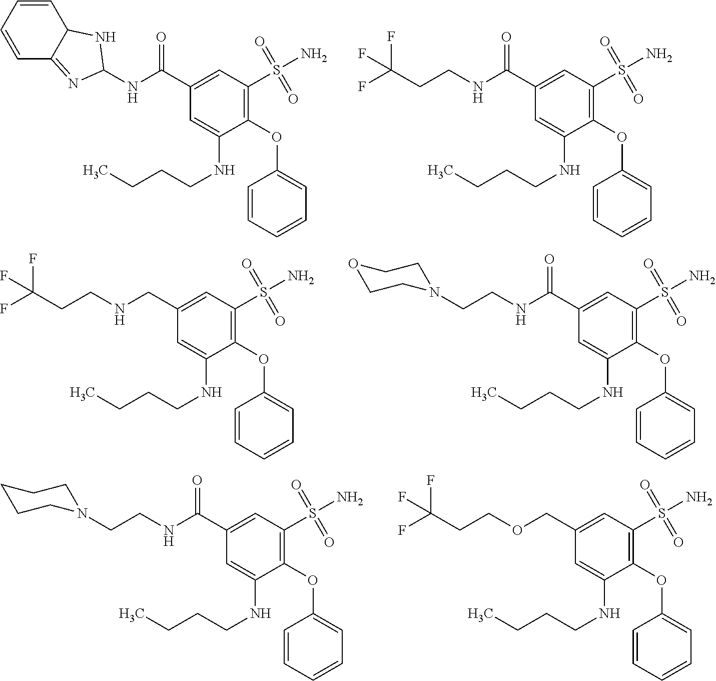

Error! Objects cannot be created from editing field codes.Error! Objects cannot be created from editing field codes.Error! Objects cannot be created from editing field codes.

For a person skilled in the field of synthetic chemistry, various ways for the preparation of the compounds of formula (I) will be readily apparent. For example, the compounds of formula (I) can be prepared in accordance with or in analogy to the synthetic routes described in the examples section.

The following definitions apply throughout the present specification, unless specifically indicated otherwise.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₆ alkyl” denotes an alkyl group having 1 to 6 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₆ alkenyl” denotes an alkenyl group having 2 to 6 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more carbon-to-carbon double bonds. The term “C₂₋₆ alkynyl” denotes an alkynyl group having 2 to 6 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₁₅ alkylene” denotes an alkylene group having 1 to 15 carbon atoms, and the term “C₀₋₁₅ alkylene” indicates that a covalent bond (corresponding to the option “Co alkylene”) or a C₁₋₁₅ alkylene is present. Preferred exemplary alkylene groups are methylene (—CH₂—), ethylene (e.g., —CH₂—CH₂— or —CH(—CH₃)-), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “alkenylene” refers to an alkenediyl group, i.e. a divalent unsaturated acyclic hydrocarbon group which may be linear or branched and comprises one or more (e.g., one or two) carbon-to-carbon double bonds while it does not comprise any carbon-to-carbon triple bond. A “C₂₋₁₅ alkenylene” denotes an alkenylene group having 2 to 15 carbon atoms. Unless defined otherwise, the term “alkenylene” preferably refers to C₂₋₄ alkenylene (including, in particular, linear C₂₋₄ alkenylene).

As used herein, the term “alkynylene” refers to an alkynediyl group, i.e. a divalent unsaturated acyclic hydrocarbon group which may be linear or branched and comprises one or more (e.g., one or two) carbon-to-carbon triple bonds and optionally one or more carbon-to-carbon double bonds. A “C₂₋₁₅ alkynylene” denotes an alkynylene group having 2 to 15 carbon atoms. Unless defined otherwise, the term “alkynylene” preferably refers to C₂₋₄ alkynylene (including, in particular, linear C₂₋₄ alkynylene).

As used herein, the term “alkylidene” refers to a divalent acyclic hydrocarbon group which may be linear or branched, which is connected to the remainder of the respective compound via a double bond, and which does not comprise any other double bond (i.e., which does not comprise any double bond except for the one that connects the alkylidene group to the remainder of the respective compound) or any triple bond. An alkylidene group may, e.g., be attached to a carbon atom or to a nitrogen atom of the remainder of the respective compound. A “C₁₋₆ alkylidene” denotes an alkylidene group having 1 to 6 carbon atoms. Preferred exemplary alkylidene groups are methylidene (═CH₂), ethylidene (═CH—CH₃), propylidene (e.g., ═CH—CH₂CH₃ or ═C(—CH₃)—CH₃), or butylidene (e.g., ═CH—CH₂CH₂CH₃, ═C(—CH₃)—CH₂CH₃, or ═CH—CH(—CH₃)—CH₃). Unless defined otherwise, the term “alkylidene” preferably refers to C₁₋₄ alkylidene.

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, β-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazolyl, thiazolyl, 1H-tetrazolyl, 2H-tetrazolyl, thienyl (i.e., thiophenyl), or pyrimidinyl.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members. Moreover, unless defined otherwise, particularly preferred examples of a “cycloalkyl” include cyclohexyl or cyclopropyl, particularly cyclohexyl.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heterocycloalkyl” include tetrahydropyranyl, piperidinyl, piperazinyl, morpholinyl, pyrrolidinyl, or tetrahydrofuranyl.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C3 cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “halogen” refers to fluoro (—F), chloro (—Cl), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is —CF₃.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula ( ).

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint. If the term “about” is used in connection with the endpoint of an open-ended range, it preferably refers to the corresponding range starting from the lower endpoint −10% or from the upper endpoint +10%, more preferably to the range starting from the lower endpoint −5% or from the upper endpoint +5%, and even more preferably to the open-ended range defined by the exact numerical value of the corresponding endpoint. If the term “about” is used in connection with a parameter that is quantified in integers, such as the number of nucleotides in a given nucleic acid, the numbers corresponding to ±10% or ±5% of the indicated numerical value are to be rounded to the nearest integer (using the tie-breaking rule “round half up”).

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The scope of the invention embraces all pharmaceutically or physiologically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Preferred pharmaceutically/physiologically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically/physiologically acceptable salt of the compound of formula (I) is a hydrochloride salt.

Moreover, the scope of the invention embraces the compounds of formula (I) in any solvated form, including, e.g., solvates with water (i.e., as a hydrate) or solvates with organic solvents such as, e.g., methanol, ethanol or acetonitrile (i.e., as a methanolate, ethanolate or acetonitrilate), or in any crystalline form (i.e., as any polymorph), or in amorphous form. It is to be understood that such solvates of the compounds of the formula (I) also include solvates of pharmaceutically acceptable salts of the compounds of the formula (I).

Furthermore, the compounds of formula (I) may exist in the form of different isomers, in particular stereoisomers (including, e.g., geometric isomers (or cis/trans isomers), enantiomers and diastereomers) or tautomers. All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds provided herein.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by 150 atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

The compounds provided herein may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the above described pharmaceutical compositions comprising a compound of formula (I) may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides (see, e.g., U.S. Pat. No. 3,773,919), copolymers of L-glutamic acid and gamma-ethyl-L-glutamate (Sidman, U. et al., Biopolymers 22:547-556 (1983)), poly(2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP133988). Sustained-release pharmaceutical compositions also include liposomally entrapped compounds. Liposomes containing a compound of the present invention can be prepared by methods known in the art, such as, e.g., the methods described in any one of: DE3218121; Epstein et al., Proc. Natl. Acad. Sci. (USA) 82:3688-3692 (1985); Hwang et al., Proc. Natl. Acad. Sci. (USA) 77:4030-4034 (1980); EP0052322; EP0036676; EP088046; EP0143949; EP0142641; JP 83-118008; U.S. Pat. Nos. 4,485,045; 4,544,545; and EP0102324.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to the emulsification/spray drying process disclosed in WO 99/16419 or WO 01/85136. Spray drying of solution formulations of the compounds of the invention can be carried out, e.g., as described generally in the “Spray Drying Handbook”, 5th ed., K. Masters, John Wiley & Sons, Inc., NY (1991), in WO 97/41833, or in WO 03/053411.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Particularly preferred routes of administration are topical administration, oral administration or parenteral administration. For the treatment or prevention of hyperhidrosis, topical administration is even more preferred.

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 2000 mg, particularly 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The compound of formula (I) or a pharmaceutical composition comprising the compound of formula (I) can be administered in monotherapy (e.g., without concomitantly administering any further therapeutic agents, or without concomitantly administering any further therapeutic agents against the same disease that is to be treated or prevented with the compound of formula (I)). However, the compound of formula (I) or a pharmaceutical composition comprising the compound of formula (I) can also be administered in combination with one or more further therapeutic agents, such as, e.g., one or more further therapeutic agents selected from phenobarbital, phenytoin, valproate (or valproic acid), carbamazepine, lamotrigine, levetiracetam, ethosuximide, and pharmaceutically acceptable salts of any of the aforementioned agents. If the compound of formula (I) is used in combination with a second therapeutic agent active against the same disease or condition, the dose of each compound may differ from that when the corresponding compound is used alone, in particular, a lower dose of each compound may be used. The combination of the compound of formula (I) with one or more further therapeutic agents (e.g., one or more of the corresponding exemplary therapeutic agents mentioned above) may comprise the simultaneous/concomitant administration of the compound of formula (I) and the further therapeutic agent(s) (either in a single pharmaceutical formulation or in separate pharmaceutical formulations), or the sequential/separate administration of the compound of formula (I) and the further therapeutic agent(s). If administration is sequential, either the compound of formula (I) according to the invention or the one or more further therapeutic agents may be administered first. If administration is simultaneous, the one or more further therapeutic agents may be included in the same pharmaceutical formulation as the compound of formula (I), or they may be administered in one or more different (separate) pharmaceutical formulations.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “treatment” of a disorder or disease as used herein (e.g., “treatment” of hyperhidrosis) is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease as used herein (e.g., “prevention” of hyperhidrosis) is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

It is to be understood that the present invention specifically relates to each and every combination of features described herein, including any combination of general and/or preferred features. In particular, the invention specifically relates to each combination of meanings (including general and/or preferred meanings) for the various groups and variables comprised in formula ( ).

In this specification, a number of documents including patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The present invention particularly relates to the following items:
1. A compound of any one of the following formulae or a pharmaceutically acceptable salt or solvate thereof:

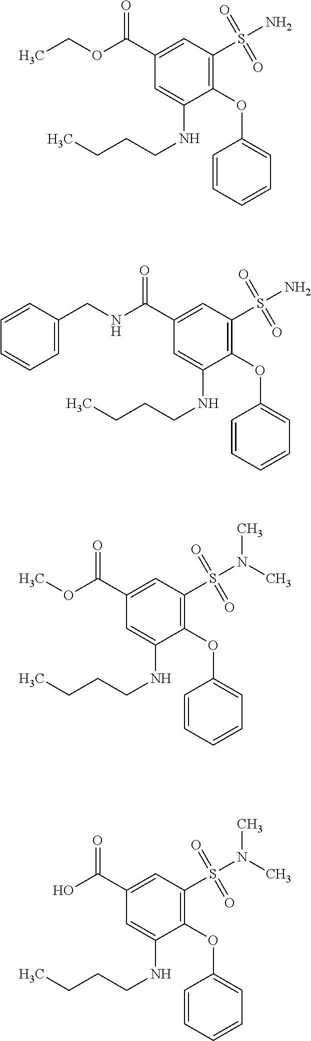

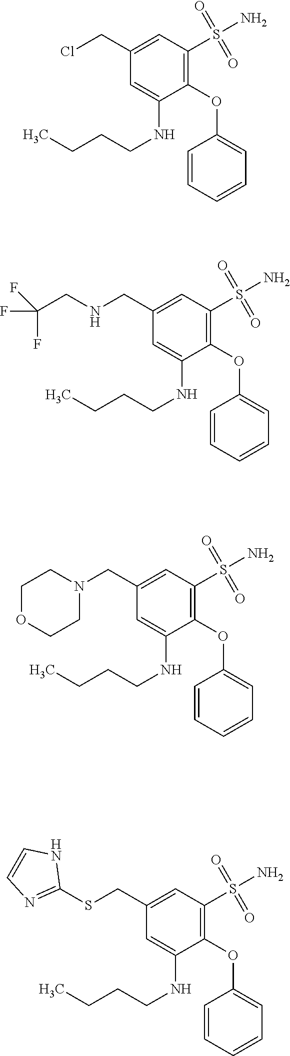 [SMILES: CCCCNc1cc(CCl)cc(S(N)(=O)=O)c1Oc1ccccc1 ; CCCCNc1cc(CNCC(F)(F)F)cc(S(N)(=O)=O)c1Oc1ccccc1 ; CCCCNc1cc(CN2CCOCC2)cc(S(N)(=O)=O)c1Oc1ccccc1 ; CCCCNc1cc(CSc2ncc[nH]2)cc(S(N)(=O)=O)c1Oc1ccccc1]

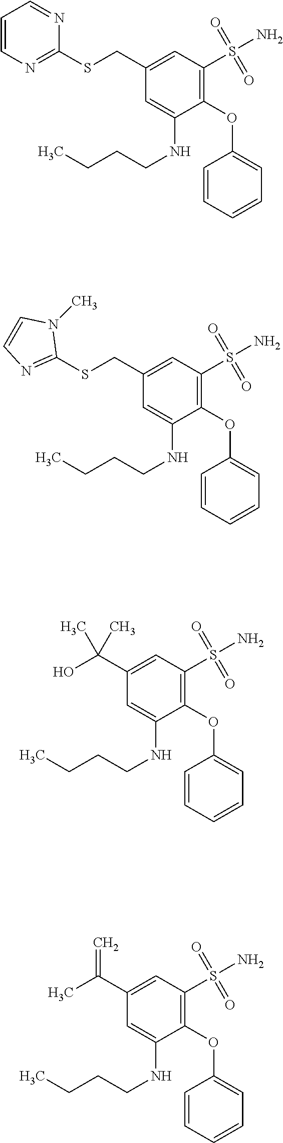 [SMILES: CCCCNc1cc(CSc2ncccn2)cc(S(N)(=O)=O)c1Oc1ccccc1 ; CCCCNc1cc(CSc2nccn2C)cc(S(N)(=O)=O)c1Oc1ccccc1 ; CCCCNc1cc(C(C)(C)O)cc(S(N)(=O)=O)c1Oc1ccccc1 ; C=C(C)c1cc(NCCCC)c(Oc2ccccc2)c(S(N)(=O)=O)c1]

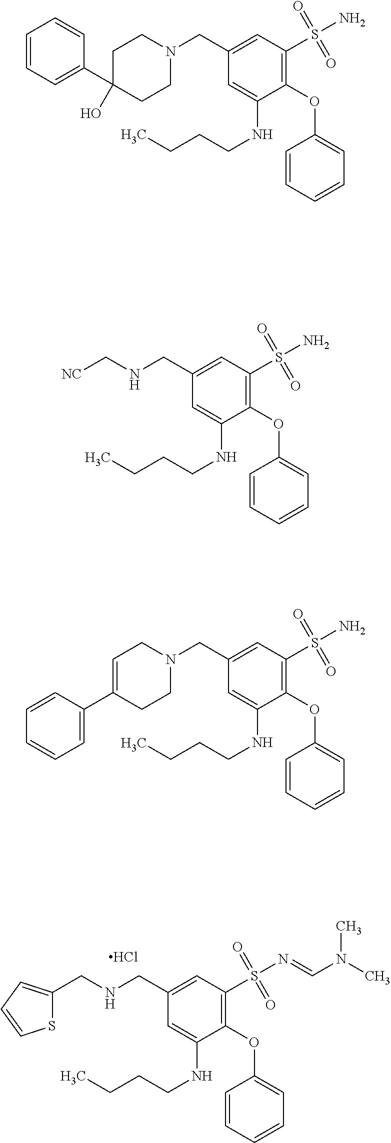 [SMILES: CCCCNc1cc(CN2CCC(O)(c3ccccc3)CC2)cc(S(N)(=O)=O)c1Oc1ccccc1 ; CCCCNc1cc(CNCC#N)cc(S(N)(=O)=O)c1Oc1ccccc1 ; CCCCNc1cc(CN2CC=C(c3ccccc3)CC2)cc(S(N)(=O)=O)c1Oc1ccccc1 ; CCCCNc1cc(CNCc2cccs2)cc(S(=O)(=O)/N=C/N(C)C)c1Oc1ccccc1 ; Cl]

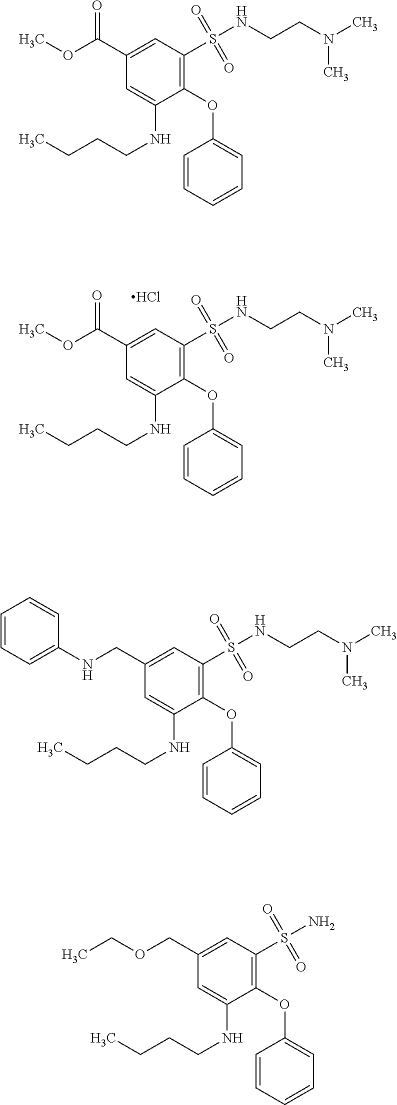

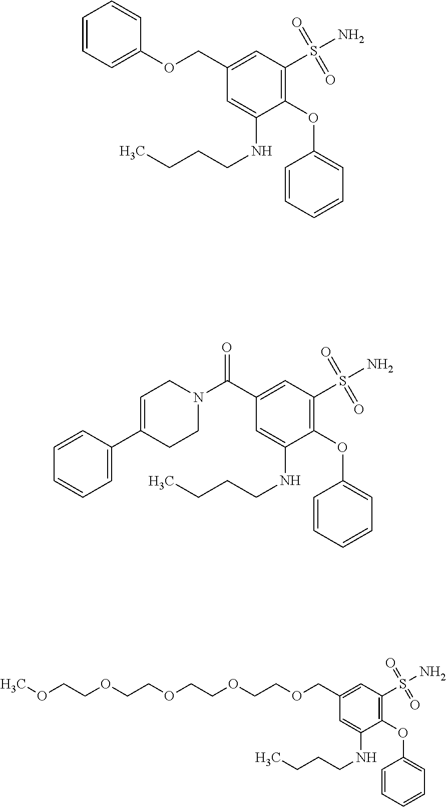 [SMILES: CCCCNc1cc(COc2ccccc2)cc(S(N)(=O)=O)c1Oc1ccccc1 ; CCCCNc1cc(C(=O)N2CC=C(c3ccccc3)CC2)cc(S(N)(=O)=O)c1Oc1ccccc1 ; CCCCNc1cc(COCCOCCOCCOCCOC)cc(S(N)(=O)=O)c1Oc1ccccc1]

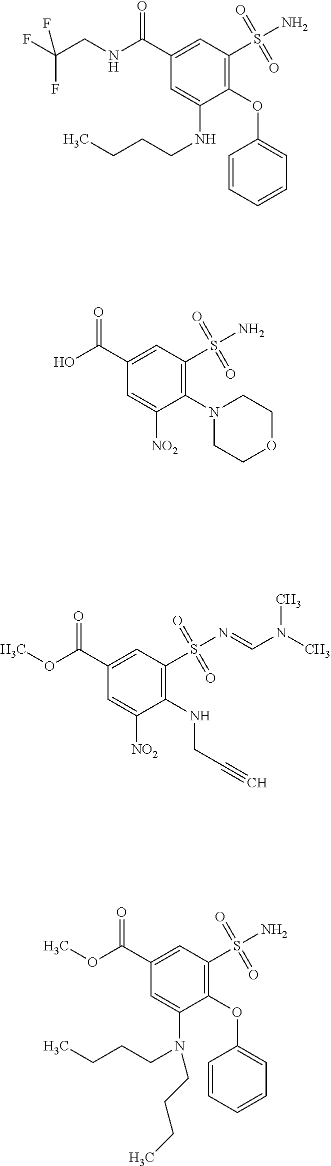

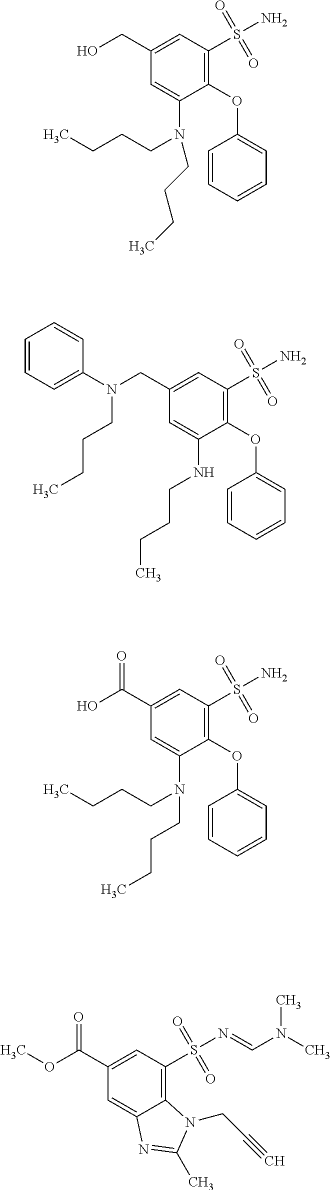

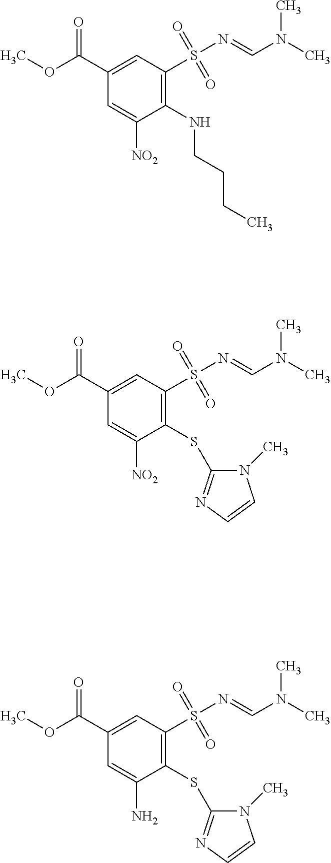

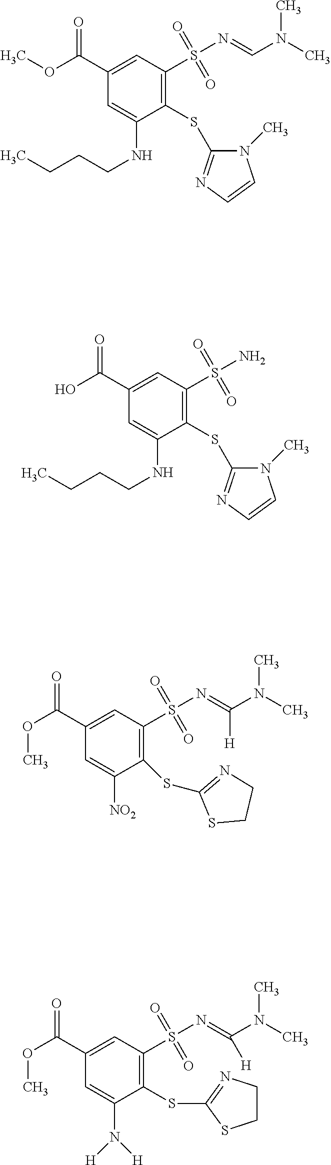

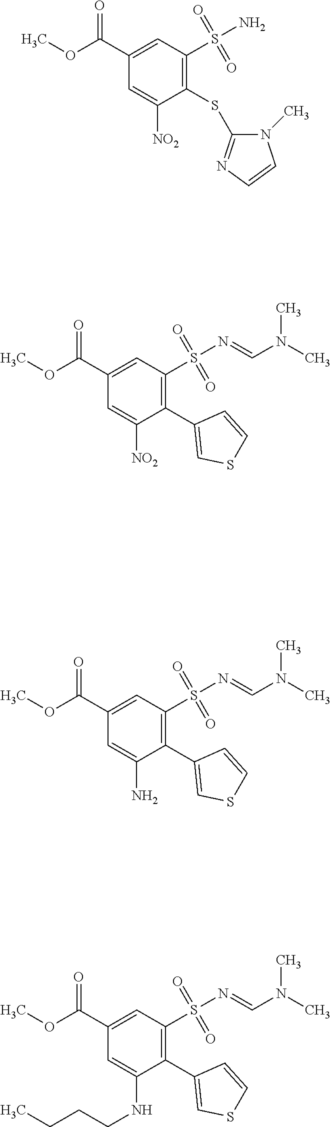

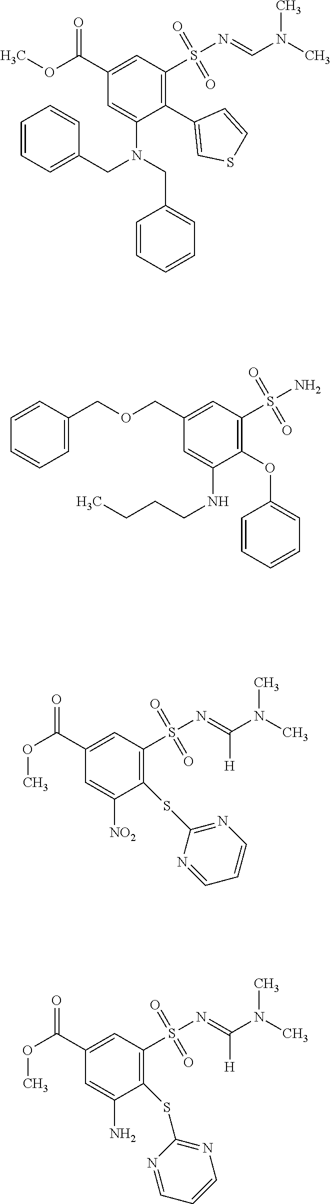

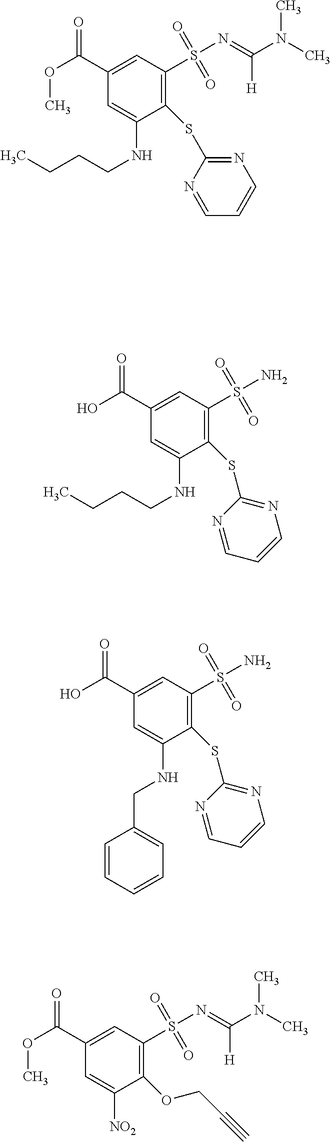

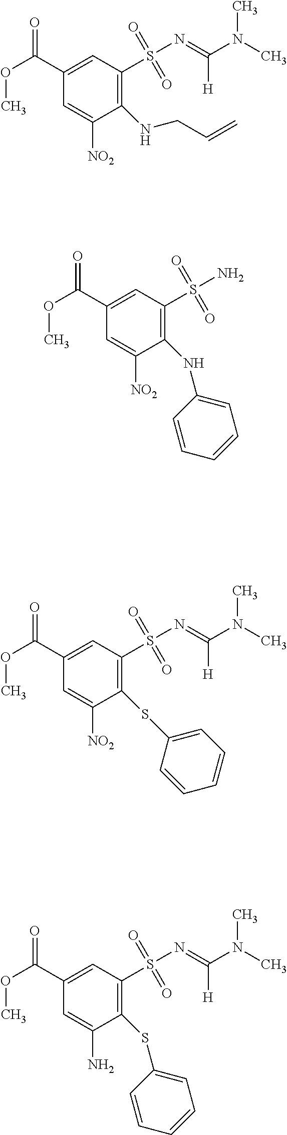 [SMILES: C=CCNc1c([N+](=O)[O-])cc(C(=O)OC)cc1S(=O)(=O)/N=C/N(C)C ; COC(=O)c1cc([N+](=O)[O-])c(Nc2ccccc2)c(S(N)(=O)=O)c1 ; COC(=O)c1cc([N+](=O)[O-])c(Sc2ccccc2)c(S(=O)(=O)/N=C/N(C)C)c1 ; COC(=O)c1cc(N)c(Sc2ccccc2)c(S(=O)(=O)/N=C/N(C)C)c1]

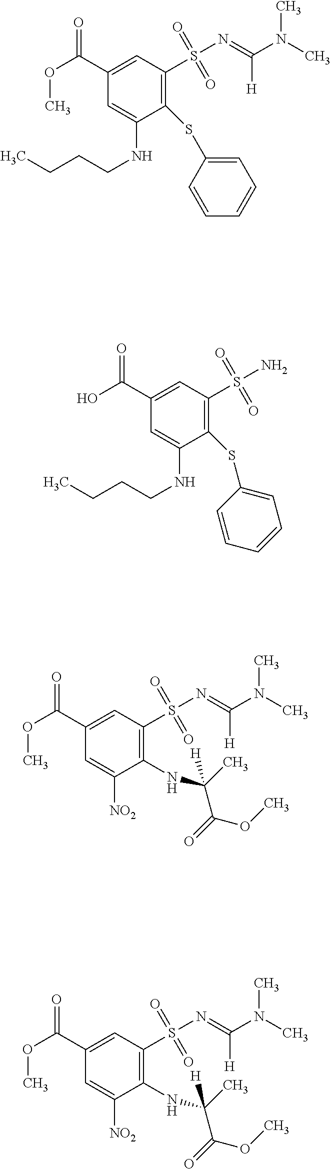

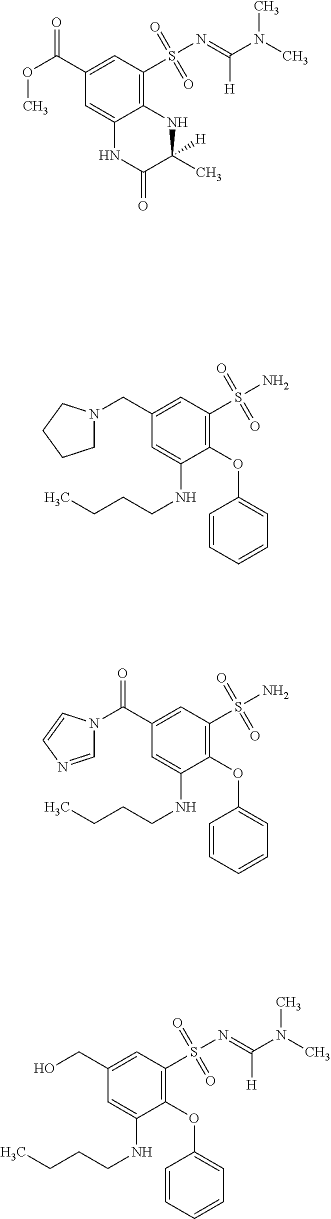

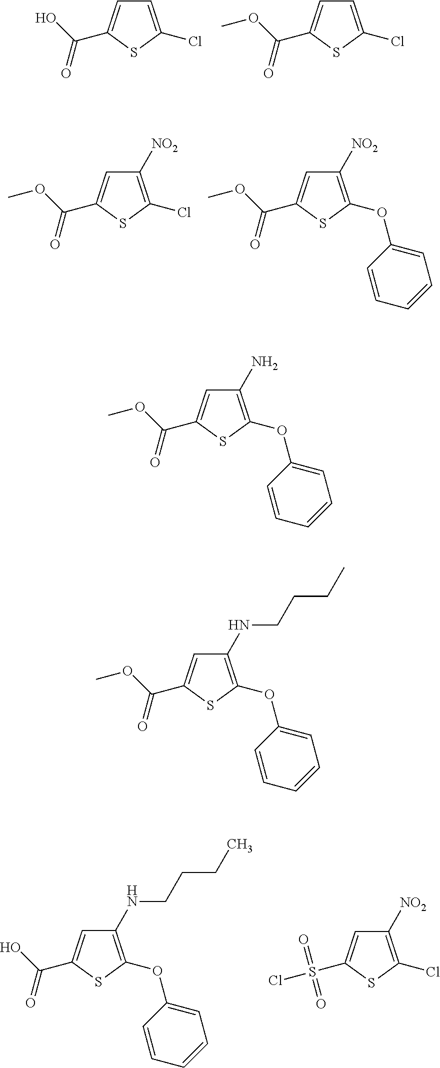

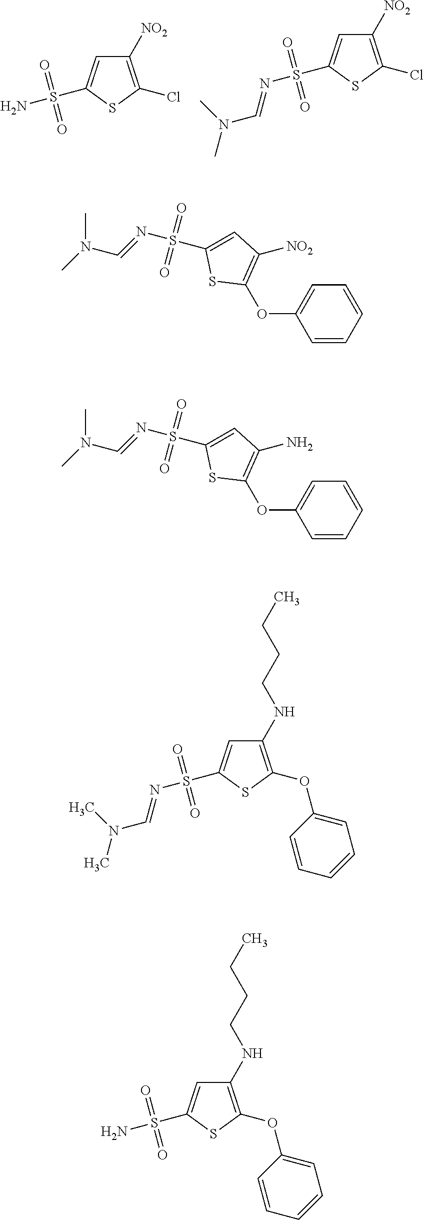

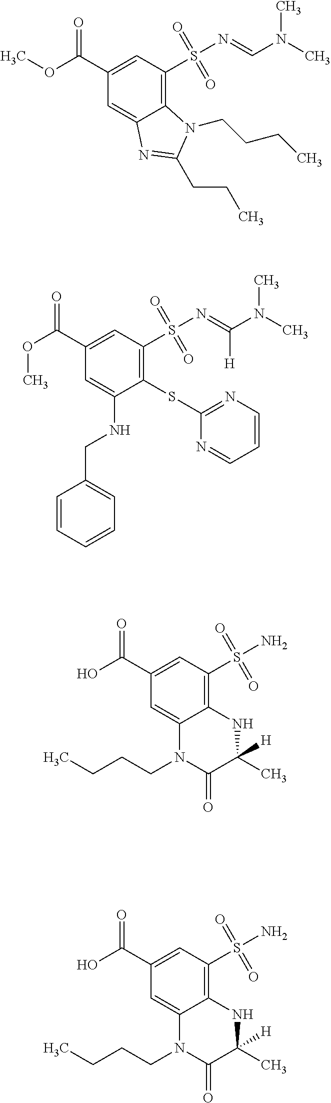

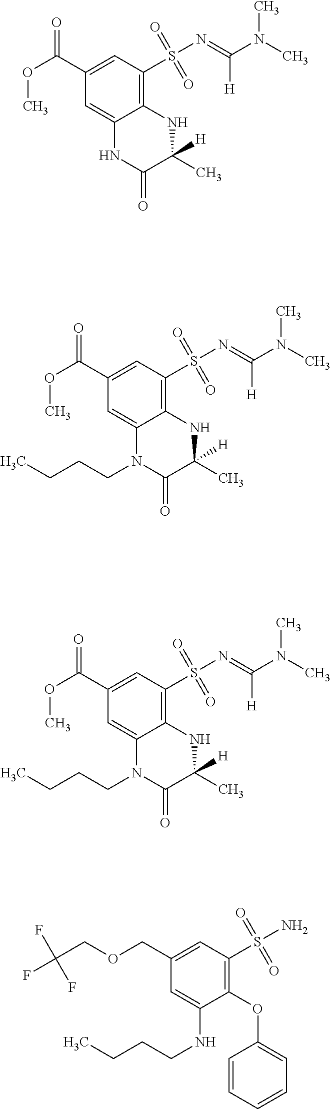

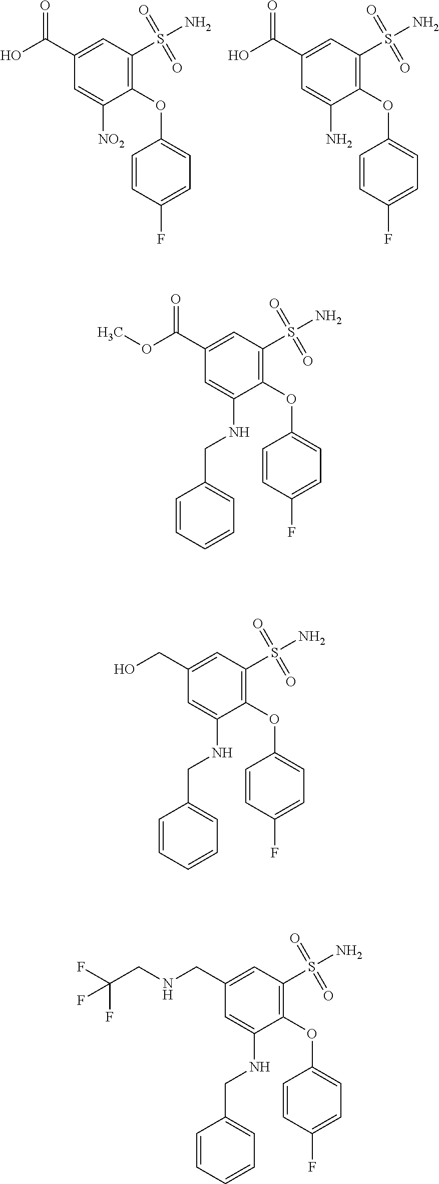 [SMILES: NS(=O)(=O)c1cc(C(=O)O)cc([N+](=O)[O-])c1Oc1ccc(F)cc1 ; Nc1cc(C(=O)O)cc(S(N)(=O)=O)c1Oc1ccc(F)cc1 ; COC(=O)c1cc(NCc2ccccc2)c(Oc2ccc(F)cc2)c(S(N)(=O)=O)c1 ; NS(=O)(=O)c1cc(CO)cc(NCc2ccccc2)c1Oc1ccc(F)cc1 ; NS(=O)(=O)c1cc(CNCC(F)(F)F)cc(NCc2ccccc2)c1Oc1ccc(F)cc1]

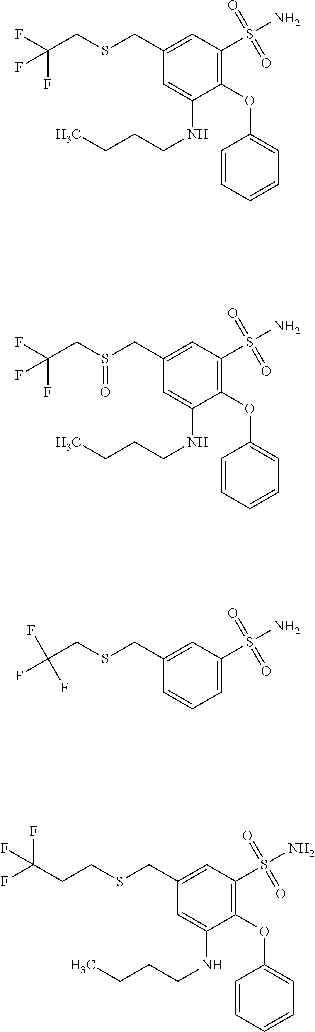 [SMILES: CCCCNc1cc(CSCC(F)(F)F)cc(S(N)(=O)=O)c1Oc1ccccc1 ; CCCCNc1cc(CS(=O)CC(F)(F)F)cc(S(N)(=O)=O)c1Oc1ccccc1 ; NS(=O)(=O)c1cccc(CSCC(F)(F)F)c1 ; CCCCNc1cc(CSCCC(F)(F)F)cc(S(N)(=O)=O)c1Oc1ccccc1]

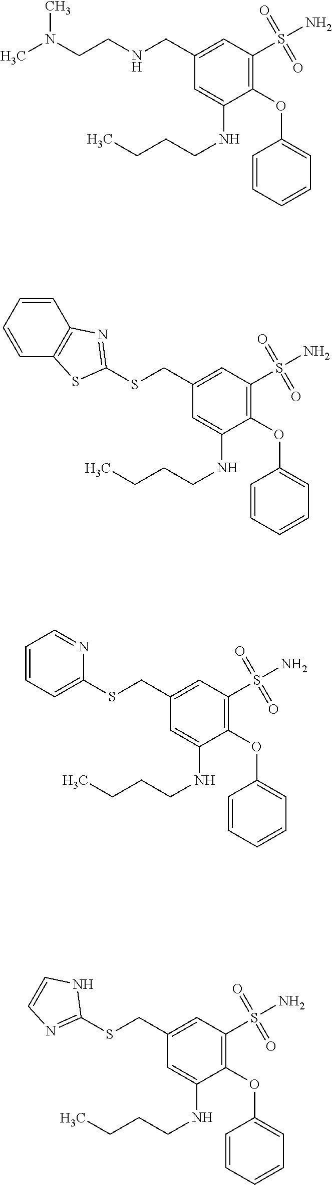 [SMILES: CCCCNc1cc(CNCCN(C)C)cc(S(N)(=O)=O)c1Oc1ccccc1 ; CCCCNc1cc(CSc2nc3ccccc3s2)cc(S(N)(=O)=O)c1Oc1ccccc1 ; CCCCNc1cc(CSc2ccccn2)cc(S(N)(=O)=O)c1Oc1ccccc1 ; CCCCNc1cc(CSc2ncc[nH]2)cc(S(N)(=O)=O)c1Oc1ccccc1]

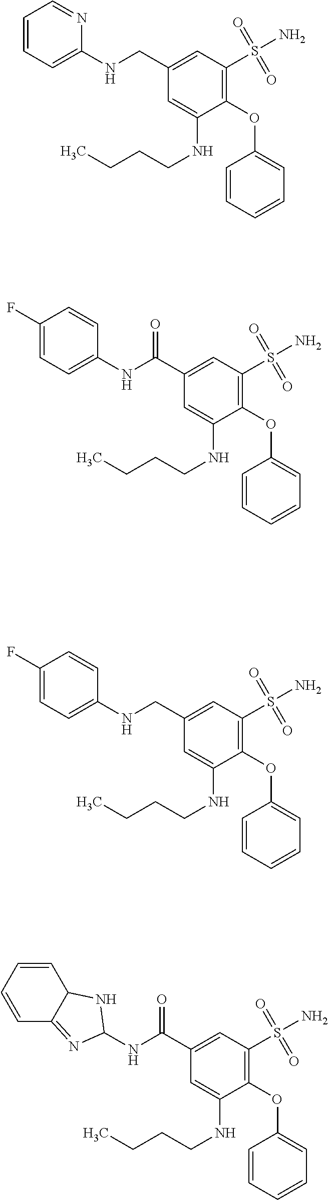 [SMILES: CCCCNc1cc(CNc2ccccn2)cc(S(N)(=O)=O)c1Oc1ccccc1 ; CCCCNc1cc(C(=O)Nc2ccc(F)cc2)cc(S(N)(=O)=O)c1Oc1ccccc1 ; CCCCNc1cc(CNc2ccc(F)cc2)cc(S(N)(=O)=O)c1Oc1ccccc1 ; CCCCNc1cc(C(=O)NC2N=C3C=CC=CC3N2)cc(S(N)(=O)=O)c1Oc1ccccc1]

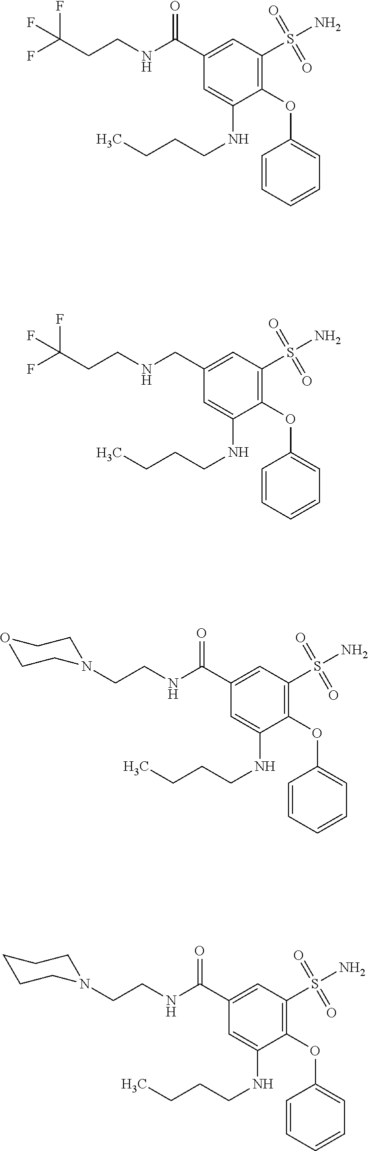 [SMILES: CCCCNc1cc(C(=O)NCCC(F)(F)F)cc(S(N)(=O)=O)c1Oc1ccccc1 ; CCCCNc1cc(CNCCC(F)(F)F)cc(S(N)(=O)=O)c1Oc1ccccc1 ; CCCCNc1cc(C(=O)NCCN2CCOCC2)cc(S(N)(=O)=O)c1Oc1ccccc1 ; CCCCNc1cc(C(=O)NCCN2CCCCC2)cc(S(N)(=O)=O)c1Oc1ccccc1]

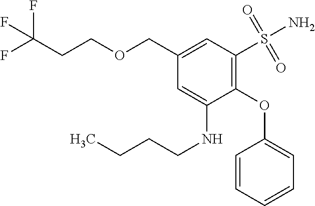 [SMILES: CCCCNc1cc(COCCC(F)(F)F)cc(S(N)(=O)=O)c1Oc1ccccc1]

Error! Objects cannot be created from editing field codes.Error! Objects cannot be created from editing field codes.Error! Objects cannot be created from editing field codes.
for use in the treatment or prevention of a disease or disorder involving an NKCC.
2. A pharmaceutical composition comprising a compound as defined in item 1 and a pharmaceutically acceptable excipient for use in the treatment or prevention of a disease or disorder involving an NKCC.
3. Use of a compound as defined in item 1 in the preparation of a medicament for the treatment or prevention of a disease or disorder involving an NKCC.
4. A method of treating or preventing a disease or disorder involving an NKCC, the method comprising administering a compound as defined in item 1 or a pharmaceutical composition as defined in item 2 to a subject in need thereof.
5. The method of item 4, wherein the subject is a human.
6. The compound for use according to item 1 or the pharmaceutical composition for use according to item 2 or the use of item 3 or the method of item 4 or 5, wherein the disease or disorder involving an NKCC is hyperhidrosis.
7. Non-therapeutic use of a compound as defined in item 1 for suppressing or reducing the sweating of a subject.
8. Non-therapeutic method of suppressing or reducing the sweating of a subject, wherein the method comprises administering a compound as defined in item 1 to the subject.
9. The non-therapeutic use of item 7 or the non-therapeutic method of item 8, wherein said compound is administered topically.
10. The non-therapeutic use of item 7 or 9 or the non-therapeutic method of item 8 or 9, wherein said compound is provided in the form of an article comprising said compound, wherein the article is a wipe, an insole or a garment.
11. An article comprising a compound as defined in item 1, wherein the article is a wipe, an insole or a garment.
12. The compound for use according to item 1 or the pharmaceutical composition for use according to item 2 or the use of item 3 or the method of item 4 or 5, wherein the disease or disorder involving an NKCC is selected from an anxiety disorder, an autism spectrum disorder, autism, Asperger syndrome, childhood disintegrative disorder, a pervasive developmental disorder as part of an autism spectrum disorder, traumatic brain injury, spinal cord injury, peripheral nerve injury, stroke, Alzheimer's disease, schizophrenia, asthma, edema, Down syndrome, mental disability in patients with Down syndrome, glaucoma, primary open angle glaucoma, angle closure glaucoma, and a parasitic infection,
   wherein said parasitic infection is preferably selected from a helminth infection, a hookworm infection, a roundworm infection, a whipworm infection, a tapeworm infection, a guinea worm infection, a pinworm infection, a toxocara infection, a Strongyloides stercoralis infection, an Ascaris lumbricoides infection, a parasitic fluke infection, Schistosomiasis, Gnathostomiasis, Paragonimiasis, Fascioliasis, Swimmer's itch, a protozoan infection, malaria, amoebiasis, giardiasis, African sleeping sickness, toxoplasmosis, Acanthamoeba keratitis, leishmaniasis, babesiosis, granulomatous amoebic encephalitis, cryptosporidiosis, cyclosporiasis, primary amoebic meningoencephalitis, an ectoparasitic infection, an infection with Sarcoptes scabiei, an infection with Pediculus humanus capitis, an infection with Phthirus pubis, an infection with human botfly maggots, an infection with Tunga penetrans, and an infection with Ixodoidea.
13. In vitro use of a compound as defined in item 1 as an NKCC inhibitor.
14. An in vitro method of inhibiting NKCC, the method comprising the application of a compound as defined in item 1.
15. A compound of any one of the following formulae or a pharmaceutically acceptable salt or solvate thereof:

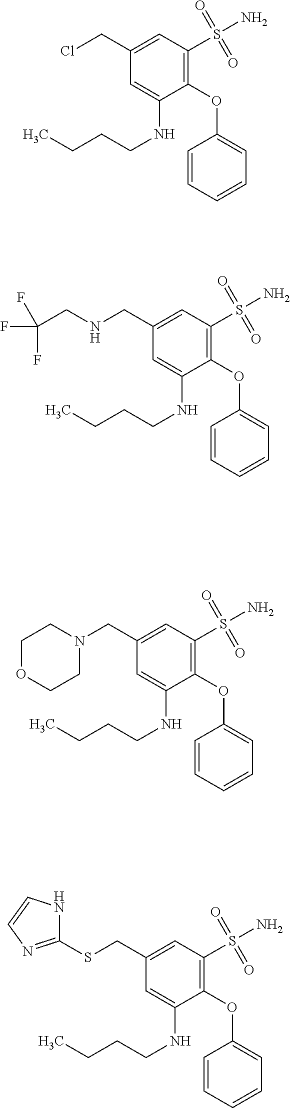 [SMILES: CCCCNc1cc(CCl)cc(S(N)(=O)=O)c1Oc1ccccc1 ; CCCCNc1cc(CNCC(F)(F)F)cc(S(N)(=O)=O)c1Oc1ccccc1 ; CCCCNc1cc(CN2CCOCC2)cc(S(N)(=O)=O)c1Oc1ccccc1 ; CCCCNc1cc(CSc2ncc[nH]2)cc(S(N)(=O)=O)c1Oc1ccccc1]

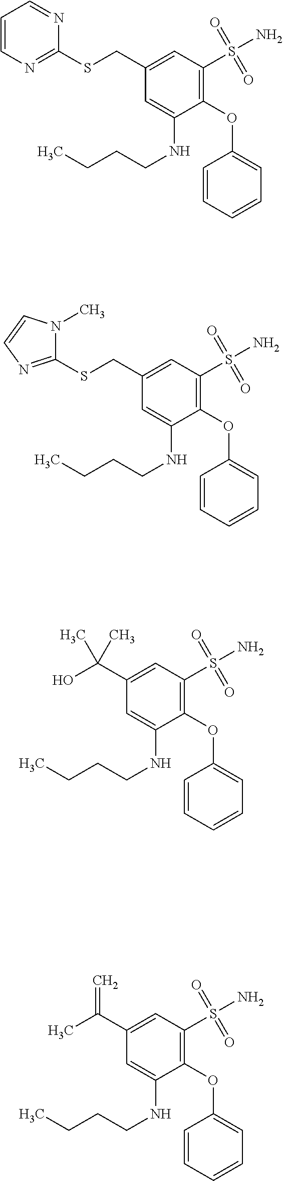 [SMILES: CCCCNc1cc(CSc2ncccn2)cc(S(N)(=O)=O)c1Oc1ccccc1 ; CCCCNc1cc(CSc2nccn2C)cc(S(N)(=O)=O)c1Oc1ccccc1 ; CCCCNc1cc(C(C)(C)O)cc(S(N)(=O)=O)c1Oc1ccccc1 ; C=C(C)c1cc(NCCCC)c(Oc2ccccc2)c(S(N)(=O)=O)c1]

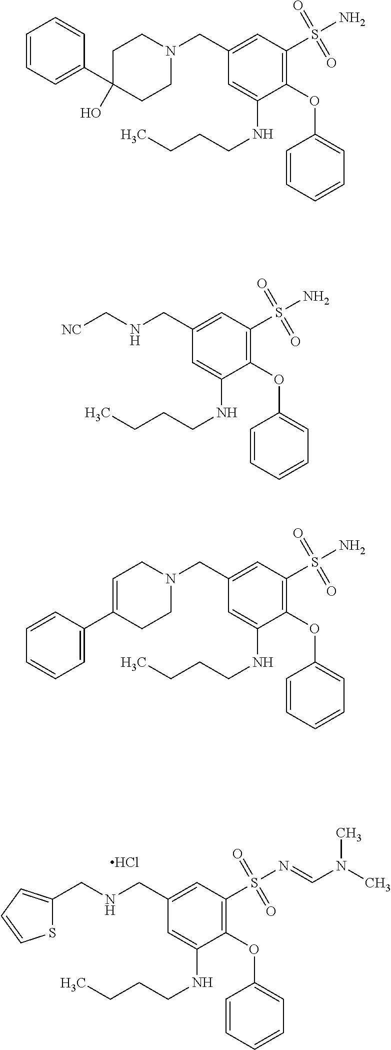 [SMILES: CCCCNc1cc(CN2CCC(O)(c3ccccc3)CC2)cc(S(N)(=O)=O)c1Oc1ccccc1 ; CCCCNc1cc(CNCC#N)cc(S(N)(=O)=O)c1Oc1ccccc1 ; CCCCNc1cc(CN2CC=C(c3ccccc3)CC2)cc(S(N)(=O)=O)c1Oc1ccccc1 ; CCCCNc1cc(CNCc2cccs2)cc(S(=O)(=O)/N=C/N(C)C)c1Oc1ccccc1 ; Cl]

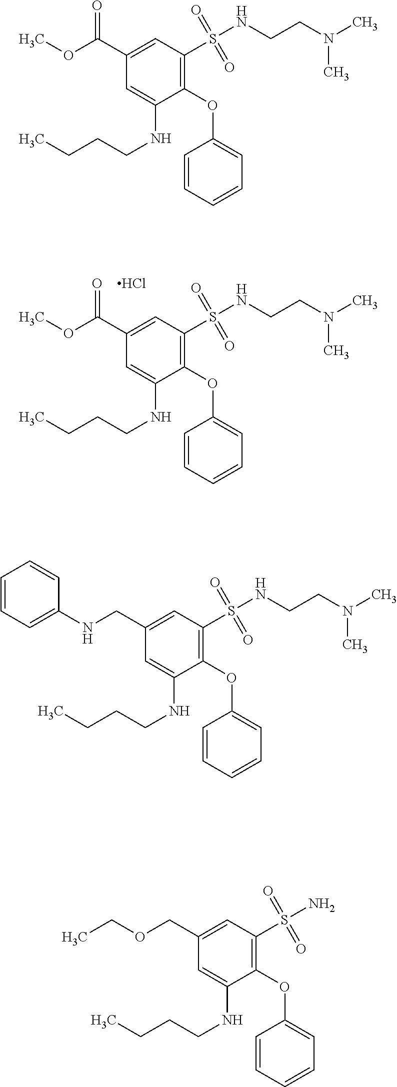

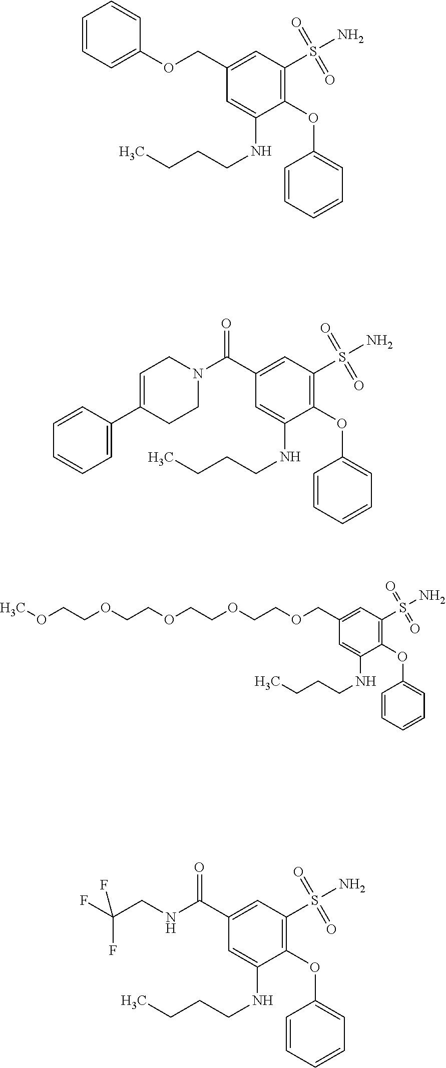 [SMILES: CCCCNc1cc(COc2ccccc2)cc(S(N)(=O)=O)c1Oc1ccccc1 ; CCCCNc1cc(C(=O)N2CC=C(c3ccccc3)CC2)cc(S(N)(=O)=O)c1Oc1ccccc1 ; CCCCNc1cc(COCCOCCOCCOCCOC)cc(S(N)(=O)=O)c1Oc1ccccc1 ; CCCCNc1cc(C(=O)NCC(F)(F)F)cc(S(N)(=O)=O)c1Oc1ccccc1]

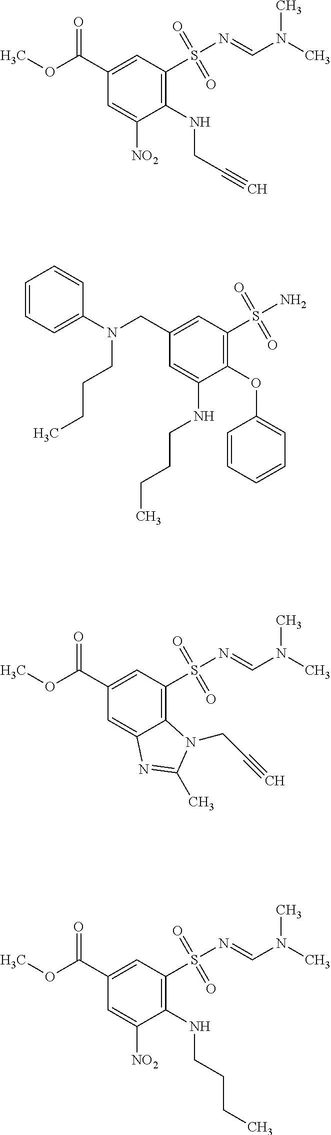

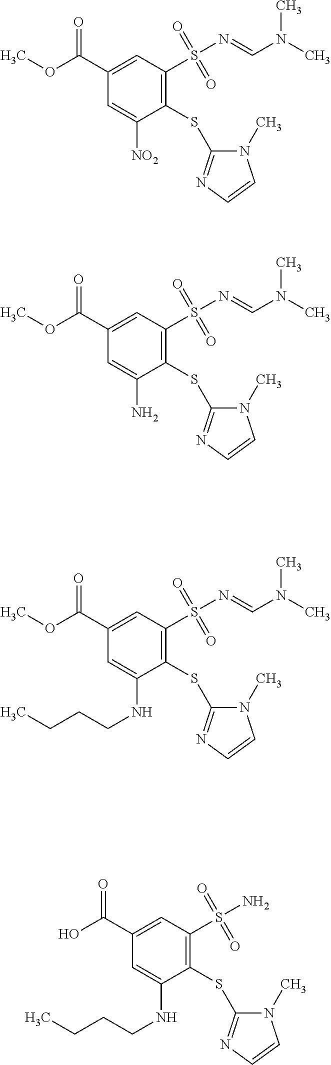

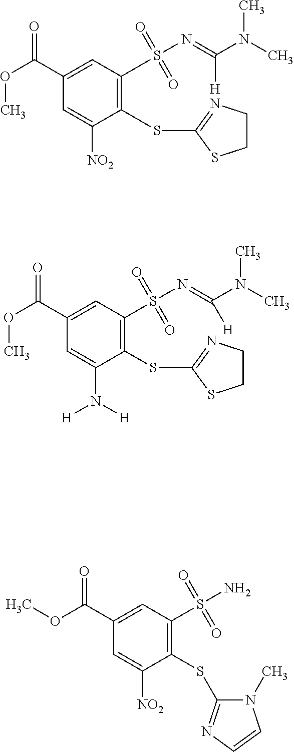

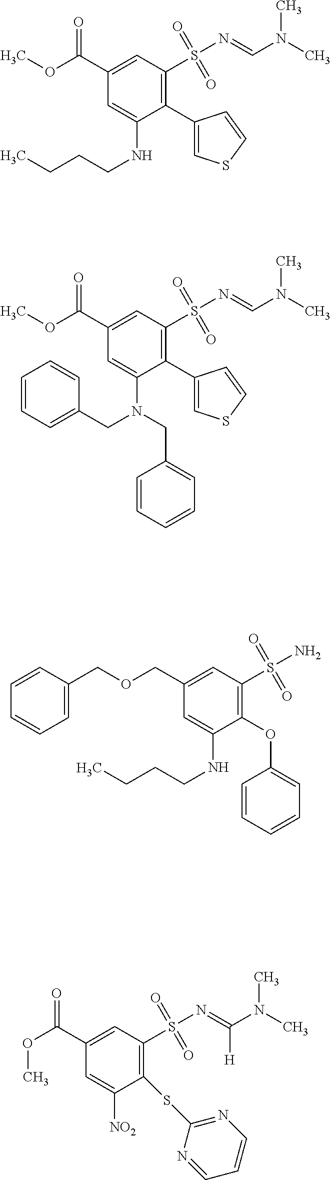

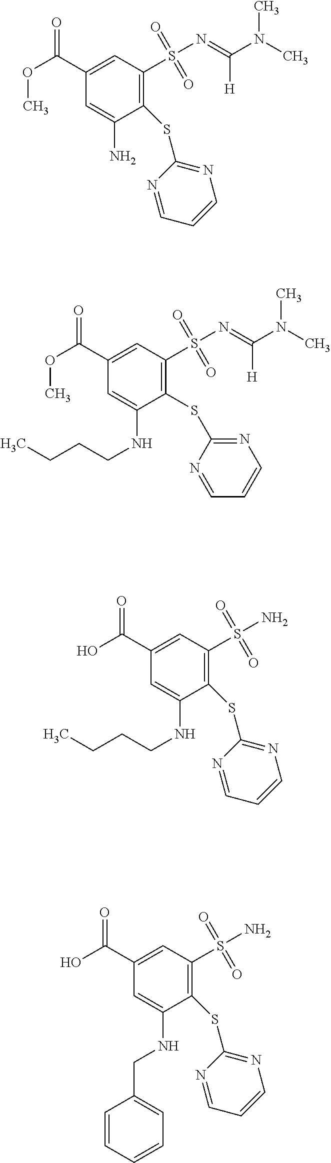

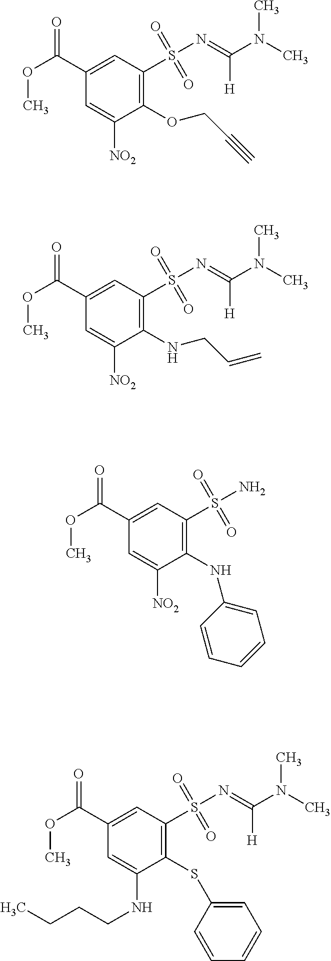

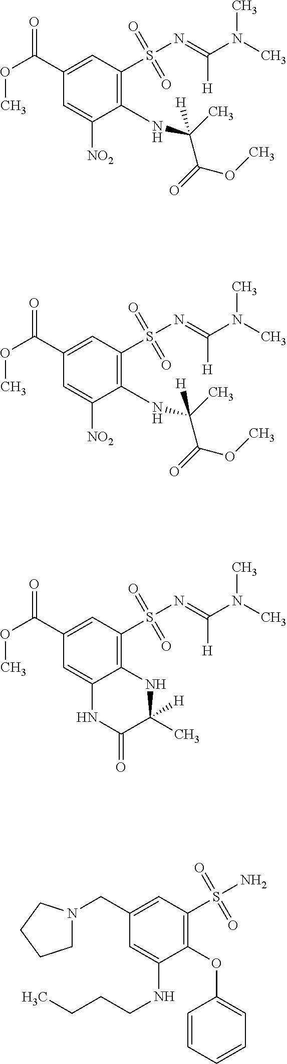 [SMILES: COC(=O)c1cc([N+](=O)[O-])c(N[C@@H](C)C(=O)OC)c(S(=O)(=O)/N=C/N(C)C)c1 ; COC(=O)c1cc([N+](=O)[O-])c(N[C@H](C)C(=O)OC)c(S(=O)(=O)/N=C/N(C)C)c1 ; COC(=O)c1cc2c(c(S(=O)(=O)/N=C/N(C)C)c1)N[C@@H](C)C(=O)N2 ; CCCCNc1cc(CN2CCCC2)cc(S(N)(=O)=O)c1Oc1ccccc1]

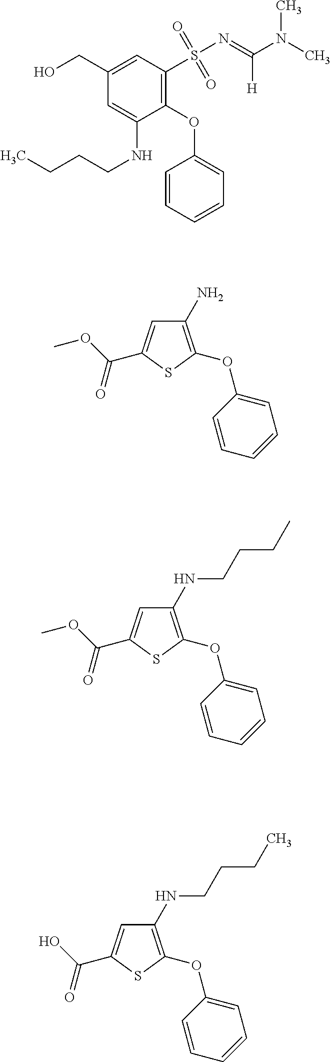

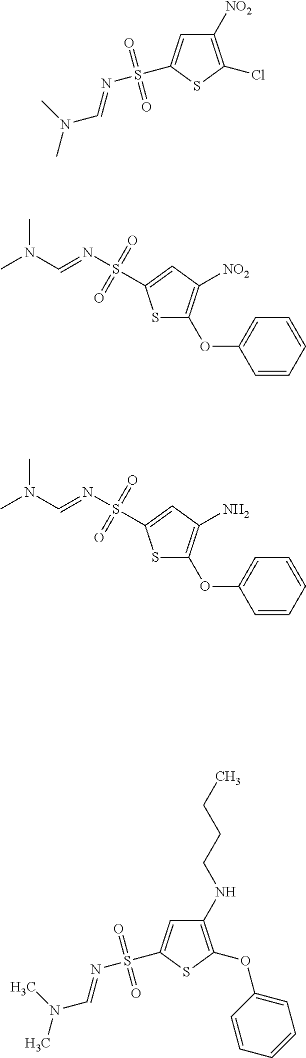 [SMILES: CN(C)/C=N/S(=O)(=O)c1cc([N+](=O)[O-])c(Cl)s1 ; CN(C)/C=N/S(=O)(=O)c1cc([N+](=O)[O-])c(Oc2ccccc2)s1 ; CN(C)/C=N/S(=O)(=O)c1cc(N)c(Oc2ccccc2)s1 ; CCCCNc1cc(S(=O)(=O)/N=C/N(C)C)sc1Oc1ccccc1]

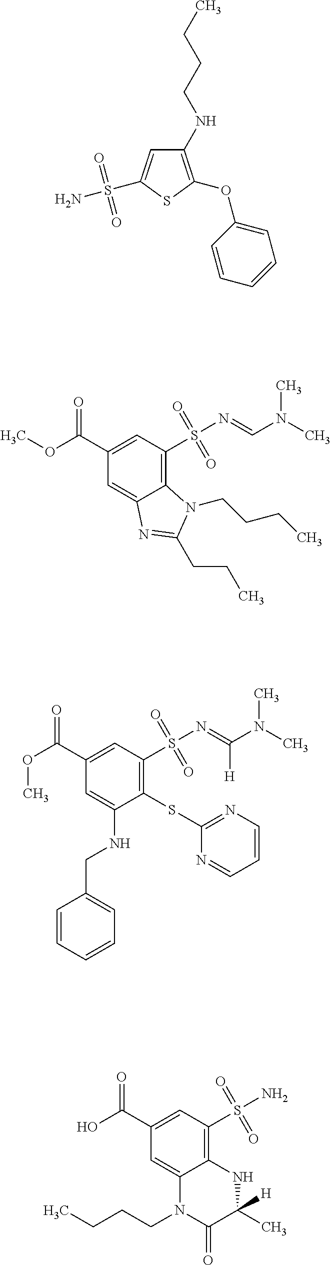

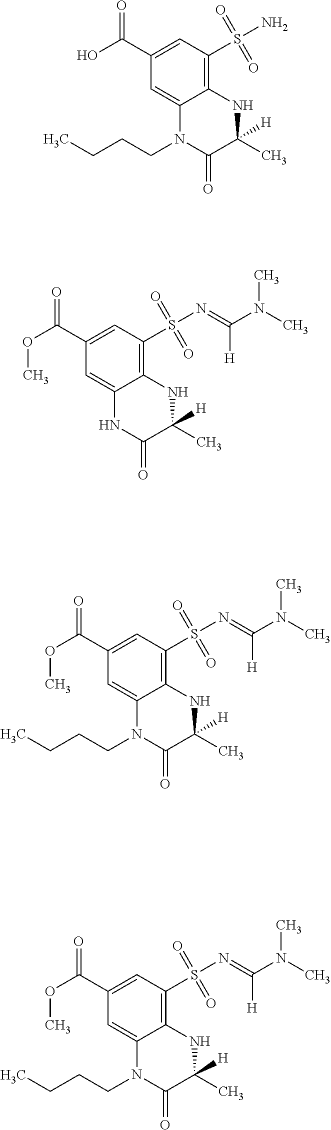

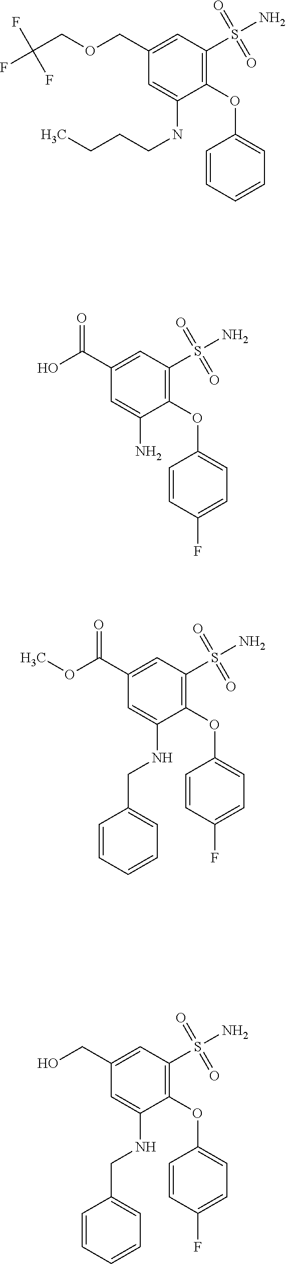 [SMILES: CCCCNc1cc(COCC(F)(F)F)cc(S(N)(=O)=O)c1Oc1ccccc1 ; Nc1cc(C(=O)O)cc(S(N)(=O)=O)c1Oc1ccc(F)cc1 ; COC(=O)c1cc(NCc2ccccc2)c(Oc2ccc(F)cc2)c(S(N)(=O)=O)c1 ; NS(=O)(=O)c1cc(CO)cc(NCc2ccccc2)c1Oc1ccc(F)cc1]

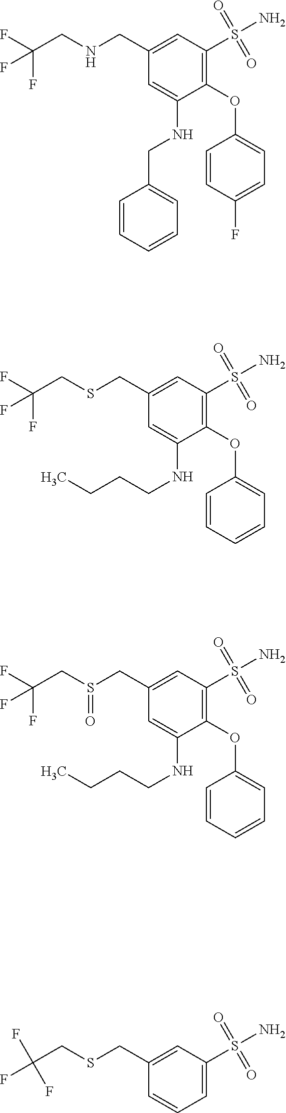 [SMILES: NS(=O)(=O)c1cc(CNCC(F)(F)F)cc(NCc2ccccc2)c1Oc1ccc(F)cc1 ; CCCCNc1cc(CSCC(F)(F)F)cc(S(N)(=O)=O)c1Oc1ccccc1 ; CCCCNc1cc(CS(=O)CC(F)(F)F)cc(S(N)(=O)=O)c1Oc1ccccc1 ; NS(=O)(=O)c1cccc(CSCC(F)(F)F)c1]

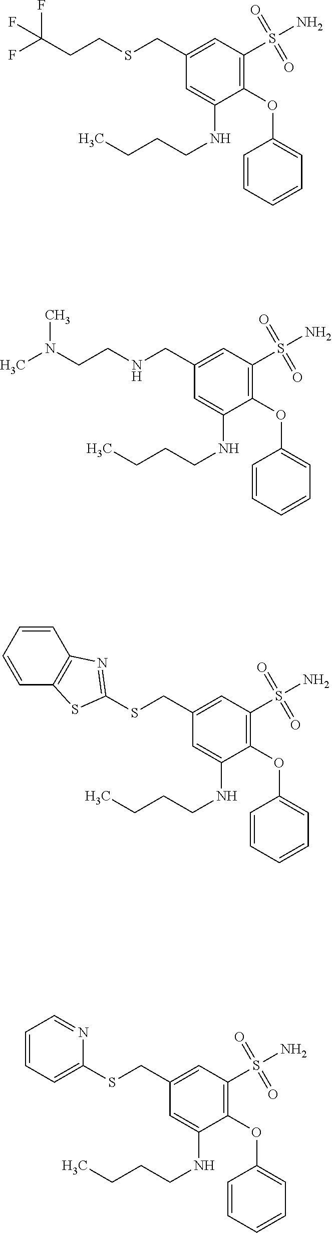 [SMILES: CCCCNc1cc(CSCCC(F)(F)F)cc(S(N)(=O)=O)c1Oc1ccccc1 ; CCCCNc1cc(CNCCN(C)C)cc(S(N)(=O)=O)c1Oc1ccccc1 ; CCCCNc1cc(CSc2nc3ccccc3s2)cc(S(N)(=O)=O)c1Oc1ccccc1 ; CCCCNc1cc(CSc2ccccn2)cc(S(N)(=O)=O)c1Oc1ccccc1]

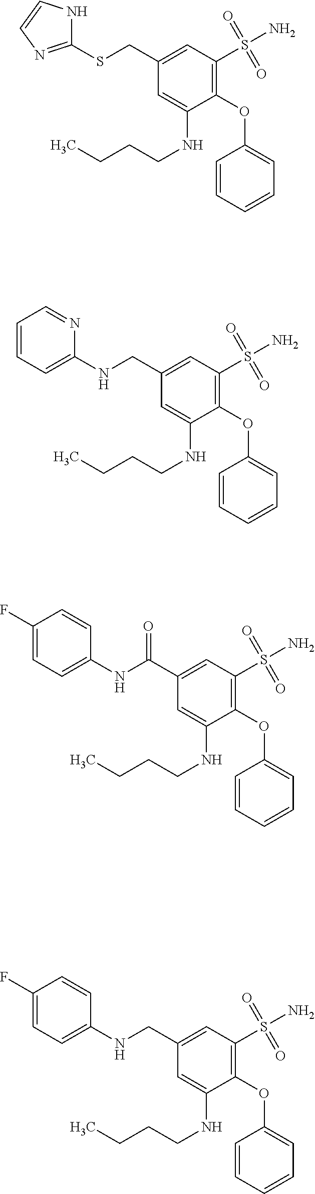 [SMILES: CCCCNc1cc(CSc2ncc[nH]2)cc(S(N)(=O)=O)c1Oc1ccccc1 ; CCCCNc1cc(CNc2ccccn2)cc(S(N)(=O)=O)c1Oc1ccccc1 ; CCCCNc1cc(C(=O)Nc2ccc(F)cc2)cc(S(N)(=O)=O)c1Oc1ccccc1 ; CCCCNc1cc(CNc2ccc(F)cc2)cc(S(N)(=O)=O)c1Oc1ccccc1]

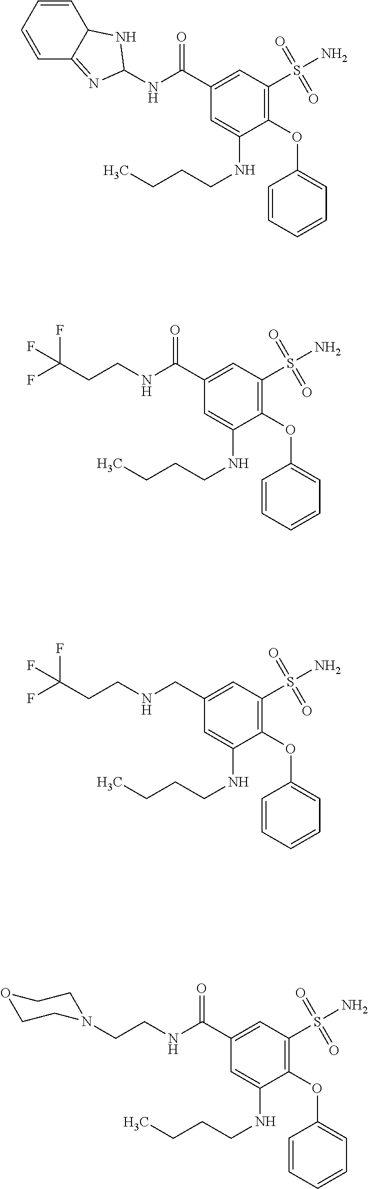 [SMILES: CCCCNc1cc(C(=O)NC2N=C3C=CC=CC3N2)cc(S(N)(=O)=O)c1Oc1ccccc1 ; CCCCNc1cc(C(=O)NCCC(F)(F)F)cc(S(N)(=O)=O)c1Oc1ccccc1 ; CCCCNc1cc(CNCCC(F)(F)F)cc(S(N)(=O)=O)c1Oc1ccccc1 ; CCCCNc1cc(C(=O)NCCN2CCOCC2)cc(S(N)(=O)=O)c1Oc1ccccc1]

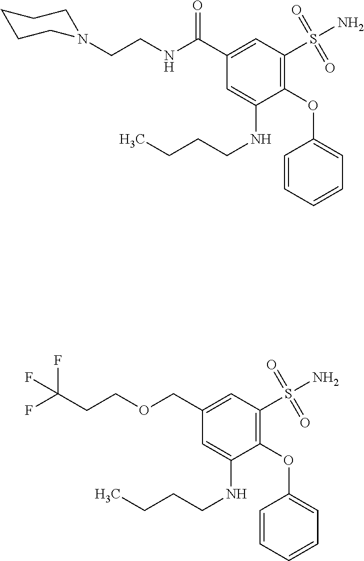 [SMILES: CCCCNc1cc(C(=O)NCCN2CCCCC2)cc(S(N)(=O)=O)c1Oc1ccccc1 ; CCCCNc1cc(COCCC(F)(F)F)cc(S(N)(=O)=O)c1Oc1ccccc1]

Error! Objects cannot be created from editing field codes.Error! Objects cannot be created from editing field codes.Error! Objects cannot be created from editing field codes.
16. A compound as defined in item 15 for use as a medicament.
17. A pharmaceutical composition comprising a compound as defined in item 15 and a pharmaceutically acceptable excipient.

The invention will now be described by reference to the following examples which are merely illustrative and are not to be construed as a limitation of the scope of the present invention.

### EXAMPLES

The compounds described in this section are defined by their chemical formulae and their corresponding chemical names. In case of conflict between any chemical formula and the corresponding chemical name indicated herein, the present invention relates to both the compound defined by the chemical formula and the compound defined by the chemical name, and particularly relates to the compound defined by the chemical formula.

### Example 1: Synthesis of Various Compounds According to the Invention

General Methods

All chemicals and solvents were purchased from commercial suppliers (Sigma Aldrich, Merck, Apollo Scientific and TCI Europe) at analytical grade. Bumetanide was obtained from OChem Inc., Des Plaines, Ill., US.

To monitor reactions via thin layer chromatography, silica gel F₂₅₄ coated aluminum sheets from Merck were used.

As a stationary phase for column chromatography silica gel 60 70-230 mesh ASTM from Merck was used.

Melting points were measured on a ThermoGalen Kofler hot stage microscope.

¹H- and ¹³C-NMR spectra were recorded on a Bruker Advance (200 and 50 MHz respectively) and chemical shifts are reported in ppm relatively to the solvent residual line or tetramethylsilane as internal standard.

Mass spectra were recorded on a Shimadzu (GC-17A; MS-QP5050A) spectrometer. The peak intensity is specified in percent relative to the biggest signal in the spectrum.

Elemental analysis were performed by Mag. Johannes Theiner at the University of Vienna and all reported values are within +/−0.4% of the calculated values.

### Ethyl 3-(butylamino)-4-phenoxy-5-sulfamoyl-benzoate (TEPS 1)

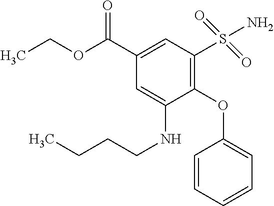 [SMILES: CCCCNc1cc(C(=O)OCC)cc(S(N)(=O)=O)c1Oc1ccccc1]

To a suspension of 5 mmol (1.82 g) bumetanide in 3 mL EtOH, 11 mmol (0.8 mL) SOC12 were added under argon atmosphere and stirred overnight. After completed conversion (monitored by TLC T/EtOAc 6+4) the mixture was extracted with 5% NaHCO₃, saturated brine, and water several times. Then the organic layer was dried over anhydrous sodium sulfate and the solvent was removed under reduced pressure. The raw product was recrystallized from ethanol to yield 1.67 g (85%) TEPS 1. ¹H NMR (200 MHz, chloroform-d) δ 8.05-7.92 (m, 1H), δ 7.66-7.54 (m, 1H), δ 7.45-7.25 (m, 2H), δ 7.21-7.05 (m, 1H), δ 7.00-6.89 (m, 2H), δ 4.96 (s, 2H), δ 4.40 (q, J=7.1 Hz, 2H), δ 4.03-3,45 (br s, 1H), δ 3.21-3.01 (m, 2H), δ 1.41 (t, J=7.1 Hz, 3H), δ 1.31-1.07 (m, 4H), δ 0.95-0.76 (m, 3H).

### N-Benzyl-3-(butylamino)-4-phenoxy-5-sulfamoyl-benzamide (TEPS 2)

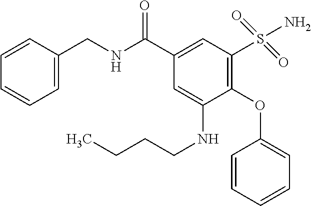 [SMILES: CCCCNc1cc(C(=O)NCc2ccccc2)cc(S(N)(=O)=O)c1Oc1ccccc1]

3 mmol (1.09 g) of bumetanide were dissolved in 20 ml dichloromethane and 3.38 mmol (0.65 g) EDC.HCl were added. After 5 min 3.37 mmol (0.52 g) HOBt were added and the reaction mixture was stirred for another 5 min. Then 3 mmol (328 μl) benzylamine were added and the reaction mixture was stirred at room temperature overnight. Once the reaction was completed, it was extracted three times with ethyl acetate and the combined organic layers were washed with brine and dried with Na₂SO₄. The solvent was then evaporated under reduced pressure and the crude product was purified by column chromatography (toluene/ethyl acetae 6+4) and by recrystallization from 50% ethanol. ¹H-NMR (200 MHz, chloroform-d): δ 7.55-7.45 (m, 2H), δ 7.40-7.20 (m, 7H), δ 7.14-7.02 (m, 1H), δ 6.96-6.80 (m, 3H), δ 5.09 (s, 2H), δ 4.59 (d, J=5.8 Hz, 2H), δ 3.07 (t, J=6.9 Hz, 2H), δ 1.49-1.30 (m, 2H), δ 1.23-1.01 (m, 2H), δ 0.79 (t, J=7.2 Hz, 3H).

### Methyl 3-(butylamino)-5-(dimethylsulfamoyl)-4-phenoxy-benzoate (TEPS 3)

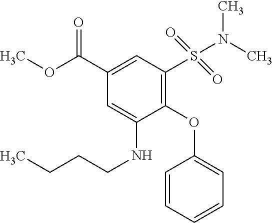 [SMILES: CCCCNc1cc(C(=O)OC)cc(S(=O)(=O)N(C)C)c1Oc1ccccc1]

3 mmol (1.09 g) of bumetanide were dissolved in 5 mL DMF and 9.9 mmol (1.37 g) K₂CO₃ and 10 mmol (0.62 mL) methyl iodide were added. The reaction mixture was stirred for 5 hours and poured into ice water. The resulting precipitate was filtered off and the crude product was purified by column chromatography (toluene/ethyl acetate 8+2). 1H-NMR (200 MHz, chloroform-d): δ 7.95 (d, J=1.9 Hz, 1H), δ 7.57 (d, J=1.9 Hz, 1H), 7.34-7.22 (m, 2H), δ 7.06 (t, J=7.3 Hz, 1H), δ 6.83 (d, J=7.9 Hz, 2H), δ 3.94 (s, 3H), δ 3.69 (s, 1H), δ 3.10 (t, J=6.9 Hz, 2H), δ 2.79 (s, 6H), 1.52-1.35 (m, 2H), δ 1.26-1.06 (m, 2H), δ 0.82 (t, J=7.2 Hz, 3H).

### 3-(Butylamino)-5-(dimethylsulfamoyl)-4-phenoxy-benzoic Acid (TEPS 4)

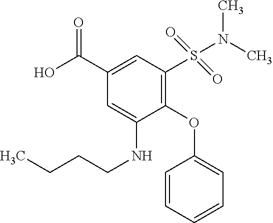 [SMILES: CCCCNc1cc(C(=O)O)cc(S(=O)(=O)N(C)C)c1Oc1ccccc1]

3 mmol (1.09 g) of bumetanide were dissolved in 20 mL of dichloromethane, then 3.38 mmol (0.65 g) EDC.HCl were added. After 5 min 3.37 mmol (0.52 g) HOBt were added and the reaction mixture was stirred for 5 min. Then 3.01 mmol (274 μl) of aniline were added and the reaction was stirred at room temperature overnight. The mixture was then extracted with ethyl acetate and washed with saturated NaHCO₃ solution and dried with Na₂SO₄. The solvent was evaporated under reduced pressure and the crude product was recrystallized from EtOH to yield 3-(butylamino)-4-phenoxy-N-phenyl-5-sulfamoyl-benzamide. 0.3 mmol (0.265 g) of 3-(butylamino)-4-phenoxy-N-phenyl-5-sulfamoyl-benzamide were dissolved in 4 mL methanol and 2 mL THF. Then 2 mL of 1M LiOH solution were added and the mixture was stirred at room temperature until the reaction was completed. The reaction mixture was acidified with 2M HCl and extracted three times with ethyl acetate. The combined organic layers were washed with brine and dried with Na₂SO₄. The solvent was then evaporated under reduced pressure. ¹H-NMR (200 MHz, chloroform-d): δ 8.05 (d, J=2.0 Hz, 1H), δ 7.63 (d, J=2.0 Hz, 1H), δ 7.37-7.24 (m, 2H), δ 7.07 (t, J=7.3 Hz, 1H), δ 6.91-6.79 (m, 2H), δ 3.13 (t, J=6.9 Hz, 2H), δ 2.81 (s, 6H), δ 1.53-1.37 (m, 2H), δ 1.29-1.08 (m, 2H), δ 0.83 (t, J=7.2 Hz, 3H).

### 3-(Butylamino)-5-(chloromethyl)-2-phenoxy-benzenesulfonamide (TEPS 76)

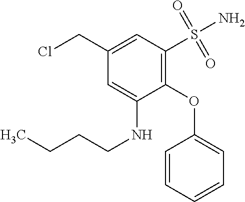 [SMILES: CCCCNc1cc(CCl)cc(S(N)(=O)=O)c1Oc1ccccc1]

1 mmol (0.35 g) of 3-(butylamino)-5-(hydroxymethyl)-2-phenoxy-benzenesulfonamide (Toellner K et al., Annals of Neurology (2014), 75(4), 550-562) was dissolved in 5 mL of thionyl chloride and heated to 80° C. for three hours. The thionyl chloride was evaporated under reduced pressure and the substance was vacuum-dried for one hour. The product was purified by recrystallization from 70% MeOH, yielding 0.34 g of brown crystals (92% yield). ¹H NMR (200 MHz, chloroform-d) δ 7.43-7-27 (m, 3H), δ 7.08 (t, J=7.3 Hz, 1H), δ 7.02-6.79 (m, 3H), δ 4.88 (s, 2H) δ 4.57 (s, 2H), δ 3.07 (t, J=6.9 Hz, 2H), δ 1.54-1.33 (m, 2H), δ 1.28-1.08 (m, 2H), δ 0.83, J=7.1 Hz (t, 3H). MS m/z: 368/370 M⁺

### 3-(Butylamino)-2-phenoxy-5-[(2,2,2-trifluoroethylamino)methyl]benzenesulfonamide (TEPS 5)

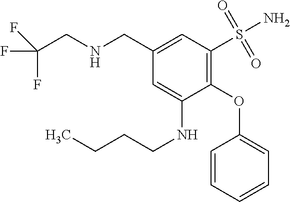 [SMILES: CCCCNc1cc(CNCC(F)(F)F)cc(S(N)(=O)=O)c1Oc1ccccc1]

General Procedure A:

1 mmol (369 mg) of 3-(butylamino)-5-(chloromethyl)-2-phenoxy-benzenesulfonamide (TEPS 76) was dissolved in 3 mL dimethylformamide (DMF). To this 2 mmol (157 μl) of 2,2,2-trifluoroethylamine were added and the mixture was stirred at room temperature overnight. After the reaction was completed, which was verified by thin layer chromatography, the fluid was evaporated under reduced pressure, yielding a white crude product. This crude product was purified by column chromatography (ethyl acetate/petroleum ether 6+4) and recrystallization from 70% MeOH, yielding 130 mg of white crystals (30% yield). ¹H NMR (200 MHz, Methanol-d4) δ 7.34-7.18 (m, 3H), δ 7.09-6.96 (m, 2H), δ 6.94-6.83 (m, 2H), δ 3.90 (s, 2H), δ 3.29-3.17 (m, 2H), δ 3.09 (t, J=6.8 Hz, 2H), δ 1.49-1.32 (m, 2H), δ 1.26-1.06 (m, 2H), δ 0.81 (t, J=7.2 Hz, 3H). MS m/z: 431 M⁺

### 3-(Butylamino)-5-(morpholinomethyl)-2-phenoxy-benzenesulfonamide (TEPS 6)

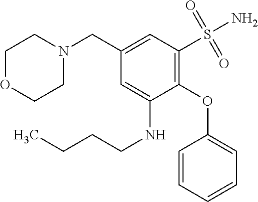 [SMILES: CCCCNc1cc(CN2CCOCC2)cc(S(N)(=O)=O)c1Oc1ccccc1]

TEPS 6 was prepared according to general procedure A, but instead of 2,2,2-trifluoroethylamine, 2 mL of morpholine were added. The crude product was purified by column chromatography (ethyl acetate/petroleum ether 1+1) and recrystallization from EtOH, yielding 130 mg of beige crystals (31% yield). ¹H NMR (200 MHz, chloroform-d) δ 7.38-7.23 (m, 3H), δ 7.20-6.80 (m, 4H), δ 5.10 (s, 2H), δ 3.98-3.66 (m, 5H), δ 3.54 (s, 2H), δ 3.16-2.98 (m, 2H), δ 2.77-2.36 (m, 4H), δ 1.52-1.30 (m, 2H), δ 1.27-1.08 (m, 2H), δ 0.82 (t, J=7.1 Hz, 3H). MS m/z: 419 M⁺

### 3-(Butylamino)-5-(1H-imidazol-2-ylsulfanylmethyl)-2-phenoxy-benzenesulfonamide (TEPS 7)

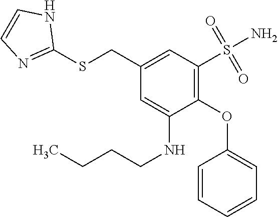 [SMILES: CCCCNc1cc(CSc2ncc[nH]2)cc(S(N)(=O)=O)c1Oc1ccccc1]

TEPS 7 was prepared according to general procedure A, but instead of 2,2,2-trifluoroethylamine, 1 mmol (100 mg) 2-mercaptoimidazole was added and the reaction was stirred for two days. The crude product was purified by column chromatography (ethyl acetate/petroleum ether/triethylamine 6+3+1) and recrystallization from 70% EtOH, yielding 0.22 g of white crystals (51% yield). ¹H NMR (200 MHz, chloroform-d) δ 7.36-7.21 (m, 2H), δ 7.14 (s, 2H), δ 7.10-6.96 (m, 2H), δ 6.94-6.80 (m, 2H), δ 6.63 (d, J=2.0 Hz, 1H), δ 4.17 (s, 2H), δ 2.95 (t, J=6.8 Hz, 2H), δ 1.42-1.26 (m, 2H), δ 1.22-1.02 (m, 2H), δ 0.80 (t, J=7.2 Hz, 3H). MS m/z: 432 M⁺

### 3-(Butylamino)-2-phenoxy-5-(pyrimidin-2-ylsulfanylmethyl)benzenesulfonamide (TEPS 8)

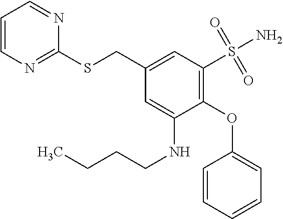 [SMILES: CCCCNc1cc(CSc2ncccn2)cc(S(N)(=O)=O)c1Oc1ccccc1]

TEPS 8 was prepared according to general procedure A, but instead of 2,2,2-trifluoroethylamine, 1 mmol (112 mg) of 2-mercaptopyrimidine were added. The crude product was purified by column chromatography (ethyl acetate/petroleum ether 7+3) and recrystallization from 70% MeOH, yielding 210 mg of white crystals (47% yield). ¹H NMR (200 MHz, chloroform-d) δ 8.55 (d, J=4.8 Hz, 2H), δ 7.38 (d, J=1.8 Hz, 1H), δ 7.35-7.23 (m, 2H), δ 7.18-6.83 (m, 5H), δ 4.90 (s, 2H), δ 4.41 (s, 2H), δ 3.80 (t, J=5.3 Hz, 1H), δ 3.17-2.91 (m, 2H), δ 1.49-1.31 (m, 2H), δ 1.26-1.05 (m, 2H), δ 0.81 (t, J=7.1 Hz, 3H). MS m/z: 444 M⁺

### 3-(Butylamino)-5-[(1-methylimidazol-2-yl)sulfanylmethyl]-2-phenoxy-benzenesulfonamide (TEPS 9)

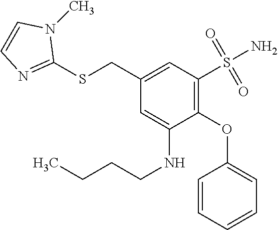 [SMILES: CCCCNc1cc(CSc2nccn2C)cc(S(N)(=O)=O)c1Oc1ccccc1]

TEPS 9 was prepared according to general procedure A, but instead of 2,2,2-trifluoroethylamine, 1 mmol (114 mg) of 2-mercapto-1-methylimidazole was added. The crude product was purified by column chromatography (ethyl acetate/petroleum ether/triethylamine 6+3+1) and recrystallization from 70% MeOH, yielding 180 mg of white crystals (40% yield). ¹H NMR (200 MHz, chloroform-d) δ 7.33-7.08 (m, 5H), δ 7.07-6.91 (m, 3H), δ 6.81 (d, J=7.7 Hz, 2H), δ 6.65 (d, J=1.7 Hz, 1H), δ 4.75, (t, J=5.7 Hz, 1H), δ 4.17 (s, 2H), δ 3.43 (s, 3H), δ 2.98-2.81 (m, 2H), δ 2.98-2.81 (m, 2H), δ 1.17-0.99 (m, 2H), δ 0.77 (t, J=7.1 Hz, 3H). MS m/z: 446 M⁺

### 3-(Butylamino)-5-(1-hydroxy-1-methyl-ethyl)-2-phenoxy-benzenesulfonamide (TEPS 10)

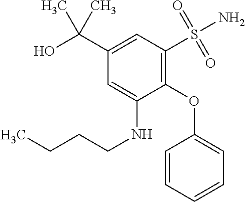 [SMILES: CCCCNc1cc(C(C)(C)O)cc(S(N)(=O)=O)c1Oc1ccccc1]

To a solution of 4 mmol of methyl 3-(butylamino)-4-phenoxy-5-sulfamoyl-benzoate (WO 2013/087090) in 10 mL dry THF 15 mL of methylmagnesium bromide solution (1.4 M in THF) were added and the mixture was stirred at room temperature. In 30 min intervals another 3 mL of the methylmagnesium bromide solution (1.4 M in THF) were added each time for 5 times. After stirring for three hours in total the mixture was quenched with 5% aqueous NH₄Cl which caused a white solid to precipitate. The mixture was then extracted with ethyl acetate and washed twice with water and once with brine. The organic layer was dried with Na₂SO₄ and concentrated under reduced pressure. The resulting crude product was purified by column chromatography (EtOAc/petroleum ether 1+1) and recrystallization from 70% EtOH yielding 1.06 g of a brown solid (70% yield). ¹H NMR (200 MHz, chloroform-d) δ 7.40-7.21 (m, 3H), CH (aromatic), δ 7.17-6.99 (m, 2H), δ 6.99-6.83 (m, 2H), δ 5.07 (s, 2H), δ 3.78 (t, J=5.1 Hz, 1H), δ 3.16-2.97 (m, 2H), δ 2.33 (s, 1H), δ 1.57 (s, 6H), δ 1.49-1.32 (m, 2H), δ 1.29-1.10 (m, 2H), δ 0.81 (t, J=7.1 Hz, 3H). MS m/z: 378 M⁺

### 3-(Butylamino)-5-isopropenyl-2-phenoxy-benzenesulfonamide (TEPS 11)

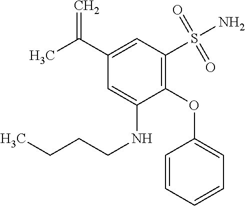 [SMILES: C=C(C)c1cc(NCCCC)c(Oc2ccccc2)c(S(N)(=O)=O)c1]

0.5 mmol of TEPS 10 were dissolved in 5 mL thionyl chloride and the mixture was stirred for one day. After the reaction was completed, the thionyl chloride was evaporated under reduced pressure. The resulting crude product was purified by column chromatography (ethyl acetate/petroleum ether 1+1) and recrystallization from 70% EtOH, yielding 120 mg of a beige solid (67% yield). ¹H NMR (200 MHz, chloroform-d) δ 7.44-7.27 (m, 3H), δ 7.12-6.90 (m, 4H), δ 5.39 (s, 1H), δ 5.23-5.10 (m, 1H), δ 4.89 (s, 2H), δ 3.78 (t, J=5.3 Hz, 1H), δ 3.16-3.00 (m, 2H), δ 2.17 (s, 3H), δ 1.51-1.35 (m, 2H), δ 1.24-1.11 (m, 2H), δ 0.83 (t, J=7.2 Hz, 3H). MS m/z: 360 M⁺

### 3-(Butylamino)-5-[(4-hydroxy-4-phenyl-1-piperidyl)methyl]-2-phenoxy-benzenesulfonamide (TEPS 12)

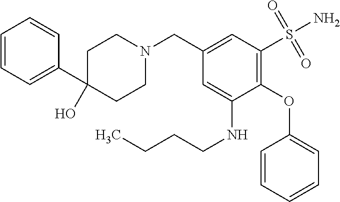 [SMILES: CCCCNc1cc(CN2CCC(O)(c3ccccc3)CC2)cc(S(N)(=O)=O)c1Oc1ccccc1]

TEPS 12 was prepared according to general procedure A, but instead of 2,2,2-trifluoroethylamine, 1.2 mmol (213 mg) of 4-hydroxy-4-phenylpiperidine were added and the mixture was stirred at room temperature for two days. The crude product was purified by column chromatography (EtOAc/petroleum ether 1+1) and recrystallization from 70% MeOH, yielding 210 mg of white crystals (41% yield). ¹H NMR (200 MHz, chloroform-d) δ 7.64-7.46 (m, 2H), δ 7.41-7.25 (m, 6H), δ 7.16-6.85 (m, 4H), δ 4.95 (s, 2H), δ 5.21-4.72 (m, 1H), δ 3.57 (s, 2H), 5=3.20-2.99 (m, 2H), δ 2.97-2.72 (m, 2H), δ 2.53 (t, J=10.8 Hz, 2H), δ 2.32-2.02 (m, 2H), δ 1.88-1.61 (m, 3H), δ 1.51-1.32 (m, 2H), δ 1.28-1.12 (m, 2H), δ 0.83 (t, J=7.1 Hz, 3H). MS m/z: 510 M⁺

### 3-(Butylamino)-5-[(cyanomethylamino)methyl]-2-phenoxy-benzenesulfonamide (TEPS 13)

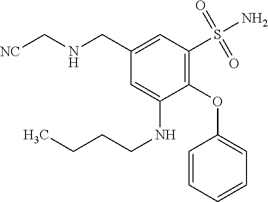 [SMILES: CCCCNc1cc(CNCC#N)cc(S(N)(=O)=O)c1Oc1ccccc1]

TEPS 13 was prepared according to general procedure A, but instead of 2,2,2-trifluoroethylamine, 1.2 mmol (167 μl) triethylamine and 1.2 mmol (71 μl) of aminoacetonitrile were added. The crude product was purified by column chromatography (EtOAc/petroleum ether 1+1) and recrystallization from 70% EtOH yielding 180 mg of beige powder (46% yield). ¹H NMR (200 MHz, chloroform-d) δ 7.40-7.22 (m, 3H), δ 7.16-6.85 (m, 4H), δ 5.02 (s, 2H), δ 3.91 (s, 2H), δ 3.88-3.78 (m, 1H), δ 3.60 (s, 2H), δ 3.20-3.00 (m, 2H), δ 2.99-2.83 (d, 1H), δ 1.49-1.34 (m, 2H), δ 1.26-1.12 (m, 2H), δ 0.90-0.74 (m, 3H). MS m/z: 388 M⁺

### 3-(Butylamino)-2-phenoxy-5-[(4-phenyl-3,6-dihydro-2H-pyridin-1-yl)methyl]benzenesulfonamide (TEPS 14)

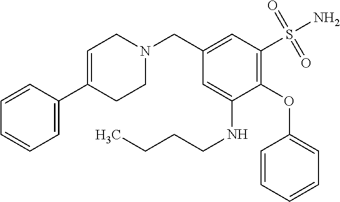 [SMILES: CCCCNc1cc(CN2CC=C(c3ccccc3)CC2)cc(S(N)(=O)=O)c1Oc1ccccc1]

TEPS 14 was prepared according to general procedure A, but instead of 2,2,2-trifluoroethylamine, 1.2 eq. 4-phenyl-1,2,3,6-tetrahydropyridine was added. The resulting crude product was purified by column chromatography (ethyl acetate/petroleum ether 3+7) to yield 110 mg of TEPS 14 (29.3% yield). ¹H NMR (200 MHz, chloroform-d) δ 7.54-7.21 (m, 9H), δ 7.07 (t, J=7.5 Hz, 1H), δ 7.00-6.83 (m, 2H), δ 6.07 (s, 1H), δ 5.06 (s, 2H), δ 4.00-3.72 (m, 3H), δ 3.50-3.28 (m, 2H), δ 3.17-3.03 (m, 2H), δ 3.00-2.80 (m, 2H), δ 2.80-2.56 (m, 2H), δ 1.54-1.32 (m, 2H), δ 1.28-1.06 (m, 2H), δ 0.81 (t, J=7.1 Hz, 3H). MS m/z: 491 M⁺

### N′-[3-(Butylamino)-2-phenoxy-5-[(2-thienylmethylamino)methyl]phenyl]sulfonyl-N,N-dimethyl-formamidine Hydrochloride (TEPS 15)

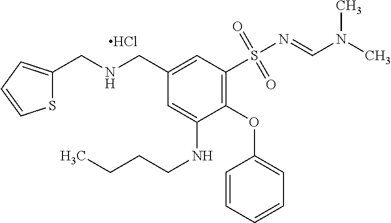 [SMILES: CCCCNc1cc(CNCc2cccs2)cc(S(=O)(=O)/N=C/N(C)C)c1Oc1ccccc1 ; Cl]

3-(Butylamino)-2-phenoxy-5-[(2-thienylmethylamino)methyl]benzenesulfonamide was prepared according to the general procedure A, but instead of aniline, 1 mmol (104 μl) of 2-Thiophenemethylamine was added. The crude product was purified by column chromatography (ethyl acetate/petroleum ether 1+1) and recrystallization from 70% EtOH, yielding 0.23 g of beige crystals (52% yield). −0.22 mmol (0.100 g) of 3-(butylamino)-2-phenoxy-5-[(2-thienylmethylamino)methyl]benzenesulfonamide were dissolved in 5 mL of dry THF and 0.48 mmol (0.068 mL) DMF-DMA were added. The reaction was stirred overnight and purified by column chromatography (ethyl acetate). The resulting brown solid was then dissolved in dry THF and 3 mL of HCl 1M in diethyl ether was added. The participating HCl salt was filtered off to yield beige crystals (37.5% yield). ¹H NMR (200 MHz, chloroform-d) δ 10.36 (s, 1H), δ 7.91 (s, 1H), δ 7.49 (d, J=11.9 Hz, 3H), δ 7.37-7.20 (m, 3H), δ 7.11-6.70 (m, 4H), δ 4.30 (s, 2H), δ 4.10 (s, 2H), δ 3.74 (t, J=6.6 Hz, 1H), δ 3.13 (s, 2H), δ 2.70 (d, J=43.2 Hz, 6H), δ 2.00-1.70 (m, 1H), δ 1.47-1.25 (m, 2H), δ 1.23-1.00 (m, 2H), δ 0.76 (t, J=6.9 Hz, 3H).

### Methyl 3-(butylamino)-5-[2-(dimethylamino)ethylsulfamoyl]-4-phenoxy-benzoate (TEPS 16)

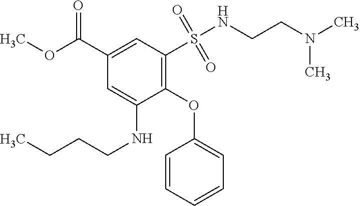 [SMILES: CCCCNc1cc(C(=O)OC)cc(S(=O)(=O)NCCN(C)C)c1Oc1ccccc1]

0.19 mmol (0.087 g) of methyl 3-(butylamino)-4-phenoxy-5-sulfamoyl-benzoate (WO 2013/087090) were dissolved in 5 mL of DMF and 1.3 mmol (0.188 g) 2-Dimethylaminoethylchlorid.HCl and 2.2 mmol (0.304 g) K₂CO₃ were added. The reaction was stirred at 40° C. for two days and the resulting crude product was purified by column chromatography (ethyl acetate/petroleum ether/triethylamine 3+6.5+0.5) to yield white powder (39.6% yield). ¹H NMR (200 MHz, chloroform-d) δ 7.95 (d, J=1.8 Hz, 1H), δ 7.67-7.53 (m, 1H), δ 7.29 (t, J=7.7 Hz, 2H), δ 7.06 (t, J=7.3 Hz, 1H), δ 6.88 (d, J=7.8 Hz, 2H), δ 3.93 (s, 3H), δ 3.88 (s, 1H), δ 3.26-3.07 (m, 2H), δ 3.05-2.90 (m, 2H), δ 2.44-2.29 (m, 2H), δ 2.16 (s, 6H), δ 1.51-1.35 (m, 2H), δ 1.21-1.05 (m, 2H), δ 0.81 (t, J=7.1 Hz, 3H). MS m/z: 449 M⁺

### Methyl 3-(butylamino)-5-[2-(dimethylamino)ethylsulfamoyl]-4-phenoxy-benzoate (TEPS 17)

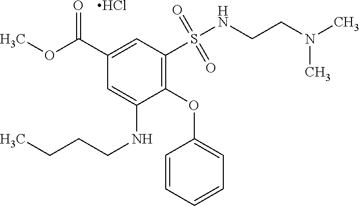 [SMILES: Cl ; CCCCNc1cc(C(=O)OC)cc(S(=O)(=O)NCCN(C)C)c1Oc1ccccc1]

TEPS 16 was dissolved in dry THF and 3 mL of HCl 1M in diethyl ether was added. The participating HCl salt was filtered off to yield white powder (92.5% yield). ¹H NMR (200 MHz, chloroform-d) δ 11.28 (s, 1H), δ 7.88 (s, 1H), δ 7.56 (s, 1H), δ 7.33-7.19 (m, 3H), δ 7.02 (t, J=8.1 Hz, 2H), δ 3.93 (s, 3H), δ 3.31 (d, J=22.4 Hz, 4H), δ 3.09 (t, J=6.8 Hz, 2H), δ 2.83 (s, 6H), δ 1.52-1.32 (m, 2H), δ 1.28-1.07 (m, 2H), δ 0.81 (t, J=7.1 Hz, 3H).

### 5-(Anilinomethyl)-3-(butylamino)-N-[2-(dimethylamino)ethyl]-2-phenoxy-benzenesulfonamide (TEPS 18)

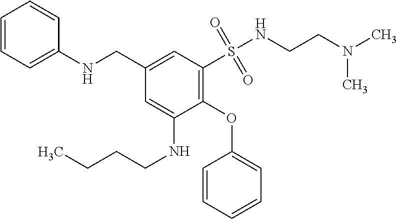 [SMILES: CCCCNc1cc(CNc2ccccc2)cc(S(=O)(=O)NCCN(C)C)c1Oc1ccccc1]

0.5 mmol (0.213 g) of 5-(anilinomethyl)-3-(butylamino)-2-phenoxy-benzenesulfonamide (Lykke K et al., British Journal of Pharmacology (2015), 172(18), 4469-4480) were dissolved in 5 mL of DMF and 2 mmol (0.288 g) 2-Dimethylaminoethylchlorid.HCl and 2 mmol (0.278 g) K₂CO₃ were added. The reaction was stirred at 40° C. overnight and the resulting crude product was purified by column chromatography (ethyl acetate/petroleum ether/triethylamine 3+6.5+0.5) to yield brown solid (38.3% yield). ¹H NMR (200 MHz, chloroform-d) δ 7.42-7.09 (m, 6H), δ 7.08-6.84 (m, 4H), δ 6.83-6.58 (m, 3H), δ 5.33 (s, 1H), δ 4.33 (s, 2H), δ 3.83 (t, J=5.3 Hz, 1H), δ 3.13-3.00 (m, 2H), δ 2.99-2.87 (m, 2H), δ 2.38-2.27 (m, 2H), δ 2.17 (s, 6H), δ 1.41-1.27 (m, 2H), δ 1.19-1.03 (m, 2H), δ 0.79 (t, J=7.1 Hz, 3H). MS m/z: 496 M⁺

### 3-(Butylamino)-5-(ethoxymethyl)-2-phenoxy-benzenesulfonamide (TEPS 19)

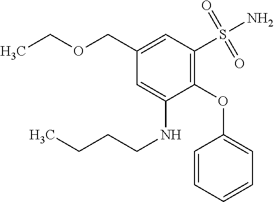 [SMILES: CCCCNc1cc(COCC)cc(S(N)(=O)=O)c1Oc1ccccc1]

0.27 mmol (100 mg) of TEPS 76 were dissolved in acetonitrile and 0.44 mmol (34 μl) sodium ethoxide were added. The reaction mixture was stirred at room temperature overnight and then additional 0.15 mmol (12 μl) of sodium ethoxide were added. After the reaction was completed 5% aqueous NaHCO₃ solution was added and the mixture was extracted with ethyl acetate. The combined organic layers were washed with brine, dried with Na₂SO₄ and concentrated under reduced pressure. The resulting crude product was purified by recrystallization from EtOH yielding 38 mg of a brown solid (22.7% yield). ¹H NMR (200 MHz, chloroform-d) δ 7.39-7.23 (m, 3H), δ 7.20-6.80 (m, 4H), δ 4.92 (s, 2H), δ 4.49 (s, 2H), δ 3.59 (q, J=7.0 Hz, 2H), δ 3.05 (t, J=7.0 Hz, 2H), δ 1.52-1.33 (m, 2H), δ 1.27 (t, J=7.0 Hz, 3H), δ 1.23-1.05 (m, 2H), δ 0.81 (t, J=6.8 Hz, 3H). MS m/z: 378 M⁺

### 3-(Butylamino)-2-phenoxy-5-(phenoxymethyl)benzenesulfonamide (TEPS 20)

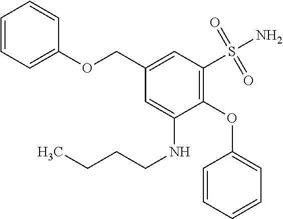 [SMILES: CCCCNc1cc(COc2ccccc2)cc(S(N)(=O)=O)c1Oc1ccccc1]

0.64 mmol (60 mg) of Phenol were dissolved in 5 mL of DMF and 0.54 mmol (200 mg) of TEPS 76 were added in three portions over 15 min. The reaction was stirred at room temperature overnight. After the reaction was completed 5% aqueous NaHCO₃ solution was added and the mixture was extracted three times with ethyl acetate. The combined organic layers were washed with brine, dried with Na₂SO₄ and concentrated under reduced pressure. The resulting crude product was purified by column chromatography (ethyl acetate/petroleum ether3+7) to yield 43 mg of TEPS 20 (15.6% yield). ¹H NMR (200 MHz, chloroform-d) δ 7.62-7.20 (m, 6H), δ 7.19-6.83 (m, 6H), δ 5.04 (s, 2H), δ 4.94 (s, 2H), δ 3.04 (t, J=6.8 Hz, 2H), δ 1.53-1.30 (m, 2H), δ 1.29-1.01 (m, 2H), δ 0.81 (t, J=6.0 Hz, 3H). MS m/z: 426 M⁺

### 3-(Butylamino)-2-phenoxy-5-(4-phenyl-3,6-dihydro-2H-pyridine-1-carbonyl)benzenesulfonamide (TEPS 21)

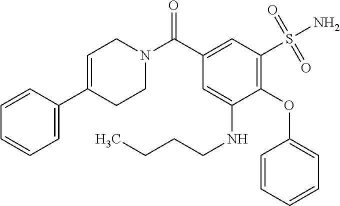 [SMILES: CCCCNc1cc(C(=O)N2CC=C(c3ccccc3)CC2)cc(S(N)(=O)=O)c1Oc1ccccc1]

1 mmol (364 mg) bumetanide and 1.1 mmol (179 mg) of CDI were added to dry THF. Once a clear solution was formed 1.3 mmol (200 mg) 4-phenyl-1,2,3,6-tetrahydropyridine were added and the reaction was stirred at room temperature overnight. After the reaction was completed 5% aqueous NaHCO₃ solution was added and the mixture was extracted three times with ethyl acetate. The combined organic layers were washed with brine, dried with Na₂SO₄ and concentrated under reduced pressure. The resulting crude product was recrystallized from EtOH to yield 294 mg of white powder (58% yield). ¹H NMR (200 MHz, chloroform-d) δ 7.59-7.21 (m, 8H), δ 7.20-6.79 (m, 4H), δ 6.39-5.78 (m, 1H), δ 5.16 (s, 2H), δ 4.47-4.07 (m, 2H), δ 4.02-3.61 (m, 2H), δ 3.05 (t, J=6.8 Hz, 2H), δ 2.63 (s-br, 2H), δ 1.52-1.29 (m, 2H), δ 1.29-1.03 (m, 2H), δ 0.80 (t, J=7.1 Hz, 3H). MS m/z: 505 M⁺

### 3-(Butylamino)-5-[2-[2-[2-(2-methoxyethoxy)ethoxy]ethoxy]ethoxymethyl]-2-phenoxy-benzenesulfonamide (TEPS 22)

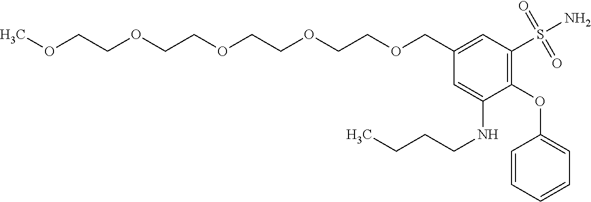 [SMILES: CCCCNc1cc(COCCOCCOCCOCCOC)cc(S(N)(=O)=O)c1Oc1ccccc1]

0.27 mmol (100 mg) of TEPS 76 were dissolved in 3 mL of dry THF, then 22 mg of NaH and 63 μl of tetraethyleneglykole were added. After the reaction was completed 5% aqueous NaHCO₃ solution was added and the mixture was extracted three times with ethyl acetate. The combined organic layers were washed with brine, dried with Na₂SO₄ and concentrated under reduced pressure. The resulting crude product was purified by column chromatography (ethyl acetate/petroleum ether 3+7) to yield 40 mg of TEPS 22 (27% yield). ¹H NMR (200 MHz, chloroform-d) δ 7.28-7.14 (m, 3H), δ 7.06-6.73 (m, 4H), δ 5.19 (s, 2H), δ 4.46 (s, 2H), δ 3.86-3.39 (m, 14H), δ 3.27 (m, 2H), δ 2.97 (t, J=6.8 Hz, 2H), δ 1.54-0.97 (m, 7H), δ 0.74 (t, J=7.2 Hz, 3H). MS m/z: 540 M⁺

### 3-(Butylamino)-4-phenoxy-5-sulfamoyl-N-(2,2,2-trifluoroethyl)benzamide (TEPS 23)

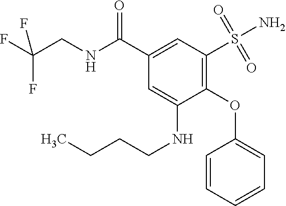 [SMILES: CCCCNc1cc(C(=O)NCC(F)(F)F)cc(S(N)(=O)=O)c1Oc1ccccc1]

To a solution of 1 mmol (364 mg) of Bumetanide in 5 mL dry THF 1.2 mmol (194 mg) of 1,1-Carbonyldiimidazole (CDI) were added and the mixture was stirred for two hours. Once TLC did not show any bumetanide remaining, 2 mmol (157 μl) of trifluoroethylamine were added and the mixture was stirred at room temperature overnight. Once the reaction was completed it was poured into 20 mL of 5% NaHCO₃ and extracted with ethyl acetate. The organic phase was then dried over Na₂SO₄ and the solvent was removed under reduced pressure. The crude product was then purified by recrystallization from EtOH to yield 159 mg of white powder (36% yield). ¹H NMR (200 MHz, Methanol-d₄) δ 7.73 (d, J=2.1 Hz, 1H), 7.44 (d, J=2.1 Hz, 1H), 7.38-7.20 (m, 2H), 7.14-6.98 (m, 1H), 6.98-6.86 (m, 2H), 4.10 (q, J=9.3 Hz, 2H), 3.13 (t, J=6.8 Hz, 2H), 1.56-1.34 (m, 2H), 1.30-1.03 (m, 3H), 0.82 (t, J=7.2 Hz, 3H). MS m/z: 445 M⁺

### 4-Morpholino-3-nitro-5-sulfamoyl-benzoic Acid (TEPS 24)

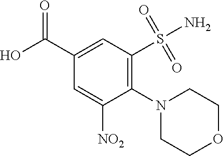 [SMILES: NS(=O)(=O)c1cc(C(=O)O)cc([N+](=O)[O-])c1N1CCOCC1]

1 mmol (280 mg) of 4-Chloro-3-nitro-5-sulfamoylbenzoic acid were dissolved in 2 mL morpholine and stirred under reflux overnight. Once the reaction was completed the crude product was purified by recrystallization from water to yield 222 mg of yellow crystals (67% yield). ¹H NMR (200 MHz, DMSO-d₆) δ 14.02 (s, 1H), δ 8.86-8.71 (m, 1H), δ 8.61-8.43 (m, 1H), δ 7.69 (s, 2H), δ 4.18-3.57 (m, 4H), δ 3.28-3.07 (m, 4H).

### Methyl 3-[(E)-dimethylaminomethyleneamino]sulfonyl-5-nitro-4-(prop-2-ynylamino)benzoate (TEPS 25)

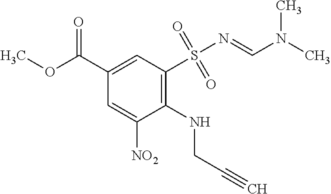

1 mmol (350 mg) of TEPS 28 was dissolved in 5 mL Acetonitrile and 2 mmol (276 mg) of K₂CO₃ and 1.1 mmol (70 μl) of propargylamine were added. The mixture was stirred overnight and after TLC showed no TEPS 28 remaining it was extracted with ethyl acetate and washed with brine. The combined organic layers were evaporated under reduced pressure and the resulting brown solid was recrystallized from 70% ethanol to yield 108 mg of brown crystals (30% yield). ¹H NMR (200 MHz, DMSO-d₆) δ 8.53-8.39 (m, 2H), δ 8.31 (s, 1H), δ 7.29 (t, J=5.6 Hz, 1H), δ 4.31-4.07 (m, 2H), δ 3.85 (s, 3H), δ 3.32 (s, 1H), δ 3.26 (t, J=2.3 Hz, 1H), δ 3.15 (s, 1H), δ 2.94 (s, 1H).

### Methyl 3-(dibutylamino)-4-phenoxy-5-sulfamoyl-benzoate (TEPS 26) (WO 2013/087090)

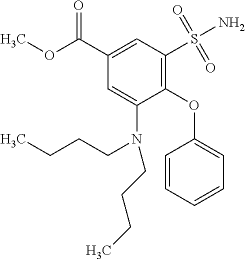 [SMILES: CCCCN(CCCC)c1cc(C(=O)OC)cc(S(N)(=O)=O)c1Oc1ccccc1]

2 mmol (228 μl) of butyraldehyde was added to a solution of 1 mmol of methyl 3-(butylamino)-4-phenoxy-5-sulfamoyl-benzoate (WO 2013/087090) in 10 mL 1,2-dichlorethane. To this solution 1.5 mmol (87 μl) of acetic acid was added. The reaction mixture was cooled to 0° C. and 3 mmol (0.64 g) of sodium triacetoxyborohydride (NaBH(OAc)3) were added over 2 hours and the mixture was stirred overnight. After the reaction was completed 10 mL of water was added and it was stirred for one hour. The reaction mixture was extracted with 20 mL dichloromethane and washed with brine. Then the organic phase was dried over Na₂SO₄ and evaporated under reduced pressure. The crude product was purified by column chromatography (ethyl acetate/petroleum ether 7+3) and recrystallized from EtOH, yielding 223 mg of white crystals (51% yield). ¹H NMR (200 MHz, chloroform-d) δ 8.23 (d, J=1.2 Hz, 1H), δ 7.87 (s, 1H), 7.37-7.15 (m, 2H), δ 7.14-6.97 (m, 1H), δ 6.93-6.73 (m, 2H), δ 4.99 (s, 2H), δ 4.12-3.79 (m, 3H), δ 3.02 (t, J=7.2 Hz, 4H), δ 1.35-1.09 (m, 4H), δ 1.10-0.92 (m, 4H), δ 0.77 (t, J=6.8 Hz, 6H). MS m/z: 434 M⁺

### 3-(Dibutylamino)-5-(hydroxymethyl)-2-phenoxy-benzenesulfonamide (TEPS 27) (WO 2013/087090)

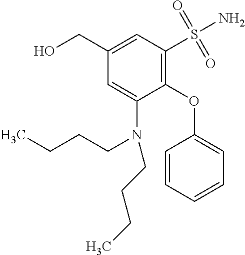 [SMILES: CCCCN(CCCC)c1cc(CO)cc(S(N)(=O)=O)c1Oc1ccccc1]

1 mmol of TEPS 26 was dissolved in 5 mL anhydrous THF and stirred at room temperature under argon atmosphere. Then 2 mL of a 1 M DIBAL-H solution in toluene were added. After one, two, three and four hours, respectively, another 1 mL of the 1M DIBAL-H solution in toluene were added each time and the reaction was stirred overnight. After TLC showed no remaining TEPS 26 the mixture was cooled to 0° C. and quenched with 5% aqueous NHCI4 solution causing a gel-like substance to precipitate. The precipitate was then dissolved in 2 N HCl and extracted three times with ethyl acetate. The combined organic layers were washed three times with water, once with brine and dried over Na2SO4. The fluids were evaporated under reduced pressure and purified by recrystallization from ethanol to yield 360 mg of beige powder (89% yield). ¹H NMR (200 MHz, Methanol-d₄) δ 7.63 (brs, 1H), δ 7.40 (brs, 1H), 7.32-7.14 (m, 2H), δ 7.00 (t, J=7.3 Hz, 1H), δ 6.82 (d, J=7.5 Hz, 2H), δ 5 4.66 (s, 2H), 3.13 (t, J=7.4 Hz, 4H), δ 1.43-0.92 (m, 8H), δ 0.78 (t, J=7.0 Hz, 6H). MS m/z: 406 M⁺

### 3-(Butylamino)-5-[(N-butylanilino)methyl]-2-phenoxy-benzenesulfonamide (TEPS 28)

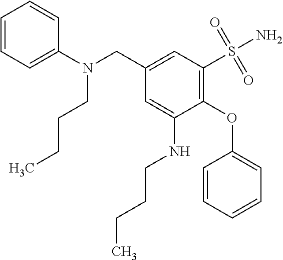 [SMILES: CCCCNc1cc(CN(CCCC)c2ccccc2)cc(S(N)(=O)=O)c1Oc1ccccc1]

2 mmol (180 μl) of butyraldehyde was added to a solution of 1 mmol of 5-(anilinomethyl)-3-(butylamino)-2-phenoxy-benzenesulfonamide (Lykke K et al., British Journal of Pharmacology (2015), 172(18), 4469-4480) in 10 mL 1,2-dichlorethane. To this solution 2 mmol (116 μl) of acetic acid and 3 mmol (0.64 g) of sodium triacetoxyborohydride (NaBH(OAc)₃) were added and the mixture was stirred overnight. The mixture was then diluted with 10 mL water and 20 mL dichloromethane. The organic phase was dried over Na₂SO₄ and then evaporated under reduced pressure. The resulting crude product was purified by column chromatography (ethyl acetate/petroleum ether 7+3), yielding 312 mg of white powder (58% yield). ¹H NMR (200 MHz, chloroform-d) δ 7.89 (s, 1H), 7.45-7.15 (m, 5H), δ 7.00 (t, J=7.3 Hz, 1H), δ 6.89-6.59 (m, 6H), δ 4.52 (s, 2H), δ 3.41 (t, J=7.6 Hz, 2H), δ 2.88 (t, J=7.0 Hz, 2H), δ 2.75 (s, 3H), δ 2.58 (s, 3H), δ 1.64 (s, 1H), δ 1.49-1.17 (m, 6H), δ 1.18-0.87 (m, 4H), δ 0.73 (t, J=7.2 Hz, 3H). MS m/z: 536 M⁺

### 3-(Dibutylamino)-4-phenoxy-5-sulfamoyl-benzoic Acid (TEPS 29) (WO 2013/087090)

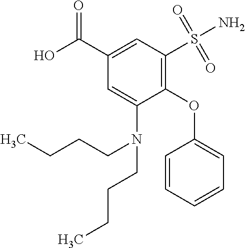 [SMILES: CCCCN(CCCC)c1cc(C(=O)O)cc(S(N)(=O)=O)c1Oc1ccccc1]

1 mmol (434 mg) of TEPS 26 was dissolved in 3 mL of MeOH and 2 mL of 2N NaOH was added. The reaction was stirred at room temperature for two hours. After TLC showed no remaining TEPS 26 the solution was acidified with 2N HCl and the resulting precipitated was filtered off and dried under vacuum to yield 380 mg of white powder (90% yield). ¹H NMR (200 MHz, Methanol-d₄) δ 8.22 (d, J=2.0 Hz, 1H), δ 7.91 (d, J=2.1 Hz, 1H), δ 7.37-7.15 (m, 2H), δ 7.02 (t, J=7.3 Hz, 1H), δ 6.83 (d, J=7.5 Hz, 2H), δ 3.10 (t, J=7.2 Hz, 4H), δ 1.38-1.15 (m, 4H), δ 1.13-0.93 (m, 4H), δ 0.78 (t, J=7.1 Hz, 6H).

### Methyl 7-[(E)-dimethylaminomethyleneamino]sulfonyl-2-methyl-1-prop-2-ynyl-benzimidazole-5-carboxylate (TEPS 30)

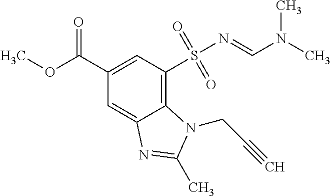

1 mmol of TEPS 25 (368 mg) was added to 15 mL of ethanol and 3 mL of dioxane, this mixture was heated to 85° C. and stirred until it was fully dissolved. Then 10 mmol of ammonium chloride (535 mg) in 6 mL water was added. 4 mmol of Iron powder (223 mg) was added in three portions 2 minutes apart. The reaction was stirred at 85° C. for another 2.5 hours until TEPS 25 could not be detected via TLC anymore. The mixture was cooled to 60° C. and then extracted three times with 25 mL of dichloromethane. The combined organic layers were washed with water and brine and dried over Na₂SO₄. The crude product was purified by flash chromatography (ethyl acetate) and recrystallization from methanol to yield 90 mg of white crystals (25% yield). ¹H NMR (200 MHz, chloroform-d) δ 8.56 (s, 1H), δ 8.48 (s, 1H), δ 8.16 (s, 1H), δ 5.80 (d, J=2.5 Hz, 2H), δ 3.95 (s, 3H), δ 3.15 (s, 3H), δ 3.12 (s, 3H), δ 2.85 (s, 3H), δ 2.38 (t, J=5.0, 2.5 Hz, 2H). MS m/z: 362 M⁺

### Methyl 4-(butylamino)-3-[(E)-dimethylaminomethyleneamino]sulfonyl-5-nitro-benzoate (TEPS 31)

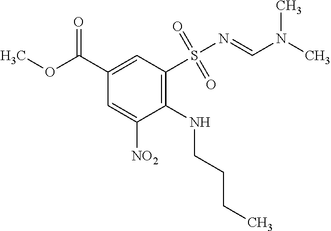

To a suspension of 3 mmol (842 mg) 4-chloro-3-nitro-5-sulfamoylbenzoic acid in 3 mL H₂O, 10.8 mmol (907 mg) NaHCO₃ was added cautiously followed by 6 mmol (595 μl) Butylamine. The resulting solution was stirred at 85° for 16 hours. After the reaction was completed, 10 mL H₂O was added and then acidified by adding 2 N HCl. The mixture was then cooled and the precipitate was filtered off to yield 825 mg of white crystals (86.6% yield). ¹H NMR (200 MHz, chloroform-d) δ 8.56 (d, J=2.2 Hz, 1H), 8.43 (d, J=2.2 Hz, 1H), 8.10 (s, 1H), 7.03 (s, 1H), 3.89 (s, 3H), 3.18 (s, 3H), 3.05 (s, 3H), 3.02-2.90 (m, 2H), 1.77-1.57 (m, 2H), 1.55-1.35 (m, 2H), 0.93 (t, J=7.1 Hz, 3H). ¹³C NMR (50 MHz, CDCl₃) δ 164.7, 159.1, 143.7, 136.8, 133.1, 132.2, 129.5, 116.1, 52.3, 46.6, 41.8, 35.8, 32.1, 19.9, 13.7. MS m/z: 387 M⁺

### Methyl 3-[(E)-dimethylaminomethyleneamino]sulfonyl-4-(1-methylimidazol-2-yl)sulfanyl-5-nitro-benzoate (TEPS 32)

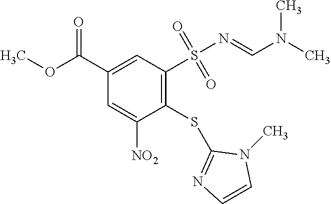

To 10 mL of Acetonitrile were added 3 mmol (1.05 g) of methyl 4-chloro-3-[(E)-dimethylaminomethyleneamino]sulfonyl-5-nitro-benzoate (WO2012/018635) 3.3 mmol (376 mg) of 2-mercapto-1-methylimidazole and 6.6 mmol (910 mg) of K₂CO₃. The solution was stirred at room temperature overnight. When TLC showed no remaining methyl 4-chloro-3-[(E)-dimethylaminomethyleneamino]sulfonyl-5-nitro-benzoate the reaction mixture was diluted with 10 mL of water and extracted with ethyl acetate. The combined organic layers were washed with brine, dried with Na₂SO₄ and evaporated under reduced pressure. The crude product was then purified by recrystallization from ethanol to yield 1.04 g of yellow crystals (81% yield). ¹H NMR (200 MHz, DMSO-d₆) δ 8.66 (d, J=1.9 Hz, 1H), δ 8.34 (d, J=1.8 Hz, 2H), δ 7.28 (s, 1H), δ 6.87 (s, 1H), δ 3.91 (s, 3H), 3.51 (s, 3H), δ 3.10 (s, 3H), δ 2.92 (s, 3H). MS m/z: not found

### Methyl 3-amino-5-[(E)-dimethylaminomethyleneamino]sulfonyl-4-(1-methylimidazol-2-yl)sulfanyl-benzoate (TEPS 33)

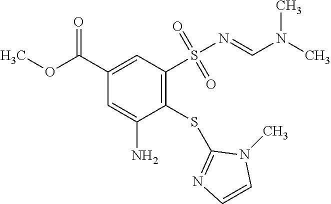

1 mmol of TEPS 32 (428 mg) was added to 15 mL of ethanol and 3 mL of dioxane, this mixture was heated to 85° C. and stirred until it was fully dissolved. Then 10 mmol of ammonium chloride (535 mg) in 6 mL water was added. 4 mmol of Iron powder (223 mg) was added in three portions 2 minutes apart. The reaction was stirred at 85° C. for another 2.5 hours until TEPS 32 could not be detected via TLC anymore. The mixture was cooled to 60° C. and then extracted three times with 25 mL of dichloromethane. The combined organic layers were washed with water and brine and dried over Na₂SO₄. The crude product was purified by flash chromatography (ethyl acetate) to yield 290 mg of yellow solid (73% yield). ¹H NMR (200 MHz, DMSO-d₆) δ 8.25 (s, 1H), δ 7.79 (d, J=1.9 Hz, 1H), δ 7.50 (d, J=2.0 Hz, 1H), δ 7.27 (d, J=1.3 Hz, 1H), δ 6.93 (d, J=1.3 Hz, 1H), δ 6.07 (s, 2H), 3.85 (s, 3H), δ 3.54 (s, 3H), δ 3.10 (s, 3H), δ 2.85 (s, 3H). MS m/z: not possible EI-MS

### Methyl 3-(butylamino)-5-[(E)-dimethylaminomethyleneamino]sulfonyl-4-(1-methylimidazol-2-yl)sulfanyl-benzoate (TEPS 34)

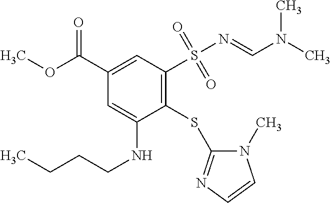

2 mmol (228 μl) of butyl iodide was added to a solution of 1 mmol of TEPS 33 in 10 mL 1,2-dichlorethane. To this solution 1.5 mmol (87 μl) of acetic acid was added. Then 3 mmol (0.64 g) of sodium triacetoxyborohydride (NaBH(OAc)₃) were added over 2 hours and the mixture was stirred overnight. After the reaction was completed it was poured into 30 mL of water. The reaction mixture was extracted twice with 20 mL dichloromethane and the combined organic layers were washed with brine. Then the organic phase was dried over Na₂SO₄ and evaporated under reduced pressure. The crude product was purified by column chromatography (ethyl acetate) yielding 234 mg of yellow powder (52% yield). ¹H NMR (200 MHz, chloroform-d) δ 8.16 (s, 1H), δ 8.09 (d, J=1.8 Hz, 1H), δ 7.36 (d, J=1.8 Hz, 1H), δ 7.10 (d, J=1.4 Hz, 1H), δ 6.96 (d, J=1.4 Hz, 1H), δ 5.79 (s, 1H), δ 3.91 (s, 3H), δ 3.59 (s, 3H), δ 3.10 (d, J=17.3 Hz, 8H), δ 1.62-1.44 (m, 2H), δ 1.43-1.15 (m, 2H), δ 0.92 (t, J=7.2 Hz, 3H). MS m/z: 453 M⁺

### 3-(Butylamino)-4-(1-methylimidazol-2-yl)sulfanyl-5-sulfamoyl-benzoic Acid (TEPS 35)

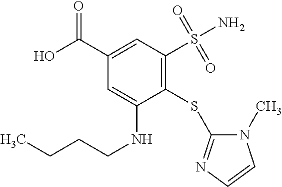 [SMILES: CCCCNc1cc(C(=O)O)cc(S(N)(=O)=O)c1Sc1nccn1C]

1 mmol (454 mg) of TEPS 34 was dissolved in 3 mL of MeOH and 2 mL of 2N NaOH was added. The reaction was stirred at room temperature for two hours. After TLC showed no remaining TEPS 34 the solution was acidified with 2N HCl and the resulting precipitate was filtered off and and recrystallized from EtOH to yield 346 mg of yellow crystals (90% yield). ¹H NMR (200 MHz, Methanol-d₄) δ 8.78 (s, 1H), δ 8.03 (dd, J=4.7, 1.7 Hz, 1H), δ 7.56 (s, 1H), δ 7.37 (s, 1H), δ 7.18 (s, 1H), δ 3.69 (s, 3H), δ 3.28-3.10 (m, 2H), δ 1.68-1.43 (m, 2H), δ 1.41-1.18 (m, 2H), δ 0.92 (t, J=6.9 Hz, 3H). MS: not possible in EI

### Methyl 4-(4,5-dihydrothiazol-2-ylsulfanyl)-3-[(E)-dimethylaminomethyleneamino]sulfonyl-5-nitro-benzoate (TEPS 36)

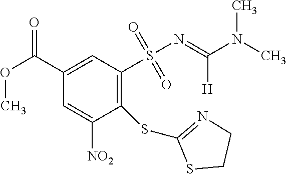 [SMILES: COC(=O)c1cc([N+](=O)[O-])c(SC2=NCCS2)c(S(=O)(=O)/N=C/N(C)C)c1]

In a three-necked flask, 1 mmol methyl 4-chloro-3-[(E)-dimethylaminomethyleneamino]sulfonyl-5-nitro-benzoate (350 mg) was dissolved in acetonitrile (10 mL). Then 1.2 mmol of 2-mercaptothiazoline (MW=119.21 g/mol; 143 mg) and 2 mmol of K₂CO₃ (MW=138 g/mol; 276 mg) were added to the flask. The reactive mixture was stirred at room temperature overnight. Water was added to the reaction and it was extracted three times with ethyl acetate washed with brine. The organic layers was dried with Na₂SO₄ and the solvent removed under reduced pressure. The crude product was then purified by column-chromatography (ethyl acetate/petroleum ether 7+3). The yield was 283 mg of a yellow, crystalline solid (78.0% yield).

### Methyl 3-[(E)-dimethylaminomethyleneamino]sulfonyl-5-nitro-4-(3-thienyl)benzoate (TEPS 39)

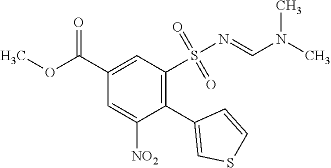

1 mmol of methyl 4-chloro-3-[(E)-dimethylaminomethyleneamino]sulfonyl-5-nitro-benzoate (320 mg) was dissolved in 3 mL of dioxane. Then 1.3 mmol (167 mg) of 3-thienyl-boronic acid, 3 mmol (414 mg) of potassium carbonate and 100 mg of tetrakis(triphenylphosphine)palladium(0) were added. The reaction vial was flooded with argon, heated to 90° C. and stirred overnight. Then the mixture was cooled to room temperature and diluted with ethyl acetate and washed with water and brine. The organic layer was dried with Na₂SO₄ and the solvent was removed under reduced pressure. The crude product was purified by flash chromatography (ethyl acetate/petroleum ether 7+3) to yield 278 mg of brown solid (70% yield). ¹H NMR (200 MHz, chloroform-d) δ 9.13 (d, J=1.8 Hz, 1H), δ 8.46 (d, J=1.8 Hz, 1H), δ 7.60-7.29 (m, 2H), δ 7.22-7.07 (m, 2H), δ 4.01 (s, 3H), δ 2.90 (s, 3H), δ 2.84 (s, 3H). MS m/z: 398 M⁺

### Methyl 3-amino-5-[(E)-dimethylaminomethyleneamino]sulfonyl-4-(3-thienyl)benzoate (TEPS 40)

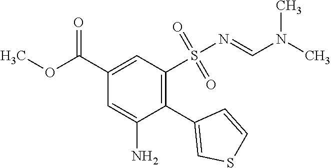

1 mmol of TEPS 39 (397 mg) was added to 15 mL of ethanol and 3 mL of dioxane and this mixture was heated to 85° C. and stirred until it was fully dissolved. Then 10 mmol of ammonium chloride (535 mg) in 6 mL water was added. 4 mmol of Iron powder (223 mg) was added in three portions 2 minutes apart. The reaction was stirred at 85° C. for another 2.5 hours until TEPS 39 could not be detected via TLC anymore. The mixture was cooled to 60° C. and then extracted three times with 25 mL of dichloromethane. The combined organic layers were washed with water and brine and dried over Na₂SO₄. The crude product was purified by flash chromatography (ethyl acetate/petroleum ether 7+3) to yield 334 mg of yellow solid (90% yield). ¹H NMR (200 MHz, chloroform-d) δ 8.43 (d, J=1.6 Hz, 1H), δ 7.73 (d, J=1.7 Hz, 1H), δ 7.47-7.35 (m, 2H), δ 7.18-7.02 (m, 2H), δ 3.93 (s, 3H), δ 2.88 (s, 3H), δ 2.84 (s, 3H). MS m/z: 367 M⁺

### Methyl 3-(butylamino)-5-[(E)-dimethylaminomethyleneamino]sulfonyl-4-(3-thienyl)benzoate (TEPS 41)

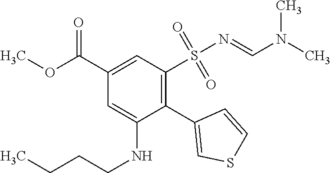

1 mmol (367 mg) of TEPS 40 is dissolved in 5 mL acetonitrile. Then 3 mmol (408 mg) of K₂CO3 and 3 mmol (3418 μl) of butyl iodide are added. The reaction mixture is stirred at 90° C. for two days. After the reaction is completed the reaction mixture is poured into 20 mL of water and extracted with ethyl acetate three times. The combined organic layers are washed with brine and dried with Na₂SO₄. The solvent is then evaporated under reduced pressure and the resulting crude product is purified by column chromatography (ethyl acetate/toluene 1+1) to yield 90 mg of white powder (21,0% yield). ¹H NMR (200 MHz, chloroform-d) δ 8.37 (d, J=1.6 Hz, 1H), δ 7.66 (d, J=1.7 Hz, 1H), δ 7.52-7.36 (m, 2H), δ 7.12 (s, 1H), δ 7.13-7.00 (m, 1H), δ 3.94 (s, 3H), δ 3.13-2.99 (m, 2H), δ 2.89 (s, 3H), δ 2.85 (s, 3H), δ 1.56-1.35 (m, 2H), δ 1.32-1.12 (m, 2H), δ 0.86 (t, J=7.2 Hz, 3H). MS m/z: 424 M⁺

### Methyl 3-(dibenzylamino)-5-[(E)-dimethylaminomethyleneamino]sulfonyl-4-(3-thienyl)benzoate (TEPS 42)

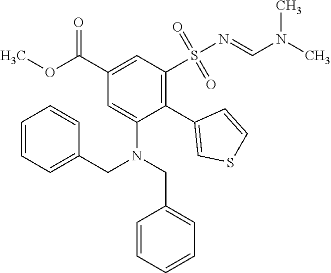

1 mmol (367 mg) of TEPS 40 is dissolved in 5 mL Acetonitrile. Then 2 mmol (276 mg) of K₂CO₃ and 2 mmol (238 μl) of benzyl bromide are added. The reaction mixture is stirred at 70° C. overnight. After the reaction is completed the reaction mixture is poured into 20 mL of water and extracted with ethyl acetate three times. The combined organic layers are washed with brine and dried with Na₂SO₄. The solvent is then evaporated under reduced pressure and the resulting crude product is purified by column chromatography (ethyl acetate/toluene 1.5+8.5) to yield 193 mg of white powder (35% yield). ¹H NMR (200 MHz, chloroform-d) δ 8.70 (d, J=1.7 Hz, 1H), δ 8.01 (d, J=1.7 Hz, 1H), δ 7.43-7.28 (m, 1H), 7.30-7.15 (m, 8H), 7.07 (s, 1H), 7.03-6.88 (m, 5H), 4.04-3.80 (m, 7H), 2.87 (s, 3H), 2.79 (s, 3H). MS m/z: 547 M⁺

### 5-(Benzyloxymethyl)-3-(butylamino)-2-phenoxy-benzenesulfonamide (TEPS 43)

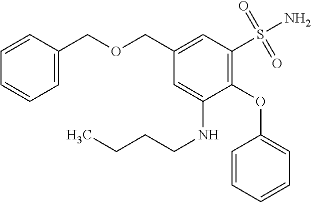 [SMILES: CCCCNc1cc(COCc2ccccc2)cc(S(N)(=O)=O)c1Oc1ccccc1]

To 10 mL of dry THF were added 1.5 mmol (37 mg) of NaH and 1.1 mmol (114 μl) of benzyl alcohol. The solution was stirred for 10 min and then 1 mmol (369 mg) of TEPS 76 were added. The reaction was stirred at room temperature overnight. When TLC showed no remaining TEPS 76, the reaction mixture was dried under reduced pressure and purified by column chromatography (ethyl acetate/petroleum ether 3+7) to yield 100 mg of white powder (22% yield). ¹H NMR (200 MHz, chloroform-d) δ 7.51-7.20 (m, 8H), δ 7.06 (t, J=7.4 Hz, 1H), δ 7.00-6.88 (m, 3H), δ 4.85 (s, 2H), 4.60 (s, 2H), δ 4.53 (s, 2H), δ 3.83 (s, 1H), δ 3.05 (t, J=6.9 Hz, 2H), δ 1.57-1.29 (m, 2H), δ 1.29-1.05 (m, 2H), δ 0.81 (t, J=7.2 Hz, 3H). MS m/z: 440 M⁺

### Methyl 3-[(E)-dimethylaminomethyleneamino]sulfonyl-5-nitro-4-pyrimidin-2-ylsulfanyl-benzoate (TEPS 44)

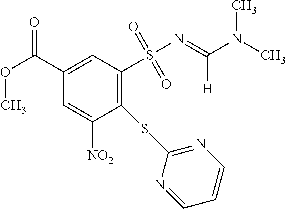 [SMILES: COC(=O)c1cc([N+](=O)[O-])c(Sc2ncccn2)c(S(=O)(=O)/N=C/N(C)C)c1]

In a three-necked flask, 1 mmol methyl 4-chloro-3-[(E)-dimethylaminomethyleneamino]sulfonyl-5-nitro-benzoate (350 mg) was dissolved in acetonitrile (10 mL). Then 1.2 mmol of 2-mercaptopyrimidine (MW=112.15 g/mol; 135 mg) and 2 mmol of K₂CO₃ (MW=138 g/mol; 276 mg) were added to the flask. The reactive mixture was stirred at room temperature overnight and controlled by thin layer chromatography (ethyl acetate/petroleum ether 7+3). The mixture was extracted three times with ethyl acetate and washed with water (2×15 mL) and brine (1×20 mL). The organic layer was dried with Na₂SO₄ and the solvent removed under reduced pressure. The crude product was then purified by column chromatography (ethyl acetate/petroleum ether 7+3). The yield was 408 mg of a yellow, crystalline solid (95.9% yield). ¹H NMR (200 MHz, DMSO-d₆) δ 8.84 (d, J=1.9 Hz, 1H), 8.68 (d, J=1.9 Hz, 1H), 8.59 (d, J=4.8 Hz, 2H), 8.24 (d, J=2.6 Hz, 2H), 7.31 (d, J=4.9 Hz, 1H), 3.96 (s, 3H), 3.33 (s, 3H), 2.99 (s, 3H). ¹³C NMR (50 MHz, CDCl₃) δ 163.6, 161.1, 154.1, 147.2, 133.0, 132.9, 128.3, 63.4, 53.3, 42.0, 36.2, 36.0. MS m/z: 426 M⁺

### Methyl 3-amino-5-[(E)-dimethylaminomethyleneamino]sulfonyl-4-pyrimidin-2-ylsulfanyl-benzoate (TEPS 45)

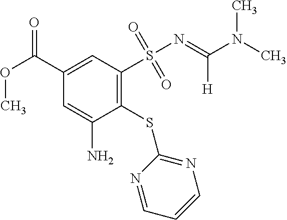 [SMILES: COC(=O)c1cc(N)c(Sc2ncccn2)c(S(=O)(=O)/N=C/N(C)C)c1]

In a three-necked flask 0.96 mmol TEPS 44 (408 mg) were dissolved under reflux in 20 mL EtOH and 10 mL dioxane. Then 9.8 mmol NH₄Cl (MW=53.49 g/mol; 524 mg) were dissolved in 12.5 mL H₂O and were added to the reaction vial. After 5 minutes 3.84 mmol Fe²⁺ (MW=55.85 g/mol; 215 mg), divided into 3 portions, were added. The reactive mixture was then stirred for 1 h under reflux. EtOH and dioxane were removed under reduced pressure, before dichloromethane and H₂O were added. The product was extracted in dichloromethane and the organic layer was washed with 2×15 mL H₂O and brine. The organic layer was dried with Na₂SO₄ and dichloromethane was removed under reduced pressure. The crude product was then purified by column-chromatography (ethyl acetate/petroleum ether 7+3) to yield 144 mg of orange crystals (37.9% yield). ¹H NMR (200 MHz, DMSO-d₆) δ 8.80 (s, 1H), 8.56 (d, J=4.8 Hz, 1H), 8.33 (d, J=4.7 Hz, 2H), 8.07 (d, J=2.0 Hz, 1H), 6.88 (t, J=4.8 Hz, 1H), 3.88 (s, 5H), 3.18 (s, 2H), 2.93 (d, J=12.3 Hz, 2H). ¹³C NMR (50 MHz, DMSO) δ 164.8, 160.8, 157.8, 146.2, 143.8, 130.7, 122.0, 114.0, 66.3, 52.7, 41.1, 35.4. MS m/z:397 M⁺

### Methyl 3-(butylamino)-5-[(E)-dimethylaminomethyleneamino]sulfonyl-4-pyrimidin-2-ylsulfanyl-benzoate (TEPS 46)

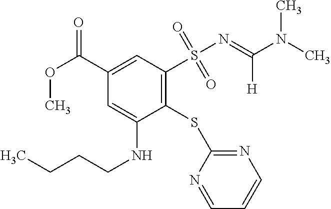 [SMILES: CCCCNc1cc(C(=O)OC)cc(S(=O)(=O)/N=C/N(C)C)c1Sc1ncccn1]

In a round-bottom flask 0.36 mmol TEPS 45 (144 mg) were dissolved in DMF. After that 0.45 mmol K₂CO₃ (63 mg) and 0.45 mmol butyliodide (51.1 μl) were added to the reaction vial. The reactive mixture was stirred at room temperature overnight. Then DMF was removed under reduced pressure. The product was extracted in ethyl acetate and the organic layer washed with H₂O and brine. The crude product was purified by column-chromatography (ethyl acetate/petroleum ether 7+3), to yield 143 mg of orange crystals (87.9% yield). ¹H NMR (200 MHz, chloroform-d) δ 9.42 (d, J=1.9 Hz, 1H), 8.98 (s, 1H), 8.68-8.44 (m, 3H), 8.34 (s, 1H), 6.87 (t, J=4.9 Hz, 1H), 3.95 (s, 3H), 3.20 (s, 3H), 3.04 (s, 2H), 2.87 (t, J=7.5 Hz, 2H), 1.60 (t, J=7.9 Hz, 1H), 1.39 (dd, J=15.1, 7.0 Hz, 2H), 0.85 (t, J=7.2 Hz, 3H). ¹³C NMR (50 MHz, CDCl₃) δ 166.0, 161.2, 159.2, 158.1, 146.4, 143.6, 131.0, 125.3, 123.6, 123.0, 113.8, 52.5, 41.6, 37.0, 35.6, 31.5, 22.2, 13.6. MS m/z: 451 M⁺

### 3-(Butylamino)-4-pyrimidin-2-ylsulfanyl-5-sulfamoyl-benzoic Acid (TEPS 47)

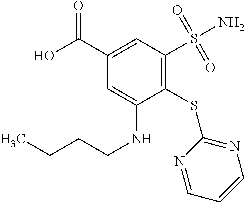 [SMILES: CCCCNc1cc(C(=O)O)cc(S(N)(=O)=O)c1Sc1ncccn1]

In a round-bottomed flask 0.28 mmol TEPS 46 (136 mg) were dissolved in 20 mL MeOH and 7 mL 2N NaOH. The reactive mixture was stirred for 2 h at 50° C. MeOH was removed under reduced pressure. The aqueous layer was poured into a beaker and acidified with 2N HCl. The resulting precipitate was afterwards extracted with ethyl acetate. The organic layer was washed with H₂O and brine and the solvent was removed under reduced pressure, to yield 104 mg of a beige powder (89% yield). ¹H NMR (200 MHz, DMSO-d₆) δ 13.43 (s, 1H), 9.36-8.88 (m, 2H), 8.59 (d, J=4.9 Hz, 2H), 8.23 (d, J=1.9 Hz, 1H), 7.52 (s, 2H), 7.01 (t, J=4.8 Hz, 1H), 2.82 (t, J=7.2 Hz, 2H), 1.46 (q, J=7.6 Hz, 2H), 1.34-1.05 (m, 3H), 0.73 (t, J=7.2 Hz, 3H). ¹³C NMR (50 MHz, DMSO) δ 166.2, 159.3, 158.5, 148.5, 143.6, 131.2, 126.0, 123.7, 121.7, 114.2, 35.8, 30.8, 21.4, 13.3. MS m/z: 382M⁺

### Methyl 3-(benzylamino)-5-[(E)-dimethylaminomethyleneamino]sulfonyl-4-pyrimidin-2-ylsulfanyl-benzoate (TEPS 83)

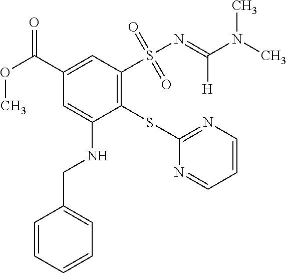 [SMILES: COC(=O)c1cc(NCc2ccccc2)c(Sc2ncccn2)c(S(=O)(=O)/N=C/N(C)C)c1]

In a round-bottom flask 0.36 mmol TEPS 45 (144 mg) were dissolved in DMF and 0.43 mmol K₂CO₃ (MG=138 g/mol; 60 mg) and 0.43 mmol benzylbromide (MG=171.04 g/mol; 5=1.44 g/cm³; 51.3 μl) were added to the reaction vial. The reactive mixture was stirred at room temperature overnight. The progress was controlled by thin-layer chromatography (ethyl acetate/petroleum ether 7+3) and DMF was removed under reduced pressure. The product was extracted in 2×15 mL ethylacetate and the organic layer washed with 2×15 mL H₂O and 1×20 mL brine. The crude product was purified by column-chromatography (ethyl acetate/petroleum ether 7+3), to yield 144 mg of beige/brown crystals (82.4% yield). ¹H NMR (200 MHz, chloroform-d) δ 9.33 (d, J=1.9 Hz, 1H), 8.69 (s, 1H), 8.57 (d, J=1.9 Hz, 1H), 8.44 (d, J=4.8 Hz, 2H), 8.34 (s, 1H), 7.23-7.00 (m, 5H), 6.81 (t, J=4.8 Hz, 1H), 4.12 (s, 2H), 3.95 (s, 3H), 3.11 (s, 3H), 3.02 (s, 3H). ¹³C NMR (50 MHz, CDCl₃) δ 166.0, 161.3, 159.1, 157.9, 146.8, 144.4, 136.7, 131.5, 129.1, 128.6, 127.3, 123.4, 123.1, 113.9, 52.6, 41.9, 41.6, 35.8, 29.8. MS m/z: 485 M⁺

### 3-(Benzylamino)-4-pyrimidin-2-ylsulfanyl-5-sulfamoyl-benzoic Acid (TEPS 48)

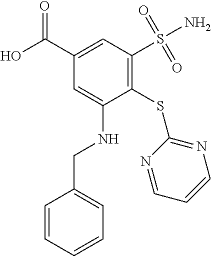 [SMILES: NS(=O)(=O)c1cc(C(=O)O)cc(NCc2ccccc2)c1Sc1ncccn1]

In a round-bottom flask 0.28 mmol TEPS 83 (136 mg) were dissolved in 20 mL MeOH and 7 mL 2N NaOH. The reactive mixture was stirred for 2 h at 50° C. MeOH was removed under reduced pressure. The aqueous layer was poured into a beaker and acidified with 2N HCl. The resulting precipitate was afterwards extracted with ethyl acetate and washed with H₂O and brine. The solvent was removed under reduced pressure, to yield 104 mg of a beige powder (89.2% yield). ¹H NMR (200 MHz, DMSO-d₆) δ 9.10 (d, J=1.9 Hz, 1H), 8.80 (s, 1H), 8.54 (d, J=4.8 Hz, 2H), 8.23 (d, J=1.9 Hz, 1H), 7.58 (s, 2H), 7.24-6.92 (m, 5H), 4.09 (s, 2H). ¹C NMR (50 MHz, DMSO) δ 166.2, 158.8, 158.2, 149.0, 144.3, 136.5, 131.8, 128.9, 128.3, 127.1, 123.3, 122.8, 121.3, 114.2, 29.0. MS m/z: not possible in EI

### Methyl 3-[(E)-dimethylaminomethyleneamino]sulfonyl-5-nitro-4-prop-2-ynoxy-benzoate (TEPS 49)

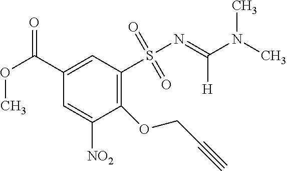 [SMILES: C#CCOc1c([N+](=O)[O-])cc(C(=O)OC)cc1S(=O)(=O)/N=C/N(C)C]

1.5 mmol (60 mg) NaH was rinsed with dry THF (6 mL) in three portions 5 minutes apart under argon atmosphere. After the dry THF was added for the third time, 1.1 mmol (64 μl) propargylalcohol and 1 mmol (320 mg) methyl 4-chloro-3-[(E)-dimethylaminomethyleneamino]sulfonyl-5-nitro-benzoate were added. The mixture was stirred at room temperature overnight. After the reaction was finished, which was controlled via thin layer chromatography, the mixture was extracted three times with ethyl acetate and washed with brine. The combined organic layers were evaporated under reduced pressure and the resulting orange solid was recrystallized from 96% ethanol to yield 83 mg of orange crystals (15% yield). ¹H NMR (200 MHz, chloroform-d) δ 8.96 (d, J=2.2 Hz, 1H), 8.70 (d, J=2.2 Hz, 1H), 8.33 (s, 1H), 5.04 (d, J=2.5 Hz, 2H), 3.98 (s, 3H), 3.23 (s, 3H), 3.04 (s, 3H), 2.69 (t, J=2.5 Hz, 1H). ¹³C NMR (50 MHz, CDCl₃) δ 163.7, 161.4, 151.7, 144.9, 139.9, 135.3, 130.5, 127.3, 77.8, 77.5, 64.7, 53.2, 42.0, 35.9. MS m/z: 370 M⁺

### Methyl 4-(allylamino)-3-[(E)-dimethylaminomethyleneamino]sulfonyl-5-nitro-benzoate (TEPS 50)

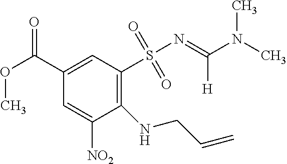 [SMILES: C=CCNc1c([N+](=O)[O-])cc(C(=O)OC)cc1S(=O)(=O)/N=C/N(C)C]

1 mmol (320 mg) of methyl 4-chloro-3-[(E)-dimethylaminomethyleneamino]sulfonyl-5-nitro-benzoate was dissolved in 5 mL Acetonitrile and 2 mmol (276 mg) of K₂CO₃ and 1.1 mmol (70 μl) of allylamine were added. The mixture was stirred overnight and after TLC showed no methyl 4-chloro-3-[(E)-dimethylaminomethyleneamino]sulfonyl-5-nitro-benzoate remaining it was extracted with ethyl acetate and washed with brine. The combined organic layers were evaporated under reduced pressure and the resulting yellow solid was recrystallized from 70% ethanol to yield 78 mg of yellow crystals (21.1% yield). ¹H NMR (200 MHz, chloroform-d) δ 8.59 (d, J=2.1 Hz, 1H), 8.46 (d, J=2.1 Hz, 1H), 8.10 (s, 1H), 7.15 (s, 1H), 6.04-5.71 (m, 1H), 5.49-5.30 (m, 1H), 5.29-5.18 (m, 1H), 3.90 (s, 3H), 3.69 (d, J=5.7 Hz, 2H), 3.18 (s, 3H), 3.04 (s, 3H). ¹³C NMR (50 MHz, CDCl₃) δ 164.6, 159.1, 143.3, 137.3, 133.1, 132.8, 132.2, 130.3, 118.8, 116.9, 52.4, 49.1, 41.8, 35.9. MS m/z: 371 M⁺

### Methyl 4-anilino-3-nitro-5-sulfamoyl-benzoate (TEPS 51)

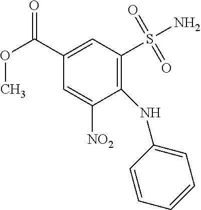 [SMILES: COC(=O)c1cc([N+](=O)[O-])c(Nc2ccccc2)c(S(N)(=O)=O)c1]

To a solution of 10 mmol (2.947 g) Methyl 3-(benzylamino)-5-[(E)-dimethylaminomethyleneamino]sulfonyl-4-phenylsulfanyl-benzoate in 50 mL acetonitrile 24 mmol aniline (2.191 mL) and 20 mmol K₂CO₃ (2.764 g) were added. The mixture was stirred for two hours at room temperature. After the reaction was complete, which was controlled by thin layer chromatography, the mixture was diluted with water and extracted with ethyl acetate. The organic phase was dried over Na₂SO₄ and then evaporated under reduced pressure. The resulting crude product was purified by recrystallization from EtOH (70%) yielding 3.40 g of orange crystals (97% yield). ¹H NMR (200 MHz, DMSO-d₆) δ 8.60 (d, J=2.1 Hz, 1H), 8.46 (s, 1H), 8.41 (d, J=2.1 Hz, 1H), 8.11 (s, 2H), 7.37-7.22 (m, 2H), 7.09 (t, J=7.3 Hz, 1H), 7.03-6.89 (m, 2H), 3.90 (s, 3H)¹³C NMR (50 MHz, DMSO) δ 163.8, 140.2, 139.3, 137.7, 133.9, 133.2, 131.1, 129.3, 124.5, 119.4, 119.3, 52.6. MS m/z: 351 M⁺

### Methyl 3-[(E)-dimethylaminomethyleneamino]sulfonyl-5-nitro-4-phenylsulfanyl-benzoate (TEPS 52)

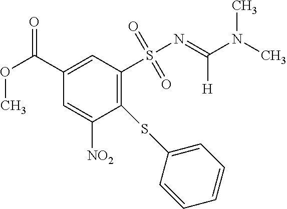 [SMILES: COC(=O)c1cc([N+](=O)[O-])c(Sc2ccccc2)c(S(=O)(=O)/N=C/N(C)C)c1]

To a solution of 2 mmol (700 mg) methyl 4-chloro-3-[(E)-dimethylaminomethyleneamino]sulfonyl-5-nitro-benzoate in 10 mL acetonitrile 4 mmol K₂CO₃ (553 mg) and 2.4 mmol of thiophenol (245 μl) were added. The mixture was stirred at room temperature for one day and after the reaction was finished, which was controlled via thin layer chromatography, the mixture was diluted with water and extracted with ethyl acetate. The organic phase was dried over Na₂SO₄ and then evaporated under reduced pressure. The resulting crude product was purified by column chromatography (first 300 mL of petroleum ether, then 300 mL of ethyl acetate) yielding 723 mg of yellow crystals (85% yield). ¹H NMR (200 MHz, chloroform-d) δ 9.10 (d, J=1.9 Hz, 1H), 8.36-8.25 (m, 2H), 7.34-7.18 (m, 3H), 7.16-7.03 (m, 2H), 3.98 (s, 3H), 2.96 (s, 3H), 2.87 (s, 3H)¹³C NMR (50 MHz, CDCl₃) δ 163.8, 161.3, 154.2, 147.0, 133.5, 132.6, 131.3, 129.90, 129.4, 128.4, 128.0, 77.2, 53.2, 41.6, 35.6. MS m/z: 423 M⁺

### Methyl 3-amino-5-[(E)-dimethylaminomethyleneamino]sulfonyl-4-phenylsulfanyl-benzoate (TEPS 53)

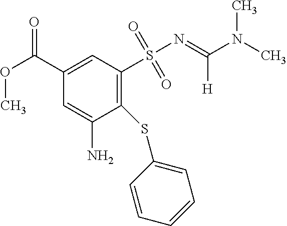 [SMILES: COC(=O)c1cc(N)c(Sc2ccccc2)c(S(=O)(=O)/N=C/N(C)C)c1]

To a solution of 1 mmol (423 mg) TEPS 52 in 10 mL EtOH a solution of 10 mmol (535 mg) NH₄Cl in 30 mL H₂O was added. The mixture was stirred and heated under reflux. After adding 10 mL dioxane 4 mmol (223 mg) iron was added in three portions at intervals of three minutes. This mixture was heated for eight hours. After the reaction was complete, which was controlled via thin layer chromatography, the mixture was diluted with water and extracted with dichloromethane. The organic phase was dried over Na₂SO₄ and then evaporated under reduced pressure, yielding 390 mg of light yellow crystals (99% of compound 4). ¹H NMR (200 MHz, chloroform-d) δ 8.39 (s, 1H), 8.22 (s, 1H), 7.65 (s, 1H), 7.26-7.06 (m, 3H), 6.99-6.84 (m, 2H), 4.25 (brs, 2H), 3.92 (s, 3H), 2.75 (s, 3H), 2.49 (s, 3H)¹³C NMR (50 MHz, CDCl₃) δ 165.9, 161.0, 150.1, 146.7, 134.7, 132.2, 129.3, 129.3, 125.7, 120.0, 119.9, 114.3, 77.2, 52.6, 41.2, 34.9. MS m/z: 493 M⁺

### Methyl 3-(butylamino)-5-[(E)-dimethylaminomethyleneamino]sulfonyl-4-phenylsulfanyl-benzoate (TEPS 54)

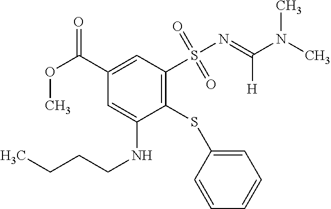 [SMILES: CCCCNc1cc(C(=O)OC)cc(S(=O)(=O)/N=C/N(C)C)c1Sc1ccccc1]

To a solution of 1.5 mmol (135 μl) butyraldehyde in 10 mL 1,2 dichloroethane 1 mmol (393 mg) TEPS 53 was added. To this solution 1 mmol acetic acid (58 μl) and 1.5 mmol (318 mg) sodium triacetoxyborohydride (NaBH(OAc)₃) were added and the mixture was stirred at room temperature overnight. After the reaction was complete, which was controlled via thin layer chromatography, the mixture was diluted with water and extracted with dichloromethane. The organic phase was dried over Na₂SO₄ and then evaporated under reduced pressure. The resulting crude product was purified by column chromatography (ethyl acetate/petroleum ether 1+1) yielding 60 mg of a beige powder (13% yield). ¹H NMR (200 MHz, chloroform-d) δ 8.31 (s, 1H), 8.22 (s, 1H), 7.49 (s, 1H), 7.33-7.04 (m, 3H), 7.01-6.83 (m, 2H), 3.93 (s, 3H), 3.11 (t, J=6.9 Hz, 2H), 2.74 (s, 3H), 2.49 (s, 3H), 1.51-1.28 (m, 2H), 1.21-0.98 (m, 2H), 0.77 (t, J=7.2 Hz, 3H)¹³C NMR (50 MHz, CDCl₃) δ 166.4, 161.0, 150.2, 146.7, 134.8, 132.3, 129.2, 129.2, 125.9, 125.8, 117.9, 114.8, 77.2, 52.6, 43.44, 41.1, 34.9, 31.0, 19.9, 13.7. MS m/z: 449 M⁺

### 3-(Butylamino)-4-phenylsulfanyl-5-sulfamoyl-benzoic Acid (TEPS 55)

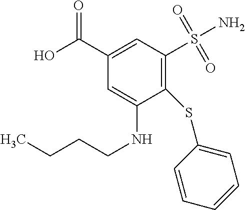 [SMILES: CCCCNc1cc(C(=O)O)cc(S(N)(=O)=O)c1Sc1ccccc1]

To a solution of 0.5 mmol (225 mg) TEPS 54 in 3 mL MeOH 2 mL 2N NaOH was added and stirred at room temperature overnight. When the reaction was complete, which was controlled by thin layer chromatography, the mixture was acidified with 2 mL 2N HCl. The precipitate was filtered off yielding 80 mg of a beige solid product (40% of compound 6). ¹H NMR (200 MHz, DMSO-d₆) δ 7.86 (s, 1H), 7.45 (s, 2H), 7.35 (s, 1H), 7.31-7.06 (m, 4H), 5.53 (t, J=5.6 Hz, 1H), 3.20-2.98 (m, 2H), 1.41-1.18 (m, 3H), 1.12-0.88 (m, 2H), 0.72 (t, J=7.2 Hz, 3H)¹³C NMR (50 MHz, DMSO) δ 166.7, 149.9, 148.8, 134.4, 133.1, 129.0, 127.4, 126.6, 114.9, 114.1, 113.5, 42.3, 39.9, 39.52, 39.1, 30.1, 19.1, 13.6. MS m/z: EI not possible

### Methyl 3-[(E)-dimethylaminomethyleneamino]sulfonyl-4-[[(1S)-2-methoxy-1-methyl-2-oxo-ethyl]amino]-5-nitro-benzoate (TEPS 56)

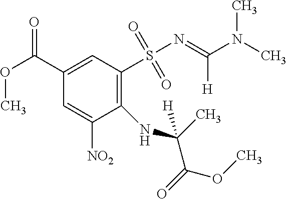 [SMILES: COC(=O)c1cc([N+](=O)[O-])c(N[C@@H](C)C(=O)OC)c(S(=O)(=O)/N=C/N(C)C)c1]

3 mmol (1049 mg) of methyl-4-chloro-3-[(E)-dimethylaminomethyleneamino]sulfonyl-5-nitro-benzoate and 6 mmol (839 mg) of L-alaninemethylester-hydrochloride were suspended in 15 mL N,N-dimethylformamide. To this mixture 1.5 mL of triethylamine were added and it was stirred at 100 C° for 2 hours. After the reaction was completed, the solution was washed with water and extracted three times with ethyl acetate. The combined organic phases were washed with brine. The organic phase was dried over sodium sulfate and the filtrate was evaporated under reduced pressure. The resulting crude product was purified by column chromatography (ethyl acetate/petroleum ether 7+3) and evaporated under reduced pressure yielding 2.21 mmol (920 mg) of yellow crystals (73.6% yield).

[α]_{D}²⁰=+67.668° (c=0.5; Methanol)

¹H NMR (200 MHz, chloroform-d) δ 8.70 (d, J=2.1 Hz, 1H), 8.48 (d, J=2.3 Hz, 1H), 8.18 (s, 1H), 7.60 (d, J=7.8 Hz, 1H), 4.34-4.11 (m, 1H), 3.91 (s, 2H), 3.75 (s, 3H), 3.18 (s, 3H), 3.07 (s, 3H), 1.45 (d, J=6.9 Hz, 3H). MS m/z: 417 M⁺

### Methyl 3-[(E)-dimethylaminomethyleneamino]sulfonyl-4-[[(1R)-2-methoxy-1-methyl-2-oxo-ethyl]amino]-5-nitro-benzoate (TEPS 57)

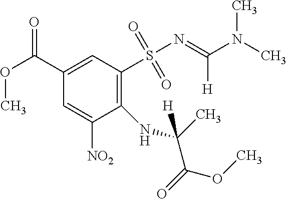 [SMILES: COC(=O)c1cc([N+](=O)[O-])c(N[C@H](C)C(=O)OC)c(S(=O)(=O)/N=C/N(C)C)c1]

1 mmol (350 mg) of methyl-4-chloro-3-[(E)-dimethylaminomethyleneamino]sulfonyl-5-nitro-benzoate and 2 mmol (280 mg) of D-alaninemethylester hydrochloride were suspended in 10 mL N,N-dimethylformamide. To this mixture 1 mL of triethylamine were added and it was stirred at 100 C° for 2 hours. After the reaction was completed, the solution was washed with water and extracted three times with ethyl acetate. The combined organic phases were washed with brine. The organic phase was dried over sodium sulfate and the filtrate was evaporated under reduced pressure. The resulting crude product was purified by column chromatography (ethyl acetate/petroleum ether 7+3) and evaporated under reduced pressure yielding 0.67 mmol (280 mg) of yellow crystals (67.0% yield).

[α]_{D}²⁰=−68.895° (c=0.5; Methanol)

¹H NMR (200 MHz, chloroform-d) δ 8.70 (d, J=2.1 Hz, 1H), 8.48 (d, J=2.2 Hz, 1H), 8.18 (s, 1H), 4.17 (dd, J=18.0, 7.0 Hz, 1H), 3.91 (s, 3H), 3.75 (s, 3H), 3.18 (s, 3H), 3.07 (s, 3H), 1.45 (d, J=6.9 Hz, 3H). ¹³C NMR (50 MHz, CDCl₃) δ 173.0, 164.6, 159.4, 141.9, 138.2, 134.0, 132.0, 131.7, 118.4, 53.5, 52.8, 52.6, 41.9, 36.0, 19.3. MS m/z: 417 M⁺

### Methyl (2S)-8-[(E)-dimethylaminomethyleneamino]sulfonyl-2-methyl-3-oxo-2,4-dihydro-1H-quinoxaline-6-carboxylate (TEPS 58)

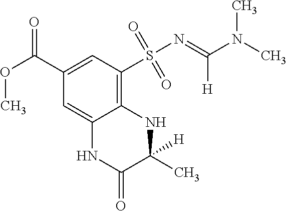 [SMILES: COC(=O)c1cc2c(c(S(=O)(=O)/N=C/N(C)C)c1)N[C@@H](C)C(=O)N2]

2 mmol (833 mg) of TEPS 56 were solved in 15 mL ethanol. 2 mL of dioxane were added and the mixture was heated to 85° C. and stirred until it was fully dissolved. Then a solution of 24 mmol of ammonium chloride (1284 mg) in 6 mL water was added. 14 mmol of Iron powder (781 mg) was added in four portions 5 minutes apart. The mixture was cooled to 60° C. and then extracted three times with 25 mL of dichloromethane.

The combined organic layers were washed with water and brine and dried over sodium sulfate. The filtrate was evaporated under reduced pressure and the resulting crude product was purified by column chromatography (with ethyl acetate/petroleum ether7+3 and ethyl acetate only) yielding 1.16 mmol (410 mg) of yellow crystals (57.8% yield).

[α]_{D}²⁰=+15.353° (c=0.5; Methanol)

¹H NMR (200 MHz, DMSO-d₆) δ 10.69 (s, 1H), 8.29 (s, 1H), 7.88 (d, J=2.0 Hz, 1H), 7.47 (d, J=2.0 Hz, 1H), 6.63 (s, 1H), 4.19 (d, J=8.8 Hz, 1H), 3.80 (s, 3H), 3.13 (s, 3H), 2.91 (s, 3H), 1.25 (d, J=6.9 Hz, 3H). ¹³C NMR (50 MHz, DMSO) δ 166.8, 165.2, 159.4, 134.6, 126.8, 124.2, 123.2, 117.7, 117.0, 51.9, 50.8, 40.8, 35.1, 29.0, 19.5. MS m/z: 354 M⁺

### 3-(Butylamino)-2-phenoxy-5-(pyrrolidin-1-ylmethyl)benzenesulfonamide (TEPS 59)

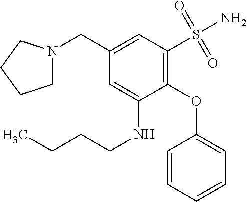 [SMILES: CCCCNc1cc(CN2CCCC2)cc(S(N)(=O)=O)c1Oc1ccccc1]

1 mmol (369 mg) of TEPS 76 was dissolved in 5 mL pyrrolidine and the mixture was stirred at room temperature overnight. After the reaction was completed, which was verified by thin layer chromatography, the fluid was evaporated under reduced pressure. The crude product was purified by column chromatography (toluene/triethylamine 8+2) and recrystallization from 70% EtOH, yielding 220 mg of white powder (55% yield). ¹H NMR (200 MHz, DMSO-d₆) δ 7.39-6.62 (m, 9H), 4.67 (s, 1H), 3.56 (s, 2H), 3.14-2.91 (m, 2H), 2.30 (s, 1H), 1.84-1.61 (m, 4H), 1.52-1.24 (m, 2H), 1.27-1.00 (m, 2H), 0.77 (t, J=7.1 Hz, 3H). ¹³C NMR (50 MHz, DMSO) δ 156.9, 142.0, 137.2, 137.0, 134.9, 129.0, 121.9, 115.5, 114.7, 113.7, 59.5, 53.6, 42.1, 30.4, 23.2, 19.3, 13.7. MS m/z: 403 M⁺

### 3-(Butylamino)-5-(imidazole-1-carbonyl)-2-phenoxy-benzenesulfonamide (TEPS 60)

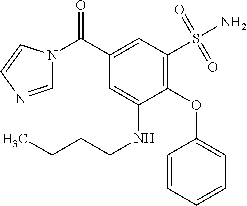 [SMILES: CCCCNc1cc(C(=O)n2ccnc2)cc(S(N)(=O)=O)c1Oc1ccccc1]

To a solution of 2.5 mmol (913 mg) of Bumetanide in 10 mL dry THF 2.75 mmol (447 mg) of 1,1-Carbonyldiimidazole (CDI) were added and the mixture was stirred at 67° C. for three hours. Then the mixture was cooled to room temperature and upon adding 15 mL of diethyl ether a white precipitate formed. The precipitate was filtered and dried under reduced pressure, yielding 0.98 g of white powder (94.30% yield). ¹H NMR (200 MHz,) δ 8.10 (d, J=1.4 Hz, 1H), 7.78 (d, J=2.0 Hz, 1H), 7.49 (d, J=2.0 Hz, 1H), 7.29-7.12 (m, 4H), 7.08-6.69 (m, 3H), 3.00 (t, J=6.7 Hz, 2H), 1.41-1.23 (m, 2H), 1.19-0.93 (m, 2H), 0.70 (t, J=7.1 Hz, 3H). ¹³C NMR (50 MHz, MeOH) δ 171.6, 158.4, 144.0, 141.1, 138.4, 133.3, 131.0, 124.3, 122.1, 117.7, 117.6, 117.1, 69.3, 44.2, 32.5, 21.3, 14.5.

### N′-[3-(Butylamino)-5-(hydroxymethyl)-2-phenoxy-phenyl]sulfonyl-N,N-dimethyl-formamidine (TEPS 61)

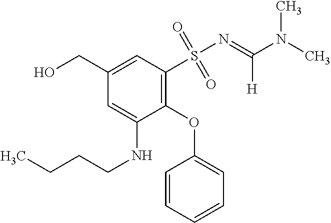 [SMILES: CCCCNc1cc(CO)cc(S(=O)(=O)/N=C/N(C)C)c1Oc1ccccc1]

To a solution of 1 mmol of 3-(butylamino)-5-(hydroxymethyl)-2-phenoxy-benzenesulfonamide (Toellner K et al., Annals of Neurology (2014), 75(4), 550-562) in 2.5 mL of DMF 1.2 mmol of (iodomethyl)cyclopropane and 1 mmol (124 mg) of AgO were added and stirred for two days. The mixture was then evaporated under reduced pressure and the resulting crude product was purified by column chromatography (toluene/triethylamine 8+2). According to the spectra the ether was not formed, but DMF bound to the sulfonamide-group resulting in the title compound, yielding 200 mg of yellow crystals (49% yield). ¹H NMR (200 MHz, DMSO-d₆) δ 7.85 (s, 1H), 7.37-7.20 (m, 2H), 7.18-6.87 (m, 3H), 6.77-6.54 (m, 2H), 5.31 (t, J=5.8 Hz, 1H), 4.72-4.56 (m, 1H), 4.51 (d, J=5.6 Hz, 2H), 4.20-3.93 (m, 1H), 3.06-2.94 (m, 2H), 2.86 (s, 3H), 1.42-1.25 (m, 2H), 1.16-1.00 (m, 2H), 0.75 (t, J=7.2 Hz, 3H). ¹³C NMR (50 MHz, DMSO) δ=160.2, 156.3, 141.7, 140.3, 135.3, 134.5, 129.3, 121.9, 115.0. 113.0, 112.7, 62.7, 42.2, 40.7, 34.5, 30.4, 19.3, 13.6. MS m/z: 406 M⁺

### 5-Chlorothiophene-2-carboxylic acid (TEPS 62) (Moni, L et al, 2016)

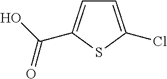

300 mmol (12 g) NaOH were solved in 60 mL H₂O and the solution was cooled down to 0-10° C. Then 90 mmol (4.65 mL) bromine were added. 30 mmol (4.8 g) 2-acetyl-5-chlorothiophene (T1) were solved in 30 mL dioxane and added at a temperature of 0-10° C. The solution was stirred for one hour at room temperature which caused that bromoform was built. Bromoform was removed by using a separatory funnel and 3 g sodium pyrosulfit in 45 mL H₂O were added to the aqueous phase. The solution was acidified with conc. HCl whereas a beige precipitate was formed. The precipitate was collected by vacuum filtration with a yield of 3.72 g. (76.1%). ¹H NMR (200 MHz, chloroform-d) δ 7.69 (d, J=4.1 Hz, 1H), δ 7.26 (s, 1H), δ 6.98 (d, J=4.1 Hz, 1H).

### Methyl 5-chlorothiophene-2-carboxylate (TEPS 63) (Hauck, S. Et al, 2016)

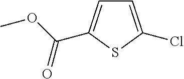

3.716 g (22.9 mmol) TEPS 62 were solved in 30 mL MeOH and 1.5 mL conc. H₂SO₄ were added. After 28 hours of stirring with reflux cooling, NaOH was added to alkalize the solution. Afterwards, the solution was extracted with ethyl acetate three times and dried over Na₂SO₄. Then the solvent was removed on a rotary evaporator, yielding a liquid brown product of 2.157 g (53.3%). ¹H NMR (200 MHz, chloroform-d) δ 7.59 (d, J=4.0 Hz, 1H) δ 6.93 (d, J=4.0 Hz, 1H) δ 3.87 (s, 3H). ¹³C NMR (50 MHz, DMSO-d6) δ 160.8, 135.6, 133.8, 131.3, 128.6, 52.5. MS m/z: 176 M⁺

### Methyl 5-chloro-4-nitro-thiophene-2-carboxylate (TEPS 64) (Park, Chan L. Et al, 2014)

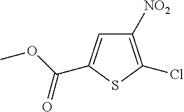

12.2 mmol (2.157 g) TEPS 63 were solved in 3.9 mL conc. H₂SO₄, and then cooled down to 0-10° C. A 0-5° C. cold mixture of 3 mL conc. H₂SO₄ and 1.7 mL conc. HNO₃ were added slowly and stirred for one hour at 0° C. Afterwards the mixture was poured on iced-water whereas a precipitate was formed. The precipitate was collected by vacuum filtration and washed three times, starting with H₂O, following by 5% -sodiumhydrogencarbonat solution and again washed with H₂O. The product was yellow with a yield of 1.502 g (54.9%). ¹H NMR (200 MHz, chloroform-d) δ 8.18 (s, 1H), δ 3.94 (s, 3H). ¹³C NMR (50 MHz, chloroform-d) δ 128.6, 53.1. MS m/z: 221 M⁺

### Methyl 4-nitro-5-phenoxy-thiophene-2-carboxylate (TEPS 65) (Consiglio, G., et al, 2002)

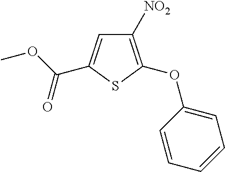 [SMILES: COC(=O)c1cc([N+](=O)[O-])c(Oc2ccccc2)s1]

1.502 g (6.8 mmol) TEPS 64 were solved in 22.4 mL dimethylfomamide, then 0.694 g phenole and 1.86 g K₂CO₃ were added. The mixture was stirred overnight by room temperature. Afterwards H₂O was added and the product was collected by extraction with ethyl acetate (three times). The organic layers were dried over Na₂SO₄ and the solvent was removed on a rotary evaporator, yielding 1.805 g of beige crystals (95.6%). ¹H NMR (200 MHz, chloroform-d) δ 8.11 (s, 1H) δ 7.64-7.16 (m, 5H) δ 3.85 (s, 3H). ¹³C NMR (50 MHz, chloroform-d) δ 161.3, 156.8, 130.8, 128.3, 127.8, 120.0, 117.3, 52.9. MS m/z: 279 M⁺

### Methyl 4-amino-5-phenoxy-thiophene-2-carboxylate (TEPS 66)

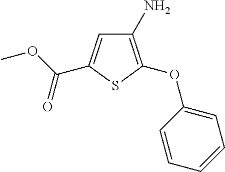 [SMILES: COC(=O)c1cc(N)c(Oc2ccccc2)s1]

1.805 g (6.5 mmol) TEPS 65 was added to 65 mL of EtOH, the mixture was heated to 85° C. and stirred until it was fully dissolved. Then 2.249 g Ammonium chloride was solved in 25 mL H₂O and added to the solution. Afterwards 0.937 g iron powder was added in three portions with few minutes apart. The solution was stirred at 85° C. for two hours until TEPS 65 could not be detected via TLC any longer. Afterwards the mixture was cooled down to 60° C. and the reaction product was collected by extraction using Dichloromethane. The organic layers were washed with H₂O and brine and were dried over Na₂SO₄. Then Dichloromethane was removed on a rotary evaporator, yielding a brown liquid of 1.25 g (76.9%). ¹H NMR (200 MHz, chloroform-d) δ 7.78-6.65 (m, 6H) δ 3.84 (s, 3H) δ 2.92 (s, 3H). ¹³C NMR (50 MHz, chloroform-d) δ 162.6, 157.9, 133.1, 129.9, 125.9, 123.8, 116.2, 62.2, 52.1. MS m/z: 249 M⁺

### Methyl 4-(butylamino)-5-phenoxy-thiophene-2-carboxylate (TEPS 67)

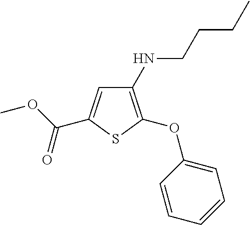 [SMILES: CCCCNc1cc(C(=O)OC)sc1Oc1ccccc1]

1.25 g (5.0 mmol) TEPS 66 were solved in 20 mL dimethylformamide, then 0.453 g K₂CO₃ and 792 μl butyl iodide were added and the mixture was stirred for three hours under reflux. Afterwards H₂O was added and the product was collected by extraction with ethyl acetate. The organic layers were dried with Na₂SO₄ and then the solvent was removed on a rotary evaporator. Afterwards the product was purified by column chromatography. (Ethyl acetate/Petroleum ether, 3+7), yielding a yellow solid of 0.702 g (4.6%). ¹H NMR (200 MHz, chloroform-d) δ 7.64-6.76 (m, 6H) δ 3.82 (s, 3H), δ 3.10 (t, J=7.0 Hz, 1H) δ 1.66-1.13 (m, 4H) δ 0.87 (t, J=7.1 Hz, 3H). ¹³C NMR (50 MHz, chloroform-d) δ 129.8, 123.7, 116.2, 52.1, 46.4, 32.1, 20.0, 13.8. MS m/z: 318 M⁺

### 4-(Butylamino)-5-phenoxy-thiophene-2-carboxylic Acid (TEPS 68)

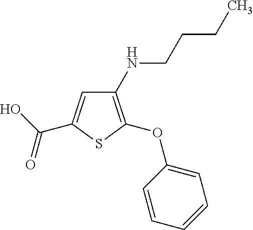 [SMILES: CCCCNc1cc(C(=O)O)sc1Oc1ccccc1]

1.98 mmol (0.7 g) TEPS 67, 2 mmol (0.218 g) KOH were dissolved in 30 mL EtOH and 10 mL H₂O. The reaction mixture was then heated to reflux for three hours. The solvent was removed under reduced pressure and the residue was diluted in H₂O. Then the solution of NaOH was added to increase the pH value to 10. Hereafter the aqueous layer was washed with ethyl acetate and acidified with concentrated HCl. Then the product was extracted with ethyl acetate, washed with water, dried over Na₂SO₄ and then removed under reduced pressure. Afterwards the product was purified by column chromatography. (Ethyl acetate/Petroleum ether, 3+7 10% CH₃COOH)) with a yield of 0.045 g (7.5%). ¹H NMR (200 MHz, chloroform-d) δ 8.20 (s, 1H) δ 7.56-6.95 (m, 6H) δ 3.13 (t, J=6.9 Hz, 1H) δ 1.42 (m, 4H) δ 0.90 (t, J=7.1 Hz, 3H). 13C NMR (50 MHz, chloroform-d) δ 167.5, 157.9, 136.5, 129.9, 125.0, 124.0, 116.5, 46.6, 31.9, 20.0, 13.8. MS m/z: 291 M⁺

### 5-Chloro-4-nitro-thiophene-2-sulfonyl Chloride (TEPS 69) (Yang, J. Et al, 2015)

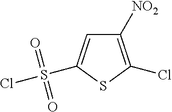 [SMILES: O=[N+]([O-])c1cc(S(=O)(=O)Cl)sc1Cl]

In a three-neck flask 6.16 mL (46.1 mmol) 5-chlorothiophene-2-sulfonylchlorid was added drowse to 55.6 mL fuming nitric acid. In the beginning the solution was cooled to provide a temperature beneath 60° C. After three hours of stirring, the reaction was completed and the solution was poured on iced-water. The product was collected by vacuum filtration yielding 11.161 g of yellow crystals (92.4%). ¹H NMR (200 MHz, chloroform-d) δ 8.30 (s, 1H). 13C NMR (50 MHz, chloroform-d) δ 129.8. MS m/z: 263 M⁺

### 5-Chloro-4-nitro-thiophene-2-sulfonamide (TEPS 70) (Kunzer, A. R., et al, 2010)

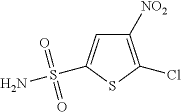 [SMILES: NS(=O)(=O)c1cc([N+](=O)[O-])c(Cl)s1]

11.31 g (42.6 mmol) TEPS 69 was added to 44.2 mL NH₄0H at 0° C. When the addition was complete, the reaction mixture was stirred at room temperature until the solid dissolved. The clear orange solution was cooled in ice, diluted with H₂O and acidified with conc. HCl to precipitate solid. The product was collected by vacuum filtration yielding 4.566 g of yellow powder (44.1%). ¹H NMR (200 MHz, DMSO-d6) δ 8.14 (s, 1H) δ 7.97 (s, 2H). 13C NMR (50 MHz, DMSO-d6) δ 141.7, 124.8. MS m/z: 242 M⁺

### N′-[(5-Chloro-4-nitro-2-thienyl)sulfonyl]-N,N-dimethyl-formamidine (TEPS 71)

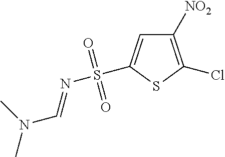 [SMILES: CN(C)/C=N/S(=O)(=O)c1cc([N+](=O)[O-])c(Cl)s1]

4.566 g (18.8 mmol) TEPS 70 was solved in 22.5 mL acetonitrile and then 2.8 mL N,N-dimehtylformamide-dimethylacetale were added slowly. The reaction was stirred overnight Then the mixture was extracted with ethyl acetate three times and the organic layer was dried with Na₂SO₄. The solvent was removed under reduced pressure, yielding a yellow product of 1.95 g (35.1%). ¹H NMR (200 MHz, DMSO-d6) δ 8.29 (s, 1H) δ 8.00 (s, 1H) δ 3.20 (s, 3H) δ 2.98 (s, 3H). ¹³C NMR (50 MHz, DMSO-d₆) δ 160.6, 124.8, 41.3, 35.4. MS m/z: 297 M⁺

### N,N-Dimethyl-N′-[(4-nitro-5-phenoxy-2-thienyl)sulfonyl]formamidine (TEPS 72)

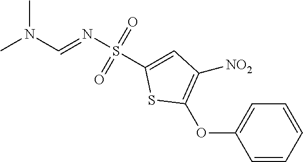 [SMILES: CN(C)/C=N/S(=O)(=O)c1cc([N+](=O)[O-])c(Oc2ccccc2)s1]

6.6 mmol (1.95 g) TEPS 71 were solved in 17 mL acetonitrile and afterwards 0.55 g phenol and 1.85 g K₂CO₃ were added. The mixture was stirred for four hours, then water was added and extracted with ethyl acetate three times and once with brine. The organic layer was dried over Na₂SO₄ and the solvent was removed under reduced pressure resulting in 0.4 g of brown crystals. The product was purified by column chromatography (Ethyl acetate/Petroleum ether 3+7) yielding 2.2 g of an orange product (92.4%). ¹H NMR (200 MHz, DMSO-d6) δ 8.22 (s, 1H) δ 7.82 (s, 1H) δ 7.70-7.37 (m, 5H) δ 3.16 (s, 3H) δ 2.92 (s, 3H). ¹³C NMR (50 MHz, chloroform-d) δ 159.5, 156.8, 130.8, 128.7, 127.8, 124.7, 119.9, 41.9, 36.0. MS m/z: 378 M+

### N′-[(4-Amino-5-phenoxy-2-thienyl)sulfonyl]-N,N-dimethyl-formamidine (TEPS 73)

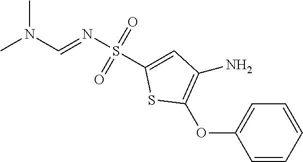 [SMILES: CN(C)/C=N/S(=O)(=O)c1cc(N)c(Oc2ccccc2)s1]

2.2 g (6.1 mmol) TEPS 72 was added to 40 mL EtOH, the mixture was heated to 85° C. and stirred until it was fully dissolved. Then 4.4 g ammonium chloride was solved in H₂O and added to the solution. Afterwards 0.61 g iron powder was added in three portions with few minutes apart. The solution was stirred at 85° C. for two hours until TEPS 72 could not be detected via TLC any longer. The mixture was cooled down to 60° C. and the reaction product was collected by extraction using Dichloromethane. The combined organic phases were washed with brine and the organic phase was dried over Na₂SO₄. Then the solvent was removed under reduced pressure. The product was purified by column chromatography (Ethyl aceteate/Petroleum ether 1+1) resulting in a yellow liquid of 0.709 g (36.1%). ¹H NMR (200 MHz, DMSO-d6) δ 8.17 (s, 1H) δ 7.46-6.92 (m, 6H) δ 4.86 (s, 2H) δ 3.16 (s, 3H) δ 2.94 (s, 3H). ¹³C NMR (50 MHz, DMSO-d₆) δ 159.6, 158.0, 135.3, 134.6, 132.2, 129.8, 123.3, 123.0, 115.8, 40.9, 35.2. MS m/z: 325 M⁺

### N′-[[4-(Butylamino)-5-phenoxy-2-thienyl]sulfonyl]-N,N-dimethyl-formamidine (TEPS 74)

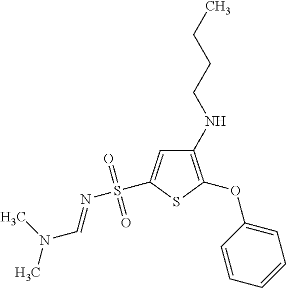 [SMILES: CCCCNc1cc(S(=O)(=O)/N=C/N(C)C)sc1Oc1ccccc1]

0.7 g (2.2 mmol) TEPS 73 were solved in 3.9 mL dimethylfomamide, then 0.31 g K₂CO₃ and 531 μl Butyl iodide were added and the mixture was stirred for three hours under reflux. Afterwards H₂O was added and the product was collected by extraction with ethyl acetate. The organic layers were dried with Na₂SO₄ and then the solvent was removed on a rotary evaporator. Afterwards the product was purified by column chromatography. (Ethyl acetate/Petroleum ether, 3+7) yielding a yellow solid of 0.101 g (11.8%). ¹H NMR (200 MHz, chloroform-d) δ 8.09 (s, 1H) δ 7.45-6.99, 6H) δ 3.20-2.98 (m, 8H) δ 1.70-1.15 (m, 4H) δ 0.89 (t, J=7.2 Hz, 1H). ¹³C NMR (50 MHz, chloroform-d) δ 159.2, 158.0, 131.7, 129.8, 123.9, 121.4, 116.3, 60.4, 46.8, 41.6, 35.7, 31.7, 19.9, 13.8. MS m/z: 381 M⁺

### Methyl 1-butyl-7-[(E)-dimethylaminomethyleneamino]sulfonyl-2-propyl-benzimidazole-5-carboxylate (TEPS 77)

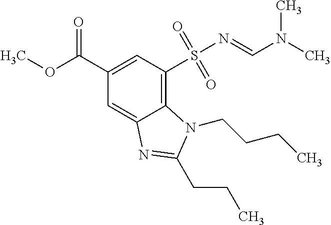

0.5 mmol (193 mg) of TEPS 31 was added to 7.5 mL of ethanol and 1.5 mL of dioxane and this mixture was heated to 85° C. and stirred until it was fully dissolved. Then 0.5 mmol ethyl acetate (25 μl), 0.5 mmol conc. acetic acid (14 μl) and 5 mmol of ammonium chloride (267.5 mg) in 3 mL water were added. 2 mmol of Iron powder (111.5 mg) were added in three portions 2 minutes apart. The reaction was stirred at 85° C. for another 2.5 hours until BUM131 could not be detected via TLC anymore. The mixture was cooled and then extracted three times with dichloromethane. The combined organic layers were washed with water and brine and dried over Na₂SO₄. The crude product was purified by column chromatography (ethyl acetate/petroleum ether 8+2) to yield 43 mg of yellow solid (21.1% yield). ¹H NMR (200 MHz, chloroform-d) δ 8.55 (s, 2H), 8.15 (s, 1H), 4.95-4.65 (m, 2H), 3.94 (s, 3H), 3.16 (s, 3H), 3.07 (s, 3H), 2.99-2.85 (m, 2H), 2.15-1.90 (m, 2H), 1.90-1.71 (m, 2H), 1.61-1.37 (m, 2H), 1.26 (s, 1H), 1.12 (t, J=7.4 Hz, 3H), 0.99 (t, J=7.3 Hz, 3H). ¹³C NMR (50 MHz, CDCl₃) δ 166.6, 159.7, 159.2, 144.3, 133.3, 126.7, 125.1, 124.2, 122.9, 52.4, 45.8, 41.8, 35.8, 33.3, 29.8, 21.1, 20.1, 14.2, 13.9. MS m/z: 408 M⁺

### Methyl (2R)-8-[(E)-dimethylaminomethyleneamino]sulfonyl-2-methyl-3-oxo-2,4-dihydro-1H-quinoxaline-6-carboxylate (TEPS78)

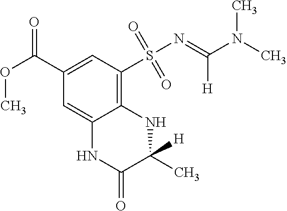 [SMILES: COC(=O)c1cc2c(c(S(=O)(=O)/N=C/N(C)C)c1)N[C@H](C)C(=O)N2]

2 mmol (833 mg) of TEPS 57 were solved in 15 mL ethanol. 2 mL of dioxane were added and the mixture was heated to 85° C. and stirred until it was fully dissolved. Then a solution of 24 mmol of ammonium chloride (1284 mg) in 6 mL water was added. 14 mmol of Iron powder (781 mg) was added in four portions 5 minutes apart. The mixture was cooled to 60° C. and then extracted three times with 25 mL of dichloromethane. The combined organic layers were washed with water and brine and dried over sodium sulfate. The filtrate was evaporated under reduced pressure and the resulting crude product was purified by column chromatography (with ethyl acetate/petroleum ether 7+3 and ethyl acetate only) yielding 1.49 mmol (530 mg) of yellow crystals (74.7% yield).

[α]_{D}²⁰=−14.159° (c=0.5; Methanol)

¹H NMR (200 MHz, DMSO-d₆) δ 10.70 (s, 1H), 8.29 (s, 1H), 7.88 (d, J=1.9 Hz, 1H), 7.47 (s, 1H), 6.64 (s, 1H), 4.19 (dd, J=6.8, 2.3 Hz, 1H), 3.80 (s, 3H), 3.56 (s, 1H), 3.13 (s, 3H), 2.91 (s, 3H), 1.25 (d, J=6.8 Hz, 3H). ¹³C NMR (50 MHz, DMSO) δ 166.8, 165.2, 159.4, 134.6, 126.8, 124.2, 123.3, 117.7, 117.0, 66.4, 51.9, 50.9, 40.9, 35.1, 19.5. MS m/z: 354 M⁺

### Methyl (2R)-4-butyl-8-[(E)-dimethylaminomethyleneamino]sulfonyl-2-methyl-3-oxo-1,2-dihydroquinoxaline-6-carboxylate (TEPS 79)

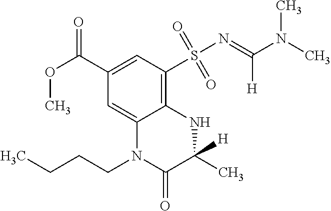 [SMILES: CCCCN1C(=O)[C@@H](C)Nc2c1cc(C(=O)OC)cc2S(=O)(=O)/N=C/N(C)C]

1 mmol (354 mg) of TEPS 78 were dissolved in 9 mL of N,N-dimethylformamide. 2 mmol (276 mg) of potassium carbonate and 6 mmol (687 μL) butyl iodide was added to the mixture and stirred at 90C° for 16 hours. After the reaction was completed, the mixture was washed with water and extracted three times with ethyl acetate.

The combined organic phases were washed with brine. The organic phase was dried over sodium sulfate and the filtrate was evaporated under reduced pressure. The resulting crude product was purified by column chromatography (ethyl acetate) and evaporated under reduced pressure yielding 0.34 mmol (139 mg) of yellow crystals (33.9% yield).

[α]_{D}²⁰=−11.163° (c=0.5; Methanol)

¹H NMR (200 MHz, chloroform-d) δ 8.37-8.02 (m, 2H), 7.66 (s, 1H), 6.62 (s, 1H), 4.19 (dd, J=6.7, 1.9 Hz, 1H), 4.07-3.76 (m, 5H), 3.15 (s, 3H), 3.03 (s, 3H), 1.67 (s, 2H), 1.74-1.57 (m, 1H), 1.56-1.31 (m, 5H), 0.97 (t, J=6.9 Hz, 3H). ¹³C NMR (50 MHz, CDCl₃) δ 166.3, 166.1, 158.9, 137.6, 128.2, 124.7, 124.5, 118.8, 118.3, 52.1, 51.4, 42.1, 41.6, 35.7, 28.9, 20.1, 19.1, 13.8. MS m/z: 410 M⁺

### (2R)-4-Butyl-2-methyl-3-oxo-8-sulfamoyl-1,2-dihydroquinoxaline-6-carboxylic Acid (TEPS 80)

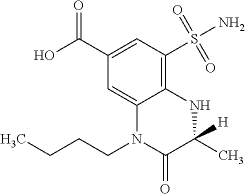 [SMILES: CCCCN1C(=O)[C@@H](C)Nc2c1cc(C(=O)O)cc2S(N)(=O)=O]

0.48 mmol (200 mg) of TEPS 79 were dissolved in 1.5 mL methanol and 1.5 mL 2N sodium hydroxide and the mixture was stirred at 40 C° for 2 hours. After the reaction was completed and the mixture was cooled down to room temperature methanol was evaporated under reduced pressure. Upon adding 1.5 mL 2N hydrochloric acid a light-yellow precipitate was formed. The precipitate was filtered and dried under reduced pressure yielding 0.43 mmol (150 mg) of light-yellow powder (91.5% yield)

[α]_{D}²⁰=−38.303° (c=0.5; Methanol)

¹H NMR (200 MHz, DMSO-d₆) δ 12.89 (s, 1H), 8.02 (s, 1H), 7.70 (s, 2H), 7.60 (s, 1H), 6.64 (s, 1H), 4.31-4.07 (m, 1H), 3.95 (t, J=7.3 Hz, 2H), 1.65-1.44 (m, 2H), 1.43-1.10 (m, 5H), 0.91 (t, J=7.1 Hz, 3H).

¹³C NMR (50 MHz, DMSO) δ 166.8, 166.6, 136.8, 128.2, 126.4, 124.8, 119.2, 118.3, 51.1, 41.2, 28.9, 19.9, 18.8, 14.1.

MS m/z: EI not possible

### Methyl (2S)-4-butyl-8-[(E)-dimethylaminomethyleneamino]sulfonyl-2-methyl-3-oxo-1,2-dihydroquinoxaline-6-carboxylate (TEPS 81)

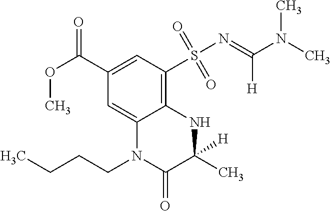 [SMILES: CCCCN1C(=O)[C@H](C)Nc2c1cc(C(=O)OC)cc2S(=O)(=O)/N=C/N(C)C]

1 mmol (354 mg) of TEPS58 were dissolved in 9 mL of N,N-dimethylformamide. 2 mmol (276 mg) of potassium carbonate and 6 mmol (687 μL) butyl iodide was added to the mixture and stirred at 90 C° for 16 hours. After the reaction was completed, the mixture was washed with water and extracted three times with ethyl acetate. The combined organic phases were washed with brine. The organic phase was dried over sodium sulfate and the filtrate was evaporated under reduced pressure. The resulting crude product was purified by column chromatography (ethyl acetate) and evaporated under reduced pressure yielding 0.32 mmol 131 mg of yellow crystals (31.9% yield).

[α]_{D}²⁰=+12.083° (c=0.5; Methanol)

¹H NMR (200 MHz, DMSO-d₆) δ 8.29 (s, 1H), 7.97 (d, J=1.7 Hz, 1H), 7.57 (d, J=1.8 Hz, 1H), 6.76 (s, 1H), 4.26 (dd, J=6.8, 1.9 Hz, 1H), 4.09-3.85 (m, 2H), 3.83 (s, 3H), 3.14 (s, 3H), 2.91 (s, 3H), 1.63-1.44 (m, 2H), 1.38-1.08 (m, 6H), 0.91 (t, J=7.1 Hz, 3H). ¹C NMR (50 MHz, DMSO) δ 166.0, 165.3, 159.4, 136.4, 127.6, 125.1, 123.5, 117.5, 117.3, 52.1, 50.8, 40.9, 35.1, 28.4, 19.4, 19.0, 13.6. MS m/z: 410 M⁺

### (2S)-4-Butyl-2-methyl-3-oxo-8-sulfamoyl-1,2-dihydroquinoxaline-6-carboxylic acid (TEPS 82)

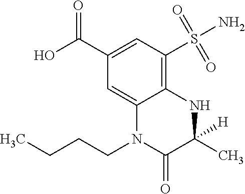 [SMILES: CCCCN1C(=O)[C@H](C)Nc2c1cc(C(=O)O)cc2S(N)(=O)=O]

0.12 mmol (50 mg) of TEPS 81 were dissolved in 1.5 mL methanol and 1.5 mL 2N sodium hydroxide and the mixture was stirred at 40 C° for 2 hours.

After the reaction was completed and the mixture was cooled down to room temperature methanol was evaporated under reduced pressure.

Upon adding 1.5 mL 2N hydrochloric acid a light-yellow precipitate was formed. The precipitate was filtered and dried under reduced pressure yielding 0.117 mmol (40 mg) of light-yellow powder (97.5% yield)

[α]_{D}²⁰=+35.405° (c=0.5; Methanol)

¹H NMR (200 MHz, DMSO-d₆) δ 12.90 (s, 1H), 8.06-7.96 (m, 1H), 7.71 (s, 1H), 7.60 (s, 1H), 6.65 (s, 1H), 4.30-4.07 (m, 1H), 3.95 (t, J=7.2 Hz, 2H), 1.65-1.42 (m, 2H), 1.30 (d, J=6.7 Hz, 5H), 0.90 (t, J=7.1 Hz, 3H). 13C NMR (50 MHz, DMSO) δ 166.3, 166.2, 136.4, 127.7, 125.9, 124.4, 118.7, 117.8, 50.7, 40.9, 28.4, 19.4, 18.3, 13.6. MS m/z: not possible EI

### 3-(Butylamino)-2-phenoxy-5-(2,2,2-trifluoroethoxymethyl)benzenesulfonamide (TEPS 83)

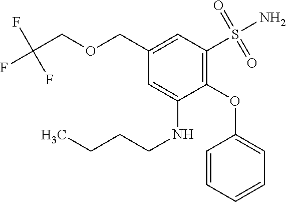 [SMILES: CCCCNc1cc(COCC(F)(F)F)cc(S(N)(=O)=O)c1Oc1ccccc1]

84 mg of a dispersion of sodium hydride in mineral oil (2 mmol) was washed two times with dry THF. 3 mL of THF was added to the sodium hydride, then 1.5 mmol (108 μl) 2,2,2-Trifluorethanol and 0.5 mmol (184 mg) of TEPS 76 were added. The reaction mixture was stirred at room temperature overnight. After the reaction was completed it was quenched with 3 mL of water. The mixture was then extracted three times with ethyl acetate, the combined organic layers are washed with brine and dried over Na₂SO₄. The organic layers are dried under reduced pressure to yield a white crude product. The crude product was further purified by column chromatography (ethyl acetate/petroleum ether 3+7) and recrystallization from EtOH to yield 78 mg of white powder (36% yield). ¹H NMR (200 MHz, DMSO-d₆) δ 7.40-7.03 (m, 5H), 7.07-6.72 (m, 4H), 4.67 (s, 2H), 4.14 (q, J=9.4 Hz, 2H), 3.03 (t, J=6.9 Hz, 2H), 1.48-1.24 (m, 2H), 1.26-0.98 (m, 2H), 0.77 (t, J=7.2 Hz, 3H). 1C NMR (50 MHz, DMSO-d₆) δ 156.7, 142.2, 137.3, 135.8, 134.7, 129.0, 124.5 (d, J=279.2 Hz), 121.9, 115.5, 113.9, 113.0, 72.9, 66.6 (q, J=32.8 Hz), 42.0, 30.3, 19.3, 13.6. MS m/z 443

### 4-(4-Fluorophenoxy)-3-nitro-5-sulfamoyl-benzoic Acid (TEPS 84) (WO 2012/018635)

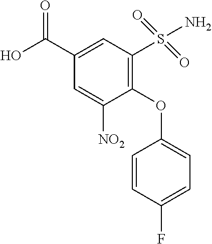 [SMILES: NS(=O)(=O)c1cc(C(=O)O)cc([N+](=O)[O-])c1Oc1ccc(F)cc1]

To a suspension of 20 mmol (5.61 g) of 4-chloro-3-nitro-5-sulfamoyl-benzoic acid (561 mg) in 30 mL water, 80 mmol NaHCO₃ (6.8 g) were added cautiously followed by 40 mmol (4.77 g) 4-fluorophenol. This solution was stirred at 85° for 16 hours. After cooling to room temperature, the precipitate was filtered off and dissolved in 10 mL of hot water. Then 6N HCl was added and the resulting precipitate was filtered off and dried to yield 4.35 g of a yellow solid (61% yield). ¹H NMR (200 MHz, DMSO) δ 14.01 (brs, 1H), 8.83-8.54 (m, 2H), 7.88 (s, 2H), 7.15 (t, J=8.8 Hz, 2H), 7.05-6.86 (m, 3H). ¹³C NMR (50 MHz, DMSO) δ 164.7, 158.56 (d, J=239.5 Hz), 153.25 (d, J=2.3 Hz), 148.2, 143.4, 140.3, 133.6, 130.9, 128.6, 118.24 (d, J=8.5 Hz), 116.56 (d, J=23.7 Hz). MS m/z 356

### 3-Amino-4-(4-fluorophenoxy)-5-sulfamoyl-benzoic Acid (TEPS 85)

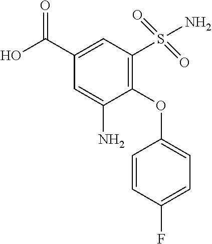 [SMILES: Nc1cc(C(=O)O)cc(S(N)(=O)=O)c1Oc1ccc(F)cc1]

To an aqueous solution of LiOH (adjusted to pH 11) 10 mmol (3.56 g) TEPS84 and 350 mg palladium on activated charcoal (5% Pd/C) were added. The resulting mixture was hydrogenated at room temperature. When the H₂ uptake became negligible, the mixture was filtered and the filtrate was acidified with 6N HCl and extracted with ethyl acetate three times. The combined organic layers were washed with brine, dried over Na₂SO₄ and dried under reduced pressure to yield 2.15 g of a brown solid (66% yield). ¹H NMR (200 MHz, DMSO) δ 7.78-7.47 (m, 2H), 7.30 (s, 2H), 7.19-7.00 (m, 2H), 6.99-6.76 (m, 2H), 5.32 (s, 2H). ¹³C NMR (50 MHz, DMSO) δ 166.9, 157.8 (d, J=236.9 Hz), 152.8 (d, J=2.0 Hz), 143.2, 139.3, 138.3, 128.3, 120.7, 117.3 (d, J=8.4 Hz), 116.1, 115.7. MS m/z 326

### Methyl 3-(benzylamino)-4-(4-fluorophenoxy)-5-sulfamoyl-benzoate (TEPS 86)

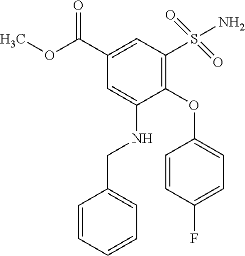 [SMILES: COC(=O)c1cc(NCc2ccccc2)c(Oc2ccc(F)cc2)c(S(N)(=O)=O)c1]

To a suspension of 2 mmol (652 mg) TEPS85 in 10 mL MeOH 5 mmol (0.6 mL) benzylbromide were added. The mixture was then refluxed for 16 hour to form a solution. After the reaction was completed, MeOH was removed under reduced pressure and 20 mL 5% NaHCO₃ were added. This mixture was extracted three times with ethyl acetate and the combined organic layers were washed with brine, dried over Na₂SO₄ and the solvent was removed under reduced pressure. The crude product was purified by column chromatography (ethyl acetate/petroleum ether 3+7) to yield 351 mg of a white solid (41% yield). ¹H NMR (200 MHz, DMSO) δ 7.67 (d, J=1.9 Hz, 1H), 7.40 (s, 2H), 7.35-7.04 (m, 8H), 6.96-6.80 (m, 2H), 6.21 (t, J=6.0 Hz, 1H), 4.35 (d, J=6.0 Hz, 2H), 3.81 (s, 3H). ¹³C NMR (50 MHz, DMSO) δ 165.8, 158.0 (d, J=237.2 Hz), 153.1 (d, J=2.0 Hz), 142.8, 140.5, 139.5, 138.3, 128.8, 127.3, 127.3, 127.1, 117.4 (d, J=8.3 Hz), 116.1, 115.7, 52.9, 46.2. MS m/z 430

### 3-(Benzylamino)-2-(4-fluorophenoxy)-5-(hydroxymethyl)benzenesulfonamide (TEPS 87)

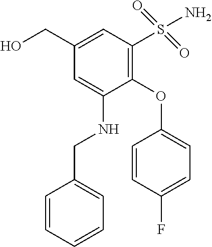 [SMILES: NS(=O)(=O)c1cc(CO)cc(NCc2ccccc2)c1Oc1ccc(F)cc1]

In a three necked flask 2 mmol of TEPS86 (860 mg) were dissolved in 8 mL anhydrous THF under argon atmosphere. Then 4 mL of a 1 M DIBAL-H solution in toluene were added. After one, two, three and four hours, respectively, another 2 mL of the 1M DIBAL-H solution in toluene were added each time and the reaction was stirred overnight. After TLC showed no remaining TEPS86 the mixture was cooled to 0° C. and quenched with 5% aqueous NH₄Cl solution causing a gel-like substance to precipitate. The precipitate was then dissolved in 2 N HCl and extracted three times with ethyl acetate. The combined organic layers were washed three times with water, once with brine and dried over Na₂SO₄. The fluids were evaporated under reduced pressure and purified by recrystallization from ethanol to yield 665 mg of beige powder (83% yield). ¹H NMR (200 MHz, DMSO) δ 7.34-7.00 (m, 10H), 6.92-6.74 (m, 3H), 5.86-5.69 (m, 1H), 4.38 (s, 2H), 4.30 (d, J=5.2 Hz, 2H). 13C NMR (50 MHz, DMSO) δ 157.7 (d, J=236.5 Hz), 153.7, 140.6, 140.0, 137.4, 135.5, 128.7, 128.1, 127.2, 117.2 (d, J=8.1 Hz), 115.7 (d, J=23.3 Hz), 113.5, 112.6, 63.0, 46.3. MS m/z 402

### 3-(Benzylamino)-2-(4-fluorophenoxy)-5-[(2,2,2-trifluoroethylamino)methyl]benzenesulfonamide (TEPS 88)

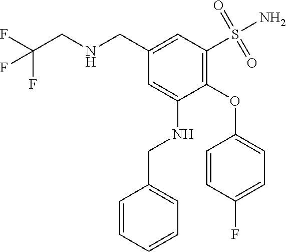 [SMILES: NS(=O)(=O)c1cc(CNCC(F)(F)F)cc(NCc2ccccc2)c1Oc1ccc(F)cc1]

1.5 mmol (604 mg) of TEPS87 were dissolved in 5 mL thionyl chloride and heated to 80 C° for three hours. The thionyl chloride was evaporated under reduced pressure. The product was purified by column chromatography (ethyl acetate/petroleum ether 7+3) to yield 470 mg of brown solid (74% yield). 1 mmol (420 mg) of this intermediate benzyl chloride was dissolved in 5 mL of DMF, to this solution 2 mmol (157 μl) of 2,2,2-trifluoroethylamine were added and the mixture was stirred at room temperature overnight in a sealed vial. After the reaction was completed, which was verified by thin layer chromatography, the fluid was evaporated under reduced pressure. This crude product was purified by column chromatography (ethyl acetate/petroleum ether 3+7) and recrystallization from ethanol, yielding 86 mg of white crystals (18% yield). ¹H NMR (200 MHz, MeOD) δ 7.32-7.11 (m, 6H), 7.09-6.71 (m, 5H), 4.34 (d, J=3.8 Hz, 2H), 3.84 (d, J=26.0 Hz, 2H), 3.05 (q, J=9.8 Hz, 2H). 13C NMR (50 MHz, MeOD) δ 142.0, 139.0, 137.3, 128.1, 126.7, 116.5, 116.4, 115.5, 115.4, 115.0, 114.2, 52.1, 46.4. MS m/z 483

### 3-(Butylamino)-2-phenoxy-5-(2,2,2-trifluoroethylsulfanylmethyl)benzenesulfonamide (TEPS89)

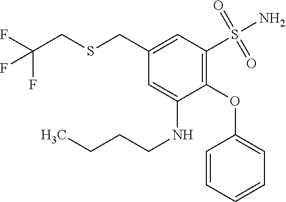 [SMILES: CCCCNc1cc(CSCC(F)(F)F)cc(S(N)(=O)=O)c1Oc1ccccc1]

84 mg of a dispersion of sodium hydride in mineral oil (2 mmol) was washed two times with dry THF. 3 mL of THF was added to the sodium hydride, then 1 mL 2,2,2-trifluoroethanthiol and 1 mmol (369 mg) of TEPS76 were added and the vial was sealed. The reaction mixture was stirred at room temperature overnight. After the reaction was completed it was quenched with 3 mL of water. The mixture was then extracted three times with ethyl acetate, the combined organic layers are washed with brine and dried over Na₂SO₄. The organic layers were dried under reduced pressure and purified by column chromatography (ethyl acetate/petroleum ether 4+6) and recrystallization from ethanol to yield 257 mg of white powder (57% yield). ¹H NMR (200 MHz, CDCl₃) δ 7.38-7.18 (m, 3H), 7.08 (t, J=7.3 Hz, 1H), 6.99-6.75 (m, 3H), 4.89 (s, 2H), 3.83 (s, 2H), 3.16-2.82 (m, 4H), 1.59-1.28 (m, 2H), 1.31-1.03 (m, 2H), 0.82 (t, J=7.1 Hz, 3H). ¹³C NMR (50 MHz, CDCl₃) δ 156.2, 142.5, 136.1, 136.0, 135.1, 130.2, 126.1 (d, J=276.9 Hz), 123.6, 116.3, 115.4, 43.2, 36.8, 33.4 (q, J=32.6 Hz), 31.2, 31.1, 19.9, 13.8. MS m/z 448

### 3-(Butylamino)-2-phenoxy-5-(2,2,2-trifluoroethylsulfinylmethyl)benzenesulfonamide (TEPS 90)

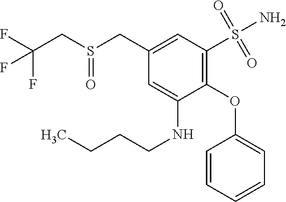 [SMILES: CCCCNc1cc(CS(=O)CC(F)(F)F)cc(S(N)(=O)=O)c1Oc1ccccc1]

To a stirred solution of 0.5 mmol TEPS89 (224 mg) in 5 mL acetonitril was added a solution of 0.35 mmol Oxone (53 mg) in 2 mL water. The reaction was stirred at room temperature for two days until no TLC showed no remaining TEPS89 and then poured into 10 mL of ice water. The solid was filtered and recrystallized from ethanol to yield 162 mg of white powder (70% yield). ¹H NMR (200 MHz, DMSO) δ 7.39-7.12 (m, 4H), 7.11-6.75 (m, 5H), 4.91 (t, J=5.8 Hz, 1H), 4.28 (q, J=12.8 Hz, 2H), 4.14-3.75 (m, 2H), 3.03 (q, J=6.5 Hz, 2H), 1.50-1.22 (m, 2H), 1.24-0.95 (m, 2H), 0.77 (t, J=7.2 Hz, 3H). ¹³C NMR (50 MHz, DMSO) δ 156.6, 142.3, 137.4, 136.2, 129.0, 128.3, 127.6, 122.0, 116.5, 115.5, 115.5, 57.4, 53.3 (d, J=26.9 Hz), 42.1, 30.2, 19.3, 13.6. MS m/z 464

### 3-(2,2,2-Trifluoroethylsulfanylmethyl)benzenesulfonamide (TEPS 91)

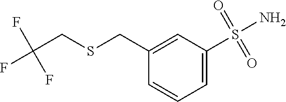 [SMILES: NS(=O)(=O)c1cccc(CSCC(F)(F)F)c1]

84 mg of a dispersion of sodium hydride in mineral oil (2 mmol) was washed two times with dry THF. 3 mL of THF was added to the sodium hydride, then 1 mL 2,2,2-trifluoroethanthiol and 1 mmol (250 mg) of 3-bromomethyl-benzenesulfonamide were added. The reaction mixture was stirred at room temperature overnight. After the reaction was completed it was quenched with 3 mL of water. The mixture was then extracted three times with ethyl acetate, the combined organic layers are washed with brine and dried over Na₂SO₄. The organic layers are dried under reduced pressure to yield a white crude product. The crude product was further purified by column chromatography (ethyl acetate/petroleum ether 1+1) and recrystallization from EtOH to yield 135 mg of white powder (61% yield). ¹H NMR (200 MHz, MeOD) δ 7.90 (s, 1H), 7.90-7.75 (m, 1H), 7.65-7.41 (m, 2H), 3.97 (s, 2H), 3.16 (q, J=10.2 Hz, 2H). ¹³C NMR (50 MHz, MeOD) δ 145.5, 139.9, 133.8, 130.4, 127.7 (d, J=275.4 Hz), 127.6, 126.2, 37.2, 33.85 (q, J=32.7 Hz). MS m/z 285

### 3-(Butylamino)-2-phenoxy-5-(3,3,3-trifluoropropylsulfanylmethyl)benzenesulfonamide (TEPS 92)

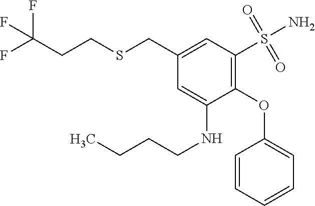 [SMILES: CCCCNc1cc(CSCCC(F)(F)F)cc(S(N)(=O)=O)c1Oc1ccccc1]

84 mg of a dispersion of sodium hydride in mineral oil (2 mmol) was washed two times with dry THF. 3 mL of THF was added to the sodium hydride, then 1 mL 2,2,2-trifluoropropanthiol and 1 mmol (369 mg) of TEPS76 were added and the vial was sealed. The reaction mixture was stirred at room temperature for two days. After the reaction was completed it was quenched with 3 mL of water. The mixture was then extracted three times with ethyl acetate, the combined organic layers are washed with brine and dried over Na₂SO₄. The organic layers were dried under reduced pressure and purified by column chromatography (ethyl acetate/petroleum ether 4+6) and recrystallization from ethanol to yield 137 mg of white powder (30% yield). ¹H NMR (200 MHz, CDCl₃) δ 7.44-7.18 (m, 3H), 7.08 (t, J=7.3 Hz, 1H), 6.98-6.80 (m, 3H), 4.89 (s, 2H), 3.73 (s, 2H), 3.05 (t, J=6.9 Hz, 2H), 2.74-2.50 (m, 2H), 2.51-2.17 (m, 2H), 1.54-1.30 (m, 2H), 1.31-1.01 (m, 2H), 0.82 (t, J=7.2 Hz, 3H). ¹³C NMR (50 MHz, MeOD) δ 156.2, 142.4, 136.2, 135.9, 135.8, 134.3, 130.1, 126.1 (d, J=277.2 Hz), 123.6, 117.4, 116.0, 115.3, 115.2, 43.2, 36.5, 34.6 (q, J=28.7 Hz), 31.1, 23.8 (q, J=3.3 Hz), 19.9, 13.8. MS m/z 462

### 3-(Butylamino)-5-[[2-(dimethylamino)-ethylamino]-methyl]-2-phenoxy-benzenesulfonamide Hydrochloride (TEPS 93)

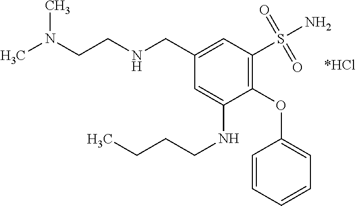 [SMILES: CCCCNc1cc(CNCCN(C)C)cc(S(N)(=O)=O)c1Oc1ccccc1]

2 mmol (740 mg) of TEPS76 were dissolved in a solution consisting of 5 mL DMF and 1 mL TEA. To this 2 mmol (440 μL) of N,N′-dimethylethylendiamine 98% were added and the mixture was stirred at room temperature overnight. After the reaction was accomplished, it was purified through column chromatography (ethyl acetate/triethylamine/EtOH 6+3+1). The crude product was dissolved in dry THF and 1 mL of a hydrogen chloride solution 1.0 M in diethyl ether was added. The resulting precipitate was filtered off to yield 123 mg of yellow powder (13% yield). ¹H NMR (200 MHz, MeOD) δ 7.42 (d, J=2.0 Hz, 1H), 7.37-7.18 (m, 3H), 7.05 (t, J=7.3 Hz, 1H), 6.91 (d, J=7.6 Hz, 2H), 4.40-4.23 (m, 2H), 3.65 (d, J=10.5 Hz, 4H), 3.15 (t, J=6.8 Hz, 2H), 3.00 (s, 6H), 1.57-1.27 (m, 2H), 1.30-1.00 (m, 2H), 0.81 (t, J=7.2 Hz, 3H). ¹³C NMR (50 MHz, MeOD) δ 156.6, 142.7, 137.9, 137.4, 129.2, 128.7, 122.6, 116.7, 115.8, 115.2, 52.9, 51.2, 42.7, 42.6, 41.9, 30.5, 19.5, 12.6. MS EI not possible

### 5-(1,3-Benzothiazolyl-2-sulfanylmethyl)-3-(butylamino)-2-phenoxy-benzenesulfonamide (TEPS 93)

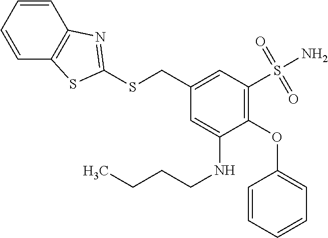 [SMILES: CCCCNc1cc(CSc2nc3ccccc3s2)cc(S(N)(=O)=O)c1Oc1ccccc1]

1 mmol (368 mg) of TEPS76 was dissolved in a solution consisting of 3 mL DMF and 1 mL TEA. Thereafter, 2 mmol (338 mg) 2-mercaptobenzothiazole were added and the mixture was stirred at room temperature overnight. After the reaction was accomplished, it was purified through column chromatography (ethyl acetate/petroleum ether 3+7). The crude product was then purified by recrystallization from EtOH to yield 140 mg of white crystal (28% yield). ¹H NMR (200 MHz, DMSO) δ 8.01 (d, J=7.8 Hz, 1H), 7.89 (d, J=7.8 Hz, 1H), 7.54-7.32 (m, 2H), 7.30-7.07 (m, 6H), 6.97 (t, J=7.3 Hz, 1H), 6.88-6.62 (m, 2H), 4.83 (t, J=5.9 Hz, 1H), 4.67 (s, 2H), 3.10-2.79 (m, 2H), 1.41-1.15 (m, 2H), 1.13-0.87 (m, 2H), 0.68 (t, J=7.1 Hz, 3H). ¹³C NMR (50 MHz, DMSO) δ 166.5, 157.1, 153.0, 142.6, 137.8, 136.0, 136.0, 135.2, 135.0, 129.5, 126.9, 125.1, 122.4, 122.3, 121.6, 115.9, 114.7, 42.5, 36.9, 30.7, 19.7, 14.0. MS m/z 499

### 3-(Butylamino)-2-phenoxy-5-(2-pyridylsulfanylmethyl)benzenesulfonamide (TEPS 95)

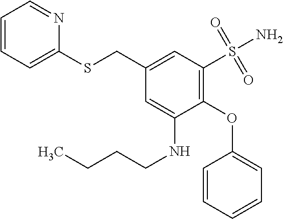 [SMILES: CCCCNc1cc(CSc2ccccn2)cc(S(N)(=O)=O)c1Oc1ccccc1]

To 3 mL DMF and 2 mL triethylamine 1 mmol (369 mg) TEPS76 and 1 mmol (114 mg) 2-mercaptopyridine were added and stirred overnight at room temperature. After the reaction was completed DMF was evaporated and water was added. The mixture was extracted three times with ethyl acetate, washed three times with water and once with brine. It was purified by column chromatography (ethyl acetate/petroleum ether 4+6). After that the product was recrystallized from 70% isopropanol yielding 221 mg of light yellow crystals. ¹H NMR (200 MHz, chloroform-d) δ 8.46 (d, J=3.4 Hz, 1H), 7.59-7.41 (m, 1H), 7.39-7.13 (m, 4H), 7.13-6.83 (m, 5H), 4.87 (s, 2H), 4.43 (s, 2H), 3.00 (t, J=6.9 Hz, 2H), 1.45-1.25 (m, 2H), 1.24-1.03 (m, 2H), 0.80 (t, J=7.2 Hz, 3H). ¹³C NMR (50 MHz, chloroform-d) δ 158.1, 156.2, 149.4, 142.1, 136.8, 136.2, 135.5, 129.9, 123.3, 122.3, 119.9, 116.5, 115.4, 115.2, 100.0, 100.0, 43.0, 34.0, 31.0, 19.8, 13.7. MS m/z 444

### 3-(Butylamino)-5-(1H-imidazol-2-ylsulfanylmethyl)-2-phenoxy-benzenesulfonamide (TEPS96)

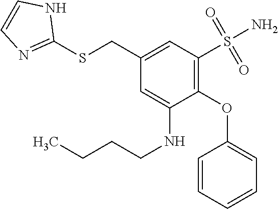 [SMILES: CCCCNc1cc(CSc2ncc[nH]2)cc(S(N)(=O)=O)c1Oc1ccccc1]

1 mmol (369 mg) of 3-(butylamino)-5-(chloromethyl)-2-phenoxy-benzenesulfonamide (TEPS 76) was dissolved in 3 mL DMF and 1 mL TEA. To this 2 mmol (205 mg) of 2-mercaptoimidazole were added and the mixture was stirred at room temperature overnight. After the reaction was accomplished, which was verified by the thin layer chromatography, it was purified through column chromatography with the mobile phase consisting of ethyl acetate and petroleum ether (9+1). Those fractions which contained the sample were united and the mobile phase was evaporated under reduced pressure, yielding 308 mg of white brown powder (71% yield). ¹H NMR (200 MHz, MeOD) δ 7.35-7.17 (m, 2H), 7.14-6.93 (m, 4H), 6.93-6.77 (m, 2H), 6.62-6.59 (m, 1H), 4.14 (s, 1H), 2.94 (t, J=6.7 Hz, 2H), 1.46-1.22 (m, 2H), 1.23-0.98 (m, 2H), 0.80 (t, J=7.1 Hz, 3H). ¹³C NMR (50 MHz, MeOD) δ 156.8, 142.2, 138.4, 136.5, 136.0, 135.8, 129.1, 122.3, 115.3, 115.1, 114.4, 42.3, 39.1, 30.6, 19.5, 12.6. MS m/z 432

### 3-(Butylamino)-2-phenoxy-5-[(2-pyridylamino)methyl]benzenesulfonamide (TEPS 97)

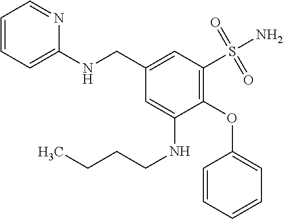 [SMILES: CCCCNc1cc(CNc2ccccn2)cc(S(N)(=O)=O)c1Oc1ccccc1]

1 mmol (348 mg) of 3-(butylamino)-5-formyl-2-phenoxy-benzenesulfonamide was dissolved in 10 mL 1,2-dichlorethane and 1 mmol (29 μL) of acetic acid and 1.2 mmol 2-aminopyridine were added. After stirring the mixture for two hours, 1.5 mmol of triacetoxyborohydride (NaBH(OAc)₃) were added and the mixture was stirred overnight. The product was diluted with 40 mL of dichloromethane and 10 mL saturated NaHCO₃. After washing it with brine it was evaporated under reduced pressure. The purification was made by column chromatography (ethyl acetate/petroleum ether 6+4) and recrystallization out of 70% EtOH, resulting in 130 mg of white crystals (30% yield). ¹H NMR (200 MHz, DMSO) δ 8.06-7.88 (m, 1H), 7.51-6.30 (m, 12H), 4.76-4.61 (m, 1H), 4.55-4.41 (m, 2H), 3.13-2.87 (m, 2H), 1.40-1.20 (m, 2H), 1.19-0.97 (m, 2H), 0.74 (t, J=7.0 Hz, 3H). ¹³C NMR (50 MHz, DMSO) δ 159.0, 157.3, 147.9, 142.4, 138.9, 137.3, 137.3, 135.2, 129.5, 122.4, 115.9, 114.2, 112.8, 112.4, 108.6, 44.4, 42.6, 30.8, 19.8, 14.1. MS m/z 427

### 3-(Butylamino)-N-(4-fluorophenyl)-4-phenoxy-5-sulfamoyl-benzamide (TEPS 98)

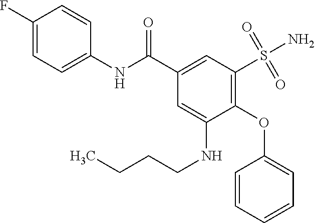 [SMILES: CCCCNc1cc(C(=O)Nc2ccc(F)cc2)cc(S(N)(=O)=O)c1Oc1ccccc1]

1 mmol (364 mg) of bumetanide was dissolved in 5 mL THF, then 1.2 mmol (194 mg) 1,1-carbonyldiimidazole were added and the mixture was stirred for three hours. Afterwards 2 mmol (222 mg) 4-Fluoroaniline were added and the mixture was stirred at room temperature overnight. After the reaction was completed, 20 mL of 5% sodium bicarbonate solution were added and the reaction was extracted three times with ethyl acetate. The collected organic phase was washed with saturated sodium chloride solution, dried over sodium sulfate and evaporated under reduced pressure. The crude product was purified via recrystallization using EtOH to yield 200 mg yellow powder (44% yield). ¹H NMR (200 MHz, DMSO) δ 10.40 (s, 1H), 7.86-7.69 (m, 2H), 7.68 (d, J=1.9 Hz, 1H), 7.45 (d, J=1.9 Hz, 1H), 7.41-7.10 (m, 6H), 7.02 (t, J=7.3 Hz, 1H), 6.96-6.77 (m, 2H), 4.97 (t, J=5.6 Hz, 1H), 3.12 (q, J=6.5 Hz, 2H), 1.49-1.28 (m, 2H), 1.23-0.97 (m, 2H), 0.78 (t, J=7.2 Hz, 3H). ¹³C NMR (50 MHz, DMSO) δ 164.7, 158.41 (d, J=240.4 Hz) 156.4, 142.2, 138.5, 137.5, 135.32 (d, J=2.5 Hz), 132.2, 129.1, 122.54 (d, J=7.9 Hz), 122.2, 115.6, 115.19 (d, J=22.2 Hz), 113.6, 42.1, 30.3, 19.3, 13.6. MS m/z 347

### 3-(Butylamino)-5-[(4-fluoroanilino)methyl]-2-phenoxy-benzenesulfonamide (TEPS 99)

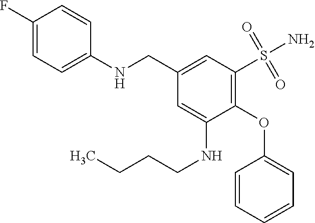 [SMILES: CCCCNc1cc(CNc2ccc(F)cc2)cc(S(N)(=O)=O)c1Oc1ccccc1]

0.33 mmol (150 mg) of TEPS98 was dissolved in 20 mL tetrahydrofuran. 1.32 mmol (4 equivalent, 100 mg) of borane dimethyl sulfide complex were added. The mixture was stirred at 86° C. overnight under reflux. After TLC showed that no starting material was present, the mixture was cooled down to room temperature and quenched with 20 mL of half-saturated aqueous amount of NaHCO₃. Afterwards the mixture was extracted three times with ethyl acetate, washed with saturated sodium chloride solution and dried over sodium sulfate. The solvent was removed under reduced pressure and the crude product was purified by recrystallization using EtOH. Yield: 56 mg (38%)¹H NMR (200 MHz, DMSO) δ 7.25 (t, J=7.7 Hz, 2H), 7.15-7.04 (m, 3H), 7.06-6.76 (m, 6H), 6.67-6.50 (m, 2H), 6.24 (t, J=6.0 Hz, 1H), 4.72 (t, J=5.7 Hz, 1H), 4.24 (d, J=5.9 Hz, 2H), 2.99 (q, J=6.2 Hz, 2H), 1.40-1.18 (m, 2H), 1.21-0.93 (m, 2H), 0.74 (t, J=7.1 Hz, 3H). ¹³C NMR (50 MHz, DMSO) δ 156.8, 154.32 (d, J=241.5 Hz), 145.3 145.29 (d, J=1.4 Hz), 137.9, 137.1, 134.9, 129.0, 121.8, 115.5, 115.20 (d, J=21.9 Hz), 113.5, 112.98 (d, J=7.3 Hz), 112.4, 46.8, 42.1, 39.5, 30.3, 19.3, 13.6. MS m/z 333

### N-(2,7α-Dihydro-1H-benzimidazol-2-yl)-3-(butylamino)-4-phenoxy-5-sulfamoyl-benzamide (TEPS 100)

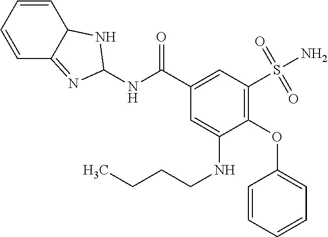 [SMILES: CCCCNc1cc(C(=O)NC2N=C3C=CC=CC3N2)cc(S(N)(=O)=O)c1Oc1ccccc1]

2 mmol (728 mg) of bumetanide was dissolved in in 5 mL dry tetrahydrofuran. 2.4 mmol (388 mg) 1,1-carbonyldiimidazole were added and the mixture was stirred for two hours. After the thin-layer chromatography showed no remaining bumetanide, 4 mmol (532 mg) trifluoropropan-1-amine were added and the mixture was stirred at room temperature overnight. After the reaction was completed 20 mL of 5% NaHCO₃ were added and it extracted three times with ethyl acetate. The collected organic phase was washed with brine and dried over Na₂SO₄. The solvent was then removed under reduced pressure. The crude product was purified via recrystallization from MeOH. Yield: 413 mg (43%). ¹H NMR (200 MHz, DMSO) δ 12.38 (brs, 1H), 7.95 (d, J=1.7 Hz, 1H), 7.74 (d, J=1.7 Hz, 1H), 7.50-7.40 (m, 2H), 7.35-7.22 (m, 4H), 7.20-7.13 (m, 2H), 7.01 (t, J=7.3 Hz, 1H), 6.88 (d, J=7.8 Hz, 2H), 4.90 (t, J=5.5 Hz, 1H), 3.13 (q, J=6.4 Hz, 2H), 1.41 (p, J=6.9 Hz, 2H), 1.25-1.04 (m, 2H), 0.79 (t, J=7.2 Hz, 3H). ¹³C NMR (50 MHz, DMSO) δ 156.5, 142.1, 138.8, 137.4, 129.2, 122.3, 121.9, 115.6, 114.8, 114.3, 42.2, 30.4, 19.4, 13.7. MS m/z 481

### 3-(Butylamino)-4-phenoxy-5-sulfamoyl-N-(3,3,3-trifluoropropyl)benzamide (TEPS 101)

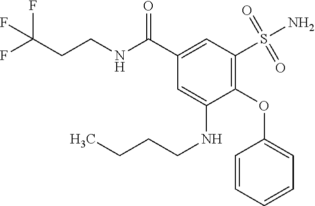 [SMILES: CCCCNc1cc(C(=O)NCCC(F)(F)F)cc(S(N)(=O)=O)c1Oc1ccccc1]

1 mmol (364 mg) of bumetanide was dissolved in in 5 mL dry tetrahydrofuran. 1.2 mmol (194 mg) 1,1-carbonyldiimidazole were added and the mixture was stirred for three hours. After the thin-layer chromatography showed that all bumetanide reacted, 2 mmol (300 mg) trifluoropropan-1-Amine were added and the mixture was stirred at room temperature overnight. After the reaction was completed 20 mL of 5% NaHCO₃ were added and it was extracted three times with ethyl acetate. The collected organic phase was washed with brine and dried over Na₂SO₄. The solvent was then removed under reduced pressure. The crude product was purified via recrystallization from EtOH. Yield: 220 mg (47%).

¹H NMR (200 MHz, MeOD) δ 7.74-7.61 (m, 2H), 7.40 (d, J=2.0 Hz, 1H), 7.29 (t, J=7.9 Hz, 2H), 7.10-7.03 (m, 2H), 6.96-6.85 (m, 2H), 3.64 (t, J=7.0 Hz, 2H), 3.12 (t, J=6.8 Hz, 2H), 2.70-2.37 (m, 2H), 1.42 (p, J=6.8 Hz, 2H), 1.24-1.05 (m, 2H), 0.81 (t, 3H). ¹³C NMR (50 MHz, MeOD) δ 169.1, 157.8, 144.0, 140.6, 138.4, 132.9, 130.7, 127.96 (d, J=276.2 Hz), 124.0, 116.6, 114.9, 114.5, 43.7, 34.60 (q, J=4.0 Hz), 34.03 (q, J=27.8 Hz), 32.0, 20.8, 14.0. MS m/z 459

### 3-(Butylamino)-2-phenoxy-5-[(3,3,3-trifluoropropylamino)methyl]benzenesulfonamide (TEPS102)

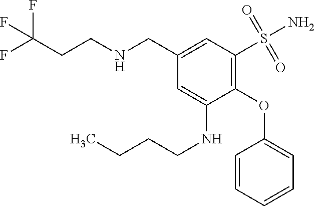 [SMILES: CCCCNc1cc(CNCCC(F)(F)F)cc(S(N)(=O)=O)c1Oc1ccccc1]

1.56 mmol (363 mg) of TEPS101 was dissolved in 20 mL of THF and 5.8 mmol (0.556 mL) borane dimethylsulfid complex was added. The reaction mixture was then stirred at 86° overnight. Once TLC showed that no starting material was present, the mixture was cooled to room temperature and then quenched with 20 mL of half-saturated aqueous NaHCO₃. It was extracted three times with 25 mL of ethyl acetate, washed brine and dried over Na₂SO₄. The solvent was removed under reduced pressure and the crude product was purified by column chromatography (ethyl acetate/petroleum ether and TEA, 1:1+20 mL of TEA) and recrystallized from 70% EtOH to yield 178 mg. (Yield 26%). ¹H NMR (200 MHz, CDCl₃) δ 7.38-7.16 (m, 3H), 7.06 (t, J=7.3 Hz, 1H), 6.91 (d, J=7.3 Hz, 3H), 4.90 (s, 1H), 3.79 (s, 2H), 3.06 (q, J=6.7 Hz, 2H), 2.91 (t, J=7.1 Hz, 2H), 2.34 (qt, J=10.9, 7.1 Hz, 2H), 1.41 (p, J=6.8 Hz, 2H), 1.17 (dq, J=13.7, 6.9 Hz, 2H), 0.82 (t, J=7.2 Hz, 3H). ¹³C NMR (50 MHz, CDCl₃) δ 156.4, 142.4, 138.2, 135.7, 135.6, 133.0, 130.1, 126.79 (d, J=276.8 Hz), 123.5, 120.5, 115.5, 115.3, 114.3, 53.4, 43.2, 42.23 (q, J=3.3 Hz), 34.41 (q, J=27.7 Hz), 31.2, 19.9, 13.8. MS m/z 445

### 3-(Butylamino)-N-(2-morpholinoethyl)-4-phenoxy-5-sulfamoyl-benzamide (TEPS 103)

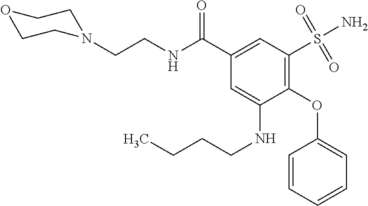 [SMILES: CCCCNc1cc(C(=O)NCCN2CCOCC2)cc(S(N)(=O)=O)c1Oc1ccccc1]

To a solution of 1 mmol (364 mg) of bumetanide in 5 mL dry THF, 1.2 mmol (194 mg) of 1,1-carbonyldiimidazole (CDI) were added and the mixture was stirred for 3 hours. Once TLC showed that all the bumetanide reacted, 2 mmol (0.262 mL) of 4-(2aminoethyl)morpholin were added and the mixture was stirred at room temperature overnight. After the reaction was completed, 20 mL of 5% NaHCO₃ were added and it was extracted with 25 mL ethyl acetate three times. The organic Phase was washed brine and then dried over Na₂SO₄. The solvent was removed under reduced pressure. The crude product was purified via flash column chromatography (TEA/ethyl acetate, 1:9). The obtained substance was dried under vacuum to yield 385 mg of white powder (80% yield). ¹H NMR (200 MHz, MeOD) δ 7.69 (d, J=2.0 Hz, 1H), 7.42 (d, J=2.0 Hz, 1H), 7.29 (t, J=7.9 Hz, 2H), 7.09-7.00 (m, 2H), 6.96-6.87 (m, 2H), 3.76-3.67 (m, 4H), 3.56 (t, J=6.6 Hz, 2H), 3.13 (t, J=6.8 Hz, 2H), 2.67-2.50 (m, 6H), 1.43 (p, J=6.9 Hz, 2H), 1.28-1.09 (m, 2H), 0.82 (t, J=7.2 Hz, 3H). ¹³C NMR (50 MHz, MeOD) δ 169.0, 157.8, 143.9, 140.4, 138.3, 130.6, 124.0, 116.6, 115.0, 114.5, 67.7, 58.6, 54.7, 43.7, 37.8, 32.0, 20.8, 14.0. MS m/z 476

### 3-(Butylamino)-4-phenoxy-N-[2-(1-piperidyl)ethyl]-5-sulfamoyl-benzamide (TEPS 104)

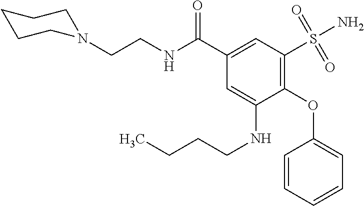 [SMILES: CCCCNc1cc(C(=O)NCCN2CCCCC2)cc(S(N)(=O)=O)c1Oc1ccccc1]

To a solution of 1 mmol (364 mg) of bumetanide in 5 mL dry THF, 1.2 mmol (194 mg) of 1,1-carbonyldiimidazole (CDI) were added and the mixture was stirred for 2 hours. Once TLC showed that all the bumetanide reacted, 2 mmol (0.285 mL) of 4-(2aminoethyl)piperidin were added and the mixture was stirred at room temperature overnight. After the reaction was completed, 20 mL of 5% NaHCO₃ were added and it was extracted with 25 mL ethyl acetate three times. The organic Phase was washed with brine and then dried over Na₂SO₄. The solvent was removed under reduced pressure. The crude Product was purified by recrystallization from 70% EtOH to yield 302 mg (Yield 67%). ¹H NMR (200 MHz, MeOD) δ 7.69 (d, J=2.0 Hz, 1H), 7.42 (d, J=2.0 Hz, 1H), 7.29 (t, J=7.9 Hz, 2H), 7.05 (t, J=7.3 Hz, 1H), 6.97-6.84 (m, 2H), 3.56 (t, J=6.8 Hz, 2H), 3.13 (t, J=6.8 Hz, 2H), 2.67-2.45 (m, 6H), 1.73-1.33 (m, 8H), 1.26-1.05 (m, 2H), 0.82 (t, J=7.2 Hz, 3H). ¹³C NMR (50 MHz, MeOD) δ 169.0, 157.8, 143.9, 140.5, 138.3, 133.3, 130.6, 124.0, 116.6, 115.0, 114.5, 58.9, 55.5, 43.7, 38.0, 32.0, 26.6, 25.1, 20.9, 14.0. MS m/z 474

### 3-(Butylamino)-2-phenoxy-5-(3,3,3-trifluoropropoxymethyl) benzenesulfonamide (TEPS 105)

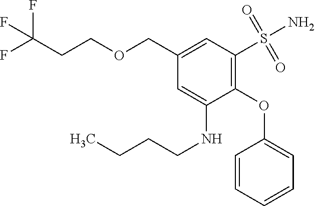 [SMILES: CCCCNc1cc(COCCC(F)(F)F)cc(S(N)(=O)=O)c1Oc1ccccc1]

In a three necked round bottom flask 4 mmol (168 mg) of a dispersion of sodium hydride in mineral oil (60%) were washed two times with dry THF. After that 5 mL of THF, 4.5 mmol (0.4 mL) 3,3,3-trifluoro-1-propanol and 1 mmol (368 mg) of TEPS76 were added. The reaction mixture was stirred at room temperature overnight. After the reaction was completed it was quenched with 10 mL water. The mixture was then extracted three times with ethyl acetate, the combined organic layers were washed with brine, dried over Na₂SO₄ and the solvent was removed under reduced pressure. The crude product was purified by column chromatography (ethyl acetate/petroleum ether 4+6) and recrystallization from toluene to yield 122 mg of white powder (27% yield). ¹H NMR (200 MHz, MeOD) δ 7.37-7.15 (m, 3H), 7.10-6.81 (m, 4H), 4.55 (s, 2H), 3.76 (t, J=6.3 Hz, 2H), 3.07 (t, J=6.8 Hz, 2H), 2.66-2.37 (m, 2H), 1.53-1.27 (m, 2H), 1.30-1.02 (m, 2H), 0.81 (t, J=7.2 Hz, 3H). ¹³C NMR (50 MHz, MeOD) δ 158.2, 143.8, 137.8, 137.5, 130.5, 123.7, 116.5, 115.4, 114.5, 73.5, 64.32 (q, J=3.6 Hz), 43.8, 35.04 (q, J=28.3 Hz), 32.1, 20.9, 14.0. MS m/z 446

### 3-[(2,2,2-Trifluoroethylamino)methyl]benzenesulfonamide

Error! Objects cannot be created from editing field codes.

5 mmol (1.41 g) of 2,2,2-trifluoro-N-[(4-sulfamoylphenyl)methyl]acetamide (Augurusa, A., et al., 2016) were dissolved in 10 mL dry tetrahydrofuran (THF). The mixture was cooled at 0-4° C. and flooded with argon gas. 25 mmol (12.5 mL) of LiAlH₄ (2.0 M in THF) were added carefully in three portions every 30 minutes, then the solution was heated to 60° C. for 3 hours. The mixture was stirred overnight at room temperature. Again, the mixture was cooled at 0-4° C. and the reaction was quenched with 5% aqueous NH₄Cl. 2 N HCl was added until the mixture was completely clear and extracted two times with ethyl acetate. The aqueous phase was neutralized by adding 2 M NaOH and again extracted two times with ethyl acetate. The second organic phase was dried over sodium sulfate and evaporated under reduced pressure. Afterwards the product was recrystallized from isopropanol. The resulting product yielded 387 mg of white crystals (28.9% yield). ¹H NMR (200 MHz, DMSO-d₆) δ 7.79 (A-part of AB system, J_{AB}=8.3 Hz, 2H), 7.52 (B-part of AB system, J_{AB}=8.3 Hz, 2H), 7.31 (s, 2H), 3.86 (d, J=5.7 Hz, 2H), 3.32-3.11 (m, 2H), 3.09-2.96 (m, 1H). ¹C NMR (50 MHz, DMSO-d₆) δ 144.3, 142.6, 126.2 (q, J=279.3 Hz), 128.1, 125.6, 51.8, 48.7 (q, J=30.3 Hz). MS m/z: 269 M⁺

### 2-(2,2,2-Trifluoroethyl)-3,4-dihydro-1H-isoquinoline-7-sulfonamide

Error! Objects cannot be created from editing field codes.

### 2-(2,2,2-Trifluoroethyl)-3,4-dihydro-1H-isoquinoline-6-sulfonamide

Error! Objects cannot be created from editing field codes.

### Example 2: NKCC1 Inhibitory Activity of the Compounds According to the Invention

The compounds of formula (I) according to the present invention are inhibitors of Na⁺—K⁺-2Cl⁻-cotransporters (NKCCs), particularly of NKCC1. The NKCC1 inhibitory activity of the compounds of the invention can be determined, for example, using the following NKCC1A activity assay.

To activate NKCC1A prior to the uptake experiment, hNKCC1A-expressing oocytes (Lykke, K., et al. 2016) or uninjected control oocytes are pre-incubated for 30 min at room temperature in a K⁺-free solution. To measure K⁺ influx, oocytes are exposed to an isosmotic test solution in which KCl is substituted for choline chloride and ⁸⁶Rb⁺ is added as a tracer for K⁺. Bumetanide (positive control), a compound of formula (I) according to the invention (“drug”), or control vehicle (negative control) are added to the test solution. The uptake assay is then performed at room temperature with mild agitation for 5 min. The influx experiments are terminated and the radioactivity present is determined by liquid scintillation β-counting with Opti-Fluor scintillation using a Liquid Scintillation Analyzer. hNKCC1A-mediated K⁺ uptake is then assessed as ([flux_{NKCC1-expressing oocytes} in presence of ×μM drug]−[flux_{uninjected oocytes} in presence of ×μM drug]), in order to correct for endogenous NKCC activity. A reduction in hNKCC1A-mediated K⁺ uptake observed with a test compound is indicative of the compound inhibiting NKCC1. When the exemplary compounds of formula (I) described in Example 1 are subjected to this assay, it can be confirmed that they exhibit NKCC1 inhibitory activity.

### REFERENCES

Alessi, D., Zhang, J., Khanna, A., Hochdorfer, T., Shang, Y., & Kahle, K. T. (2014). The WNKSPAK/OSR1 pathway: Master regulator of cation-chloride cotransporters. Science Signaling(334).
Ares G., Caceres P., Ortiz P. (2011) Molecular regulation of NKCC2 in the thick ascending limb. Am J Physiol Renal Physiol 301:F1143-F1159
Augurusa, A., Mehta, M., Perez, M., Zhu, J., Stephan, D. (2016) Catalytic Reduction of Amides by Electrophilic Phosphonium Cations via FLP Hydrosilylation. The Royal Society of Chemistry 52: 12195-12198
Cho Y., et al. (2011) Drug Repositioning and Pharmacophore Identification in the Discovery of Hookworm MIF Inhibitors. Chemistry & Biology 18, 1089-1101
Consiglio, Giovanni; Frenna, Vincenzo; Guernelli, Susanna; Macaluso, Gabriella. (2002). Analysis of substituent effects in the reactions of some 3-nitro-5-X-thiophenes with aniline in benzene. A new interpretation of the ‘element effect’. ARK/VOC (Gainesville, Fla., United States), Issue 11, Pages 104-117
Deidda, G., et al. (2015) Reversing excitatory GABAAR signaling restores synaptic plasticity and memory in a mouse model of Down syndrome. Nature Medicine 21, 318-326.
Deng Y., et al. (2016) Progress in Drug Treatment of Cerebral Edema. Mini-Reviews in Medicinal Chemistry 16: 917-925
Denys D., de Geus F. (2005) Predictors of pharmacotherapy response in anxiety disorders. Curr Psychiatry Rep 7: 252-257.
Doolittle J., et al. (2016) Hyperhidrosis: an update on prevalence and severity in the United States. Archives of Dermatological Research 1-7)
Dzhala, V. I., Talos, D. M., Sdrulla, D. A., Brumback, A. C., Mathews, G. C., Benke, T. A., & Staley, K. J. (November 2005). NKCC1 transporter facilitates seizures in the developing brain. Nature Medicine(11), pp. 1205-1213.
Hadjikhani N., et al. (2015) Improving emotional face perception in autism with diuretic bumetanide: A proof-of-concept behavioral and functional brain imaging pilot study. Autism Vol. 19(2) 149-157)
Hauck, Stefanie; Hiesinger, Kerstin; Khageh Hosseini, Sabrina; Achenbach, Janosch; Biond, Ricardo M.; Proschak, Ewgenij; Zoernig, Martin; Odadzic, Dalibor. (2016). Pyrazolo[1,-5a]-pyrimidines as a new class of FUSE binding protein 1 (FUBP1) inhibitors. Bloorganic & Medicinal Chemistry Volume 24, Issue 22, Pages 5717-5729.
Hirschfeld, R., 2003 Long-term side effects of SSRIs: sexual dysfunction and weight gain. The Journal of Clinical Psychiatry 64 Suppl 18: 20-24
Huang Y., et al. (2016) Acute spinal cord injury (SCI) transforms how GABA affects nociceptive sensitization. Experimental Neurology 285 82-95)
Kahle, K. T., & Staley, K. J. (2008, September). The bumetanide-sensitive Na—K-2C cotransporter NKCC1 as a potential target of a novel mechanism-based treatment strategy for neonatal seizures. Neurosurg Focus, pp. 1-7.
Kunzer, Aaron R.; Elmore, Steven W.; Hexamer, Laura; Park, Cheol-Min; Souers, Andrew J.; Sullivan, Gerard M.; Wang, Gary T.; Wang, Xilu; Wendt, Michael D. (2010). Preparation of heterocyclic apoptosis-inducing agents for treatment of cancer and immune disorders. PCT Int. Appl. 175 pp., Patent 2010, WO 2010083442
Krystal A D, Sutherland J, Hochman D W (2012) Loop Diuretics Have Anxiolytic Effects in Rat Models of Conditioned Anxiety. PLoS ONE 7(4): e35417).
Le'pine, J., (2002) The epidemiology of anxiety disorders: prevalence and societal costs. The Journal of Clinical Psychiatry 63 Suppl 14: 4-8
Louie J C et al., Physiol Rep. 2016, 4(22). pii: e13024)
Lykke, K., et al. (2016) The search for NKCC1-selective drugs for the treatment of epilepsy: Structure-function relationship of bumetanide and various bumetanide derivatives in inhibiting the human cation-chloride cotransporter NKCC1A. Epilepsy & Behavior, 59:42-49
Maa, E. H., Kahle, K. T., Walcott, B. P., Spitz, M. C., & Staley, K. J. (2011). Diuretics and epilepsy: Will the past and present meet? Epilepsia(52), pp. 1559-1669.
Markadieu, N., Delpire, E. (2014) Physiology and pathophysiology of SLC12A1/2 transporters Pflugers Arch-Eur J Physiol 466:91-105
Merner, N. D., et al. (2016) Gain-of-function missense variant in SLC12A2, encoding the bumetanidesensitive NKCC1 cotransporter, identified in human schizophrenia. Journal of Psychiatric Research 77 22-26
Merner, N. D., et al. (2015) Regulatory domain or CPG site variation in SLC12A5, encoding the chloride transporter KCC2, in human autism and schizophrenia. Frontiers in Cellular Neurosience 9, 386
Moni, L.; Banfi, L.; Basso, A.; Bozzano, A.; Spallarossa, M.; Wessjohann, L.; Riva R. (2016). Molecules, Volume 21, Issue 9, Pages 1153/1-1153/9
Munoz, A., DeFelipe, J., & Alvarez-Leefmans, F. J. (2007). Cation-Chloride Cotransporters and GABAergic Innervation in the Human Epileptic Hippocampus. Epilepsia(48), pp. 663-673. O'Brien, O. G., et al. (2005) Treatment of edema. American Family Physician 71, 2111-2117.
Owens, D. F., & Kriegstein, A. R. (September 2002). Is there more to GABA than synaptic inhibition. Nature Reviews Neuroscience(3), pp. 715-727.
Park, Chan Hee; Lee, Sang Hwi; Im, Junhwan; Lee, Soon Ok; Kim, Hoe Moon; Moon, Sung Hyun; Kim, Seunghee; Kim, Jongmin; Ko, Kwang Seok; Choi, Bu Young; Kim, Byungho (2014). Heterocyclic derivatives as STAT3 protein inhibitors and their preparation. PCT Int. Appl. 293 pp., Patent 2014. WO 2014196793
Payne, J. A., Rivera, C., Voipio, J., & Kaila, K. (April 2003). Cation-chloride co-transporters in neuronal communication, development and trauma. Trends in Neurosciences (26), pp. 199-206.
Peti-Peterdi J., Harris R. (2010) Macula densa sensing and signaling mechanisms of renin release. J Am Soc Nephrol 21:1093-1096
Tyzio R. et al. (2014) Oxytocin-Mediated GABA Inhibition During Delivery Attenuates Autism Pathogenesis in Rodent Offspring Science 343 (6171), 675-679)
Xu W., et al. (2016) Chloride Co-transporter NKCC1 Inhibitor Bumetanide Enhances Neurogenesis and Behavioral Recovery in Rats After Experimental Stroke. Mol Neurobiol DOI 10.1007/s12035-016-9819-0)
Yang, Jing; Sun, Hao; Zhao, Jing (2015). Preparation of piperazine derivatives as antitumor agents. Faming Zhuanli Shenqing 14 pp. Patent 2015, CN 104926804

1. A compound of formula (I) 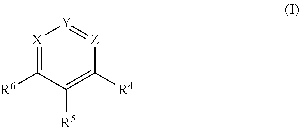
wherein:
the ring moiety is or 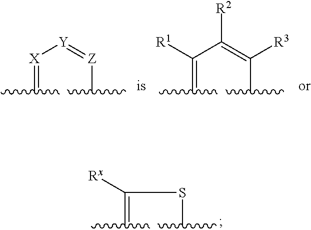
R^{x} is R¹ or R³;
R¹ is selected from —COOH, —COO—(C₁₋₁₅ alkyl), —COO—(C₀₋₁₅ alkylene)-carbocyclyl, —COO—(C₀₋₁₅ alkylene)-heterocyclyl, —O—CHO, —O—CO—(C₁₋₁₅ alkyl), —O—CO—(C₁₋₁₅ alkylene)-carbocyclyl, —O—CO—(C₀₋₁₅ alkylene)-heterocyclyl, —CHO, —CO—(C₁₋₁₅ alkyl), —CO—(C₀₋₁₅ alkylene)-carbocyclyl, —CO—(C₀₋₁₅ alkylene)-heterocyclyl, —CO—NH₂, —CO—N(R¹¹)—(C₁₋₁₅ alkyl), —CO—N(R¹¹)—(C₀₋₁₅ alkylene)-carbocyclyl, —CO—N(R¹¹)—(C₀₋₁₅ alkylene)-heterocyclyl, —N(R¹¹)—CHO, —N(R¹¹)—CO—(C₁₋₁₅ alkyl), —N(R¹¹)—CO—(C₀₋₁₅ alkylene)-carbocyclyl, —N(R¹¹)—CO—(C₀₋₁₅ alkylene)-heterocyclyl, C₁₋₁₅ alkyl, —(C₀₋₁₅ alkylene)-carbocyclyl, —(C₀₋₁₅ alkylene)-heterocyclyl, C₂₋₁₅ alkenyl, —(C₂₋₁₅ alkenylene)-carbocyclyl, —(C₂₋₁₅ alkenylene)-heterocyclyl, C₂₋₁₅ alkynyl, —(C₂₋₁₅ alkynylene)-carbocyclyl and —(C₂₋₁₅ alkynylene)-heterocyclyl,
wherein the alkyl moiety of any of the aforementioned groups, the alkylene moiety of any of the aforementioned groups, the alkenylene moiety of any of the aforementioned groups, the alkynylene moiety of any of the aforementioned groups, said C₁₋₁₅ alkyl, said C₂₋₁₅ alkenyl and said C₂₋₁₅ alkynyl are each optionally substituted with one or more groups independently selected from halogen, —CF₃, —CN, —NO₂, —N(R¹¹)(R¹¹), —O(R¹¹), —S(R¹¹) and —SO₃H,
wherein one or more —CH₂— units comprised in the alkyl moiety of any of the aforementioned groups, in the alkylene moiety of any of the aforementioned groups, in the alkenylene moiety of any of the aforementioned groups, in the alkynylene moiety of any of the aforementioned groups, in said C₁₋₁₅ alkyl, in said C₂₋₁₅ alkenyl, or in said C₂₋₁₅ alkynyl are each optionally replaced by a group independently selected from —O—, —CO—, —COO—, —O—CO—, —N(R)—, —N(R¹¹)—CO—, —CO—N(R¹¹)—, —S—, —SO—, —SO₂—, —SO₂—N(R¹¹)— and —N(R¹¹)—SO₂—,
and further wherein the carbocyclyl moiety of any of the aforementioned groups and the heterocyclyl moiety of any of the aforementioned groups are each optionally substituted with one or more groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹), —S(R¹), —SO₃H, carbocyclyl and heterocyclyl;
each R¹¹ is independently hydrogen or C₁₋₆ alkyl;
R² is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl);
R³ is selected from —SO₂—NH₂, —SO₂—NH(C₁₋₆ alkyl), —SO₂—N(C₁₋₆ alkyl)(C₁₋₆ alkyl), —SO₂—N═(C₁₋₆ alkylidene) and —SO₂-halogen, wherein the alkyl moiety of said —SO₂—NH(C₁₋₆ alkyl), one or both of the alkyl moieties of said —SO₂—N(C₁₋₆ alkyl)(C₁₋₆ alkyl), and the alkylidene moiety of said —SO₂—N═(C₁₋₆ alkylidene) are each optionally substituted with one or more groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl);
R⁴ is a group R^{4a}, and R⁵ is a group R^{5a}, or R⁴ and R⁵ are mutually linked to form a group —R^{5b}—;
R^{4a} is selected from —O—R⁴¹, —S—R⁴¹, —NH—R⁴¹, —N(C₁₋₆ alkyl)-R⁴¹, halogen, hydrogen, carbocyclyl and heterocyclyl, wherein said carbocyclyl and said heterocyclyl are each optionally substituted with one or more groups R⁴²;
R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₀₋₄ alkylene)-carbocyclyl and the heterocyclyl moiety of said —(C₁₋₄ alkylene)-heterocyclyl are each optionally substituted with one or more groups R⁴², and wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₁₋₄ alkylene)-heterocyclyl are each optionally substituted with one or more groups R⁴³;
each R⁴² is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl);
each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃, —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl) and —N(C₁₋₆ alkyl)-CO—(C₁₋₆ alkyl);
R^{5a} is selected from —NH₂, —NH(C₁₋₆ alkyl), —N(C₁₋₆ alkyl)(C₁₋₆ alkyl), —NO₂ and hydrogen, wherein the alkyl moiety of said —NH(C₁₋₆ alkyl) and one or both of the alkyl moieties of said —N(C₁₋₆ alkyl)(C₁₋₆ alkyl) are each optionally substituted with one or more groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH, —S(C₁₋₆ alkyl), carbocyclyl and heterocyclyl, wherein said carbocyclyl and said heterocyclyl are each optionally substituted with one or more groups R⁵¹;
each R⁵¹ is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl);
R^{5b} is selected from —R^{5b1}—R^{5b2}—R^{5b1}, —N═C(R⁵³)—R^{5b3}—R^{5b1}, —R^{5b1}—R^{5b3}—C(R⁵³)═N—, and —N═C(R⁵³)—R^{5b4}—C(R⁵³)═N—;
each R^{5b1} is independently selected from —N(R⁵²)—, —O— and —S—;
R^{5b2} is selected from —C(R⁵³)(R⁵³)—, —C(R⁵³)(R⁵³)—C(R⁵³)(R⁵³)—, —C(R⁵³)═C(R⁵³)—, —C(R⁵³)(R⁵³)—C(R⁵³)═C(R⁵³)— and —C(R⁵³)═C(R⁵³)—C(R⁵³)(R⁵³)—;
R^{5b3} is selected from a covalent bond, —C(R⁵³)(R⁵³), —C(R⁵³⁾(R⁵³)—C(R⁵³)(R⁵³)— and —C(R⁵³)═C(R⁵³)—;
R^{5b4} is selected from a covalent bond and —C(R⁵³)(R⁵³)—;
each R⁵² is independently selected from hydrogen, C₁₋₆ alkyl, C₂₋₆ alkenyl, C₂₋₆ alkynyl, —(C₀₋₄ alkylene)-aryl and —(C₀₋₄ alkylene)-heteroaryl;
each R⁵³ is independently selected from hydrogen, C₁₋₆ alkyl, C₂₋₆ alkenyl, C₂₋₆ alkynyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl), —N(C₁₋₆ alkyl)-SO₂—(C₁₋₆ alkyl), —(C₀₋₄ alkylene)-aryl and —(C₀₋₄ alkylene)-heteroaryl, and any two groups R⁵³ that are attached to the same carbon atom may also together form a group ═O, and any two groups R⁵³ that are attached to adjacent carbon atoms connected by a double bond may also be mutually linked to form a group —C(R⁵⁴)═C(R⁵⁴)—C(R⁵)═C(R⁵⁴)—;
each R⁵⁴ is independently selected from hydrogen, C₁₋₆ alkyl, C₂₋₆ alkenyl, C₂₋₆ alkynyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl); and
R⁶ is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆alkyl);
or R¹ and R⁶ are mutually linked to form a group —R¹⁶—, wherein:
R¹⁶ is a group —C(R¹⁶¹)(R¹⁶¹)—C(R¹⁶¹)(R¹⁶¹)—C(R¹⁶¹)(R¹⁶¹)—C(R¹⁶¹)(R¹⁶¹)—, wherein one or two —C(R¹⁶¹)(R¹⁶¹)— units comprised in said group are each replaced by —R¹⁶³—;
each R¹⁶¹ is independently selected from hydrogen, C₁₋₆ alkyl, C₂₋₆ alkenyl, C₂₋₆ alkynyl, halogen, C₁₋₆ haloalkyl, —(C₀₋₆ alkylene)-CF₃, —(C₀₋₆ alkylene)-CN, —(C₀₋₆ alkylene)-NO₂, —(C₀₋₆ alkylene)-N(R¹⁶²)(R¹⁶²), —(C₀₋₆ alkylene)-O(R¹⁶²), —(C₀₋₆ alkylene)-S(R¹⁶²), —(C₀₋₆ alkylene)-SO₃H, —(C₀₋₆ alkylene)-carbocyclyl and —(C₀₋₆ alkylene)-heterocyclyl;
each R¹⁶² is independently hydrogen or C₁₋₆ alkyl; and
each R¹⁶³ is independently selected from —N(R¹⁶¹)—, —O— and —S—;
with the proviso that, if the ring moiety 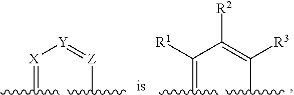
R² is hydrogen, R³ is —SO₂—NH₂, R⁴ is —O-phenyl, R⁵ is —NH—CH₂CH₂CH₂CH₃ and R⁶ is hydrogen, then R¹ is different from —COOH;
or a pharmaceutically acceptable salt or solvate thereof
for use in the treatment or prevention of hyperhidrosis.

2. The compound for use according to claim 1, wherein the compound of formula (I) is a compound of formula (Ia) or a pharmaceutically acceptable salt or solvate thereof: 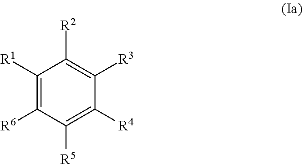
wherein R¹, R², R³, R⁴, R⁵ and R⁶ are as defined in claim 1.

3. The compound for use according to claim 2, wherein R¹ is —COO—(C₁₋₅ alkyl), wherein the alkyl moiety of said —COO—(C₁₋₁₅ alkyl) is optionally substituted with one or more groups independently selected from halogen, —CF₃, —CN, —NO₂, —NH₂, —NH(C₁₋₄ alkyl), —N(C₁₋₄ alkyl)(C₁₋₄ alkyl), —OH, —O(C₁₋₄ alkyl), —SH and —S(C₁₋₄ alkyl), and further wherein one or two —CH₂— units comprised in the alkyl moiety of said —COO—(C₁₋₁₅ alkyl) are each optionally replaced by a group independently selected from —O—, —CO—, —COO—, —O—CO—, —NH—, —N(C₁₋₄ alkyl)-, —NH—CO—, —N(C₁₋₄ alkyl)-CO—, —CO—NH—, —CO—N(C₁₋₄ alkyl)-, —S—, —SO—, —SO₂—, —SO₂—NH—, —SO₂—N(C₁₋₄ alkyl)-, —NH—SO₂— and —N(C₁₋₄ alkyl)-SO₂—.

4. The compound for use according to claim 2 or 3, wherein R¹ is —COO—CH₃.

5. The compound for use according to claim 2, wherein R¹ is —COOH.

6. The compound for use according to claim 2, wherein R¹ is selected from —(C₁₋₄ alkylene)-NH—(C₁₋₄ alkylene)-R¹², —COO—(C₁₋₄ alkylene)-R¹², —O—CO—(C₁₋₄ alkylene)-R¹², —CO—(C₁₋₄ alkylene)-R¹², —CO—NH—(C₁₋₄ alkylene)-R¹², —CO—N(C₁₋₄ alkyl)-(C₁₋₄ alkylene)-R¹², —NH—CO—(C₁₋₄ alkylene)-R¹² and —N(C₁₋₄ alkyl)-CO—(C₁₋₄ alkylene)-R¹², wherein R¹² is independently selected from —CF₃, —CN and halogen.

7. The compound for use according to claim 2 or 6, wherein R¹ is —(C₁₋₄ alkylene)-NH—(C₁₋₄ alkylene)-CF₃.

8. The compound for use according to any one of claims 2 to 7, wherein R² is hydrogen.

9. The compound for use according to any one of claims 2 to 8, wherein R³ is selected from —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), and further wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one group selected from —NH₂, —NH(C₁₋₄ alkyl) and —N(C₁₋₄ alkyl)(C₁₋₄ alkyl).

10. The compound for use according to any one of claims 2 to 9, wherein R⁴ is a group R^{4a} which is selected from —O-aryl, —O-heteroaryl, —S-aryl, —S-heteroaryl, —NH-aryl, —NH— heteroaryl, —N(C₁₋₄ alkyl)-aryl, —N(C₁₋₄ alkyl)-heteroaryl, aryl and heteroaryl, and wherein the aryl moiety of any of the aforementioned groups, the heteroaryl moiety of any of the aforementioned groups, said aryl and said heteroaryl are each optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN.

11. The compound for use according to any one of claims 2 to 10, wherein R⁵ is a group R^{5a} which is selected from —NH₂, —NH(C₁₋₆ alkyl), —N(C₁₋₆ alkyl)(C₁₋₆ alkyl), and —NO₂.

12. The compound for use according to any one of claims 2 to 11, wherein R⁶ is hydrogen.

13. The compound for use according to claim 1, wherein the compound of formula (I) is a compound of formula (Ib) or a pharmaceutically acceptable salt or solvate thereof: 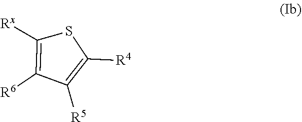
wherein R^{x}, R⁴, R⁵ and R⁶ are as defined in claim 1.

14. The compound for use according to claim 13, wherein R is selected from —COOH, —COO—(C₁₋₆ alkyl), —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), and further wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one group selected from —NH₂, —NH(C₁₋₄ alkyl) and —N(C₁₋₄ alkyl)(C₁₋₄ alkyl).

15. The compound for use according to claim 13 or 14, wherein R⁴ is a group R^{4a} which is —O-aryl or halogen, and wherein the aryl moiety of said —O-aryl is optionally substituted with one or more groups independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl and —CN.

16. The compound for use according to any one of claims 13 to 15, wherein R⁵ is a group R^{5a} which is selected from —NH₂, —NH(C₁₋₆ alkyl), —N(C₁₋₆ alkyl)(C₁₋₆ alkyl), and —NO₂.

17. The compound for use according to any one of claims 13 to 16, wherein R⁶ is hydrogen.

18. The compound for use according to claim 1, wherein the compound of formula (I) is a compound of any one of the following formulae, or a pharmaceutically acceptable salt or solvate thereof: 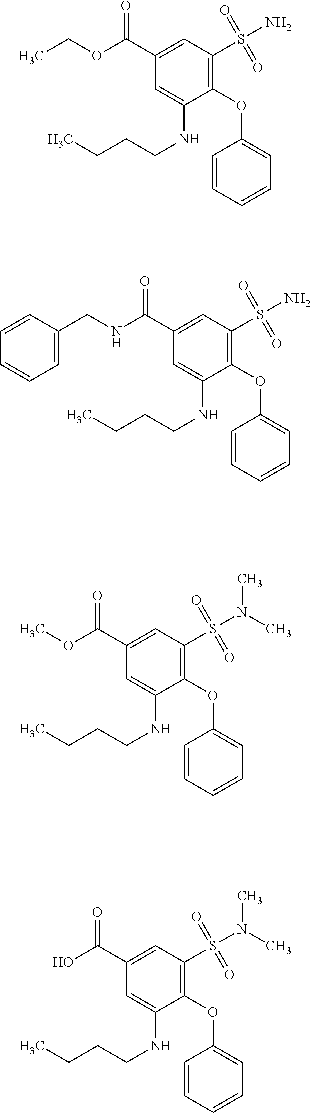 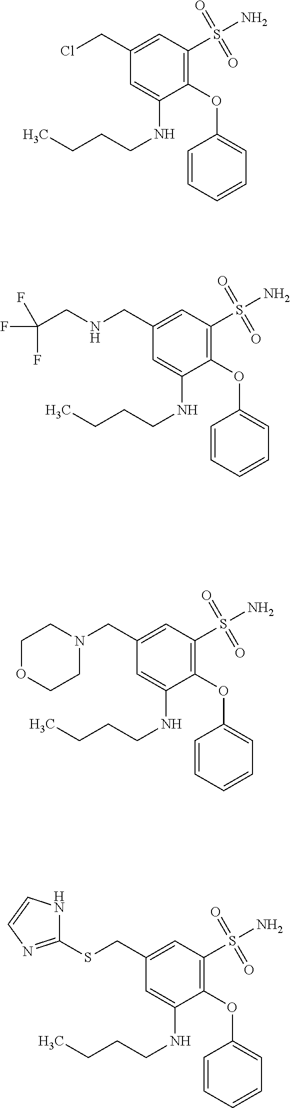 [SMILES: CCCCNc1cc(CCl)cc(S(N)(=O)=O)c1Oc1ccccc1 ; CCCCNc1cc(CNCC(F)(F)F)cc(S(N)(=O)=O)c1Oc1ccccc1 ; CCCCNc1cc(CN2CCOCC2)cc(S(N)(=O)=O)c1Oc1ccccc1 ; CCCCNc1cc(CSc2ncc[nH]2)cc(S(N)(=O)=O)c1Oc1ccccc1] 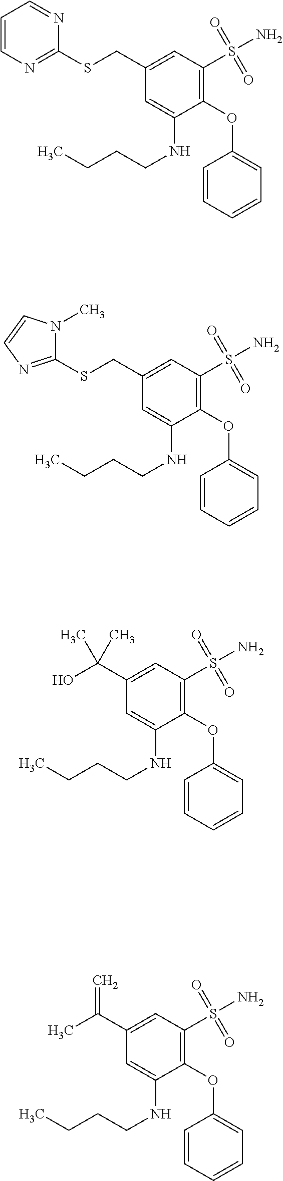 [SMILES: CCCCNc1cc(CSc2ncccn2)cc(S(N)(=O)=O)c1Oc1ccccc1 ; CCCCNc1cc(CSc2nccn2C)cc(S(N)(=O)=O)c1Oc1ccccc1 ; CCCCNc1cc(C(C)(C)O)cc(S(N)(=O)=O)c1Oc1ccccc1 ; C=C(C)c1cc(NCCCC)c(Oc2ccccc2)c(S(N)(=O)=O)c1] 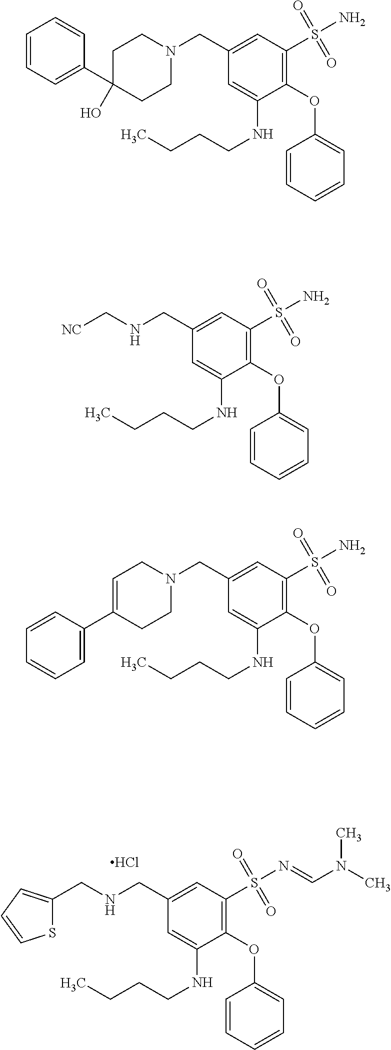 [SMILES: CCCCNc1cc(CN2CCC(O)(c3ccccc3)CC2)cc(S(N)(=O)=O)c1Oc1ccccc1 ; CCCCNc1cc(CNCC#N)cc(S(N)(=O)=O)c1Oc1ccccc1 ; CCCCNc1cc(CN2CC=C(c3ccccc3)CC2)cc(S(N)(=O)=O)c1Oc1ccccc1 ; CCCCNc1cc(CNCc2cccs2)cc(S(=O)(=O)/N=C/N(C)C)c1Oc1ccccc1 ; Cl] 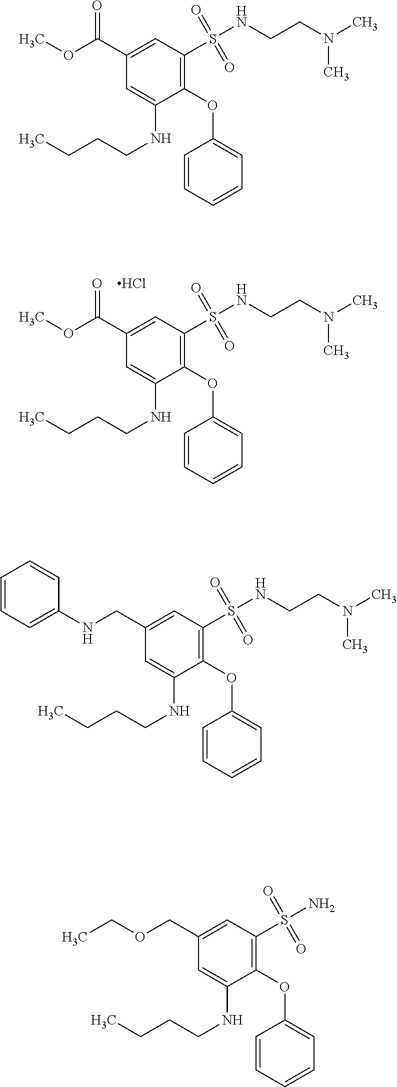 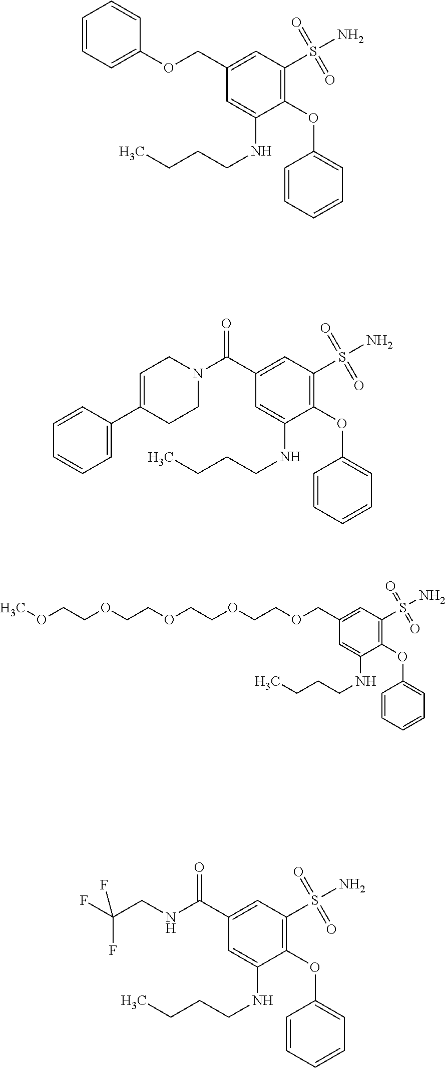 [SMILES: CCCCNc1cc(COc2ccccc2)cc(S(N)(=O)=O)c1Oc1ccccc1 ; CCCCNc1cc(C(=O)N2CC=C(c3ccccc3)CC2)cc(S(N)(=O)=O)c1Oc1ccccc1 ; CCCCNc1cc(COCCOCCOCCOCCOC)cc(S(N)(=O)=O)c1Oc1ccccc1 ; CCCCNc1cc(C(=O)NCC(F)(F)F)cc(S(N)(=O)=O)c1Oc1ccccc1] 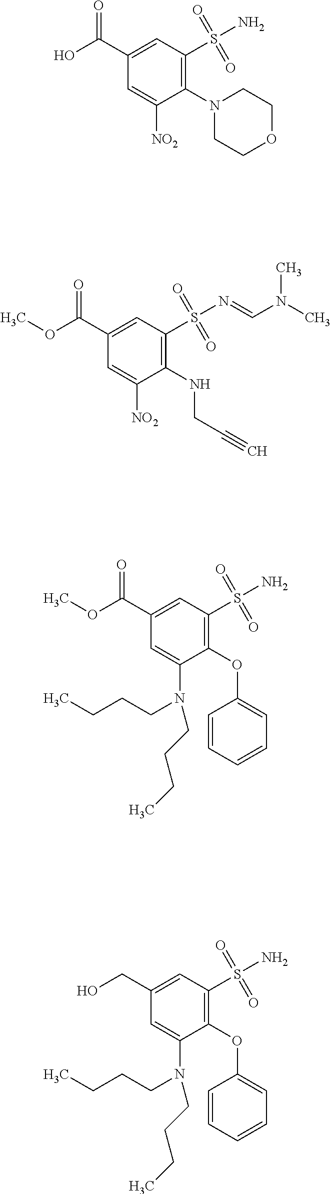 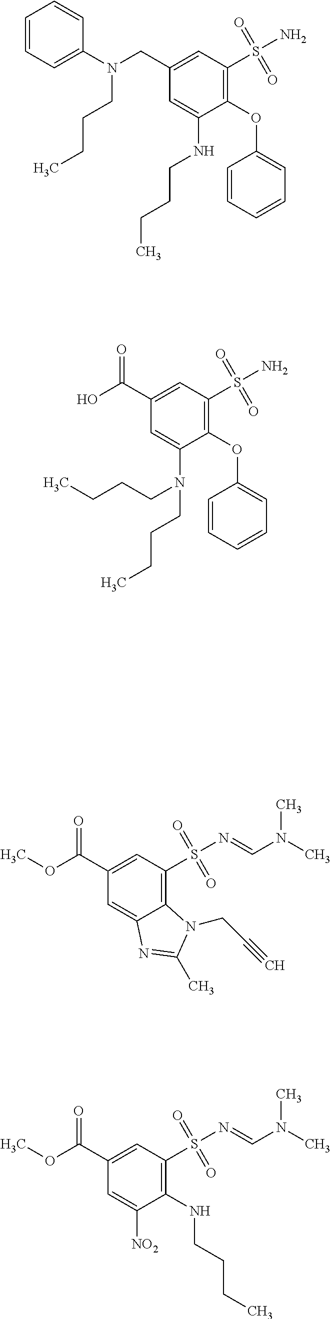 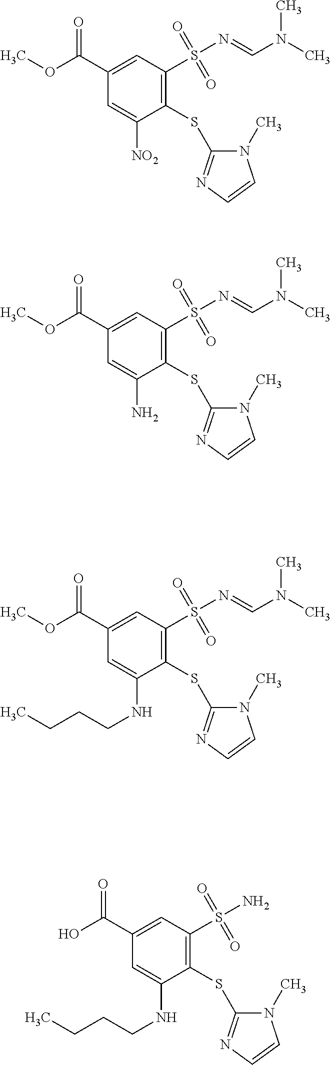 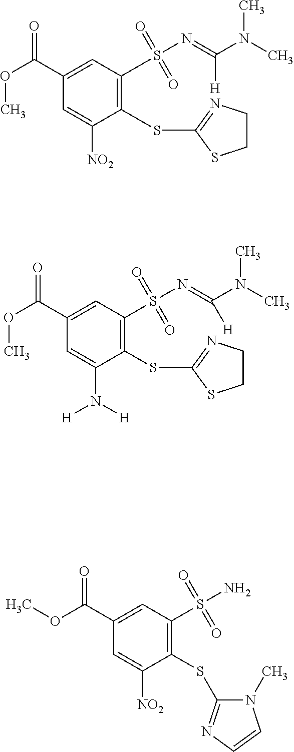 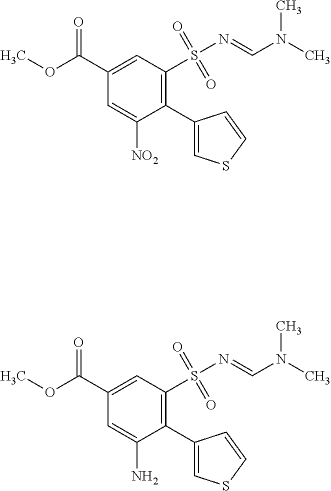 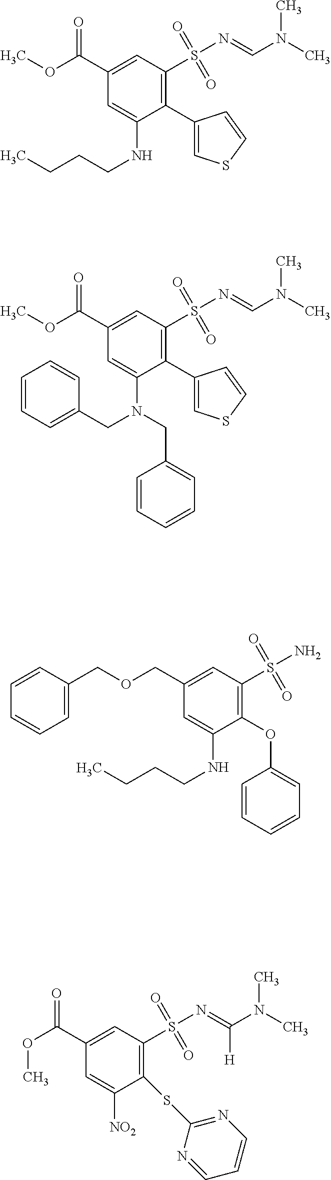 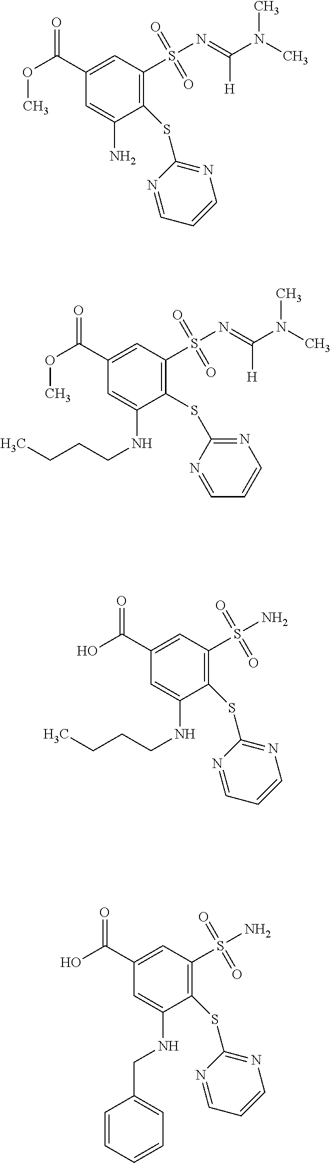 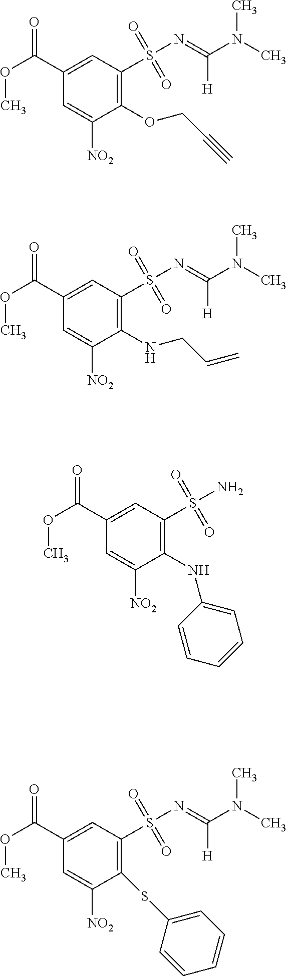 [SMILES: C#CCOc1c([N+](=O)[O-])cc(C(=O)OC)cc1S(=O)(=O)/N=C/N(C)C ; C=CCNc1c([N+](=O)[O-])cc(C(=O)OC)cc1S(=O)(=O)/N=C/N(C)C ; COC(=O)c1cc([N+](=O)[O-])c(Nc2ccccc2)c(S(N)(=O)=O)c1 ; COC(=O)c1cc([N+](=O)[O-])c(Sc2ccccc2)c(S(=O)(=O)/N=C/N(C)C)c1] 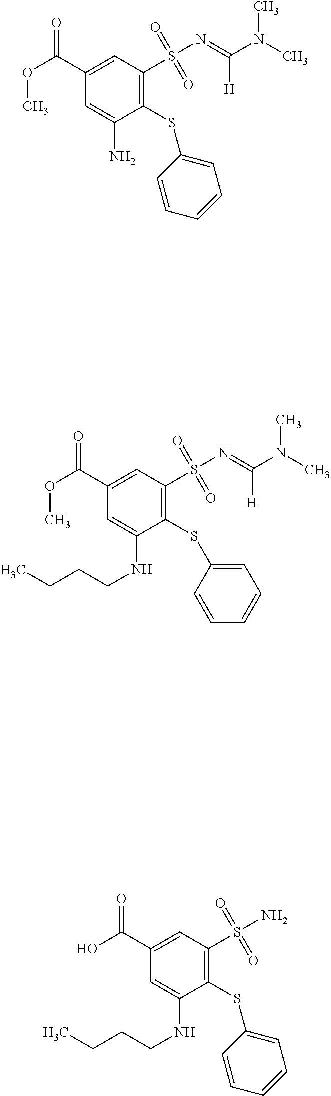 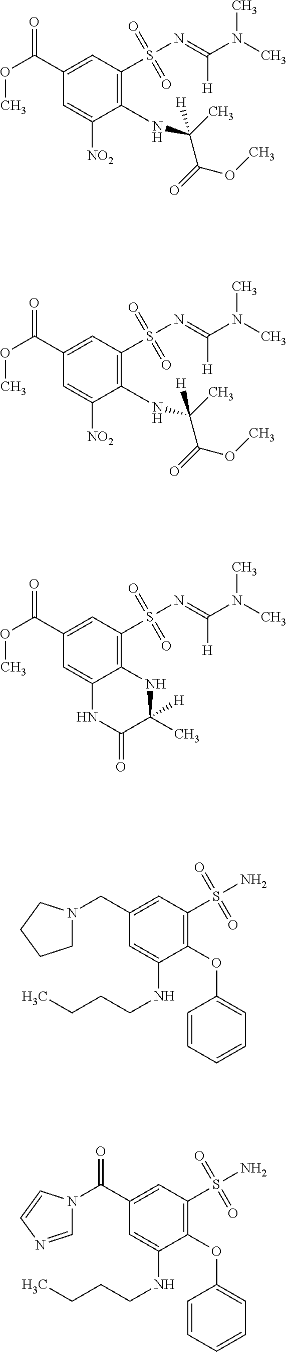 [SMILES: COC(=O)c1cc([N+](=O)[O-])c(N[C@@H](C)C(=O)OC)c(S(=O)(=O)/N=C/N(C)C)c1 ; COC(=O)c1cc([N+](=O)[O-])c(N[C@H](C)C(=O)OC)c(S(=O)(=O)/N=C/N(C)C)c1 ; COC(=O)c1cc2c(c(S(=O)(=O)/N=C/N(C)C)c1)N[C@@H](C)C(=O)N2 ; CCCCNc1cc(CN2CCCC2)cc(S(N)(=O)=O)c1Oc1ccccc1 ; CCCCNc1cc(C(=O)n2ccnc2)cc(S(N)(=O)=O)c1Oc1ccccc1] 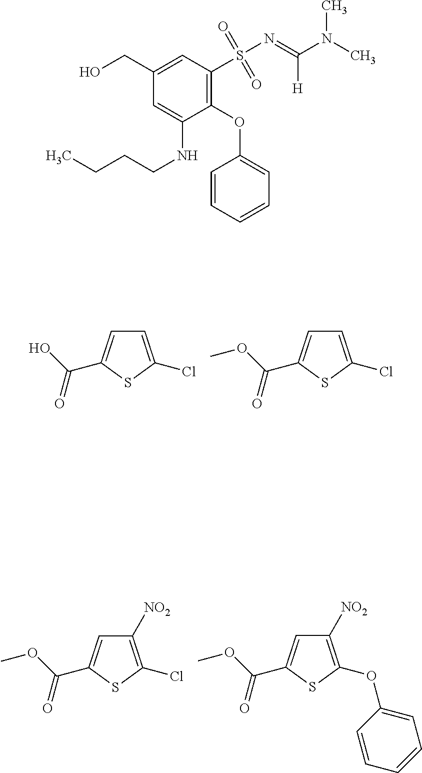 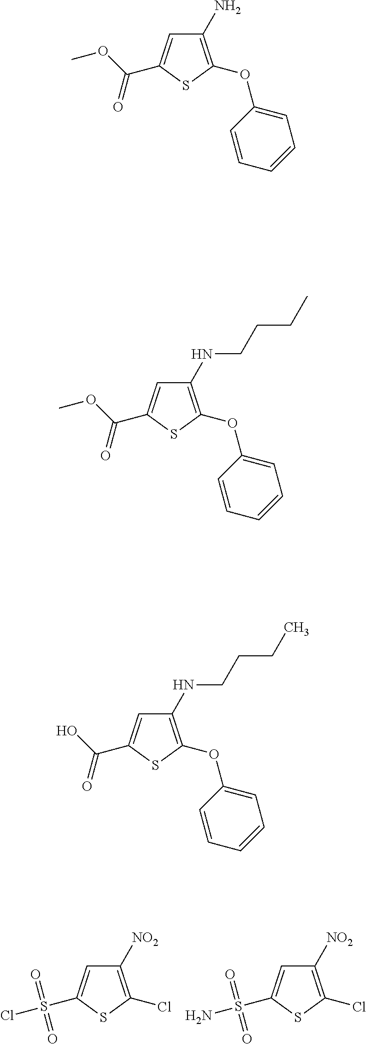 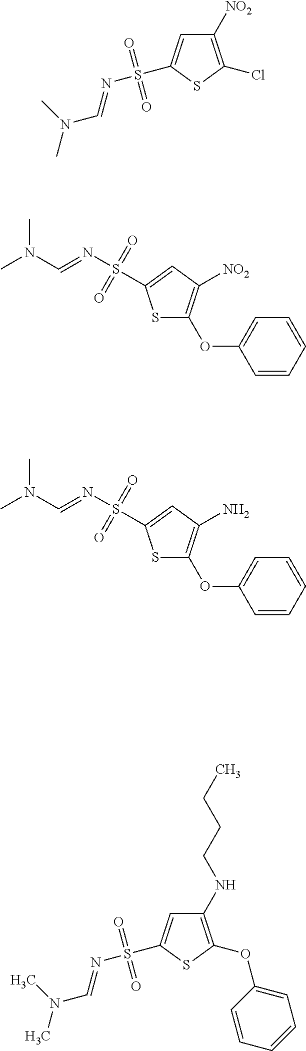 [SMILES: CN(C)/C=N/S(=O)(=O)c1cc([N+](=O)[O-])c(Cl)s1 ; CN(C)/C=N/S(=O)(=O)c1cc([N+](=O)[O-])c(Oc2ccccc2)s1 ; CN(C)/C=N/S(=O)(=O)c1cc(N)c(Oc2ccccc2)s1 ; CCCCNc1cc(S(=O)(=O)/N=C/N(C)C)sc1Oc1ccccc1] 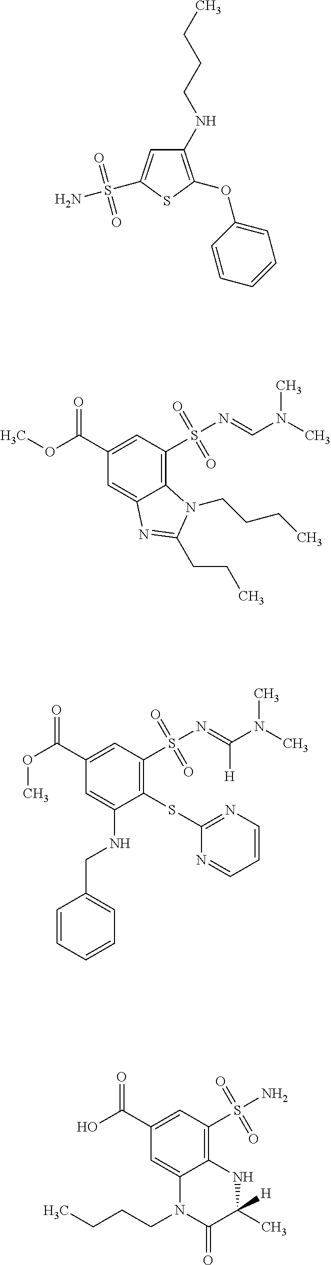 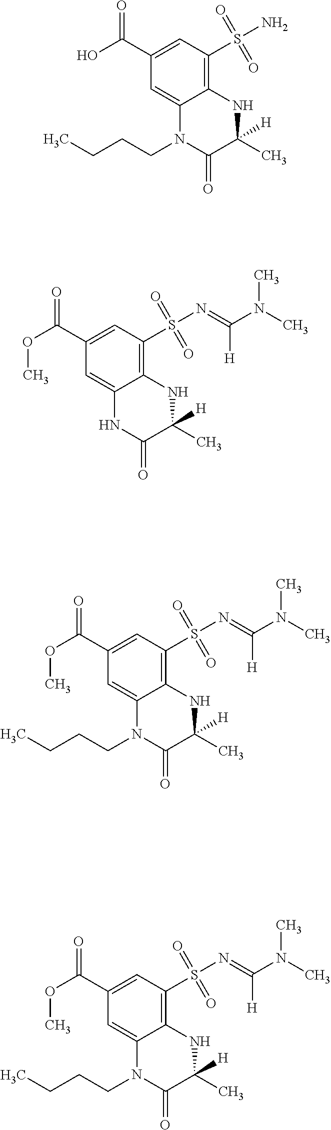 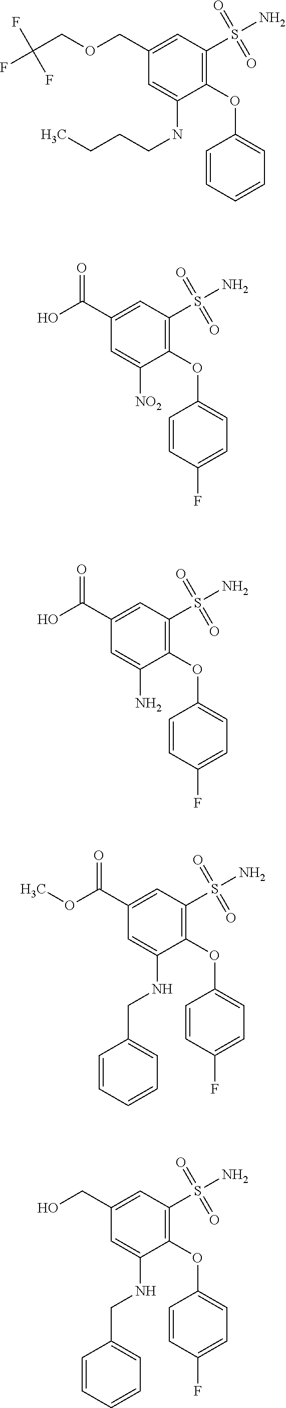 [SMILES: CCCCNc1cc(COCC(F)(F)F)cc(S(N)(=O)=O)c1Oc1ccccc1 ; NS(=O)(=O)c1cc(C(=O)O)cc([N+](=O)[O-])c1Oc1ccc(F)cc1 ; Nc1cc(C(=O)O)cc(S(N)(=O)=O)c1Oc1ccc(F)cc1 ; COC(=O)c1cc(NCc2ccccc2)c(Oc2ccc(F)cc2)c(S(N)(=O)=O)c1 ; NS(=O)(=O)c1cc(CO)cc(NCc2ccccc2)c1Oc1ccc(F)cc1] 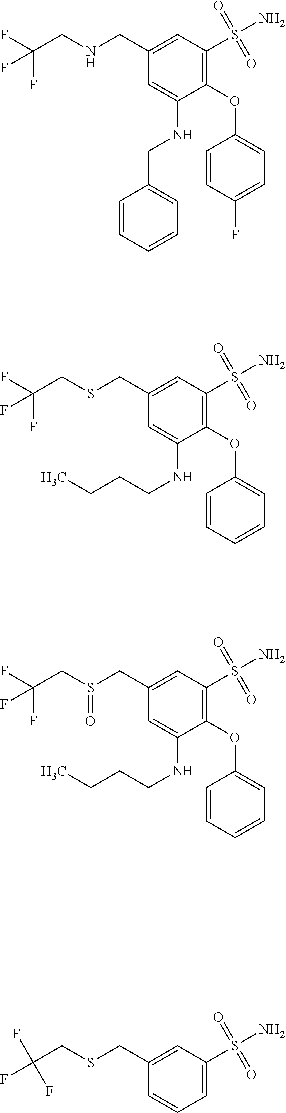 [SMILES: NS(=O)(=O)c1cc(CNCC(F)(F)F)cc(NCc2ccccc2)c1Oc1ccc(F)cc1 ; CCCCNc1cc(CSCC(F)(F)F)cc(S(N)(=O)=O)c1Oc1ccccc1 ; CCCCNc1cc(CS(=O)CC(F)(F)F)cc(S(N)(=O)=O)c1Oc1ccccc1 ; NS(=O)(=O)c1cccc(CSCC(F)(F)F)c1] 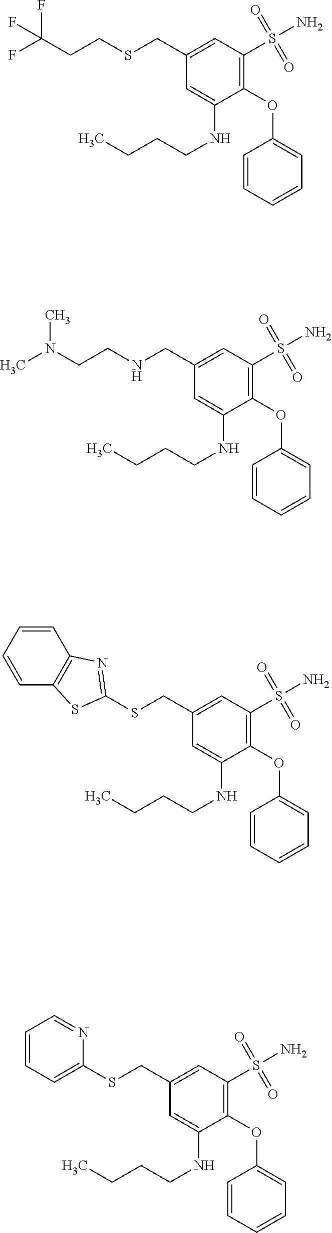 [SMILES: CCCCNc1cc(CSCCC(F)(F)F)cc(S(N)(=O)=O)c1Oc1ccccc1 ; CCCCNc1cc(CNCCN(C)C)cc(S(N)(=O)=O)c1Oc1ccccc1 ; CCCCNc1cc(CSc2nc3ccccc3s2)cc(S(N)(=O)=O)c1Oc1ccccc1 ; CCCCNc1cc(CSc2ccccn2)cc(S(N)(=O)=O)c1Oc1ccccc1] 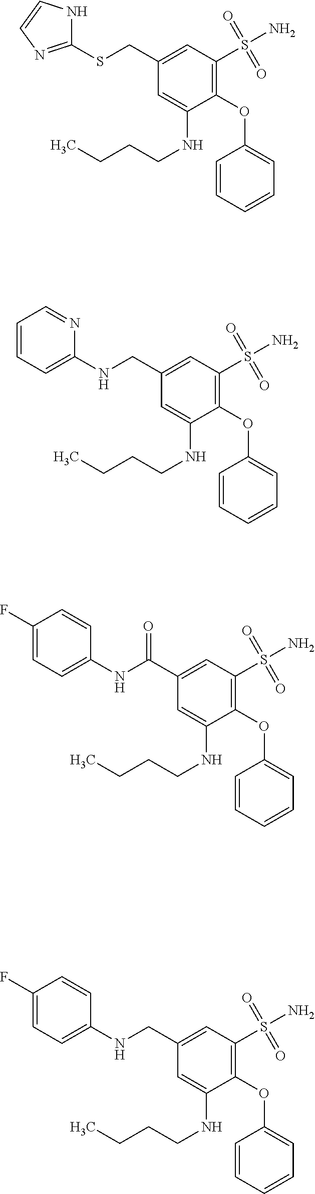 [SMILES: CCCCNc1cc(CSc2ncc[nH]2)cc(S(N)(=O)=O)c1Oc1ccccc1 ; CCCCNc1cc(CNc2ccccn2)cc(S(N)(=O)=O)c1Oc1ccccc1 ; CCCCNc1cc(C(=O)Nc2ccc(F)cc2)cc(S(N)(=O)=O)c1Oc1ccccc1 ; CCCCNc1cc(CNc2ccc(F)cc2)cc(S(N)(=O)=O)c1Oc1ccccc1] 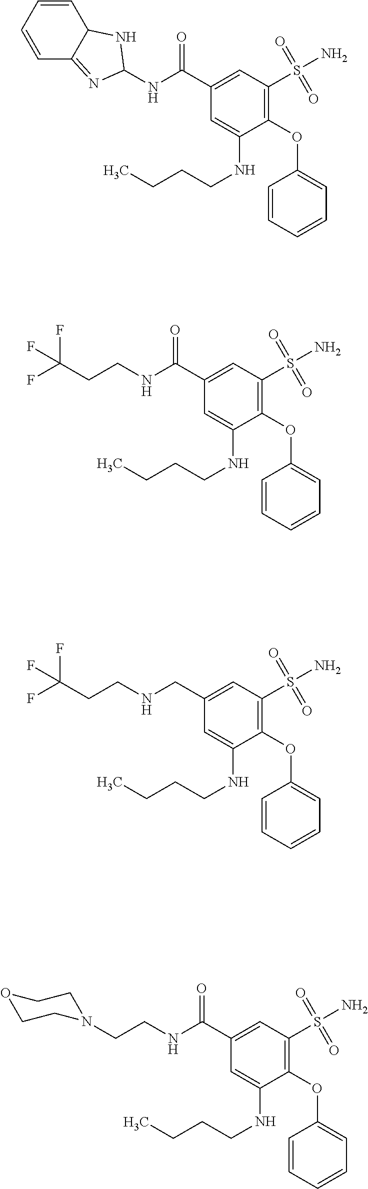 [SMILES: CCCCNc1cc(C(=O)NC2N=C3C=CC=CC3N2)cc(S(N)(=O)=O)c1Oc1ccccc1 ; CCCCNc1cc(C(=O)NCCC(F)(F)F)cc(S(N)(=O)=O)c1Oc1ccccc1 ; CCCCNc1cc(CNCCC(F)(F)F)cc(S(N)(=O)=O)c1Oc1ccccc1 ; CCCCNc1cc(C(=O)NCCN2CCOCC2)cc(S(N)(=O)=O)c1Oc1ccccc1] 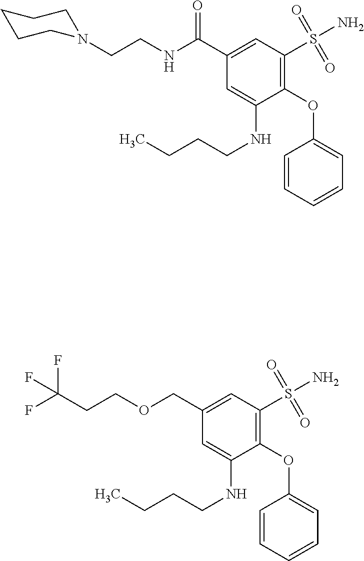 [SMILES: CCCCNc1cc(C(=O)NCCN2CCCCC2)cc(S(N)(=O)=O)c1Oc1ccccc1 ; CCCCNc1cc(COCCC(F)(F)F)cc(S(N)(=O)=O)c1Oc1ccccc1]
Error! Objects cannot be created from editing field codes.Error! Objects cannot be created from editing field codes.Error! Objects cannot be created from editing field codes.

19. A pharmaceutical composition comprising a compound as defined in any one of claims 1 to 18 and a pharmaceutically acceptable excipient for use in the treatment or prevention of hyperhidrosis.

20. Use of a compound as defined in any one of claims 1 to 18 in the preparation of a medicament for the treatment or prevention of hyperhidrosis.

21. A method of treating or preventing hyperhidrosis, the method comprising administering a compound as defined in any one of claims 1 to 18 or a pharmaceutical composition as defined in claim 19 to a subject in need thereof.

22. The method of claim 21, wherein the subject is a human.

23. Non-therapeutic use of a compound as defined in any one of claims 1 to 18 for suppressing or reducing the sweating of a subject.

24. Non-therapeutic method of suppressing or reducing the sweating of a subject, wherein the method comprises administering a compound as defined in any one of claims 1 to 18 to the subject.

25. The non-therapeutic use of claim 23 or the non-therapeutic method of claim 24, wherein said compound is administered topically.

26. The non-therapeutic use of claim 23 or 25 or the non-therapeutic method of claim 24 or 25, wherein said compound is provided in the form of an article comprising said compound, wherein the article is a wipe, an insole or a garment.

27. An article comprising a compound as defined in any one of claims 1 to 18, wherein the article is a wipe, an insole or a garment.

28. A compound as defined in any one of claims 1 to 18 for use in the treatment or prevention of a disease or disorder selected from an anxiety disorder, an autism spectrum disorder, autism, Asperger syndrome, childhood disintegrative disorder, a pervasive developmental disorder as part of an autism spectrum disorder, traumatic brain injury, spinal cord injury, peripheral nerve injury, stroke, Alzheimer's disease, schizophrenia, asthma, edema, Down syndrome, mental disability in patients with Down syndrome, glaucoma, primary open angle glaucoma, angle closure glaucoma, and a parasitic infection,
wherein said parasitic infection is preferably selected from a helminth infection, a hookworm infection, a roundworm infection, a whipworm infection, a tapeworm infection, a guinea worm infection, a pinworm infection, a toxocara infection, a Strongyloides stercoralis infection, an Ascaris lumbricoides infection, a parasitic fluke infection, Schistosomiasis, Gnathostomiasis, Paragonimiasis, Fascioliasis, Swimmer's itch, a protozoan infection, malaria, amoebiasis, giardiasis, African sleeping sickness, toxoplasmosis, Acanthamoeba keratitis, leishmaniasis, babesiosis, granulomatous amoebic encephalitis, cryptosporidiosis, cyclosporiasis, primary amoebic meningoencephalitis, an ectoparasitic infection, an infection with Sarcoptes scabiei, an infection with Pediculus humanus capitis, an infection with Phthirus pubis, an infection with human botfly maggots, an infection with Tunga penetrans, and an infection with Ixodoidea.

29. A pharmaceutical composition comprising a compound as defined in any one of claims 1 to 18 and a pharmaceutically acceptable excipient for use in the treatment or prevention of a disease or disorder selected from an anxiety disorder, an autism spectrum disorder, autism, Asperger syndrome, childhood disintegrative disorder, a pervasive developmental disorder as part of an autism spectrum disorder, traumatic brain injury, spinal cord injury, peripheral nerve injury, stroke, Alzheimer's disease, schizophrenia, asthma, edema, Down syndrome, mental disability in patients with Down syndrome, glaucoma, primary open angle glaucoma, angle closure glaucoma, and a parasitic infection,
wherein said parasitic infection is preferably selected from a helminth infection, a hookworm infection, a roundworm infection, a whipworm infection, a tapeworm infection, a guinea worm infection, a pinworm infection, a toxocara infection, a Strongyloides stercoralis infection, an Ascaris lumbricoides infection, a parasitic fluke infection, Schistosomiasis, Gnathostomiasis, Paragonimiasis, Fascioliasis, Swimmer's itch, a protozoan infection, malaria, amoebiasis, giardiasis, African sleeping sickness, toxoplasmosis, Acanthamoeba keratitis, leishmaniasis, babesiosis, granulomatous amoebic encephalitis, cryptosporidiosis, cyclosporiasis, primary amoebic meningoencephalitis, an ectoparasitic infection, an infection with Sarcoptes scabiei, an infection with Pediculus humanus capitis, an infection with Phthirus pubis, an infection with human botfly maggots, an infection with Tunga penetrans, and an infection with Ixodoidea.

30. Use of a compound as defined in any one of claims 1 to 18 in the preparation of a medicament for the treatment or prevention of a disease or disorder selected from an anxiety disorder, an autism spectrum disorder, autism, Asperger syndrome, childhood disintegrative disorder, a pervasive developmental disorder as part of an autism spectrum disorder, traumatic brain injury, spinal cord injury, peripheral nerve injury, stroke, Alzheimer's disease, schizophrenia, asthma, edema, Down syndrome, mental disability in patients with Down syndrome, glaucoma, primary open angle glaucoma, angle closure glaucoma, and a parasitic infection,
wherein said parasitic infection is preferably selected from a helminth infection, a hookworm infection, a roundworm infection, a whipworm infection, a tapeworm infection, a guinea worm infection, a pinworm infection, a toxocara infection, a Strongyloides stercoralis infection, an Ascaris lumbricoides infection, a parasitic fluke infection, Schistosomiasis, Gnathostomiasis, Paragonimiasis, Fascioliasis, Swimmer's itch, a protozoan infection, malaria, amoebiasis, giardiasis, African sleeping sickness, toxoplasmosis, Acanthamoeba keratitis, leishmaniasis, babesiosis, granulomatous amoebic encephalitis, cryptosporidiosis, cyclosporiasis, primary amoebic meningoencephalitis, an ectoparasitic infection, an infection with Sarcoptes scabiei, an infection with Pediculus humanus capitis, an infection with Phthirus pubis, an infection with human botfly maggots, an infection with Tunga penetrans, and an infection with Ixodoidea.

31. A method of treating or preventing a disease/disorder, the method comprising administering a compound as defined in any one of claims 1 to 18 or a pharmaceutical composition comprising said compound and a pharmaceutically acceptable excipient to a subject in need thereof,
wherein said disease/disorder selected from the group consisting of an anxiety disorder, an autism spectrum disorder, autism, Asperger syndrome, childhood disintegrative disorder, a pervasive developmental disorder as part of an autism spectrum disorder, traumatic brain injury, spinal cord injury, peripheral nerve injury, stroke, Alzheimer's disease, schizophrenia, asthma, edema, Down syndrome, mental disability in patients with Down syndrome, glaucoma, primary open angle glaucoma, angle closure glaucoma, and a parasitic infection,
wherein said parasitic infection is preferably selected from the group consisting of a helminth infection, a hookworm infection, a roundworm infection, a whipworm infection, a tapeworm infection, a guinea worm infection, a pinworm infection, a toxocara infection, a Strongyloides stercoralis infection, an Ascaris lumbricoides infection, a parasitic fluke infection, Schistosomiasis, Gnathostomiasis, Paragonimiasis, Fascioliasis, Swimmer's itch, a protozoan infection, malaria, amoebiasis, giardiasis, African sleeping sickness, toxoplasmosis, Acanthamoeba keratitis, leishmaniasis, babesiosis, granulomatous amoebic encephalitis, cryptosporidiosis, cyclosporiasis, primary amoebic meningoencephalitis, an ectoparasitic infection, an infection with Sarcoptes scabiei, an infection with Pediculus humanus capitis, an infection with Phthirus pubis, an infection with human botfly maggots, an infection with Tunga penetrans, and an infection with lxodoidea.

32. The method of claim 31, wherein the subject is a human.

33. In vitro use of a compound as defined in any one of claims 1 to 18 as an NKCC inhibitor.

34. An in vitro method of inhibiting NKCC, the method comprising the application of a compound as defined in any one of claims 1 to 18.

35. A compound of any one of the following formulae or a pharmaceutically acceptable salt or solvate thereof: 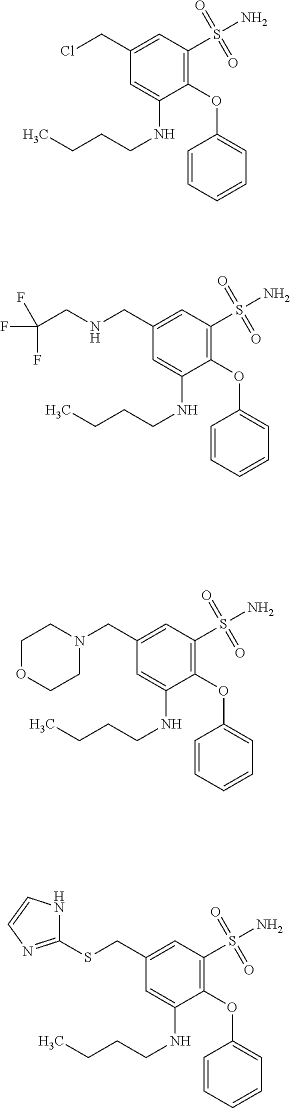 [SMILES: CCCCNc1cc(CCl)cc(S(N)(=O)=O)c1Oc1ccccc1 ; CCCCNc1cc(CNCC(F)(F)F)cc(S(N)(=O)=O)c1Oc1ccccc1 ; CCCCNc1cc(CN2CCOCC2)cc(S(N)(=O)=O)c1Oc1ccccc1 ; CCCCNc1cc(CSc2ncc[nH]2)cc(S(N)(=O)=O)c1Oc1ccccc1] 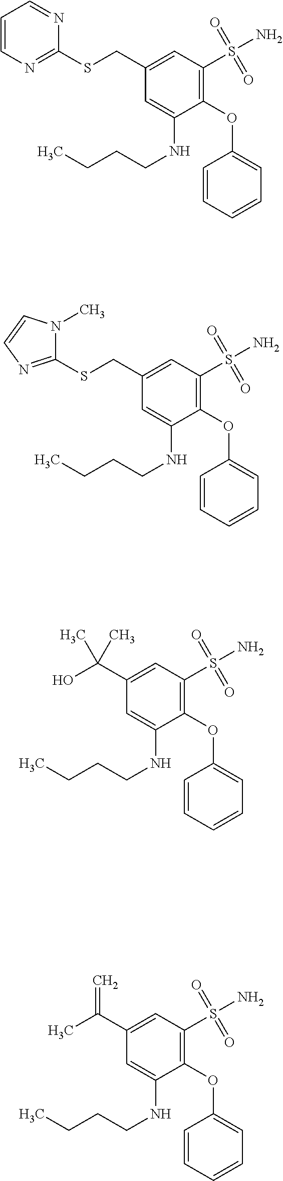 [SMILES: CCCCNc1cc(CSc2ncccn2)cc(S(N)(=O)=O)c1Oc1ccccc1 ; CCCCNc1cc(CSc2nccn2C)cc(S(N)(=O)=O)c1Oc1ccccc1 ; CCCCNc1cc(C(C)(C)O)cc(S(N)(=O)=O)c1Oc1ccccc1 ; C=C(C)c1cc(NCCCC)c(Oc2ccccc2)c(S(N)(=O)=O)c1] 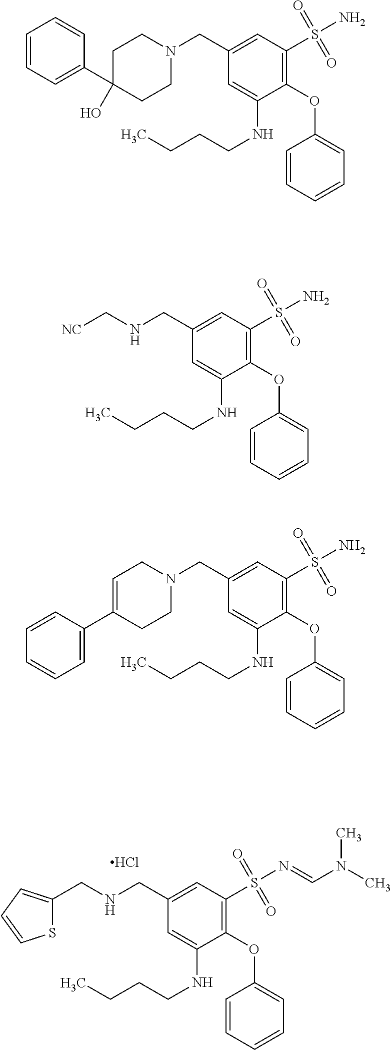 [SMILES: CCCCNc1cc(CN2CCC(O)(c3ccccc3)CC2)cc(S(N)(=O)=O)c1Oc1ccccc1 ; CCCCNc1cc(CNCC#N)cc(S(N)(=O)=O)c1Oc1ccccc1 ; CCCCNc1cc(CN2CC=C(c3ccccc3)CC2)cc(S(N)(=O)=O)c1Oc1ccccc1 ; CCCCNc1cc(CNCc2cccs2)cc(S(=O)(=O)/N=C/N(C)C)c1Oc1ccccc1 ; Cl] 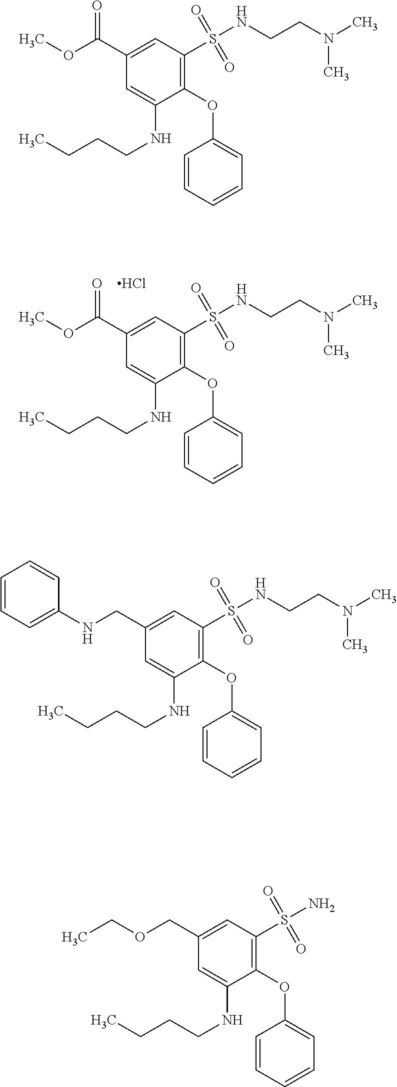 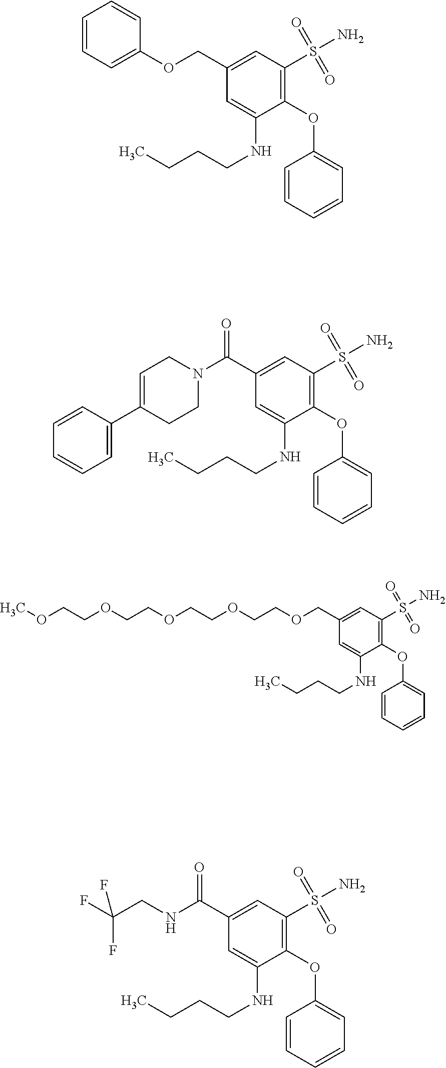 [SMILES: CCCCNc1cc(COc2ccccc2)cc(S(N)(=O)=O)c1Oc1ccccc1 ; CCCCNc1cc(C(=O)N2CC=C(c3ccccc3)CC2)cc(S(N)(=O)=O)c1Oc1ccccc1 ; CCCCNc1cc(COCCOCCOCCOCCOC)cc(S(N)(=O)=O)c1Oc1ccccc1 ; CCCCNc1cc(C(=O)NCC(F)(F)F)cc(S(N)(=O)=O)c1Oc1ccccc1] 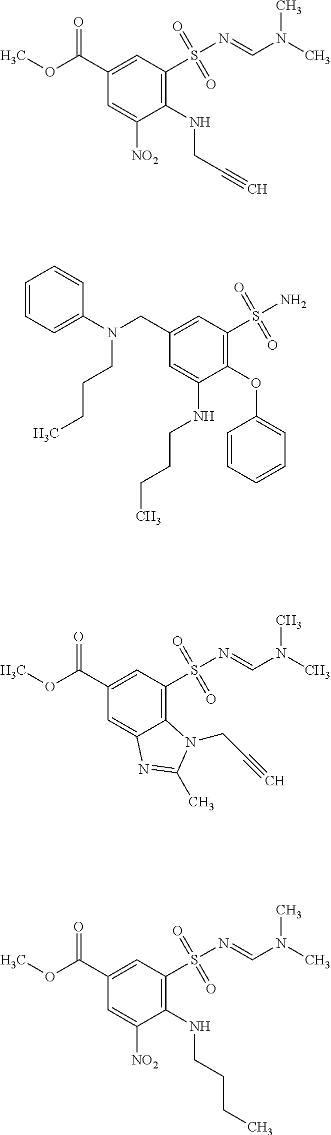 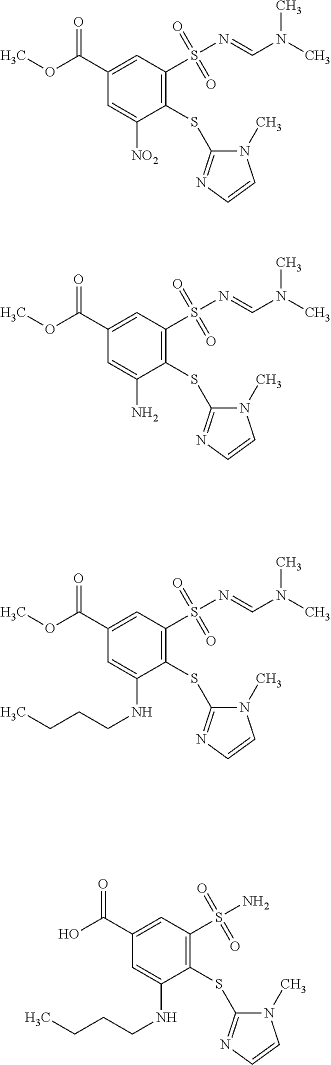 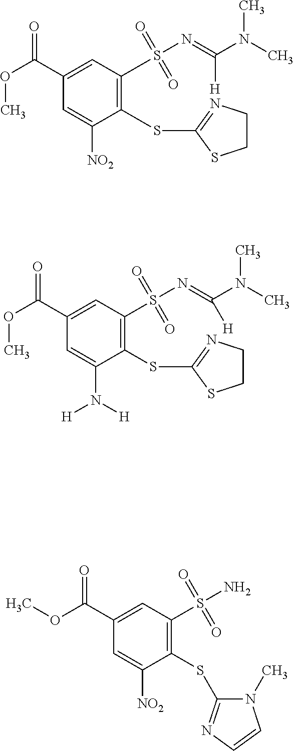 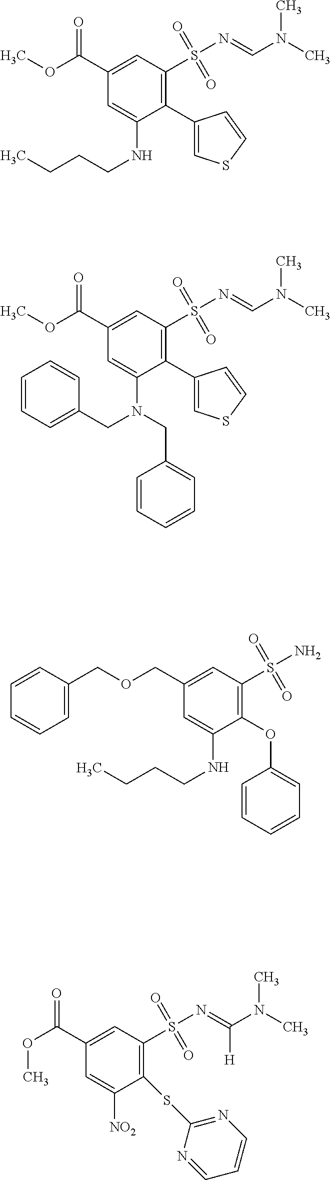 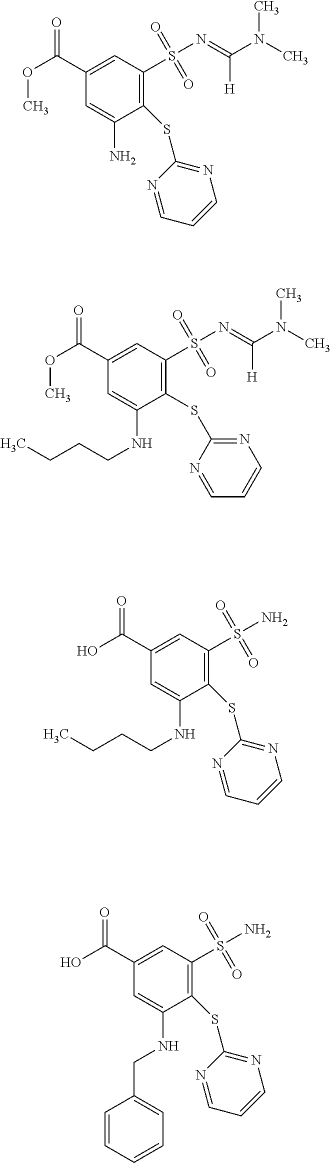 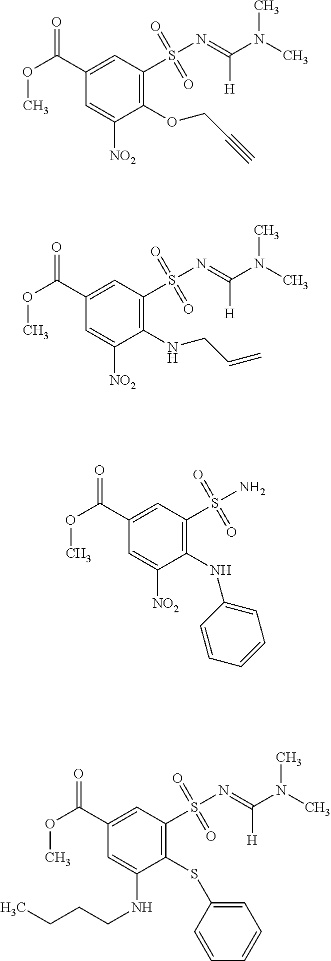 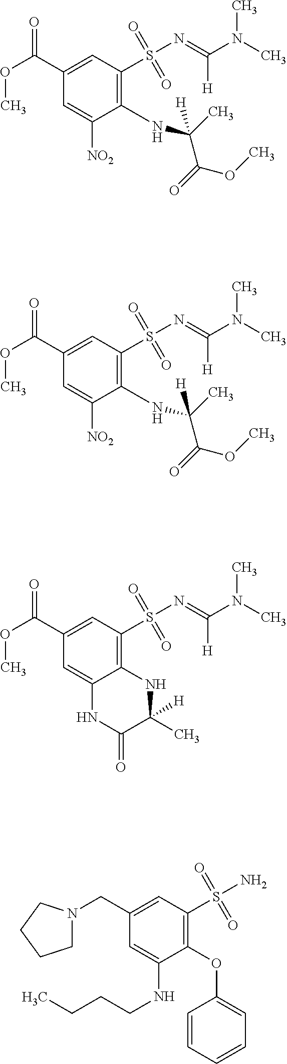 [SMILES: COC(=O)c1cc([N+](=O)[O-])c(N[C@@H](C)C(=O)OC)c(S(=O)(=O)/N=C/N(C)C)c1 ; COC(=O)c1cc([N+](=O)[O-])c(N[C@H](C)C(=O)OC)c(S(=O)(=O)/N=C/N(C)C)c1 ; COC(=O)c1cc2c(c(S(=O)(=O)/N=C/N(C)C)c1)N[C@@H](C)C(=O)N2 ; CCCCNc1cc(CN2CCCC2)cc(S(N)(=O)=O)c1Oc1ccccc1] 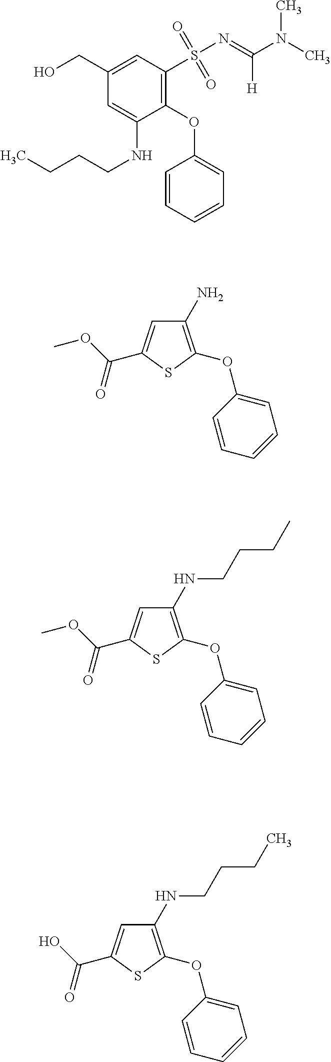 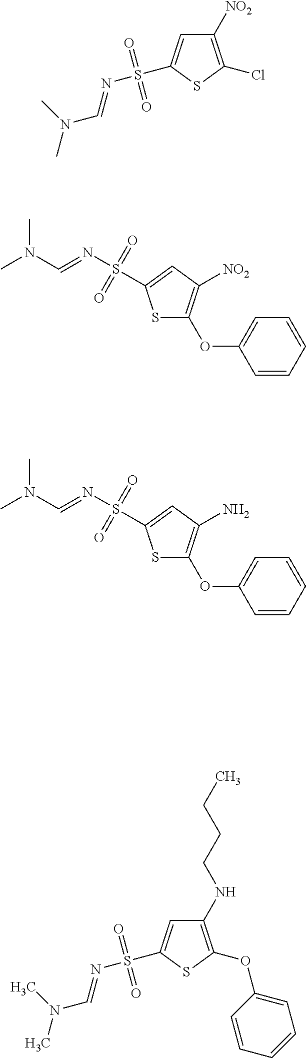 [SMILES: CN(C)/C=N/S(=O)(=O)c1cc([N+](=O)[O-])c(Cl)s1 ; CN(C)/C=N/S(=O)(=O)c1cc([N+](=O)[O-])c(Oc2ccccc2)s1 ; CN(C)/C=N/S(=O)(=O)c1cc(N)c(Oc2ccccc2)s1 ; CCCCNc1cc(S(=O)(=O)/N=C/N(C)C)sc1Oc1ccccc1] 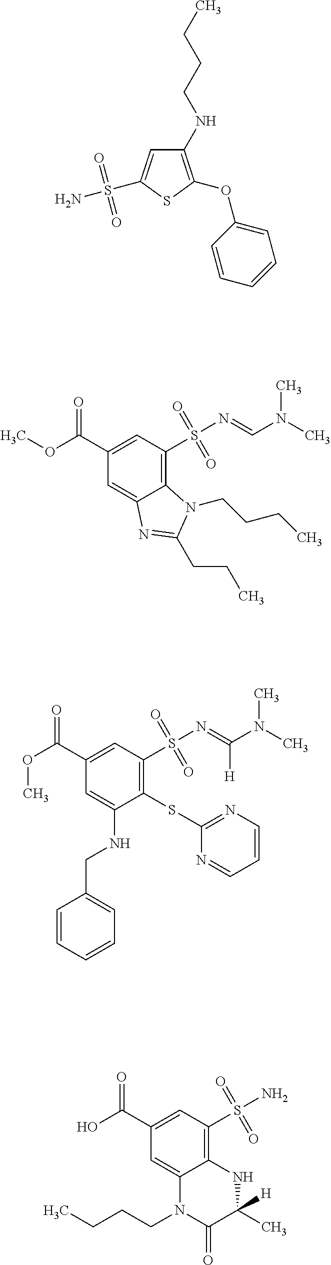 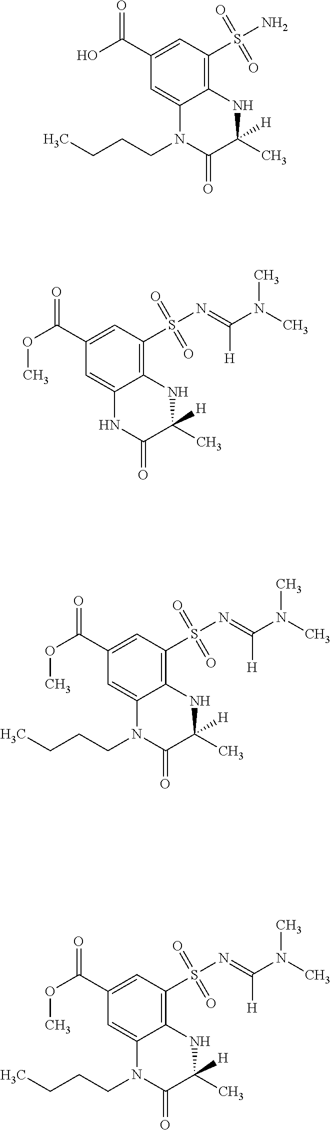 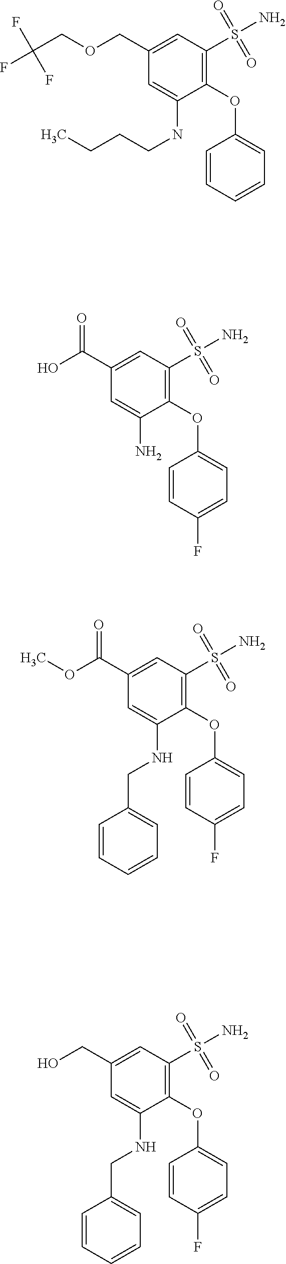 [SMILES: CCCCNc1cc(COCC(F)(F)F)cc(S(N)(=O)=O)c1Oc1ccccc1 ; Nc1cc(C(=O)O)cc(S(N)(=O)=O)c1Oc1ccc(F)cc1 ; COC(=O)c1cc(NCc2ccccc2)c(Oc2ccc(F)cc2)c(S(N)(=O)=O)c1 ; NS(=O)(=O)c1cc(CO)cc(NCc2ccccc2)c1Oc1ccc(F)cc1] 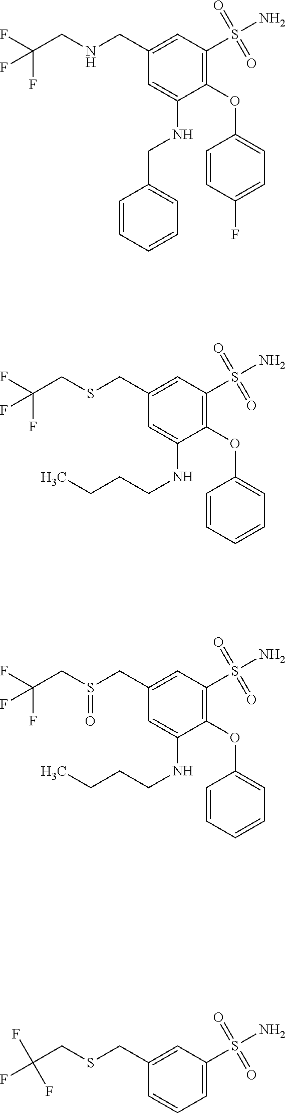 [SMILES: NS(=O)(=O)c1cc(CNCC(F)(F)F)cc(NCc2ccccc2)c1Oc1ccc(F)cc1 ; CCCCNc1cc(CSCC(F)(F)F)cc(S(N)(=O)=O)c1Oc1ccccc1 ; CCCCNc1cc(CS(=O)CC(F)(F)F)cc(S(N)(=O)=O)c1Oc1ccccc1 ; NS(=O)(=O)c1cccc(CSCC(F)(F)F)c1] 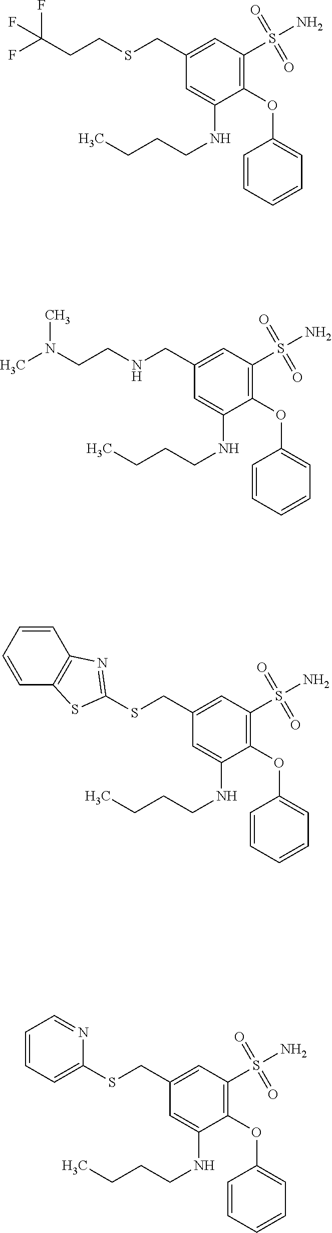 [SMILES: CCCCNc1cc(CSCCC(F)(F)F)cc(S(N)(=O)=O)c1Oc1ccccc1 ; CCCCNc1cc(CNCCN(C)C)cc(S(N)(=O)=O)c1Oc1ccccc1 ; CCCCNc1cc(CSc2nc3ccccc3s2)cc(S(N)(=O)=O)c1Oc1ccccc1 ; CCCCNc1cc(CSc2ccccn2)cc(S(N)(=O)=O)c1Oc1ccccc1] 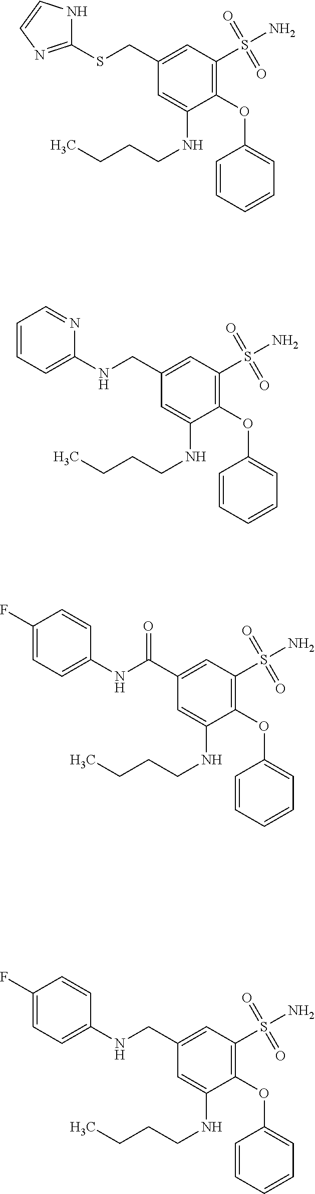 [SMILES: CCCCNc1cc(CSc2ncc[nH]2)cc(S(N)(=O)=O)c1Oc1ccccc1 ; CCCCNc1cc(CNc2ccccn2)cc(S(N)(=O)=O)c1Oc1ccccc1 ; CCCCNc1cc(C(=O)Nc2ccc(F)cc2)cc(S(N)(=O)=O)c1Oc1ccccc1 ; CCCCNc1cc(CNc2ccc(F)cc2)cc(S(N)(=O)=O)c1Oc1ccccc1] 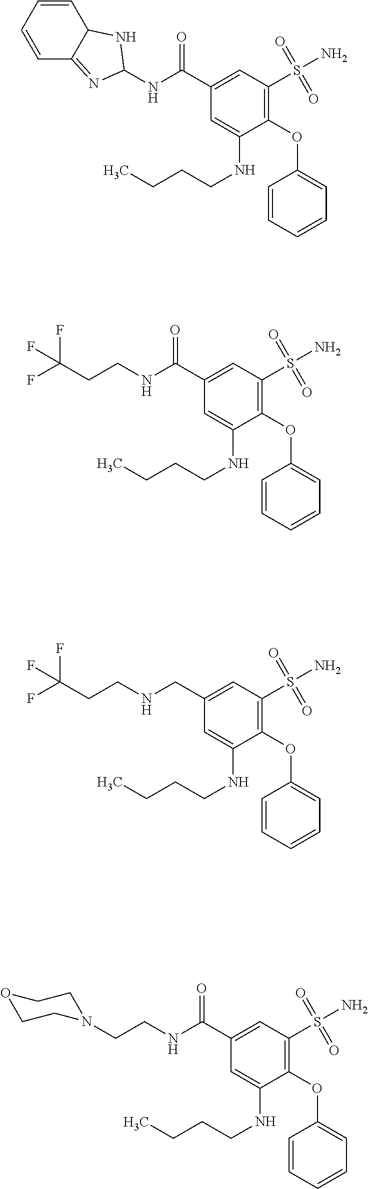 [SMILES: CCCCNc1cc(C(=O)NC2N=C3C=CC=CC3N2)cc(S(N)(=O)=O)c1Oc1ccccc1 ; CCCCNc1cc(C(=O)NCCC(F)(F)F)cc(S(N)(=O)=O)c1Oc1ccccc1 ; CCCCNc1cc(CNCCC(F)(F)F)cc(S(N)(=O)=O)c1Oc1ccccc1 ; CCCCNc1cc(C(=O)NCCN2CCOCC2)cc(S(N)(=O)=O)c1Oc1ccccc1] 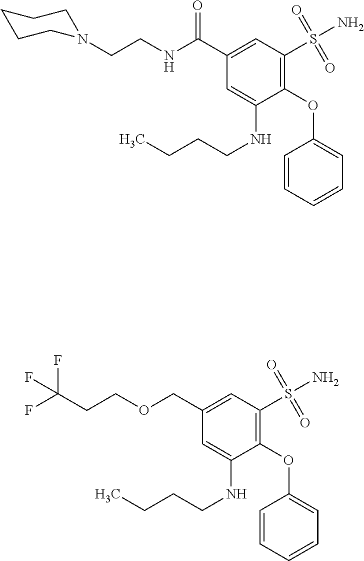 [SMILES: CCCCNc1cc(C(=O)NCCN2CCCCC2)cc(S(N)(=O)=O)c1Oc1ccccc1 ; CCCCNc1cc(COCCC(F)(F)F)cc(S(N)(=O)=O)c1Oc1ccccc1]
Error! Objects cannot be created from editing field codes.Error! Objects cannot be created from editing field codes.Error! Objects cannot be created from editing field codes.

36. A compound as defined in claim 35 for use as a medicament.

37. A pharmaceutical composition comprising a compound as defined in claim 35 and a pharmaceutically acceptable excipient.

38. A compound of formula (Ia) 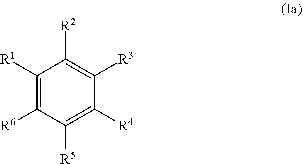
wherein:
R¹ is selected from —COOH, —COO—(C₁₋₁₅ alkyl), —COO—(C₀₋₁₅ alkylene)-carbocyclyl, —COO—(C₀₋₁₅ alkylene)-heterocyclyl, —O—CHO, —O—CO—(C₁₋₁₅ alkyl), —O—CO—(C₀₋₁₅ alkylene)-carbocyclyl, —O—CO—(C₀₋₁₅ alkylene)-heterocyclyl, —CHO, —CO—(C₁₋₁₅ alkyl), —CO—(C₀₋₅ alkylene)-carbocyclyl, —CO—(C₀₋₁₅ alkylene)-heterocyclyl, —CO—NH₂, —CO—N(R¹¹)—(C₁₋₁₅ alkyl), —CO—N(R¹¹)—(C₀₋₁₅ alkylene)-carbocyclyl, —CO—N(R¹¹)—(C₀₋₁₅ alkylene)-heterocyclyl, —N(R¹¹)—CHO, —N(R¹¹)—CO—(C₁₋₁₅ alkyl), —N(R¹¹)—CO—(C₀₋₁₅ alkylene)-carbocyclyl, —N(R¹¹)—CO—(C₀₋₁₅ alkylene)-heterocyclyl, C₁₋₁₅ alkyl, —(C₀₋₁₅ alkylene)-carbocyclyl, —(C₀₋₁₅ alkylene)-heterocyclyl, C₂₋₁₅ alkenyl, —(C₂₋₁₅ alkenylene)-carbocyclyl, —(C₂₋₁₅ alkenylene)-heterocyclyl, C₂₋₁₅ alkynyl, —(C₂₋₁₅ alkynylene)-carbocyclyl and —(C₂₋₁₅ alkynylene)-heterocyclyl,
wherein the alkyl moiety of any of the aforementioned groups, the alkylene moiety of any of the aforementioned groups, the alkenylene moiety of any of the aforementioned groups, the alkynylene moiety of any of the aforementioned groups, said C₁₋₁₅ alkyl, said C₂₋₁₅ alkenyl and said C₂₋₁₅ alkynyl are each optionally substituted with one or more groups independently selected from halogen, —CF₃, —CN, —NO₂, —N(R¹¹)(R¹¹), —O(R¹¹), —S(R¹¹) and —SO₃H,
wherein one or more —CH₂— units comprised in the alkyl moiety of any of the aforementioned groups, in the alkylene moiety of any of the aforementioned groups, in the alkenylene moiety of any of the aforementioned groups, in the alkynylene moiety of any of the aforementioned groups, in said C₁₋₁₅ alkyl, in said C₂₋₁₅ alkenyl, or in said C₂₋₁₅ alkynyl are each optionally replaced by a group independently selected from —O—, —CO—, —COO—, —O—CO—, —N(R¹¹)—, —N(R¹¹)—CO—, —CO—N(R¹¹)—, —S—, —SO—, —SO₂—, —SO₂—N(R¹¹)— and —N(R¹¹)—SO₂—, and further wherein the carbocyclyl moiety of any of the aforementioned groups and the heterocyclyl moiety of any of the aforementioned groups are each optionally substituted with one or more groups independently selected from C₁₋₆ alkyl, C₂₋₆ alkenyl, C₂₋₆ alkynyl, halogen, C₁₋₆ haloalkyl, —CN, —NO₂, —N(R¹¹)(R¹¹), —O(R¹¹), —S(R¹¹), —SO₃H, carbocyclyl and heterocyclyl;
each R¹¹ is independently hydrogen or C₁₋₆ alkyl;
R² is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl);
R³ is selected from —SO₂—NH₂, —SO₂—NH(C₁₋₆ alkyl), —SO₂—N(C₁₋₆ alkyl)(C₁₋₆ alkyl), —SO₂—N═(C₁₋₆ alkylidene) and —SO₂-halogen, wherein the alkyl moiety of said —SO₂—NH(C₁₋₆ alkyl), one or both of the alkyl moieties of said —SO₂—N(C₁₋₆ alkyl)(C₁₋₆ alkyl), and the alkylidene moiety of said —SO₂—N═(C₁₋₆ alkylidene) are each optionally substituted with one or more groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH and —S(C₁₋₆ alkyl);
R⁴ and R⁵ are mutually linked to form a group —R^{5b}—;
R^{5b} is selected from —R^{5b1}—R^{5b2}—R^{5b1}, —N═C(R⁵³)—R^{5b3}—R^{5b1}, —R^{5b1}—R^{5b3}—C(R⁵³)═N—, and —N═C(R⁵³)—R^{5b4}—C(R⁵³)═N—;
each R^{5b1} is independently selected from —N(R⁵²)—, —O— and —S—;
R^{5b2} is selected from —C(R⁵³)(R⁵³)—, —C(R⁵³)(R⁵³)—C(R⁵³)(R⁵³)—, —C(R⁵³)═C(R⁵³)—, —C(R⁵³)(R⁵³)—C(R⁵³)═C(R⁵³)— and —C(R⁵³)═C(R⁵³)—C(R⁵³)(R⁵³)—;
R^{5b3} is selected from a covalent bond, —C(R⁵³)(R⁵³), —C(R⁵³⁾(R⁵³)—C(R⁵³)(R⁵³)— and —C(R⁵³)═C(R⁵³)—;
R^{5b4} is selected from a covalent bond and —C(R⁵³)(R⁵³)—;
each R⁵² is independently selected from hydrogen, C₁₋₆ alkyl, C₂₋₆ alkenyl, C₂₋₆ alkynyl, —(C₀₋₄ alkylene)-aryl and —(C₀₋₄ alkylene)-heteroaryl;
each R⁵³ is independently selected from hydrogen, C₁₋₆ alkyl, C₂₋₆ alkenyl, C₂₋₆ alkynyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl), —N(C₁₋₆ alkyl)-SO₂—(C₁₋₆ alkyl), —(C₀₋₄ alkylene)-aryl and —(C₀₋₄ alkylene)-heteroaryl, and any two groups R⁵³ that are attached to the same carbon atom may also together form a group ═O, and any two groups R⁵³ that are attached to adjacent carbon atoms connected by a double bond may also be mutually linked to form a group —C(R⁵⁴)═C(R⁵⁴)—C(R⁵⁴)═C(R⁵⁴)—;
each R⁵⁴ is independently selected from hydrogen, C₁₋₆ alkyl, C₂₋₆ alkenyl, C₂₋₆ alkynyl, —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl); and
R⁶ is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆alkyl);
or a pharmaceutically acceptable salt or solvate thereof.

39. A compound as defined in claim 38 for use as a medicament.

40. A pharmaceutical composition comprising a compound as defined in claim 38 and a pharmaceutically acceptable excipient.

41. A compound of formula (Ib) 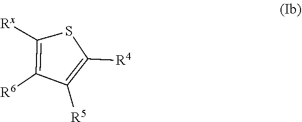
wherein:
R^{x} is selected from —COOH, —COO—(C₁₋₆ alkyl), —SO₂—NH₂, —SO₂—NH(C₁₋₄ alkyl), —SO₂—N(C₁₋₄ alkyl)(C₁₋₄ alkyl), and —SO₂—N═(C₁₋₄ alkylidene), wherein the alkyl moiety of said —SO₂—NH(C₁₋₄ alkyl), one or both of the alkyl moieties of said —SO₂—N(C₁₋₄ alkyl)(C₁₋₄ alkyl), and the alkylidene moiety of said —SO₂—N═(C₁₋₄ alkylidene) are each optionally substituted with one group selected from —NH₂, —NH(C₁₋₄ alkyl) and —N(C₁₋₄ alkyl)(C₁₋₄ alkyl);
R⁴ is a group R^{4a}, and R⁵ is a group R^{5a};
R^{4a} is selected from —O—R⁴¹, —S—R⁴¹, —NH—R⁴¹, —N(C₁₋₆ alkyl)-R⁴¹, halogen, carbocyclyl and heterocyclyl, wherein said carbocyclyl and said heterocyclyl are each optionally substituted with one or more groups R⁴²;
R⁴¹ is selected from —(C₀₋₄ alkylene)-carbocyclyl, —(C₀₋₄ alkylene)-heterocyclyl, C₁₋₆ alkyl, C₂₋₆ alkenyl and C₂₋₆ alkynyl, wherein the carbocyclyl moiety of said —(C₀₋₄ alkylene)-carbocyclyl and the heterocyclyl moiety of said —(C₁₋₄ alkylene)-heterocyclyl are each optionally substituted with one or more groups R⁴², and wherein said C₁₋₆ alkyl, said C₂₋₆ alkenyl, said C₂₋₆ alkynyl, the alkylene moiety of said —(C₀₋₄ alkylene)-carbocyclyl, and the alkylene moiety of said —(C₁₋₄ alkylene)-heterocyclyl are each optionally substituted with one or more groups R⁴³;
each R⁴² is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl);
each R⁴³ is independently selected from —OH, —O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, —CF₃, —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl) and —N(C₁₋₆ alkyl)-CO—(C₁₋₆ alkyl);
R^{5a} is selected from —NH₂, —NH(C₁₋₆ alkyl) and —N(C₁₋₆ alkyl)(C₁₋₆ alkyl), wherein the alkyl moiety of said —NH(C₁₋₆ alkyl) and one or both of the alkyl moieties of said —N(C₁₋₆ alkyl)(C₁₋₆ alkyl) are each optionally substituted with one or more groups independently selected from halogen, —CF₃, —CN, —NO₂, —NH₂, —NH(C₁₋₆ alkyl), —N(C₁₋₆ alkyl)(C₁₋₆ alkyl), —OH, —O(C₁₋₆ alkyl), —SH, —S(C₁₋₆ alkyl), carbocyclyl and heterocyclyl, wherein said carbocyclyl and said heterocyclyl are each optionally substituted with one or more groups R⁵¹;
each R⁵¹ is independently selected from C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆ alkyl); and
R⁶ is selected from hydrogen, C₁₋₆ alkyl, C₂₋₆ alkenyl, C₂₋₆ alkynyl, —OH, —O(C₁₋₆ alkyl), —O(C₁₋₆ alkylene)-OH, —O(C₁₋₆ alkylene)-O(C₁₋₆ alkyl), —SH, —S(C₁₋₆ alkyl), —NH₂, —NH(C₁₋₆ alkyl), —N(C₁₋₆ alkyl)(C₁₋₆ alkyl), halogen, C₁₋₆ haloalkyl, —O—(C₁₋₆ haloalkyl), —CN, —NO₂, —CHO, —CO—(C₁₋₆ alkyl), —COOH, —COO—(C₁₋₆ alkyl), —O—CO—(C₁₋₆ alkyl), —CO—NH₂, —CO—NH(C₁₋₆ alkyl), —CO—N(C₁₋₆ alkyl)(C₁₋₆ alkyl), —NH—CO—(C₁₋₆ alkyl), —N(C₁₋₆ alkyl)-CO—(C₁₋₆ alkyl), —SO₂—NH₂, —SO₂—NH(C₁₋₆ alkyl), —SO₂—N(C₁₋₆ alkyl)(C₁₋₆ alkyl), —NH—SO₂—(C₁₋₆ alkyl) and —N(C₁₋₆ alkyl)-SO₂—(C₁₋₆alkyl);
or a pharmaceutically acceptable salt or solvate thereof.

42. A compound as defined in claim 41 for use as a medicament.

43. A pharmaceutical composition comprising a compound as defined in claim 41 and a pharmaceutically acceptable excipient.
